(19) United States
(12) Patent Application Publication
(10) Pub. No.: US 2012/0244155 A1
(43) Pub. Date: Sep. 27, 2012

(54) Dendritic Cells (DCs) Targeting for Tuberculosis (TB) Vaccine

(75) Inventors: Patrick Lecine, Dallas, TX (US); Yves Levy, Paris (FR); Gerard Zurawski, Midlothian, TX (US); Jacques F. Banchereau, Montclair, NJ (US)

(73) Assignees: BAYLOR RESEARCH INSTITUTE, Dallas, TX (US); Agence Nationale de Recherches Sur le Sida, Paris (FR); Institut National de la Sante et de la Recherche Medicale (INSERM), Paris (FR)

(21) Appl. No.: 13/424,582

(22) Filed: Mar. 20, 2012

Related U.S. Application Data
(60) Provisional application No. 61/466,292, filed on Mar. 22, 2011.

(51) Int. Cl. A61K 39/395; A61P 37/04; C12N 5/078

(52) U.S. Cl. 424/134.1; 435/375; 424/178.1

(57) ABSTRACT

Compositions and methods comprising high affinity monoclonal antibody conjugates against several DC receptors are described herein. The inventors prepared fusion proteins with antigens for DC-targeting vaccine generation by conjugating several M. tuberculosis protein antigens with high affinity monoclonal antibodies against several DC receptors with a view to developing novel human vaccines based on in vivo DC-targeting. The findings of studies described herein indicate that vaccines bearing TB antigens can recall a potent memory antigen-specific T cell response in vitro resulting in IFNγ secretion.

### CROSS-REFERENCE TO RELATED APPLICATIONS

This application claims priority to U.S. Provisional Application Ser. No. 61/466,292, filed Mar. 22, 2011, the entire contents of which are incorporated herein by reference.

### TECHNICAL FIELD OF THE INVENTION

The present invention relates in general to tuberculosis (TB) treatment and control, and more particularly, to a composition and a method of developing a TB vaccine by targeting antigens to human dendritic cells and their subtypes.

### STATEMENT OF FEDERALLY FUNDED RESEARCH

None.

### REFERENCE TO A SEQUENCE LISTING

The present application includes a Sequence Listing which has been submitted in ASCII format via EFS-Web and is hereby incorporated by reference in its entirety. Said ASCII copy, created on Mar. 12, 2012, is named BHCS1119.txt and is 882,499 bytes in size.

### BACKGROUND OF THE INVENTION

Without limiting the scope of the invention, its background is described in connection with design of fusion proteins and targeting antigens to dendritic cells (DCs) for the purpose of vaccination and to evoke potent humoral and cellular immune responses.

U.S. Patent Application No. 20040146948 (Britton et al. 2003) provides single-chain Fv (scFv) fragment-based compositions and methods for targeting antigens (including Mycobacterial antigens) to antigen-presenting cells (APCs) such as, for example, dendritic cells (DC). The Britton invention further discloses compositions and methods useful in the treatment of diseases including infectious diseases and cancer.

U.S. Pat. No. 7,560,534 issued to Deo et al. (2009) describes molecular conjugates comprising an antigen linked to a human monoclonal antibody that specifically binds to dendritic cells. The '534 patent further discloses pharmaceutical compositions comprising the molecular conjugates and therapeutic methods for using the conjugates.

U.S. Pat. No. 7,261,897 issued to Skeiky et al. (2007) relates to compositions and fusion proteins containing at least two Mycobacterium sp. antigens, and nucleic acids encoding such compositions and fusion proteins. The compositions of the Skeiky invention increase serological sensitivity of sera from individuals infected with tuberculosis, and methods for their use in the diagnosis, treatment, and prevention of tuberculosis infection.

### SUMMARY OF THE INVENTION

The present invention describes a tuberculosis (TB) vaccine comprising selected TB proteins domains fused to heavy and light chain of antibodies that is used to target and activate dendritic cells (DCs). The vaccine composition described herein delivers antigen specifically to DCs for the purpose of vaccination to evoke potent humoral and cellular immune responses. The vaccine composition of the present invention promotes efficient recall memory induced in blood from TB patients. Targeting. DCs with TB-antigen fused to anti-DC antibodies enables recognition of both human and monkey proteins.

A method for increasing the effectiveness of antigen presentation by an antigen presenting cell (APC) is disclosed in one embodiment of the present invention. The method of the present invention comprises the steps of: isolating and purifying a dendritic cell (DC)-specific antibody or fragment thereof to which one or more antigens or recombinant antigens are attached or conjugated to form an antibody-antigen complex, and contacting the APC with the antibody-antigen complex under conditions wherein the antibody-antigen complex is processed and presented for T cell recognition.

In one aspect of the method described hereinabove the APC comprises an isolated dendritic cell (DC), a peripheral blood mononuclear cell (PBMC), a monocyte, a B cell, a myeloid dendritic cell and combinations thereof that have been cultured in vitro with GM-CSF and IL-4, interferon alpha, antigen and combinations thereof. In one aspect, the one or more antigens or recombinant antigens comprise M. tuberculosis antigens comprises at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211. In another aspect, the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR. In another aspect, the method of claim 1, wherein a nucleotide sequence for the DC-specific antibody or the antigen is selected from SEQ ID NOS: 6 to 70, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, or 220.

Another embodiment described herein relates to an immunostimulatory composition for generating an immune response, for prophylaxis, for therapy or any combination thereof against tuberculosis in a human or animal subject comprising: (i) an anti-dendritic cell (DC)-specific antibody or fragment thereof fused or conjugated to at least a portion of one or more M. tuberculosis antigens or antigenic peptides and (ii) a pharmaceutically acceptable carrier, wherein the conjugate is comprised in an amount effective to generate the immune response against tuberculosis. In one aspect of the composition the one or more M. tuberculosis antigens are selected from the group consisting of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, and any combinations thereof. In another aspect the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR. In yet another aspect the DC-specific antibody or fragment comprises αDCIR, αLangerin, αLOX-1 or αCD40. In a related aspect the DC-specific antibody is humanized. In another aspect the composition is administered to the human or animal subject by an oral route, a nasal route, topically or as an injection, wherein the injection is selected from the group consisting of subcutaneous, intravenous, intraperitoneal:

In yet another embodiment the instant invention discloses a vaccine composition comprising: an anti-dendritic cell (DC)-specific antibody or fragment thereof fused or conjugated to at least a portion of one or more recombinant or natural M. tuberculosis antigens or antigenic peptides, wherein the antigenic peptides are representative of one or more epitopes of the one or more antigens implicated in tuberculosis and an optional pharmaceutically acceptable carrier or an adjuvant. The vaccine composition of the present invention further comprises a flexible linker between the DC-specific antibody or fragment thereof and the one or more antigens, wherein the one or more antigens or antigenic peptides are conjugated to a heavy-chain, a light-chain or both of the anti-dendritic cell (DC)-specific antibody.

In one aspect of the vaccine composition the one or more M. tuberculosis antigens are selected from the group consisting of SEQ ID NO: 1, SEQ ID NO: 2, SEQ ID NO: 3, SEQ ID NO: 4, SEQ ID NO: 5, and any combinations thereof. In another aspect the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR. In a specific aspect the DC-specific antibody or fragment comprises αDCIR, αLangerin, αLOX-1 or αCD40. In a related aspect the DC-specific antibody is humanized and the composition is administered to the human or animal subject by an oral route, a nasal route, topically or as an injection. In one aspect the injection is selected from the group consisting of subcutaneous, intravenous, intraperitoneal, intramuscular, and intravenous.

A tuberculosis vaccine composition is disclosed in one embodiment of the instant invention. The composition comprises a dendritic cell (DC)-specific antibody or fragment thereof comprising αDCIR, αLangerin, αLOX-1 or αCD40 fused to one or more antigens or antigenic peptides selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, wherein the antigens or the antigenic peptides are conjugated to a heavy chain of the DC-specific antibody via one or more flexible linkers and an optional pharmaceutically acceptable carrier or an adjuvant.

Another embodiment of the instant invention describes a tuberculosis vaccine comprising: a dendritic cell (DC)-specific antibody or fragment thereof comprising αDCIR, αLangerin, αLOX-1 or αCD40 fused to one or more antigens selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, wherein the antigens or the antigenic peptides are conjugated to a heavy chain of the DC-specific antibody via one or more flexible linkers and an optional pharmaceutically acceptable carrier or an adjuvant. A tuberculosis vaccine comprising a dendritic cell (DC)-specific antibody or fragment thereof comprising αDCIR, αLangerin, αLOX-1 or αCD40 fused to one or more antigens or antigenic peptide comprising SEQ ID NO: 3, wherein the antigen or the antigenic peptide is conjugated to a light chain of the DC-specific antibody via one or more flexible linkers and an optional pharmaceutically acceptable carrier or an adjuvant is also disclosed herein.

In yet another embodiment the instant invention provides a method for increasing effectiveness of one or more dendritic cells (DCs) comprising the steps of: a) isolating the DCs from a patient, b) exposing the DCs to activating amounts of a vaccine comprising a dendritic cell (DC)-specific antibody or fragment thereof loaded, fused or conjugated to one or more antigens selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, and any combinations thereof wherein the antigens or the antigenic peptides are conjugated to a heavy chain, a light chain or both of the DC-specific antibody via one or more flexible linkers; and an optional pharmaceutically acceptable carrier or an adjuvant, and c) reintroducing the antigen-loaded and activated DCs into the patient. In one aspect of the method the DC-specific antibody or fragment comprises αDCIR, αLangerin, αLOX-1 or αCD40.

A method of providing immunostimulation by activation of one or more dendritic cells (DCs) to a human subject for a prophylaxis, a therapy or a combination thereof against one or more viral, bacterial, fungal, parasitic, protozoal, and parasitic diseases, and allergic disorders is disclosed in one embodiment of the present invention. The immunostimulation method comprises the steps of: (i) identifying the human subject in need of immunostimulation for the prophylaxis, the therapy or a combination thereof against the one or more diseases or disorders; (ii) isolating one or more DCs from the human subject; (iii) activating the isolated DCs with an amount of a composition effective to form activated DCs comprising a dendritic cell (DC)-specific antibody or fragment thereof loaded, fused or conjugated to one or more antigens or antigenic peptides, wherein the antigens or the antigenic peptides are conjugated to a heavy chain, a light chain or both of the DC-specific antibody via one or more flexible linkers; and an optional pharmaceutically acceptable carrier or an adjuvant; and (iv) reintroducing the activated DCs into the human subject. In a specific aspect the bacterial disease is tuberculosis. In one aspect of the method the antigens selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, and any combinations thereof. In another aspect of the method the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR. In yet another aspect the DC-specific antibody or fragment comprises αDCIR, αLangerin, αLOX-1 or αCD40.

In another embodiment the instant invention describes a method for a treatment, a prophylaxis or a combination thereof against tuberculosis in a human subject comprising the steps of: identifying the human subject in need of the treatment, the prophylaxis or a combination thereof against tuberculosis; and administering a vaccine composition comprising an anti-dendritic cell (DC)-specific antibody or fragment thereof fused or conjugated to at least a portion of one or more recombinant or natural M. tuberculosis antigens or antigenic peptides, wherein the antigenic peptides are representative of one or more epitopes of the one or more antigens implicated in tuberculosis and an optional pharmaceutically acceptable carrier or an adjuvant.

The method as described hereinabove further comprises a flexible linker between the DC-specific antibody or fragment thereof and the one or more antigens. In one aspect the one or more antigens or antigenic peptides are conjugated to a heavy-chain, a light-chain or both of the anti-dendritic cell (DC)-specific antibody. In another aspect the one or more M. tuberculosis antigens are selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, and any combinations thereof. In yet another aspect the DC-specific antibody or fragment is selected from an anti-DCIR, αDCIR, MHC class 1, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR. In a related aspect the humanized DC-specific antibody or fragment comprises αDCIR, αLangerin, αLOX-1 or αCD40. In another aspect the composition is administered to the human or animal subject by an oral route, a nasal route, topically or as an injection. In another aspect the injection is selected from the group consisting of subcutaneous, intravenous, intraperitoneal, intramuscular, and intravenous.

An immunostimulatory composition for generating an immune response is disclosed in one embodiment of the present invention. The composition provides a prophylaxis, a therapy or any combination thereof against tuberculosis in a human or animal subject and comprises: a) an anti-dendritic cell (DC)-specific antibody or fragment thereof fused or conjugated to at least a portion of one or more M. tuberculosis antigens or antigenic peptides, b) at least one Toll-Like Receptor (TLR) agonist which is selected from the group consisting of TLR1, TLR2, TLR3, TLR4, TLR5, TLR6, TLR7, and TLR8 agonists and c) a pharmaceutically acceptable carrier, wherein the conjugate is comprised in an amount effective to generate the immune response against tuberculosis. In one aspect the one or more M. tuberculosis antigens are selected from the group consisting of SEQ ID NO: 1, SEQ ID NO: 2, SEQ ID NO: 3, SEQ ID NO: 4, SEQ ID NO: 5, and any combinations thereof. In another aspect the DC-specific antibody or fragment comprises αDCIR, αLangerin, αLOX-1 or αCD40.

The present invention also discloses A tuberculosis vaccine comprising: an anti-dendritic cell (DC)-specific antibody or fragment thereof fused or conjugated to at least a portion of one or more recombinant or natural M. tuberculosis antigens or antigenic peptides, wherein the antigenic peptides are representative of one or more epitopes of the one or more antigens implicated in tuberculosis, at least one Toll-Like Receptor (TLR) agonist which is selected from the group consisting of TLR1, TLR2, TLR3, TLR4, TLR5, TLR6, TLR7, and TLR8 agonists, and one or more optional pharmaceutically acceptable carriers and adjuvants. In one aspect the one or more M. tuberculosis antigens are selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, and any combinations thereof. In another aspect the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44; CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR. In yet another aspect the DC-specific antibody is humanized. In another aspect the composition is administered to the human or animal subject by an oral route, a nasal route, topically or as an injection, wherein the injection is selected from the group consisting of subcutaneous, intravenous, intraperitoneal, intramuscular, and intravenous.

In one embodiment the present invention discloses a method for a treatment, a prophylaxis or a combination thereof against tuberculosis in a human subject comprising the steps of: (i) identifying the human subject in need of the treatment, the prophylaxis or a combination thereof against tuberculosis, and (ii) administering a vaccine composition comprising: a) an anti-dendritic cell (DC)-specific antibody or fragment thereof fused or conjugated to at least a portion of one or more recombinant or natural M. tuberculosis antigens or antigenic peptides, wherein the antigenic peptides are representative of one or more epitopes of the one or more antigens implicated in tuberculosis; b) at least one Toll-Like Receptor (TLR) agonist which is selected from the group consisting of TLR1, TLR2, TLR3, TLR4, TLR5, TLR6, TLR7, and TLR8 agonists; and c) one or more optional pharmaceutically acceptable carriers and adjuvants.

In one aspect the vaccine described hereinabove further comprises a flexible linker between the DC-specific antibody or fragment thereof and the one or more antigens. In another aspect the one or more antigens or antigenic peptides are conjugated to a heavy-chain, a light-chain or both of the anti-dendritic cell (DC)-specific antibody. In yet another aspect the one or more M. tuberculosis antigens are selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, and any combinations thereof. In another aspect the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR. In related aspects the DC-specific antibody or fragment comprises αDCIR, αLangerin, αLOX-1 or αCD40 and further the DC-specific antibody is humanized.

### BRIEF DESCRIPTION OF THE DRAWINGS

For a more complete understanding of the features and advantages of the present invention, reference is now made to the detailed description of the invention along with the accompanying figures and in which:

FIG. 1 shows the results of IFNγ measurement from the peripheral blood mononuclear cells (PBMCs) of tuberculosis (TB) patients, active and latent, that were incubated for 48 h with recombinant antigens;

FIG. 2 is a schematic showing TB antigens expressed as fusion proteins with the heavy chain of each antibody;

FIG. 3 is a schematic showing the fusion of Rv0475 to the light chain of each antibody;

FIG. 4 is a schematic representation of the chimeric recombinant antibody used as vaccine according to an embodiment of the present invention; and

FIGS. 5A-5F demonstrate the ability of recombinant anti-DCIR and anti-CD40 antibodies fused to individual TB antigen to elicit expansion of antigen-specific CD4+ T cells from TB patients: Delivering ESAT6 to DCs through CD40 and DCIR induces IFNγ-TNFα-producing ESAT6-specific CD4+ T cells. Monocytes-derived DCs (GM-CSF & IFNγ) were loaded ON with 10 (FIGS. 5A-5C) or 100 pM (FIGS. 5D-5F) of each targeting antibody. T cells were then added (1:40 ratio) and cultivated for 8 days with addition of 100 U of IL-2 at day 2 and day 5. At day 9, DCs were thawed and loaded ON with 1 nM of targeting mAbs (IgG4, αCD40 and αDCIR). On day 9.5 50,000 loaded-DCs (IgG4, αCD40 and αDCIR) or unloaded were then added. After 6 h, frequency of peptide-specific CD4+ and CD8+ T cells with intracellular IFNγ+TNFα+ was assessed by FACS analysis.

FIGS. 6A (non-reduced) and 6B (reduced) are gels that show the expression of the constructs listed as follows: In FIG. 6A—Lane 1. hIgG4-Flex-v1-m.tbRv1980-f4-m.tbRv0288-f2-m.tbAg85BDel41; Lane 2. manti-CD40_{—}12E12.3F3-hIgG4HC-Flex-v1-m.tbRv1980-f4-m.tbRv0288-f2-m.tbAg85BDel41; Lane 3. mAnti-DCIR_{—}9E8-hIgG4-C-Flex-v1-m.tbRv1980-f4-m.tbRv0288-f2-m.tbAg85BDel41; Lane 4. hIgG4-Flex-v1-m.tbAg85BDel41-f2-m.tbESAT6-f4-m.tbRv1980; Lane 5. manti-CD40_{—}12E12-hIgG4-C-Flex-v1-m.tbAg85BDel41-f2-m.tbESAT6-f4-m.tbRv1980; Lane 6. hIgG4-Flex-v1-m.tbRv1980-f4-m.tbRv0288-f2-m.tbAg85BDel41; Lane 7. manti-CD40_{—}12E12.3F3-hIgG4HC-Flex-v1-m.tbRv1980-f4-m.tbRv0288-f2-m.tbAg85BDel41; Lane 8. mAnti-DCIR_{—}9E8-hIgG4-C-Flex-v1-m.tbRv1980-f4-m.tbRv0288-f2-m.tbAg85BDel41; Lane 9. hIgG4-Flex-v1-m.tbAg85BDel41-f2-m.tbESAT6-f4-m.tbRv1980; Lane 10. manti-CD40_{—}12E12-hIgG4-C-Flex-v1-m.tbAg85BDel141-f2-m.tbESAT6-f4-m.tbRv1980. In FIG. 6B—Lane 1. hIgG4-Flex-v1-m.tbRv1980-f4-m.tbRv028842-m.tbAg85BDel41; Lane 2. manti-CD40_{—}12E12.3F3-hIgG4HC-Flex-v1-m.tbRv1980-f4-m.tbRv0288-f2-m.tbAg85BDel41; Lane 3. mAnti-DCIR_{—}9E8-hIgG4-C-Flex-v1-m.tbRv1980-f4-m.tbRv0288-f2-m.tbAg85BDel41; Lane 4. hIgG4-Flex-v1-m.tbAg85BDel41-f2-m.tbESAT6-f4-m.tbRv1980; Lane 5. manti-CD40_{—}12E12-hIgG4-C-Flex-v1-m.tbAg85BDel41-f2-m.tbESAT6-f4-m.tbRv1980; Lane 6. mAnti-DCIR_{—}9E8-hIgG4-C-Flex-v1-m.tbAg85BDel41-f2-m.tbESAT6-f4-m.tbRv1980; Lane 7. manti-CD40_{—}12E12-hIgG4-C-Flex-v1m.tbCFP10-f2-m.tbESAT6; Lane 8. hIgG4-Flex-v1-m.tbCFP10-f2-m.tbESAT6; Lane 9. mAnti-DCIR_{—}9E8-hIgG4-C-Flex-v1-m.tbCFP10-f2-m.tbESAT6; Lane 10. mAnti-DCIR_{—}9E8-hIgG4-C-Flex-v1-m.tbHspX. The sequences for each of these constructs is detailed hereinbelow.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Antigen Presenting Cells” (APC) refers to cells that are capable of activating T cells, and include, but are not limited to, certain macrophages, B cells and dendritic cells. “Dendritic cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refers to any protein for which receptors are expressed on a dendritic cell. Examples include GM-CSF, IL-1, TNF, IL-4, CD40L, CTLA4, CD28, and FLT-3 ligand.

For the purpose of the present invention, the term “vaccine composition” is intended to mean a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes, or both. As used herein, the term “antigen” refers to any antigen which can be used in a vaccine, whether it involves a whole microorganism or a subunit, and whatever its nature: peptide, protein, glycoprotein, polysaccharide, glycolipid, lipopeptide, etc. They may be viral antigens, bacterial antigens, or the like; the term “antigen” also comprises the polynucleotides, the sequences of which are chosen so as to encode the antigens whose expression by the individuals to which the polynucleotides are administered is desired, in the case of the immunization technique referred to as DNA immunization. They may also be a set of antigens, in particular in the case of a multivalent vaccine composition which comprises antigens capable of protecting against several diseases, and which is then generally referred to as a vaccine combination, or in the case of a composition which comprises several different antigens in order to protect against a single disease, as is the case for certain vaccines against whooping cough or the flu, for example. The term “antibodies” refers to immunoglobulins, whether natural or partially or wholly produced artificially, e.g. recombinant. An antibody may be monoclonal or polyclonal. The antibody may, in some cases, be a member of one, or a combination immunoglobulin classes, including: IgG, IgM, IgA, IgD, and IgE.

The term “adjuvant” refers to a substance that enhances, augments or potentiates the host's immune response to a vaccine antigen.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used in this application, the term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “in vivo” refers to being inside the body. The term “in vitro” used as used in the present application is to be understood as indicating an operation carried out in a non-living system.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes a novel tuberculosis (TB) human vaccine based on in vivo dendritic cell (DC)-targeting. The vaccine comprises a conjugate of one or more high affinity monoclonal antibodies against several DC receptors with several M. tuberculosis protein antigens. The vaccine composition described herein delivers antigen specifically to DCs for the purpose of vaccination to evoke potent humoral and cellular immune responses. The vaccine composition of the present invention promotes efficient recall memory induced in blood from TB patients.

TB is a leading cause of death: in 2006, there were 9.2 million new cases of active TB and an estimated 1.5 million deaths in the world, according to the World Health Organization. It is estimated that one-third of the world's population harbors Mycobacterium tuberculosis (M. tuberculosis) in the form of non-replicating bacilli (latent tuberculosis) (1). In addition, an increasing proportion of TB cases results from infection with M. tuberculosis resistant to first line chemotherapy (multi-drug resistant TB or MDR-TB) (2). M. bovis BCG, the only vaccine against TB currently available, prevents 80% of childhood military disease and meningitis, but its efficacy in preventing pulmonary disease in adulthood is rather low (3). Therefore, efficient therapeutic vaccines are urgently needed to cure active and latent TB patients. Furthermore, according to the World Health Organization, M. tuberculosis is also the major opportunistic pathogen associated with HIV disease, contributing significantly to HIV-associated mortality (almost 25% of HIV-related deaths are from TB).

Dendritic cells (DCs) are leukocytes specialized in the uptake, processing and presentation of antigens and play fundamental roles in regulating both innate and adaptive immunity functions and in maintaining immunological tolerance. They are mainly localized at the interface between body surfaces and the environment, continuously scrutinizing incoming antigen for the potential threat it may represent for the body. To control immune responses against a large variety of microorganisms including viruses and bacteria they exhibit at their surface a large number of receptors, which have been shown to be crucial in DC biological functions in vivo by sensing pathogen-associated molecular patterns (PAMPs). These specialized pathogen recognition receptors (PRRs) whose engagement modulates the functional activity of DCs, include C-type lectin receptors (CLRs) and Toll-like receptors (TLRs) (4). Following pathogen contact, PRRs are endocytosed by immature DCs, which induce the DC to enter a developmental program called maturation, which transforms DCs from sentinels into efficient APCs and T cell stimulators (5). With the induction of maturation, DCs undergo massive morphological and functional changes. Antigen internalization is down modulated due to repression of most antigen-receptors and to an overall reduction in phagocytosis and macropinocytosis. In addition, MHC molecules are redistributed from intracellular endocytic compartments to the cell surface (6,7). Peptide loading as well as the half-life of MHC molecules is increased (6,7). Finally, the surface expression of T cell costimulatory molecules also rises. Concomitant with the modifications of their antigen presentation abilities, maturation induces migration of DCs out of peripheral tissues (8). Modifications in the expression of chemokine receptors and adhesion molecules, as well as profound changes of the cytoskeleton organization, contribute to the migration of DCs, through the lymph, towards secondary lymphoid organs (9). By linking antigen uptake, peptide loading, and cell migration to the encounter of a pathogen, DCs restrict antigen presentation to those antigens internalized during maturation, thus favoring the stimulation of T cells specific for potentially pathogenic antigens.

All these biological events result in the activation of specific signaling pathways that lead to the induction of specific immune responses against these pathogens, in part through expression of effector cytokines. The resulting T cell responses following these interactions between PRRs and PAMPs have been extensively studied. However, little is known about the way the PPRs are internalized and how from inside the cell, they are able to dictate and orientate immune responses. The present inventors have previously produced high affinity monoclonal antibodies against several DC receptors, and prepared fusion proteins with antigens for DC-targeting vaccine generation. Targeting antigens to DCs using these fusion proteins recognizing different CLRs resulted in different biological responses. The inventors hypothesize is that subsets of human DCs express distinct receptors that need to be mobilized in order to mount a durable immune response adapted to each microbial invasion, depending on the pathogen to be eliminated. To test this hypothesis, the inventors have prepared high affinity monoclonal antibodies against several DC receptors to which antibodies have been conjugated to several M. tuberculosis protein antigens to develop novel human vaccines based on in vivo DC-targeting.

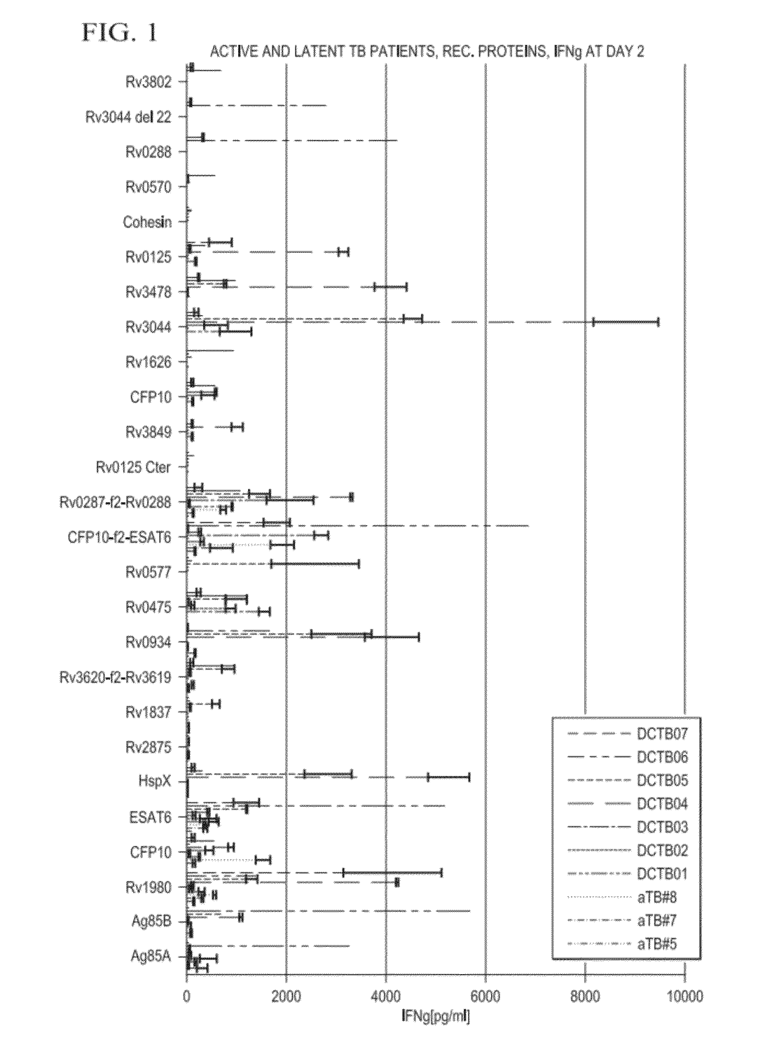

Construction of TB vaccine, Antigen selection: M. tuberculosis genome encodes for more than 4000 genes. Antigens selected were tested with PBMCs from active or latent TB patients. IFNγ secretion was measured to identify antigens recognized (FIG. 1). The data showed that several antigens are broadly recognized and therefore constitute potential antigens that will be fused to recombinant mAb. These include: ESAT6, CFP10, Rv1980 and combination of antigens: CFP10-f2-ESAT6 and Rv0287-f2-Rv0288. One antigen, Rv0475, is recognized mainly by latent TB patient as already reported in the literature (10-12). Because ESAT6 and CFP10 are used as a diagnostic tool to identify TB patients from environmental mycobacteria-infected person, clinical trials have focused on using Rv0288 instead of ESAT6. The inventors also chose Ag85B as a potential antigen even though its immunogenicity was not detected in the study described above. The inventors chose 5 antigens: ESAT6, Rv0288, Rv0475, Rv1980 and Ag85B. The sequences of the selected antigens are presented herein below. The underlined amino acids within parentheses are sequences that have been removed from the protein for better expression.

ESAT6 (95 amino acids):

(SEQ ID NO: 1)

MTEQQWNFAGIEAAASAIQGNVTSIHSLLDEGKQSLTKLAAAWGGSGSEA
YQGVQQKWDATATELNNALQNLARTISEAGQAMASTEGNVTGMFA
Rv0288 (96 amino acids):

(SEQ ID NO: 2)

MSQIMYNYPAMLGHAGDMAGYAGTLQSLGAEIAVEQAALQSAWQGDTGIT
YQAWQAQWNQAMEDLVRAYHAMSSTHEANTMAMMARDTAEAAKWGG
Rv0475 (199 amino acids):

(SEQ ID NO: 3)

MAENSNIDDIKAPLLAALGAADLALATVNELITNLRERAEETRTDTRSRV
EESRARLTKLQEDLPEQLTELREKFTAEELRKAAEGYLEAATSRYNELVE
RGEAALERLRSQQSFEEVSARAEGYVDQAVELTQEALGTVASQTRAVG
ERAAKLVGIELPKKAAPAKKAAPAKKAAPAKKAAAKKAPAKKAAAKKV
TQK
Rv1980Δ24 (228 amino acids):

(SEQ ID NO: 4)

(MRIKIFMLVTAVVLLCCSGVATAA)PKTYCEELKGTDTGQACQIQMSDP
AYNINISLPSYYPDQKSLENYIAQTRDKFLSAATSSTPREAPYELNITSA
TYQSAIPPRGTQAVVLKVYQNAGGTHTTTYKAFDWDQAYRKPITYDTL
WQADTDPLPVVFPIVQGELSKQTGQQVSIAPNAGLDPVNYQNFAVTN
DGVIFFFNPGELLPEAAGPTQVLVPRSAIDSMLA
Ag85BΔ41 (228 amino acids):

(SEQ ID NO: 5)

(MTDVSRKIRAWGRRLMIGTAAAVVLPGLVGLAGGAATAGAF)SRPGLPV
EYLQVPSPSMGRDIKVQFQSGGNNSPAVYLLDGLRAQDDYNGWDINTPAF
EWYYQSGLSIVMPVGGQSSFYSDWYSPACGKAGCQTYKWETFLTSELPQW
LSANRAVKPTGSAAIGLSMAGSSAMILAAYHPQQFIYAGSLSALLDPSQG
MGPSLIGLAMGDAGGYKAADMWGPSSDPAWERNDPTQQIPKLVANNTRLW
VYCGNGTPNELGGANIPAEFLENFVRSSNLKFQDAYNAAGGHNAVFNFPP
NGTHSWEYWGAQLNAMKGDLQSSLGAG

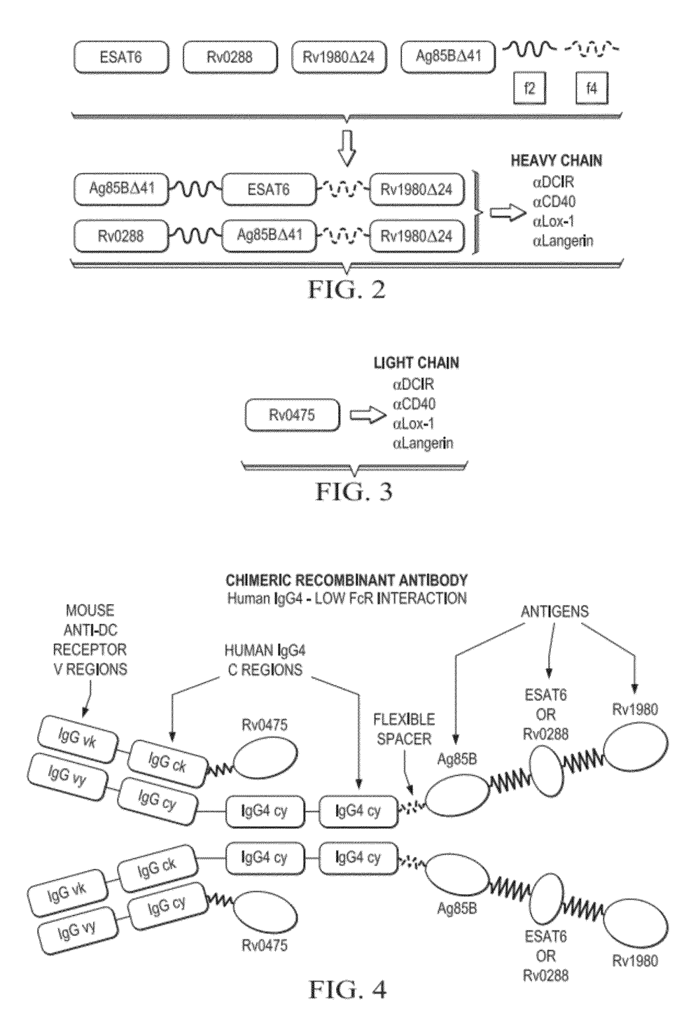

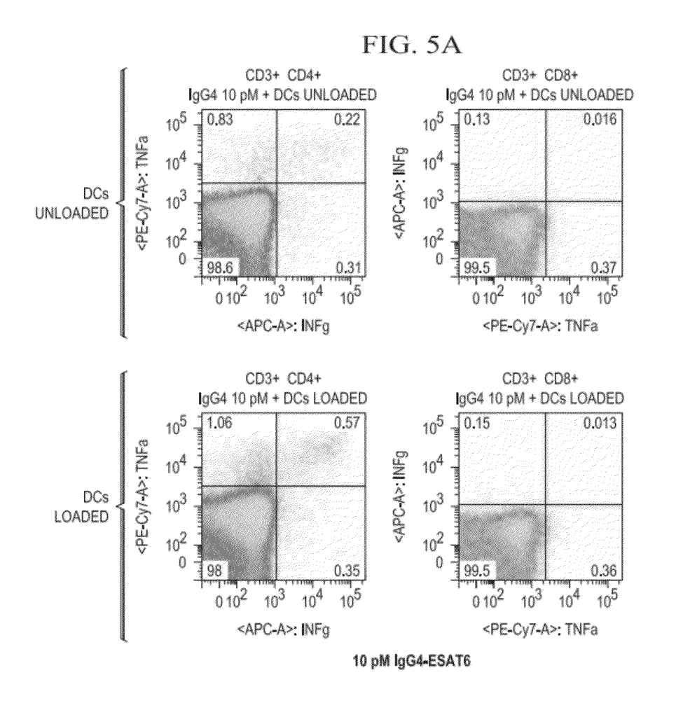

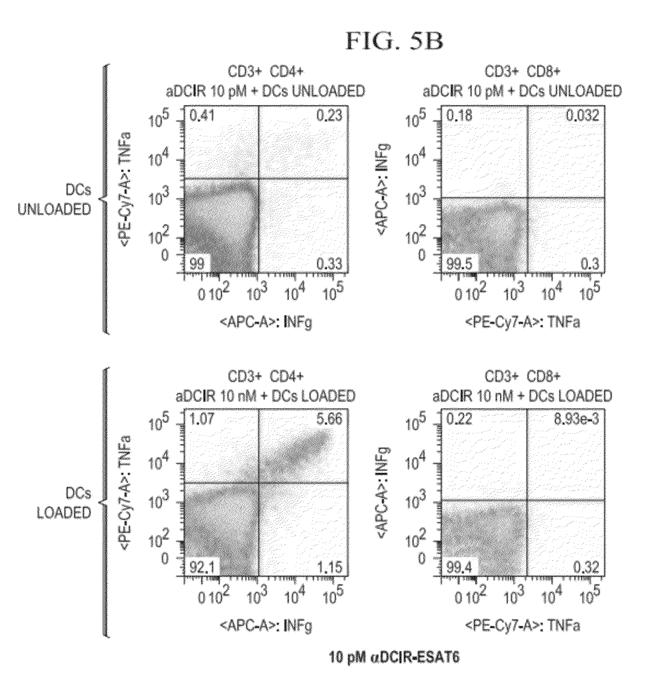

Vaccine description: Vaccines are recombinant monoclonal antibodies recognizing specific receptors expressed at the surface of DCs. These recombinant mAb are then conjugated to selected antigens (fusion to the C terminus of the heavy chain or the light chain). The inventors selected four recombinant antibodies: αCD40, αDCIR, αLox-1 and αLangerin. These antibodies have been conjugated to the selected antigens Ag85BΔ41, ESAT6, Rv0288 and Rv1980Δ24 from M. tuberculosis (FIG. 2). To cover as many potential epitopes from different antigens as possible, the inventors fused three selected antigens to the heavy chain of different recombinant mAbs, resulting in 2 constructs (FIG. 2). One antigen, Rv0475, was also fused to the light chain of each antibody (FIG. 3). The vaccine harbors three antigens fused to the heavy chain (Ag85B, Rv1980 and ESAT6 or Rv0288) and one to the light chain (Rv0475) as depicted in FIG. 4.

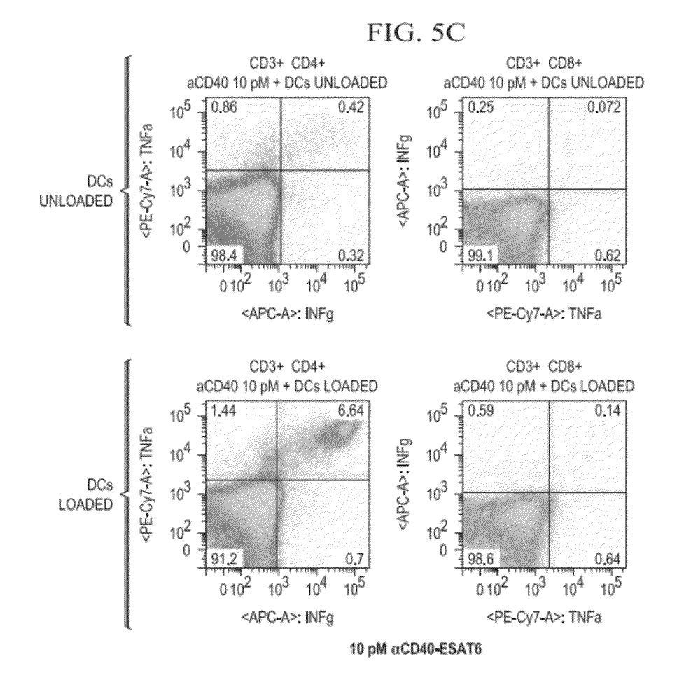

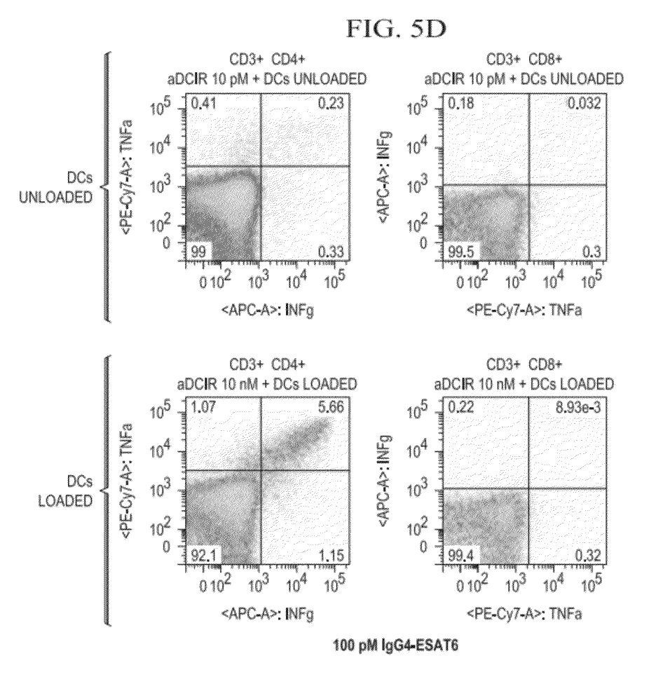

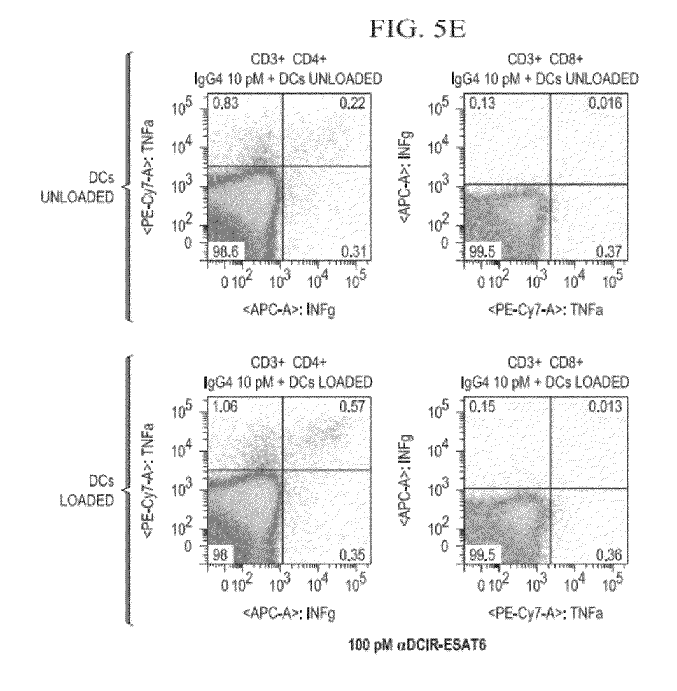

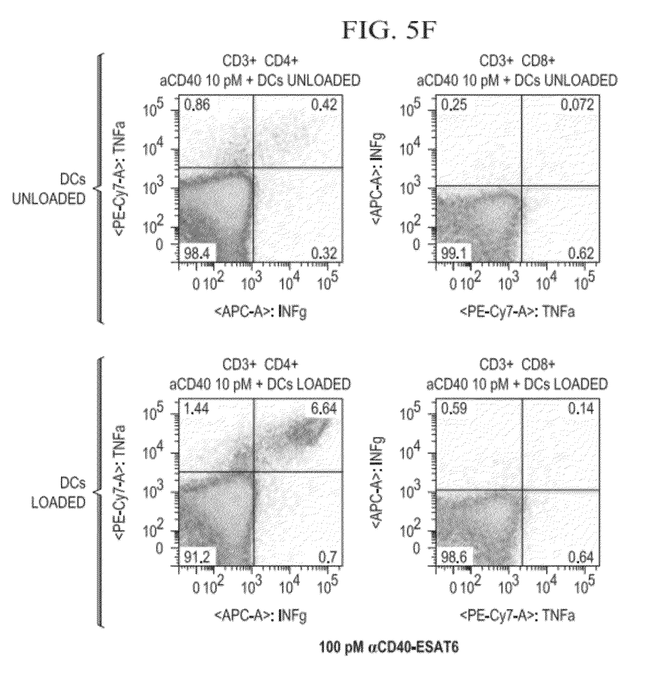

Vaccine evaluation: Vaccine candidates were tested in targeting studies by incubating vaccines with peripheral blood mononuclear cells (PBMCs) from TB patients (latent or active) or by loading monocyte-derived DCs with the recombinant mAb follow by addition of T-lymphocytes. After 11 days of culture, the efficiency of each vaccine in eliciting an immune recall response was assessed by cytokines secretion (in the culture supernatant or following intracellular staining) as seen in FIGS. 5A-5F and Table 1.

FIGS. 5A-5F demonstrate the ability of recombinant anti-DCIR and anti-CD40 antibodies fused to individual TB antigen to elicit expansion of antigen-specific CD4+ T cells from TB patients: Delivering ESAT6 to DCs through CD40 and DCIR induces IFNγ-TNFα-producing ESAT6-specific CD4+ T cells. Monocytes-derived DCs (GM-CSF and IFNγ) were loaded ON with 10 (FIGS. 5A-5C) or 100 pM (FIGS. 5D-5F) of each targeting antibody. T cells were then added (1:40 ratio) and cultivated for 8 days with addition of 100 U of IL-2 at day 2 and day 5. At day 9, DCs were thawed and loaded ON with 1 nM of targeting mAbs (IgG4, αCD40 and αDCIR). On day 9.5 50,000 loaded-DCs (IgG4, αCD40 and αDCIR) or unloaded were then added. After 6 h, frequency of peptide-specific CD4+ and CD8+ T cells with intracellular IFNγ+TNFα+ was assessed by FACS analysis. Thus, the data shows that αCD40 and αDCIR vaccines bearing a TB antigen can recall a potent memory antigen-specific anti-CD4+ T cell response in vitro. In this in vitro culture system αCD40 and αDCIR are equally potent vaccines (DCs used in this assay express both receptors).

TABLE 1
Demonstration of the ability of TB antigen vaccines to induce IFNα secretion at low doses.

| mAb | aTB#5 | aTB#7 | aTB#8 | DCTB#02 |
|---|---|---|---|---|
| IgG4-ESAT6 | 0.1 nM (~600) | 0.1 nM (~30) | 1 nM (~300) | 1 nM (~300) |
| | 0.01 nM (~50) | 0.01 nM (~10) | 0.1 nM (~60) | 0.1 nM (~60) |
| aCD40- | 0.1 nM (~600) | 0.1 nM (~140) | 1 nM (~3500) | 1 nM (~3500) |
| ESAT6 | 0.01 nM (~1000) | 0.01 nM (~500) | 0.1 nM (~2500) | 0.1 nM (~2500) |
| aDCIR- | 0.1 nM (~600) | 0.1 nM (~300) | 1 nM (~2500) | 1 nM (~2500) |
| ESAT6 | 0.01 nM (~1000) | 0.01 nM (~175) | 0.1 nM (~900) | 0.1 nM (~900) |
| IgG4-CFP10 | 0.01 nM (~470) | 0.1 nM (~20) | 0.1 nM (~700) | 0.01 nM (~2300) |
| | 0.001 nM (~80) | 0.01 nM (~10) | 0.01 nM (~50) | 0.001 n89 (~50 |
| aCD40- | 0.01 nM (~4800) | 0.1 nM (~350) | 0.1 nM (~3200) | 0.01 nM (~3000) |
| CFP10 | 0.001 nM (~2900) | 0.01 nM (~280) | 0.01 nM (~6000) | 0.001 nM (~3500) |
| aDCIR- | 0.01 nM (~1500) | 0.1 nM (~350) | 0.1 nM (~2000) | 0.01 nM (~3000) |
| CFP10 | 0.001 nM (~7300) | 0.01 nM (~260) | 0.01 nM (~3000) | 0.001 nM (~3900) |
| IgG4-Ag85B | 0.01 nM (~190) | ND | ND | ND |
| | 0.001 nM (~40) | | | |
| aCD40- | 0.01 nM (~6000) | ND | ND | ND |
| Ag85B | 0.001 nM (~10000) | | | |
| aDCIR- | 0.1 nM (~14000) | ND | ND | ND |
| Ag85B | 0.01 nM (~6000) | | | |
| IgG1-Rv1980 | 0.1 nM (~500) | ND | ND | ND |
| | 0.01 nM (~60) | | | |
| | 0.001 nM (~15) | | | |
| aCD40- | 0.01 nM (~2000) | ND | ND | ND |
| Rv1980 | 0.001 nM (~11000) | | | |
| aDCIR- | 0.1 nM (~3000) | ND | ND | ND |
| Rv1980 | 0.01 nM (~19000) | | | |

ESAT6 and CFP-10 were fused to IgG4, αCD40 and αDCIR and incubated with PBMCs from TB patients at different concentration (from 10 nM to 1 pM). Cells were cultivated for 8 days with addition of 100 U of IL-2 at day 2 and day 5. At day 8, cells were washed in DPBS and rested for 36 h. At day 9.5, cluster of peptides (10 μM each peptide) were used to stimulate cells for another 36 h. Supernatants were recovered and IFNγ secretion measured. IFNγ secretion is indicated in pg/ml.

The data presented hereinabove shows that both αCD40 and αDCIR vaccines bearing TB antigens can recall a potent memory antigen-specific T cell response in vitro resulting in IFNγ secretion. Specific immune responses were observed with low doses of vaccines, i.e., between 1 and 100 pM. Both constructs induced similar levels of IFNγ secretion in different patients.

Non-limiting examples different DC-specific antibodies or fragments (both nucleotide and protein sequences) that may be used in the preparation of the TB vaccine of the present invention are shown herein below:

Anti_CLEC_6_9B9.2G12_Hv-V-hIgG4H-C (SEQ ID NO: 6):
ATGGGCAGGCTTACTTCTTCATTCTTGCTACTGATTGTCCCTGCATATGTCCTGTCCCAGGTTAC
TCTGAAAGAGTCTGGCCCTGGGATATTGCAGCCCTCCCAGACCCTCAGTCTGACCTGTTCTTTC
TCTGGGTTTTCACTGAGCACTTCTGGTATGAGTGTAGGCTGGATTCGTCAGCCTTCAGGGAAGG
GTCTGGAGTGGCTGGCTCACATTTGGTGGAATGATGATAAGTACTATAATCCAGTCCTGAAAA
GCCGGCTCACAATCTCCAAGGAGACCTCCAACAACCAGGTATTCCTCAAGATCGCCAGTGTGG
TCTCTGCAGATACTGCCACATACTACTGTGCTCGATTCTATGGTAACTGTCTTGACTACTGGGG
CCAAGGCACCACTCTCACAGTCTCCTCGGCCAAAACAaagggcccATCCGTCTTCCCCCTGGCGCC
CTGCTCCAGGAGCACCTCCGAGAGCACAGCCGCCCTGGGCTGCCTGGTCAAGGACTACTTCCC
CGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTG
GGCACGAAGACCTACACCTGCAACGTAGATCACAAGCCCAGCAACACCAAGGTGGACAAGAG
AGTTGAGTCCAAATATGGTCCCCCATGCCCACCCTGCCCAGCACCTGAGTTCGAAGGGGGACC
ATCAGTCTTCCTGTTCCCCCCAAAACCCAAGGACACTCTCATGATCTCCCGGACCCCTGAGGTC
ACGTGCGTGGTGGTGGACGTGAGCCAGGAAGACCCCGAGGTCCAGTTCAACTGGTACGTGGAT
GGCGTGGAGGTGCATAATGCCAAGACAAAGCCGCGGGAGGAGCAGTTCAACAGCACGTACCG
TGTGGTCAGCGTCCTCACCGTCCTGCACCAGGACTGGCTGAACGGCAAGGAGTACAAGTGCAA
GGTCTCCAACAAAGGCCTCCCGTCCTCCATCGAGAAAACCATCTCCAAAGCCAAAGGGCAGCC
CCGAGAGCCACAGGTGTACACCCTGCCCCCATCCCAGGAGGAGATGACCAAGAACCAGGTCA
GCCTGACCTGCCTGGTCAAAGGCTTCTACCCCAGCGACATCGCCGTGGAGTGGGAGAGCAATG
GGCAGCCGGAGAACAACTACAAGACCACGCCTCCCGTGCTGGACTCCGACGGCTCCTTCTTCC
TCTACAGCAGGCTAACCGTGGACAAGAGCAGGTGGCAGGAGGGGAATGTCTTCTCATGCTCCG
TGATGCATGAGGCTCTGCACAACCACTACACACAGAAGAGCCTCTCCCTGTCTCTGGGTAAAG
CTAGCTGATTAATTAA
Anti_CLEC_6_9B9.2G12_Hv-V-hIgG4H-C (SEQ ID NO: 71):
MGRLTSSFLLLIVPAYVLSQVTLKESGPGILQPSQTLSLTCSFSGFSLSTSGMSVGWIRQPSGKGLEW
LAHIWWNDDKYYNPVLKSRLTISKETSNNQVFLKIASVVSADTATYYCARFYGNCLDYWGQGTTL
TVSSAKTKGPSVFPLAPCSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPPCPAPEFEGGPSVFLFPPKPKDTL
MISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHYTQKSLSLSLGK
AS
Anti_CLEC_6_9B9.2G12_Kv-V-hIgGK-C (SEQ ID NO: 7):
ATGATGTCCTCTGCTCAGTTCCTTGGTCTCCTGTTGCTCTGTTTTCAAGGTACCAGATGTGATAT
CCAGATGACACAGACTACATCCTCCCTGTCTGCCTCTCTGGGAGACAGAGTCACCATCAGTTGC
AGGGCAAGTCAGGACATTAGCAATTATTTAAACTGGTATCAGCAGAAACCAGATGGAACTGTT
AAACTCCTGATCTACTACACATCAATATTACAATTAGGAGTCCCATCAAGATTCAGTGGCAGT
GGGTCTGAAACAGATTATTCTCTCACCATTAGCAACCTGGAGCAAGAAGATATTGCCACTTAC
TTTTGCCAACAGGGTGATTCGCTTCCATTCACGTTCGGCTCGGGGACAAAGCTCGAGATCAAA
CGAACTGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACACAAAGT
CTATGCCTGCGAAGTCACCCATCAGGGCCTGAGCTCGCCCGTCACAAAGAGCTTCAACAGGGG
AGAGTGTTAG
Anti_CLEC_6_9B9.2G12_Kv-V-hIgGK-C (SEQ ID NO: 72):
MMSSAQFLGLLLLCFQGTRCDIQMTQTTSSLSASLGDRVTISCRASQDISNYLNWYQQKPDGTVKL
LIYYTSILQLGVPSRFSGSGSETDYSLTISNLEQEDIATYFCQQGDSLPFTFGSGTKLEIKRTVAAPSVF
IFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
Anti-ASGPR_49C11_7H-LV-hIgG4H-C (SEQ ID NO: 8):
ATGAGAGCGCTGATTCTTTTGTGCCTGTTCACAGCCTTTCCTGGTATCCTGTCTGATGTGCAGCT
TCAGGAGTCAGGACCTGACCTGGTGAAACCTTCTCAGTCACTTTCACTCACCTGCACTGTCACT
GGCTACTCCATCACCAGTGGTTATAGCTGGCACTGGATCCGGCAGTTTCCAGGAAACAAACTG
GAATGGATGGGCTACATACTCTTCAGTGGTAGCACTAACTACAACCCATCTCTGAAAAGTCGA
ATCTCTATCACTCGAGACACATCCAAGAACCAGTTCTTCCTGCAGTTGAATTCTGTGACTACTG
AGGACACAGCCACATATTTCTGTGCAAGATCTAACTATGGTTCCTTTGCTTCCTGGGGCCAAGG
GACTCTGGTCACTGTCTCTGCAGCCAAAACAACGGGCCCATCCGTCTTCCCCCTGGCGCCCTGC
TCCAGGAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCA
CGAAGACCTACACCTGCAACGTAGATCACAAGCCCAGCAACACCAAGGTGGACAAGAGAGTT
GAGTCCAAATATGGTCCCCCATGCCCACCCTGCCCAGCACCTGAGTTCGAAGGGGGACCATCA
GTCTTCCTGTTCCCCCCAAAACCCAAGGACACTCTCATGATCTCCCGGACCCCTGAGGTCACGT
GCGTGGTGGTGGACGTGAGCCAGGAAGACCCCGAGGTCCAGTTCAACTGGTACGTGGATGGC
GTGGAGGTGCATAATGCCAAGACAAAGCCGCGGGAGGAGCAGTTCAACAGCACGTACCGTGT
GGTCAGCGTCCTCACCGTCCTGCACCAGGACTGGCTGAACGGCAAGGAGTACAAGTGCAAGGT
CTCCAACAAAGGCCTCCCGTCCTCCATCGAGAAAACCATCTCCAAAGCCAAAGGGCAGCCCCG
AGAGCCACAGGTGTACACCCTGCCCCCATCCCAGGAGGAGATGACCAAGAACCAGGTCAGCC
TGACCTGCCTGGTCAAAGGCTTCTACCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCT
ACAGCAGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTAAAGCT
AGCTGATTAATTAA
Anti-ASGPR_49C11_7H-LV-hIgG4H-C (SEQ ID NO: 73):
MRALILLCLFTAFPGILSDVQLQESGPDLVKPSQSLSLTCTVTGYSITSGYSWHWIRQFPGNKLEWM
GYILFSGSTNYNPSLKSRISITRDTSKNQFFLQLNSVTTEDTATYFCARSNYGSFASWGQGTLVTVSA
AKTTGPSVFPLAPCSRSTSESTAALGCLVKDYFPEPVTVSWNSGALTSGVHTFPAVLQSSGLYSLSS
VVTVPSSSLGTKTYTCNVDHKPSNTKVDKRVESKYGPPCPPCPAPEFEGGPSVFLFPPKPKDTLMIS
RTPEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWESNG
QPENNYKTTPPVLDSDGSFFLYSRLTVDKSRWQEGNVFSCSVMHEALHNHYTQKSLSLSLGKAS
Anti-ASGPR_49C11_7K-LV-hIgGK-C (SEQ ID NO: 9):
ATGGATTTTCAAGTGCAGATTTTCAGCTTCCTGCTAATCAGTGCCTCAGTCATAATATCCAGAG
GACAAATTGTTCTCACCCAGTCTCCAGCAATCATGTCTGCATCTCCAGGGGAGAAGGTCACCA
TGACCTGCAGTGCCAGCTCAAGTGTAAGTCACATGCACTGGTACCAGCAGAAGTCAGGCACTT
CCCCCAAAAGATGGATTTATGACACATCCAGACTGGCTTCTGGAGTCCCTGCTCGCTTCAGTGG
CAGTGGGTCTGGGACCTCTTACTCTCTCACAATCAGCAGCATGGAGGCTGAAGATGCTGCCAC
TTATTACTGCCAGCAGTGGAGTAGTCACCCATGGTCGTTCGGTGGAGGCACCAAACTCGAGAT
CAAACGAACTGTGGCTGCACCATCTGTCTTCATCTTCCCGCCATCTGATGAGCAGTTGAAATCT
GGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAA
GGACAGCACCTACAGCCTCAGCAGCACCCTGACGCTGAGCAAAGCAGACTACGAGAAACACA
AAGTCTATGCCTGCGAAGTCACCCATCAGGGCCTGAGCTCGCCCGTCACAAAGAGCTTCAACA
GGGGAGAGTGTTAG
Anti-ASGPR_49C11_7K-LV-hIgGK-C (SEQ ID NO: 74):
MDFQVQIFSFLLISASVIISRGQIVLTQSPAIMSASPGEKVTMTCSASSSVSHMHWYQQKSGTSPKR
WIYDTSRLASGVPARFSGSGSGTSYSLTISSMEAEDAATYYCQQWSSHPWSFGGGTKLEIKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Anti-ASGPR_4G2.2_Hv-V-hIgG4H-C (SEQ ID NO: 10):
ATGGCTTGGGTGTGGACCTTGCTATTCCTGATGGCAGCTGCCCAAAGTGCCCAAGCACAGATC
CAGTTGGTGCAGTCTGGACCTGAGCTGAAGAAGCCTGGAGAGACAGTCAAGATCTCCTGCAAG
GCTTCTGGGTATACCTTCACAAACTATGGAATGAACTGGGTGAAGCAGGTTCCAGGAAAAGGT
TTAAGGTGGATGGGCTGGATGGACACCTTCACTGGAGAGCCAACATATGCTGATGACTTCAAG
GGACGGTTTGCCTTCTCTTTGGAAACCTCTGCCAGCACTGCCTATTTGCAGATCAACAGCCTCA
AAAATGAGGACACGGCTACTTATTTCTGTGCAAGAGGGGGGATTTTACGACTCAACTACTTTG
ACTACTGGGGCCAAGGCACCACTCTCACAGTCTCCTCAGCCAAAACGAAGGGCCCATCCGTCT
TCCCCCTGGCGCCCTGCTCCAGGAGCACCTCCGAGAGCACAGCCGCCCTGGGCTGCCTGGTCA
AGGACTACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGACCGTGCC
CTCCAGCAGCTTGGGCACGAAGACCTACACCTGCAACGTAGATCACAAGCCCAGCAACACCAA
GGTGGACAAGAGAGTTGAGTCCAAATATGGTCCCCCATGCCCACCCTGCCCAGCACCTGAGTT
CGAAGGGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCAGTTCAA
CTGGTACGTGGATGGCGTGGAGGTGCATAATGCCAAGACAAAGCCGCGGGAGGAGCAGTTCA
ACAGCACGTACCGTGTGGTCAGCGTCCTCACCGTCCTGCACCAGGACTGGCTGAACGGCAAGG
AGTACAAGTGCAAGGTCTCCAACAAAGGCCTCCCGTCCTCCATCGAGAAAACCATCTCCAAAG
CCAAAGGGCAGCCCCGAGAGCCACAGGTGTACACCCTGCCCCCATCCCAGGAGGAGATGACC
AAGAACCAGGTCAGCCTGACCTGCCTGGTCAAAGGCTTCTACCCCAGCGACATCGCCGTGGAG
TGGGAGAGCAATGGGCAGCCGGAGAACAACTACAAGACCACGCCTCCCGTGCTGGACTCCGA
CGGCTCCTTCTTCCTCTACAGCAGGCTAACCGTGGACAAGAGCAGGTGGCAGGAGGGGAATGT
CTTCTCATGCTCCGTGATGCATGAGGCTCTGCACAACCACTACACACAGAAGAGCCTCTCCCTG
TCTCTGGGTAAAGCTAGCTGATTAATTAA
Anti-ASGPR_4G2.2_Hv-V-hIgG4H-C (SEQ ID NO: 75):
MAWVWTLLFLMAAAQSAQAQIQLVQSGPELKKPGETVKISCKASGYTFTNYGMNWVKQVPGKG
LRWMGWMDTFTGEPTYADDFKGRFAFSLETSASTAYLQINSLKNEDTATYFCARGGILRLNYFDY
WGQGTTLTVSSAKTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQ
KSLSLSLGKAS
Anti-ASGPR_4G2.2_Kv-V-hIgGK-C (SEQ ID NO: 11):
ATGAAGTTTCCTTCTCAACTTCTGCTCTTACTGCTGTTTGGAATCCCAGGCATGATATGTGACAT
CCAGATGACACAATCTTCATCCTCCTTTTCTGTATCTCTAGGAGACAGAGTCACCATTACTTGC
AAGGCAAGTGAGGACATATATAATCGGTTAGGCTGGTATCAGCAGAAACCAGGAAATGCTCCT
AGGCTCTTAATATCTGGTGCAACCAGTTTGGAAACTGGGGTTCCTTCAAGATTCAGTGGCAGTG
GATCTGGAAAGGATTACGCTCTCAGCATTACCAGTCTTCAGACTGAAGATCTTGCTACTTATTA
CTGTCAACAGTGTTGGACTTCTCCGTACACGTTCGGAGGGGGGACCAAGCTCGAGATCAAACG
AACTGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCT
ATGCCTGCGAAGTCACCCATCAGGGCCTGAGCTCGCCCGTCACAAAGAGCTTCAACAGGGGAG
AGTGTTAG
Anti-ASGPR_4G2.2_Kv-V-hIgGK-C (SEQ ID NO: 76):
MKFPSQLLLLLLFGIPGMICDIQMTQSSSSFSVSLGDRVTITCKASEDIYNRLGWYQQKPGNAPRLLI
SGATSLETGVPSRFSGSGSGKDYALSITSLQTEDLATYYCQQCWTSPYTFGGGTKLEIKRTVAAPSV
FIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
Anti-ASGPR_5F10H-LV-hIgG4H-C (SEQ ID NO: 12):
ATGGGATGGAGCTGGATCTTTCTCTTTCTCTTGTCAGGAACTGGAGGTGTCCTCTCTGAGGTCC
AGCTGCAACAGTCTGGACCTGAGCTGGTGAAGCCTGGGGCTTCAGTGAAGATGTCCTGCAAGG
CTTCTGGATACACCTTCACTGACTACTACATGAAGTGGGTGAAGCAGAGCCATGGAAAGAGCC
TTGAGTGGATTGGAGATATTAATCCTAACTATGGTGATACTTTCTACAACCAGAAGTTCGAGG
GCAAGGCCACATTGACTGTAGACAAATCCTCCAGGACAGCCTACATGCAGCTCAACAGCCTGA
CATCTGAGGACTCTGCAGTCTATTATTGTGGAAGAGGGGACTATGGATACTTCGATGTCTGGG
GCGCAGGGACCACGGTCACCGTCTCCTCAGCCAAAACAAAGGGCCCATCCGTCTTCCCCCTGG
CGCCCTGCTCCAGGAGCACCTCCGAGAGCACAGCCGCCCTGGGCTGCCTGGTCAAGGACTACT
TCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGGTGGACA
AGAGAGTTGAGTCCAAATATGGTCCCCCATGCCCACCCTGCCCAGCACCTGAGTTCGAAGGGG
GACCATCAGTCTTCCTGTTCCCCCCAAAACCCAAGGACACTCTCATGATCTCCCGGACCCCTGA
GGTCACGTGCGTGGTGGTGGACGTGAGCCAGGAAGACCCCGAGGTCCAGTTCAACTGGTACGT
GGATGGCGTGGAGGTGCATAATGCCAAGACAAAGCCGCGGGAGGAGCAGTTCAACAGCACGT
ACCGTGTGGTCAGCGTCCTCACCGTCCTGCACCAGGACTGGCTGAACGGCAAGGAGTACAAGT
GCAAGGTCTCCAACAAAGGCCTCCCGTCCTCCATCGAGAAAACCATCTCCAAAGCCAAAGGGC
AGCCCCGAGAGCCACAGGTGTACACCCTGCCCCCATCCCAGGAGGAGATGACCAAGAACCAG
GTCAGCCTGACCTGCCTGGTCAAAGGCTTCTACCCCAGCGACATCGCCGTGGAGTGGGAGAGC
AATGGGCAGCCGGAGAACAACTACAAGACCACGCCTCCCGTGCTGGACTCCGACGGCTCCTTC
TTCCTCTACAGCAGGCTAACCGTGGACAAGAGCAGGTGGCAGGAGGGGAATGTCTTCTCATGC
TCCGTGATGCATGAGGCTCTGCACAACCACTACACACAGAAGAGCCTCTCCCTGTCTCTGGGT
AAAGCTAGCTGA
Anti-ASGPR_5F10H-LV-hIgG4H-C (SEQ ID NO: 77):
MGWSWIFLFLLSGTGGVLSEVQLQQSGPELVKPGASVKMSCKASGYTFTDYYMKWVKQSHGKSL
EWIGDINPNYGDIFYNQKFEGKATLTVDKSSRTAYMQLNSLTSEDSAVYYCGRGDYGYFDVWGA
GTTVTVSSAKTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSVFLFPPKP
KDTLMISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLL
SLGKAS
Anti-ASGPR_5F10K-LV-hIgGK-C (SEQ ID NO: 13):
ATGGAGACACATTCTCAGGTCTTTGTATACATGTTGCTGTGGTTGTCTGGTGTTGAAGGAGACA
TTGTGATGACCCAGTCTCACAAATTCATGTCCACATCAGTAGGAGACAGGGTCAGCATCACCT
GCAAGGCCAGTCAGGATGTGGGTACTGCTGTAGCCTGGTATCAACAGAAACCAGGGCAATCTC
CTAAACTACTGATTTACTGGGCATCCACCCGGCACACTGGAGTCCCTGATCGCTTCACAGGCA
GTGGATCTGGGACAGATTTCACTCTCACCATTAACAATGTGCAGTCTGAAGACTTGGCAGATT
ATTTCTGTCAGCAATATAGCAGCAATCCGTACATGTTCGGAGGGGGGACCAAGCTCGAGATCA
AACGAACTGTGGCTGCACCATCTGTCTTCATCTTCCCGCCATCTGATGAGCAGTTGAAATCTGG
AACTGCCTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAACACAAA
GTCTATGCCTGCGAAGTCACCCATCAGGGCCTGAGCTCGCCCGTCACAAAGAGCTTCAACAGG
GGAGAGTGTTAG
Anti-ASGPR_5F10K-LV-hIgGK-C (SEQ ID NO: 78):
METHSQVFVYMLLWLSGVEGDIVMTQSHKFMSTSVGDRVSITCKASQDVGTAVAWYQQKPGQSP
KLLIYWASTRHTGVPDRFTGSGSGTDFTLTINNVQSEDLADYFCQQYSSNPYMFGGGTKLEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-ASGPR1H11_H-V-hIgG4H-C (SEQ ID NO: 14):
ATGGGATGGAGCTGGATCTTTCTCTTTCTCCTGTCAGGAACTGCAGGTGTCCTCTCTGAGGTCC
AGCTGCAACAGTCTGGACCTGAGTTGGTGAAGCCTGGGGCTTCAGTGAAGATATCCTGCAAGA
CTTCTGGATACACATTCACTGAATACACCATGCACTGGGTGAGGCAGAGCCATGGAAAGAGCC
TTGAGTGGATTGGAGGTATTAATCCTATCAATGGTGGTCCTACCTACAACCAGAAGTTCAAGG
GCAAGGCCACATTGACTGTTGACAAGTCCTCCAGCACAGCCTACATGGAGCTCCGCAGCCTGA
CATCTGAGGACTCTGCAGTCTATTACTGTGCAAGATGGGACTATGGTAGTCGAGATGTTATGG
ACTACTGGGGTCAAGGAACCTCAGTCACCGTCTCCTCAGCCAAAACGAAGGGCCCATCCGTCT
TCCCCCTGGCGCCCTGCTCCAGGAGCACCTCCGAGAGCACAGCCGCCCTGGGCTGCCTGGTCA
AGGACTACTTCCCCGAACCGGTACCGGTGACGGTGTCGTGGAACTCAGGCGCCCTGACCAGCG
GCGTGCACACCTTCCCGGCTGTCCTACAGTCCTCAGGACTCTACTCCCTCAGCAGCGTGGTGAC
CGTGCCCTCCAGCAGCTTGGGCACGAAGACCTACACCTGCAACGTAGATCACAAGCCCAGCAA
CACCAAGGTGGACAAGAGAGTTGAGTCCAAATATGGTCCCCCATGCCCACCCTGCCCAGCACC
TGAGTTCGAAGGGGGACCATCAGTCTTCCTGTTCCCCCCAAAACCCAAGGACACTCTCATGAT
CTCCCGGACCCCTGAGGTCACGTGCGTGGTGGTGGACGTGAGCCAGGAAGACCCCGAGGTCCA
GTTCAACTGGTACGTGGATGGCGTGGAGGTGCATAATGCCAAGACAAAGCCGCGGGAGGAGC
AGTTCAACAGCACGTACCGTGTGGTCAGCGTCCTCACCGTCCTGCACCAGGACTGGCTGAACG
GCAAGGAGTACAAGTGCAAGGTCTCCAACAAAGGCCTCCCGTCCTCCATCGAGAAAACCATCT
CCAAAGCCAAAGGGCAGCCCCGAGAGCCACAGGTGTACACCCTGCCCCCATCCCAGGAGGAG
ATGACCAAGAACCAGGTCAGCCTGACCTGCCTGGTCAAAGGCTTCTACCCCAGCGACATCGCC
GTGGAGTGGGAGAGCAATGGGCAGCCGGAGAACAACTACAAGACCACGCCTCCCGTGCTGGA
CTCCGACGGCTCCTTCTTCCTCTACAGCAGGCTAACCGTGGACAAGAGCAGGTGGCAGGAGGG
GAATGTCTTCTCATGCTCCGTGATGCATGAGGCTCTGCACAACCACTACACACAGAAGAGCCT
CTCCCTGTCTCTGGGTAAAGCTAGCTGA
Anti-ASGPR1H11_H-V-hIgG4H-C (SEQ ID NO: 79):
MGWSWIFLFLLSGTAGVLSEVQLQQSGPELVKPGASVKISCKTSGYTFTEYTMHWVRSHGKSLEW
IGGINPINGGPTYNQKFKGKATLTVDKSSSTAYMELRSLTSEDSAVYYCARWDYGSRDVMDYWG
QGTSVTVSSAKTKGPSVFPLAPCSRSTSESTAALGCLVKDYFPEPVPVTVSWNSGALTSGVHTFPAV
LQSSGLYSLSSVVTVPSSSLGTKTYTCNVDHKPSNTKVDKRVESKYGPPCPPCPAPEFEGGPSVFLFP
PKPKDTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMHEALHNHYTQKS
LSLSLGKAS
Anti-ASGPR1H11K-LV-var2-hIgGK-C (SEQ ID NO: 15):
ATGGAATCACAGACTCTGGTCTTCATATCCATACTGCTCTGGTTATATGGTGCTGATGGGAACA
TTGTAATGACTCAATCTCCCAAATCCATGTCCATGTCAGTAGGGGAGAGGGTCACCTTGAGCT
GCAAGGCCAGTGAGAATGTGGGAACTTATGTATCCTGGTATCAACAGAGACCAGAACAGTCTC
CAAAACTGCTGATATACGGGGCATCCAACCGGTACACTGGGGTCCCCGATCGCTTCACAGGCA
GTGGATCTGCAACAGATTTCACTCTGACCATCAGCAGTGTGCAGGCTGAGGACCTTGCAGATT
ATCACTGTGGACAGACTTACAGCTATATATTCACGTTCGGCTCGGGGACAAAGCTCGAGATCA
AACGAACTGTGGCTGCACCATCTGTCTTCATCTTCCCGCCATCTGATGAGCAGTTGAAATCTGG
AACTGCCTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAACACAAA
GTCTATGCCTGCGAAGTCACCCATCAGGGCCTGAGCTCGCCCGTCACAAAGAGCTTCAACAGG
GGAGAGTGTTAG
Anti-ASGPR1H11K-LV-var2-hIgGK-C (SEQ ID NO: 80):
METHSQVFVYMLLWLSGVEGNIVMTQSPKSMSMSVGERVTLSCKASENVGTYVSWYQQRPEQSP
KLLIYGASNRYTGVPDRFTGSGSATDFTLTISSVQAEDLADYHCGQTYSYIFTFGSGTKLEIKRTVA
APSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Anti-CD1d_2B5.3G10_H-V-hIgG4H-C (SEQ ID NO: 16):
ATGGGATGGAGCCGGATCTTTCTCTTCCTCCTGTCAATAACTGCAGGTGTCCATTGCCAGGTCC
AGGTGCAGCAGTCGGGACCTGAGTTGGTGAAGCCTGGGGCCTCAGTGAAGATTTCCTGCAAAG
CCTCTGGCGACGCATTCAGTAGTTCTTGGATGAACTGGGTGAAGCAGAGGCCTGGACAGGGTC
TTGAGTGGATTGGACGGATTTATCTTGGAGATGGAGATATTAATTACAATGGGAAGTTCAAGG
GCAGGGCCACACTGACTGCAGACAAATCCTCCAGCACAGCCTACATGCAGCTCAGCAGCCTGA
CCTCTGTGGACTCTGCGGTCTATTTCTGCGCGAGGCAGCTCGGGCTATGGTATGTTATGGACTA
CTGGGGTCAAGGAACCTCAGTCACCGTCTCCTCAGCCAAAACAAAGGGCCCATCCGTCTTCCC
CCTGGCGCCCTGCTCCAGGAGCACCTCCGAGAGCACAGCCGCCCTGGGCTGCCTGGTCAAGGA
CTACTTCCCCGAACCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGGTGACCGTGCCCTCC
AGCAGCTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAGTTCGA
AGGGGGACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTTCAACTG
GTACGTGGATGGCGTGGAGGTGCATAATGCCAAGACAAAGCCGCGGGAGGAGCAGTTCAACA
GCACGTACCGTGTGGTCAGCGTCCTCACCGTCCTGCACCAGGACTGGCTGAACGGCAAGGAGT
ACAAGTGCAAGGTCTCCAACAAAGGCCTCCCGTCCTCCATCGAGAAAACCATCTCCAAAGCCA
AAGGGCAGCCCCGAGAGCCACAGGTGTACACCCTGCCCCCATCCCAGGAGGAGATGACCAAG
AACCAGGTCAGCCTGACCTGCCTGGTCAAAGGCTTCTACCCCAGCGACATCGCCGTGGAGTGG
GAGAGCAATGGGCAGCCGGAGAACAACTACAAGACCACGCCTCCCGTGCTGGACTCCGACGG
CTCCTTCTTCCTCTACAGCAGGCTAACCGTGGACAAGAGCAGGTGGCAGGAGGGGAATGTCTT
CTCATGCTCCGTGATGCATGAGGCTCTGCACAACCACTACACACAGAAGAGCCTCTCCCTGTCT
CTGGGTAAAGCTAGCTGA
Anti-CD1d_2B5.3G10_H-V-hIgG4H-C (SEQ ID NO: 81):
MGWSRIFLFLLSITAGVHCQVQVQQSGPELVKPGASVKISCKASGDAFSSSWMNWVKQRPGQGLE
WIGRIYLGDGDINYNGKFKGRATLTADKSSSTAYMQLSSLTSVDSAVYFCARQLGLWYVMDYWG
QGTSVTVSSAKTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFEGGPSVFLFPPK
PKDTLMISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLS
LSLGKAS
Anti-CD1d_2B5.3G10_K-V-hIgGK-C (SEQ ID NO: 17):
ATGAGTGTGCCCACTCAGGTCCTGGGGTTGCTGCTGCTGTGGCTTACAGGTGCCAGATGTGAC
ATCCAGATGGCTCAGTCTCCAGCCTCCCTATCTGCATCTGTGGGAGAAACTGTCACCATCACAT
GTCGAGCAAGTGAGAATATTTACAGTTATTTAGCATGGTATCAGCAGAAACAGGGAAAATCTC
CTCAGCTCCTGGTCTATAATGCAAAAACCTTAGCAGAAGGTGTGCCATCAAGGTTCAGTGGCA
GTGGATCAGGCACACAGTTTTCTCTGAAGATCAACAGCCTGCAGCCTGAAGATTTTGGGAGTT
ATTACTGTCAACATCATTATGGTTTTCCGTGGACGTTCGGTGGAGGCACCAAGCTCGAGATCAA
ACGAACTGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACACAAAG
TCTATGCCTGCGAAGTCACCCATCAGGGCCTGAGCTCGCCCGTCACAAAGAGCTTCAACAGGG
GAGAGTGTTAG
Anti-CD1d_2B5.3G10_K-V-hIgGK-C (SEQ ID NO: 82):
MSVPTQVLGLLLLWLTGARCDIQMAQSPASLSASVGETVTITCRASENIYSYLAWYQQKQGKSPQL
LVYNAKTLAEGVPSRFSGSGSGTQFSLKINSLQPEDFGSYYCQHHYGFPWTFGGGTKLEIKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Anti-CD1d_2H11.2G5_H-V-hIgG4H-C (SEQ ID NO: 18):
ATGAACTTCGGGCTCAGCTTGATTTTCCTTGTCCTCATTTTAAAAGGTGTCCAGTGTGAGGTGC
AGCTGGTGGAGTCTGGGGGAGACTTAGTGAAGCCTGGAGGGTCCCTGAAACTCTCCTGTGCAG
CCTCTGGATTCACTTTCAGTAGCTATGGCATGTCTTGGGTTCGCCAGACTCCAGACAAGAGGCT
GGAGTGGGTCGCAGTCATTAGTAGTGGTGGAAGTTCCACCTTCTATCCAGACAGTGTGAAGGG
GCGATTCACCATCTCCAGAGACAATGCCAAGAACACCCTGTACCTGCAAATGAGCAGTCTGAA
GTCTGAGGACACAGCCGTGTATTACTGTTCAAGAGGAGGTTACTACTTTGACTACTGGGGCCA
AGGCACCACTCTCACAGTCTCCGCAGCCAAAACAAAGGGCCCATCCGTCTTCCCCCTGGCGCC
CTGCTCCAGGAGCACCTCCGAGAGCACAGCCGCCCTGGGCTGCCTGGTCAAGGACTACTTCCC
CGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTG
GGCACGAAGACCTACACCTGCAACGTAGATCACAAGCCCAGCAACACCAAGGTGGACAAGAG
AGTTGAGTCCAAATATGGTCCCCCATGCCCACCCTGCCCAGCACCTGAGTTCGAAGGGGGACC
ATCAGTCTTCCTGTTCCCCCCAAAACCCAAGGACACTCTCATGATCTCCCGGACCCCTGAGGTC
ACGTGCGTGGTGGTGGACGTGAGCCAGGAAGACCCCGAGGTCCAGTTCAACTGGTACGTGGAT
GGCGTGGAGGTGCATAATGCCAAGACAAAGCCGCGGGAGGAGCAGTTCAACAGCACGTACCG
TGTGGTCAGCGTCCTCACCGTCCTGCACCAGGACTGGCTGAACGGCAAGGAGTACAAGTGCAA
GGTCTCCAACAAAGGCCTCCCGTCCTCCATCGAGAAAACCATCTCCAAAGCCAAAGGGCAGCC
CCGAGAGCCACAGGTGTACACCCTGCCCCCATCCCAGGAGGAGATGACCAAGAACCAGGTCA
GCCTGACCTGCCTGGTCAAAGGCTTCTACCCCAGCGACATCGCCGTGGAGTGGGAGAGCAATG
GGCAGCCGGAGAACAACTACAAGACCACGCCTCCCGTGCTGGACTCCGACGGCTCCTTCTTCC
TCTACAGCAGGCTAACCGTGGACAAGAGCAGGTGGCAGGAGGGGAATGTCTTCTCATGCTCCG
TGATGCATGAGGCTCTGCACAACCACTACACACAGAAGAGCCTCTCCCTGTCTCTGGGTAAAG
CTAGCTGA
Anti-CD1d_2H11.2G5_H-V-hIgG4H-C (SEQ ID NO: 83):
MNFGLSLIFLVLILKGVQCEVQLVESGGDLVKPGGSLKLSCAASGFTFSSYGMSWVRQTPDKRLEW
VAVISSGGSSTFYPDSVKGRFTISRDNAKNTLYLQMSSLKSEDTAVYYCSRGGYYFDYWGQGTTLT
VSAAKTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFEGGPSVFLFPPKPKDTL
MISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHYTQKSLSLSLGK
AS
Anti-CD1d_2H11.2G5_K-V-hIgGK-C (SEQ ID NO: 19):
ATGAGGTTCCAGGTTCAGGTTCTGGGGCTCCTTCTGCTCTGGATATCAGGTGCCCAGTGTGATG
TCCAGATAACCCAGTCTCCATCTTATCTTGCTGCATCTCCTGGAGAAACCATTACTATTAATTG
CAGGGCAAGCAAGACCATTAGCAAATATTTAGCCTGGTATCAAGAGAAACCTGAGAAAACTG
ATAAGCTTCTTATCTACTCTGGATCCACTTTGCAATCTGGAATTCCATCAAGGTTCAGTGGCAG
TGGATCTGGTACAGATTTCACTCTCACCATCAGTGGCCTGGAGCCTGAAGATTTTGCAATGTAT
TACTGTCAACAGCATAATGAATACCCGTGGACGTTCGGTGGAGGCACCAAGCTCGAGATCAAA
CGAACTGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACACAAAGT
CTATGCCTGCGAAGTCACCCATCAGGGCCTGAGCTCGCCCGTCACAAAGAGCTTCAACAGGGG
AGAGTGTTAG
Anti-CD1d_2H11.2G5_K-V-hIgGK-C (SEQ ID NO: 84):
MRFQVQVLGLLLLWISGAQCDVQITQSPSYLAASPGETITINCRASKTISKYLAWYQEKPEKTDKLL
IYSGSTLQSGIPSRFSGSGSGTDFTLTISGLEPEDFAMYYCQQHNEYPWTFGGGTKLEIKRTVAAPSV
FIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
Anti-CD40_11B6.1C3_H-LV-hIgG4H-C (SEQ ID NO: 20):
ATGGGATGGAGCTGGATCTTTCTCTTTCTCCTGTCAGGAACTGCAGGTGTCCTCTCTGAGGTCC
AGCTGCAACAGTCTGGACCTGAGCTGGTGAAGCCTGGGGCTTCAGTGAAGATATCCTGCAAGG
CTTCTGGTTACTCATTCACTGGCTACTACATGCACTGGGTGAAGCAAAGCCATGTAAAGAGCCT
TGAGTGGATTGGACGTATTAATCCTTACAATGGTGCTACTAGCTACAACCAGAATTTCAAGGA
CAAGGCCAGCTTGACTGTAGATAAGTCCTCCAGCACAGCCTACATGGAGCTCCACAGCCTGAC
ATCTGAGGACTCTGCAGTCTATTACTGTGCAAGAGAGGACTACGTCTACTGGGGCCAAGGCAC
CACTCTCACAGTCTCCTCAGCCAAAACGAAGGGCCCATCCGTCTTCCCCCTGGCGCCCTGCTCC
AGGAGCACCTCCGAGAGCACAGCCGCCCTGGGCTGCCTGGTCAAGGACTACTTCCCCGAACCG
GTGACGGTGTCGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAGCTTGGGCACG
AAGACCTACACCTGCAACGTAGATCACAAGCCCAGCAACACCAAGGTGGACAAGAGAGTTGA
GTCCAAATATGGTCCCCCATGCCCACCCTGCCCAGCACCTGAGTTCGAAGGGGGACCATCAGT
CTTCCTGTTCCCCCCAAAACCCAAGGACACTCTCATGATCTCCCGGACCCCTGAGGTCACGTGC
GTGGTGGTGGACGTGAGCCAGGAAGACCCCGAGGTCCAGTTCAACTGGTACGTGGATGGCGTG
GAGGTGCATAATGCCAAGACAAAGCCGCGGGAGGAGCAGTTCAACAGCACGTACCGTGTGGT
CAGCGTCCTCACCGTCCTGCACCAGGACTGGCTGAACGGCAAGGAGTACAAGTGCAAGGTCTC
CAACAAAGGCCTCCCGTCCTCCATCGAGAAAACCATCTCCAAAGCCAAAGGGCAGCCCCGAG
AGCCACAGGTGTACACCCTGCCCCCATCCCAGGAGGAGATGACCAAGAACCAGGTCAGCCTG
ACCTGCCTGGTCAAAGGCTTCTACCCCAGCGACATCGCCGTGGAGTGGGAGAGCAATGGGCAG
CCGGAGAACAACTACAAGACCACGCCTCCCGTGCTGGACTCCGACGGCTCCTTCTTCCTCTACA
GCAGGCTAACCGTGGACAAGAGCAGGTGGCAGGAGGGGAATGTCTTCTCATGCTCCGTGATGC
ATGAGGCTCTGCACAACCACTACACACAGAAGAGCCTCTCCCTGTCTCTGGGTAAAGCTAGCT
GA
Anti-CD40_11B6.1C3_H-LV-hIgG4H-C (SEQ ID NO: 85):
MGWSWIFLFLLSGTAGVLSEVQLQQSGPELVKPGASVKISCKASGYSFTGYYMHWVKQSHVKSLE
WIGRINPYNGATSYNQNFKDKASLTVDKSSSTAYMELHSLTSEDSAVYYCAREDYVYWGQGTTLT
VSSAKTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFEGGPSVFLFPPKPKDTL
MISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHYTQKSLSLSLGK
AS
Anti-CD40_11B6.1C3_K-LV-hIgGK-C (SEQ ID NO: 21):
ATGAAGTTGCCTGTTAGGCTGTTGGTGCTGATGTTCTGGATTCCTGCTTCCAGCAGTGATGTTG
TGATGACCCAAACTCCACTCTCCCTGCCTGTCAGTCTTGGAGATCAAGCCTCCATCTCTTGCAG
ATCTAGTCAGAGCCTTGTACACAGTAATGGAAACACCTATTTACATTGGTACCTGCAGAAGCC
AGGCCAGTCTCCAAAGCTCCTGATCTACAAAGTTTCCAACCGATTTTCTGGGGTCCCAGACAG
GTTCAGTGGCAGTGGATCAGGGACAGATTTCGCACTCAAGATCAGTAGAGTGGAGGCTGAGG
ATCTGGGAGTTTATTTCTGCTCTCAAAGTACACATGTTCCGTGGACGTTCGGTGGAGGCACCAA
GCTCGAGATCAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGA
GAAACACAAAGTCTATGCCTGCGAAGTCACCCATCAGGGCCTGAGCTCGCCCGTCACAAAGAG
CTTCAACAGGGGAGAGTGTTAG
Anti-CD40_11B6.1C3_K-LV-hIgGK-C (SEQ ID NO: 86):
MKLPVRLLVLMFWIPASSSDVVMTQTPLSLPVSLGDQASISCRSSQSLVHSNGNTYLHWYLQKPGQ
SPKLLIYKVSNRFSGVPDRFSGSGSGTDFALKISRVEAEDLGVYFCSQSTHVPWTFGGGTKLEIKRT
VAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-CD40_12B4.2C10_H-LV-hIgG4H-C (SEQ ID NO: 22):
ATGGAATGGAGTTGGATATTTCTCTTTCTTCTGTCAGGAACTGCAGGTGTCCACTCTGAGGTCC
AGCTGCAGCAGTCTGGACCTGAGCTGGTAAAGCCTGGGGCTTCAGTGAAGATGTCCTGCAAGG
CTTCTGGATACACATTCACTGACTATGTTTTGCACTGGGTGAAACAGAAGCCTGGGCAGGGCC
TTGAGTGGATTGGATATATTAATCCTTACAATGATGGTACTAAGTACAATGAGAAGTTCAAAG
GCAAGGCCACACTGACTTCAGACAAATCCTCCAGCACAGCCTACATGGAGCTCAGCAGCCTGA
CCTCTGAGGACTCTGCGGTCTATTACTGTGCAAGGGGCTATCCGGCCTACTCTGGGTATGCTAT
GGACTACTGGGGTCAAGGAACCTCAGTCACCGTCTCCTCAGCCAAAACGAAGGGCCCATCCGT
CTTCCCCCTGGCGCCCTGCTCCAGGAGCACCTCCGAGAGCACAGCCGCCCTGGGCTGCCTGGT
CAAGGACTACTTCCCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCAGCGTGGTGACCGTG
CCCTCCAGCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGCACCTGAG
TTCGAAGGGGGACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGAGGTCCAGTTCA
ACTGGTACGTGGATGGCGTGGAGGTGCATAATGCCAAGACAAAGCCGCGGGAGGAGCAGTTC
AACAGCACGTACCGTGTGGTCAGCGTCCTCACCGTCCTGCACCAGGACTGGCTGAACGGCAAG
GAGTACAAGTGCAAGGTCTCCAACAAAGGCCTCCCGTCCTCCATCGAGAAAACCATCTCCAAA
GCCAAAGGGCAGCCCCGAGAGCCACAGGTGTACACCCTGCCCCCATCCCAGGAGGAGATGAC
CAAGAACCAGGTCAGCCTGACCTGCCTGGTCAAAGGCTTCTACCCCAGCGACATCGCCGTGGA
GTGGGAGAGCAATGGGCAGCCGGAGAACAACTACAAGACCACGCCTCCCGTGCTGGACTCCG
ACGGCTCCTTCTTCCTCTACAGCAGGCTAACCGTGGACAAGAGCAGGTGGCAGGAGGGGAATG
TCTTCTCATGCTCCGTGATGCATGAGGCTCTGCACAACCACTACACACAGAAGAGCCTCTCCCT
GTCTCTGGGTAAAGCTAGCTGA
Anti-CD40_12B4.2C10_H-LV-hIgG4H-C (SEQ ID NO: 87):
MEWSWIFLFLLSGTAGVHSEVQLQQSGPELVKPGASVKMSCKASGYTFTDYVLHWVKQKPGQGL
EWIGYINPYNDGTKYNEKFKGKATLTSDKSSSTAYMELSSLTSEDSAVYYCARGYPAYSGYAMDY
WGQGTSVTVSSAKTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQ
KSLSLSLGKAS
Anti-CD40_12B4.2C10_K-LV-v2-hIgGK-C (SEQ ID NO: 23):
ATGATGTCCTCTGCTCAGTTCCTTGGTCTCCTGTTGCTCTGTTTTCAAGGTACCAGATGTGATAT
CCAGATGACACAGACTACATCCTCCCTGTCTGCCTCTCTGGGAGACAGAGTCACCATCAGTTGC
AGGGCAAGTCAGGACATTAGCAATTATTTAAACTGGTATCAGCAGAAACCAGATGGAACTGTT
AAACTCCTGATCTACTACACATCAAGATTACACTCAGGAGTCCCATCAAGGTTCAGTGGCAGT
GGGTCTGGAACAGATTATTCTCTCACCATTAGCAACCTGGAGCAAGAAGATATTGCCACTTAC
TTTTGCCATCATGGTAATACGCTTCCGTGGACGTTCGGTGGAGGCACCAAGCTCGAGATCAAA
CGAACTGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGG
TGGATAACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACACAAAGT
CTATGCCTGCGAAGTCACCCATCAGGGCCTGAGCTCGCCCGTCACAAAGAGCTTCAACAGGGG
AGAGTGTTAG
Anti-CD40_12B4.2C10_K-LV-v2-hIgGK-C (SEQ ID NO: 88):
MMSSAQFLGLLLLCFQGTRCDIQMTQTTSSLSASLGDRVTISCRASQDISNYLNWYQQKPDGTVKL
LIYYTSRLHSGVPSRFSGSGSGTDYSLTISNLEQEDIATYFCHHGNTLPWTFGGGTKLEIKRTVAAPS
VFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Anti-CD40_12E12.3F3_H-V-hIgG4H-C (SEQ ID NO: 24):
ATGAACTTGGGGCTCAGCTTGATTTTCCTTGTCCTTGTTTTAAAAGGTGTCCAGTGTGAAGTGA
AGCTGGTGGAGTCTGGGGGAGGCTTAGTGCAGCCTGGAGGGTCCCTGAAACTCTCCTGTGCAA
CCTCTGGATTCACTTTCAGTGACTATTACATGTATTGGGTTCGCCAGACTCCAGAGAAGAGGCT
GGAGTGGGTCGCATACATTAATTCTGGTGGTGGTAGCACCTATTATCCAGACACTGTAAAGGG
CCGATTCACCATCTCCAGAGACAATGCCAAGAACACCCTGTACCTGCAAATGAGCCGGCTGAA
GTCTGAGGACACAGCCATGTATTACTGTGCAAGACGGGGGTTACCGTTCCATGCTATGGACTA
TTGGGGTCAAGGAACCTCAGTCACCGTCTCCTCAGCCAAAACGAAGGGCCCATCCGTCTTCCC
CCTGGCGCCCTGCTCCAGGAGCACCTCCGAGAGCACAGCCGCCCTGGGCTGCCTGGTCAAGGA
CTACTTCCCCGAACCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGGTGACCGTGCCCTCC
AGCAGCTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAGTTCGA
AGGGGGACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTTCAACTG
GTACGTGGATGGCGTGGAGGTGCATAATGCCAAGACAAAGCCGCGGGAGGAGCAGTTCAACA
GCACGTACCGTGTGGTCAGCGTCCTCACCGTCCTGCACCAGGACTGGCTGAACGGCAAGGAGT
ACAAGTGCAAGGTCTCCAACAAAGGCCTCCCGTCCTCCATCGAGAAAACCATCTCCAAAGCCA
AAGGGCAGCCCCGAGAGCCACAGGTGTACACCCTGCCCCCATCCCAGGAGGAGATGACCAAG
AACCAGGTCAGCCTGACCTGCCTGGTCAAAGGCTTCTACCCCAGCGACATCGCCGTGGAGTGG
GAGAGCAATGGGCAGCCGGAGAACAACTACAAGACCACGCCTCCCGTGCTGGACTCCGACGG
CTCCTTCTTCCTCTACAGCAGGCTAACCGTGGACAAGAGCAGGTGGCAGGAGGGGAATGTCTT
CTCATGCTCCGTGATGCATGAGGCTCTGCACAACCACTACACACAGAAGAGCCTCTCCCTGTCT
CTGGGTAAAGCTAGCTGA
Anti-CD40_12E12.3F3_H-V-hIgG4H-C (SEQ ID NO: 89):
MNLGLSLIFLVLVLKGVQCEVKLVESGGGLVQPGGSLKLSCATSGFTFSDYYMYWVRQTPEKRLE
WVAYINSGGGSTYYPDTVKGRFTISRDNAKNTLYLQMSRLKSEDTAMYYCARRGLPFHAMDYWG
QGTSVTVSSAKTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFEGGPSVFLFPPK
PKDTLMISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLS
LSLGKAS
Anti-CD40_12E12.3F3_K-LV-hIgGK-C (SEQ ID NO: 25):
ATGATGTCCTCTGCTCAGTTCCTTGGTCTCCTGTTGCTCTGTTTTCAAGGTACCAGATGTGATAT
CCAGATGACACAGACTACATCCTCCCTGTCTGCCTCTCTAGGAGACAGAGTCACCATCAGTTGC
AGTGCAAGTCAGGGCATTAGCAATTATTTAAACTGGTATCAGCAGAAACCAGATGGAACTGTT
AAACTCCTGATCTATTACACATCAATTTTACACTCAGGAGTCCCATCAAGGTTCAGTGGCAGTG
GGTCTGGGACAGATTATTCTCTCACCATCGGCAACCTGGAACCTGAAGATATTGCCACTTACTA
TTGTCAGCAGTTTAATAAGCTTCCTCCGACGTTCGGTGGAGGCACCAAACTCGAGATCAAACG
AACTGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCT
ATGCCTGCGAAGTCACCCATCAGGGCCTGAGCTCGCCCGTCACAAAGAGCTTCAACAGGGGAG
AGTGTTAG
Anti-CD40_12E12.3F3_K-LV-hIgGK-C (SEQ ID NO: 90):
MMSSAQFLGLLLLCFQGTRCDIQMTQTTSSLSASLGDRVTISCSASQGISNYLNWYQQKPDGTVKL
LIYYTSILHSGVPSRFSGSGSGTDYSLTIGNLEPEDIATYYCQQFNKLPPTFGGGTKLEIKRTVAAPSV
FIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
Anti-DCIR_24A5.4A5_H-V-hIgG4H-C (SEQ ID NO: 26):
ATGGATTGGCTGTGGAACTTGCTATTCCTGATGGCAGCTGCCCAAAGTGCCCAAGCACAGATC
CAGTTGGTGCAGTCTGGACCTGAGCTGAAGAAGCCTGGAGAGACAGTCAAGATCTCCTGCAAG
GCTTCTGGGTATTCCTTCACAAACTATGGAATGAACTGGGTGAAACAGGCTCCAGGAAAGGGT
TTAAAGTGGATGGGCTGGATAAACACCTACACTGGAGAGTCAACATATGCTGATGACTTCAAG
GGACGGTTTGCCTTCTCTTTGGAAACCTCTGCCAGCACTGCCTATTTGCAGATCAGTAACCTCA
AAAATGAGGACATGGCTACATATTTCTGTGCTAGAGGGGACTTTAGGTACTACTATTTTGACTA
CTGGGGCCAAGGCACCACTCTCACAGGCTCCTCAGCCAAAACGAAGGGCCCATCCGTCTTCCC
CCTGGCGCCCTGCTCCAGGAGCACCTCCGAGAGCACAGCCGCCCTGGGCTGCCTGGTCAAGGA
CTACTTCCCCGAACCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGGTGACCGTGCCCTCC
AGCAGCTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAGTTCGA
AGGGGGACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTTCAACTG
GTACGTGGATGGCGTGGAGGTGCATAATGCCAAGACAAAGCCGCGGGAGGAGCAGTTCAACA
GCACGTACCGTGTGGTCAGCGTCCTCACCGTCCTGCACCAGGACTGGCTGAACGGCAAGGAGT
ACAAGTGCAAGGTCTCCAACAAAGGCCTCCCGTCCTCCATCGAGAAAACCATCTCCAAAGCCA
AAGGGCAGCCCCGAGAGCCACAGGTGTACACCCTGCCCCCATCCCAGGAGGAGATGACCAAG
AACCAGGTCAGCCTGACCTGCCTGGTCAAAGGCTTCTACCCCAGCGACATCGCCGTGGAGTGG
GAGAGCAATGGGCAGCCGGAGAACAACTACAAGACCACGCCTCCCGTGCTGGACTCCGACGG
CTCCTTCTTCCTCTACAGCAGGCTAACCGTGGACAAGAGCAGGTGGCAGGAGGGGAATGTCTT
CTCATGCTCCGTGATGCATGAGGCTCTGCACAACCACTACACACAGAAGAGCCTCTCCCTGTCT
CTGGGTAAAGCTAGCTGAT
Anti-DCIR_24A5.4A5_H-V-hIgG4H-C (SEQ ID NO: 91):
MDWLWNLLFLMAAAQSAQAQIQLVQSGPELKKPGETVKISCKASGYSFTNYGMNWVKQAPGKG
LKWMGWINTYTGESTYADDFKGRFAFSLETSASTAYLQISNLKNEDMATYFCARGDFRYYYFDY
WGQGTTLTGSSAKTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQ
KSLSLSLGKAS
Anti-DCIR_24A5.4A5_K-V-hIgGK-C (SEQ ID NO: 27):
ATGAGTGTGCTCACTCAGGTCCTGGCGTTGCTGCTGCTGTGGCTTACAGGTGCCAGATGTGACA
TCCAGATGACTCAGTCTCCAGCCTCCCTATCTGCATCTGTGGGAGAAACTGTCACCATCACGTG
TCGAGCAAGTGGGAATATTCACAATTATTTAGCATGGTATCAGCAGAAACAGGGAAAATCTCC
TCAGCTCCTGGTCTATAATGCAAAAACCTTGGCAGATGGTGTGCCATCAAGGTTCAGTGGCAG
TGGATCAGGAACACAATATTCTCTCAAGATCAACACCCTGCAGCCTGAAGATTTTGGGAGTTA
TTACTGTCAACATTTTTGGGATTCTTGGACGTTCGGTGGAGGCACCAAGCTCGAGATCAAACG
AACTGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAG
CACCTACAGCCTCAGCAGCACCCTGACGCTGAGCAAAGCAGACTACGAGAAACACAAAGTCT
ATGCCTGCGAAGTCACCCATCAGGGCCTGAGCTCGCCCGTCACAAAGAGCTTCAACAGGGGAG
AGTGTTAG
Anti-DCIR_24A5.4A5_K-V-hIgGK-C (SEQ ID NO: 92):
MSVLTQVLALLLLWLTGARCDIQMTQSPASLSASVGETVTITCRASGNIHNYLAWYQQKQGKSPQ
LLVYNAKTLADGVPSRFSGSGSGTQYSLKINTLQPEDFGSYYCQHFWDSWTFGGGTKLEIKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Anti-DCIR_24E7.3H9_H-V-hIgG4H-C (SEQ ID NO: 28):
ATGGAATGGACCTGGGTCTTTCTCTTCCTCCTGTCAGTAACTGCAGGTGTCCACTCCCAGGTTC
AGCTGCAGCAGTCTGGAGCTGAGCTGATGAAGCCTGGGGCCTCAGTGAAGATATCCTGCAAGG
CTACTGGCTACACATTCAGTAGCTACTGGATAGAGTGGGTAAAGCAGAGGCCTGGACATGGCC
TTGAGTGGATTGGAGAGATTTTACCTGGAAGTGGTAGGACTAACGACAATGAGAAGTTCAAGG
GCAAGGCCACATTCACTGCAGATACATCCTCCAAGAAAGCCTACATGCAACTCAGCAGCCTGA
CATCTGAGGACTCTGCCGTCTATTATTGTGCAAGAAGGGGTGGTTACTCCTTTGCTTACTGGGG
CCAAGGGACTCTGGTCACTGTCTCTGCAGCCAAAACAAAGGGCCCATCCGTCTTCCCCCTGGC
GCCCTGCTCCAGGAGCACCTCCGAGAGCACAGCCGCCCTGGGCTGCCTGGTCAAGGACTACTT
CCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGC
TTGGGCACGAAGACCTACACCTGCAACGTAGATCACAAGCCCAGCAACACCAAGGTGGACAA
GAGAGTTGAGTCCAAATATGGTCCCCCATGCCCACCCTGCCCAGCACCTGAGTTCGAAGGGGG
ACCATCAGTCTTCCTGTTCCCCCCAAAACCCAAGGACACTCTCATGATCTCCCGGACCCCTGAG
GTCACGTGCGTGGTGGTGGACGTGAGCCAGGAAGACCCCGAGGTCCAGTTCAACTGGTACGTG
GATGGCGTGGAGGTGCATAATGCCAAGACAAAGCCGCGGGAGGAGCAGTTCAACAGCACGTA
CCGTGTGGTCAGCGTCCTCACCGTCCTGCACCAGGACTGGCTGAACGGCAAGGAGTACAAGTG
CAAGGTCTCCAACAAAGGCCTCCCGTCCTCCATCGAGAAAACCATCTCCAAAGCCAAAGGGCA
GCCCCGAGAGCCACAGGTGTACACCCTGCCCCCATCCCAGGAGGAGATGACCAAGAACCAGG
TCAGCCTGACCTGCCTGGTCAAAGGCTTCTACCCCAGCGACATCGCCGTGGAGTGGGAGAGCA
ATGGGCAGCCGGAGAACAACTACAAGACCACGCCTCCCGTGCTGGACTCCGACGGCTCCTTCT
TCCTCTACAGCAGGCTAACCGTGGACAAGAGCAGGTGGCAGGAGGGGAATGTCTTCTCATGCT
CCGTGATGCATGAGGCTCTGCACAACCACTACACACAGAAGAGCCTCTCCCTGTCTCTGGGTA
AAGCTAGCTGA
Anti-DCIR_24E7.3H9_H-V-hIgG4H-C (SEQ ID NO: 93):
MEWTWVFLFLLSVTAGVHSQVQLQQSGAELMKPGASVKISCKATGYTFSSYWIEWVKQRPGHGL
EWIGEILPGSGRTNDNEKFKGKATFTADTSSKKAYMQLSSLTSEDSAVYYCARRGGYSFAYWGQG
TLVTVSAAKTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFEGGPSVFLFPPKP
KDTLMISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLS
LSLGKAS
Anti-DCIR_24E7.3H9_K-V-hIgGK-C (SEQ ID NO: 29):
ATGACCATGTTCTCACTAGCTCTTCTCCTCAGTCTTCTTCTCCTCTGTGTCTCTGATTCTAGGGC
AGAAACAACTGTGACCCAGTCTATGACCATGTTCTCACTAGCTCTTCTCCTCAGTCTTCTTCTCC
TCTGTGTCTCTGATTCTAGGGCAGAAACAACTGTGACCCAGTCTCCAGCATCCCTGTCCATGGC
TATAGGGGAAAAAGTCACCATCAGATGCGTAACCAGCACTGATATTGATGATGATGTGAACTG
GTACCAGCAGAAGCCAGGGGAACCTCCTAAACTCCTTATTTCAGAAGGCAATACTCTTCGTCC
TGGAGTCCCATCCCGATTCTCCAGCAGTGGCTATGGTACAGATTTTGTTTTTACAATTGAGAAC
ATGCTCTCAGAAGATGTTGCAGATTACTACTGTTTGCAAAGTGGTAACTTGCCGTACACGTTCG
GAGGGGGGACCAAGCTCGAGATCAAACGAACTGTGGCTGCACCATCTGTCTTCATCTTCCCGC
CATCTGATGAGCAGTTGAAATCTGGAACTGCCTCTGTTGTGTGCCTGCTGAATAACTTCTATCC
CAGAGAGGCCAAAGTACAGTGGAAGGTGGATAACGCCCTCCAATCGGGTAACTCCCAGGAGA
GTGTCACAGAGCAGGACAGCAAGGACAGCACCTACAGCCTCAGCAGCACCCTGACGCTGAGC
AAAGCAGACTACGAGAAACACAAAGTCTATGCCTGCGAAGTCACCCATCAGGGCCTGAGCTC
GCCCGTCACAAAGAGCTTCAACAGGGGAGAGTGTTAGCCAGCATCCCTGTCCATGGCTATAGG
GGAAAAAGTCACCATCAGATGCGTAACCAGCACTGATATTGATGATGATGTGAACTGGTACCA
GCAGAAGCCAGGGGAACCTCCTAAACTCCTTATTTCAGAAGGCAATACTCTTCGTCCTGGAGT
CCCATCCCGATTCTCCAGCAGTGGCTATGGTACAGATTTTGTTTTTACAATTGAGAACATGCTC
TCAGAAGATGTTGCAGATTACTACTGTTTGCAAAGTGGTAACTTGCCGTACACGTTCGGAGGG
GGGACCAAGCTCGAGATCAAACGAACTGTGGCTGCACCATCTGTCTTCATCTTCCCGCCATCTG
ATGAGCAGTTGAAATCTGGAACTGCCTCTGTTGTGTGCCTGCTGAATAACTTCTATCCCAGAGA
GGCCAAAGTACAGTGGAAGGTGGATAACGCCCTCCAATCGGGTAACTCCCAGGAGAGTGTCA
CAGAGCAGGACAGCAAGGACAGCACCTACAGCCTCAGCAGCACCCTGACGCTGAGCAAAGCA
GACTACGAGAAACACAAAGTCTATGCCTGCGAAGTCACCCATCAGGGCCTGAGCTCGCCCGTC
ACAAAGAGCTTCAACAGGGGAGAGTGTTAG
Anti-DCIR_24E7.3H9_K-V-hIgGK-C (SEQ ID NO: 94):
MTMFSLALLLSLLLLCVSDSRAETTVTQSPASLSMAIGEKVTIRCVTSTDIDDDVNWYQQKPGEPPK
LLISEGNTLRPGVPSRFSSSGYGTDFVFTIENMLSEDVADYYCLQSGNLPYTFGGGTKLEIKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Anti-DCIR_29E9.2E2_H-VhIgG4H-C (SEQ ID NO: 30):
ATGGCTTGGGTGTGGACCTTGCTATTCCTGATGGCAGCTGCCCAAAGTGCCCAAGCACAGATC
CAGTTGGTGCAGTCTGGACCTGAGCTGAAGAAGCCTGGAGAGACAGTCAAGATCTCCTGCAAG
GCTTCTGGGTATACCTTCACAAACTATGGAATGAACTGGGTGAAGCAGGCTCCAGGAAAGGGT
TTAAAGTGGGTGGGCTGGATAAACACCTTCACTGGAGAGCCAACATATGTTGATGACTTCAAG
GGACGGTTTGCCTTCTCTTTGGAAACCTCTGCCAGCACTGCCTATTTGCAGATCAACAACCTCA
AAAATGAGGACACGGCTACATATTTCTGTGCAAGAGGGAATTTTAGGTACTACTACTTTGACT
ACTGGGGCCAAGGCACCACTCTCACAGTCTCCTCAGCCAAAACAAAGGGCCCATCCGTCTTCC
CCCTGGCGCCCTGCTCCAGGAGCACCTCCGAGAGCACAGCCGCCCTGGGCTGCCTGGTCAAGG
ACTACTTCCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCGTGCCCTC
CAGCAGCTTGGGCACGAAGACCTACACCTGCAACGTAGATCACAAGCCCAGCAACACCAAGG
TGGACAAGAGAGTTGAGTCCAAATATGGTCCCCCATGCCCACCCTGCCCAGCACCTGAGTTCG
AAGGGGGACCATCAGTCTTCCTGTTCCCCCCAAAACCCAAGGACACTCTCATGATCTCCCGGA
CCCCTGAGGTCACGTGCGTGGTGGTGGACGTGAGCCAGGAAGACCCCGAGGTCCAGTTCAACT
GGTACGTGGATGGCGTGGAGGTGCATAATGCCAAGACAAAGCCGCGGGAGGAGCAGTTCAAC
AGCACGTACCGTGTGGTCAGCGTCCTCACCGTCCTGCACCAGGACTGGCTGAACGGCAAGGAG
TACAAGTGCAAGGTCTCCAACAAAGGCCTCCCGTCCTCCATCGAGAAAACCATCTCCAAAGCC
AAAGGGCAGCCCCGAGAGCCACAGGTGTACACCCTGCCCCCATCCCAGGAGGAGATGACCAA
GAACCAGGTCAGCCTGACCTGCCTGGTCAAAGGCTTCTACCCCAGCGACATCGCCGTGGAGTG
GGAGAGCAATGGGCAGCCGGAGAACAACTACAAGACCACGCCTCCCGTGCTGGACTCCGACG
GCTCCTTCTTCCTCTACAGCAGGCTAACCGTGGACAAGAGCAGGTGGCAGGAGGGGAATGTCT
TCTCATGCTCCGTGATGCATGAGGCTCTGCACAACCACTACACACAGAAGAGCCTCTCCCTGTC
TCTGGGTAAAGCTAGCTGA
Anti-DCIR_29E9.2E2_H-VhIgG4H-C (SEQ ID NO: 95):
MAWVWTLLFLMAAAQSAQAQIQLVQSGPELKKPGETVKISCKASGYTFTNYGMNWVKQAPGKG
LKWVGWINTFTGEPTYVDDFKGRFAFSLETSASTAYLQINNLKNEDTATYFCARGNFRYYYFDYW
GQGTTLTVSSAKTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGGPSVFLFPP
KPKDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSL
SLSLGKAS
Anti-DCIR_29E9.2E2_K-V-hIgGK-C (SEQ ID NO: 31):
ATGAGTGTGCTCACTCAGGTCCTGGCGTTGCTGCTGCTGTGGCTTACAGGTGCCAGATGTGACA
TCCAGATGACTCAGTCCCCAGCCTCCCTATCTGCATCTGTGGGAGAAACTGTCACCATCACATG
TCGAACAAGTGGGAATATTCGCAATTATTTAGCATGGTATCAGCAGAAACAGGGAAAATCTCC
TCAACTCCTGGTCTATAATGCAAAAACCTTAGCAGATGGTGTGCCATCAAGGTTCGGTGGCAG
TGGATCAGGAACACAATATTCTCTCAAGATCAACAGCCTGCAGCCTGAAGATTTTGGGAATTA
TTACTGTCAACATTTTTGGAGTAGTCCGTACACGTTCGGAGGGGGGACCAAGCTCGAGATCAA
ACGAACTGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACACAAAG
TCTATGCCTGCGAAGTCACCCATCAGGGCCTGAGCTCGCCCGTCACAAAGAGCTTCAACAGGG
GAGAGTGTTAG
Anti-DCIR_29E9.2E2_K-V-hIgGK-C (SEQ ID NO: 96):
MSVLTQVLALLLLWLTGARCDIQMTQSPASLSASVGETVTITCRTSGNIRNYLAWYQQKQGKSPQ
LLVYNAKTLADGVPSRFGGSGSGTQYSLKINSLQPEDFGNYYCQHFWSSPYTFGGGTKLEIKRTVA
APSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Anti-DCIR_29G10.3D9_H-V-hIgG4H-C (SEQ ID NO: 32):
ATGATGGGATGGAGCTATATCATCCTCTTTTTGGTAGCAACAGCTACAGATGTCCACTCCCAGG
TCCAACTGCAGCAGCCTGGGGCTGAACTGGTGAAGCCTGGGGCTTCAGTGAAGCTGTCCTGCA
AGGCTTCTGGCTACACCTTCACCAGCTACTGGATGCACTGGGTGAAGCAGAGGCCTGGAGAAG
GCCTTGAGTGGATTGGAGAGATTAATCCTAGCTACGGTCGTACTGACTACAATGAGAAGTTCA
AGAACAAGGCCACACTGACTGTAGCCAAATCCTCCAGCACAGCCTACATGCAACTCAGCAGCC
TGACATCTGAGGACTCTGCGGTCTATTACTGTGCAAGAGGAGATTACTACGGTAGTAGCTCGTT
TGCTTACTGGGGCCAAGGGACTCTGGTCACTGTCTCTGCAGCCAAAACAAAGGGCCCATCCGT
CTTCCCCCTGGCGCCCTGCTCCAGGAGCACCTCCGAGAGCACAGCCGCCCTGGGCTGCCTGGT
CAAGGACTACTTCCCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCAGCGTGGTGACCGTG
CCCTCCAGCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGCACCTGAG
TTCGAAGGGGGACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGAGGTCCAGTTCA
ACTGGTACGTGGATGGCGTGGAGGTGCATAATGCCAAGACAAAGCCGCGGGAGGAGCAGTTC
AACAGCACGTACCGTGTGGTCAGCGTCCTCACCGTCCTGCACCAGGACTGGCTGAACGGCAAG
GAGTACAAGTGCAAGGTCTCCAACAAAGGCCTCCCGTCCTCCATCGAGAAAACCATCTCCAAA
GCCAAAGGGCAGCCCCGAGAGCCACAGGTGTACACCCTGCCCCCATCCCAGGAGGAGATGAC
CAAGAACCAGGTCAGCCTGACCTGCCTGGTCAAAGGCTTCTACCCCAGCGACATCGCCGTGGA
GTGGGAGAGCAATGGGCAGCCGGAGAACAACTACAAGACCACGCCTCCCGTGCTGGACTCCG
ACGGCTCCTTCTTCCTCTACAGCAGGCTAACCGTGGACAAGAGCAGGTGGCAGGAGGGGAATG
TCTTCTCATGCTCCGTGATGCATGAGGCTCTGCACAACCACTACACACAGAAGAGCCTCTCCCT
GTCTCTGGGTAAAGCTAGCGGATGGAGCTATATCATCCTCTTTTTGGTAGCAACAGCTACAGAT
GTCCACTCCCAGGTCCAACTGCAGCAGCCTGGGGCTGAACTGGTGAAGCCTGGGGCTTCAGTG
AAGCTGTCCTGCAAGGCTTCTGGCTACACCTTCACCAGCTACTGGATGCACTGGGTGAAGCAG
AGGCCTGGAGAAGGCCTTGAGTGGATTGGAGAGATTAATCCTAGCTACGGTCGTACTGACTAC
AATGGGAAGTTCAAGAACAAGGCCACACTGACTGTAGCCAAATCCTCCAGCACAGCCTACATG
CAACTCAGCAGCCTGACATCTGAGGACTCTGCGGTCTATTACTGTGCAAGAGGAGATTACTAC
GGTAGTAGCTCGTTTGCTTACTGGGGCCAAGGGACTCTGGTCACTGTCTCTGCAGCCAAAACA
AAGGGCCCATCCGTCTTCCCCCTGGCGCCCTGCTCCAGGAGCACCTCCGAGAGCACAGCCGCC
CTGGGCTGCCTGGTCAAGGACTACTTCCCCGAACCGGTGACGGTGTCGTGGAACTCAGGCGCC
CTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCT
GCCCAGCACCTGAGTTCGAAGGGGGACCATCAGTCTTCCTGTTCCCCCCAAAACCCAAGGACA
CTCTCATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCCCATCCC
AGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTACACACAG
AAGAGCCTCTCCCTGTCTCTGGGTAAAGCTAGCTGA
Anti-DCIR_29G10.3D9_H-V-hIgG4H-C (SEQ ID NO: 97):
MMGWSYIILFLVATATDVHSQVQLQQPGAELVKPGASVKLSCKASGYTFTSYWMHWVKQRPGEG
LEWIGEINPSYGRTDYNEKFKNKATLTVAKSSSTAYMQLSSLTSEDSAVYYCARGDYYGSSSFAY
WGQGTLVTVSAAKTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQ
KSLSLSLGKAS
Anti-DCIR_29G10.3D9_K-Var1-V-hIgGK-C (SEQ ID NO: 33):
ATGGATTTTCAAGTGCAGATTTTCAGCTTCCTGCTAATGAGTGCCTCAGTCATAATGTCCAGGG
GACAAATTGTTCTCACCCAGTCTCCAGCACTCATGTCTGCATCTCCAGGGGAGAAGGTCACCAT
GACCTGCAGTGCCAGCTCAAATATAAGTTACATGTACTGGTACCAGCAGAAGCCAAGATCCTC
CCCCAAACCCTGGATTTATCTCACATCCAACCTGGCTTCTGGAGTCCCTGCTCGCTTCAGTGGC
AGTGGGTCTGGGACCTCTTACTCTCTCACAACCAGCAGCATGGAGGCTGAAGATGCTGCCACT
TATTGCTGCCAGCAGTGGAGTAGTAACCCACCCACGTTCGGTGCTGGGACCAAGCTCGAGATC
AAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAA
AGTCTATGCCTGCGAAGTCACCCATCAGGGCCTGAGCTCGCCCGTCACAAAGAGCTTCAACAG
GGGAGAGTGTTAG
Anti-DCIR_29G10.3D9_K-Var1-V-hIgGK-C (SEQ ID NO: 98):
MDFQVQIFSFLLMSASVIMSRGQIVLTQSPALMSASPGEKVTMTCSASSNISYMYWYQQKPRSSPK
PWIYLTSNLASGVPARFSGSGSGTSYSLTTSSMEAEDAATYCCQQWSSNPPTFGAGTKLEIKRTVA
APSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Anti-DCIR_29G10.3D9_K-Var2-V-hIgGK-C (SEQ ID NO: 34):
ATGGATTTTCGAGTGCAGATTTTCAGCTTCCTGCTAATGAGTGCCTCAGTCATAATGTCCAGGG
GACAAATTGTTCTCACCCAGTCTCCAGCACTCATGTCTGCATCTCCAGGGGAGAAGGTCACCAT
GACCTGCAGTGCCAGCTCAAATATAAGTTACATGTACTGGTACCAGCAGAAGCCAAGATCCTC
CCCCAAACCCTGGATTTATCTCACATCCAACCTGGCTTCTGGAGTCCCTGCTCGCTTCAGTGGC
AGTGGGTCTGGGACCTCTTACTCTCTCACAATCAGCAGCATGGAGGCTGAAGATGCTGCCACT
TATTACTGCCAGCAGTGGAGTAGTAACCCACCCACGTTCGGTGCTGGGACCAAGCTCGAGATC
AAACGAACTGTGGCTGCACCATCTGTCTTCATCTTCCCGCCATCTGATGAGCAGTTGAAATCTG
GAACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAA
AGTCTATGCCTGCGAAGTCACCCATCAGGGCCTGAGCTCGCCCGTCACAAAGAGCTTCAACAG
GGGAGAGTGTTAG
Anti-DCIR_29G10.3D9_K-Var2-V-hIgGK-C (SEQ ID NO: 99):
MDFRVQIFSFLLMSASVIMSRGQIVLTQSPALMSASPGEKVTMTCSASSNISYMYWYQQKPRSSPKP
WIYLTSNLASGVPARFSGSGSGTSYSLTISSMEAEDAATYYCQQWSSNPPTFGAGTKLEIKRTVAAP
SVFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Anti-DCIR_2C9K-V-hIgGK-C (SEQ ID NO: 35):
ATGGAGACAGACACACTCCTGCTATGGGTGCTGCTGCTCTGGGTTCCAGGTTCCACAGGTGAC
ATTGTGCTGATCCAATCTCCAGCTTCTTTGGCTGTGTCTCTAGGGCAGAGGGCCACCATATCCT
GCAGAGCCAGTGAAAGTGTTGATAGTTATGTCAATAGTTTTATGCACTGGTACCAGCAGAAAC
CAGGACAGCCACCCAAACTCCTCATCTATCGTGTATCCAACCTAGAATCTGGGATCCCTGCCA
GGTTCAGTGGCAGTGGGTCTAGGACAGACTTCACCCTCACCATTAATCCTGTGGAGGCTGATG
ATGTTGCAACCTATTACTGTCAGCAAAGTAATGAGGATCCATTCACGTTCGGCTCGGGGACAA
AGCTCGAGATCAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACG
AGAAACACAAAGTCTATGCCTGCGAAGTCACCCATCAGGGCCTGAGCTCGCCCGTCACAAAGA
GCTTCAACAGGGGAGAGTGTTAG
Anti-DCIR_2C9K-V-hIgGK-C (SEQ ID NO: 100):
METDTLLLWVLLLWVPGSTGDIVLIQSPASLAVSLGQRATISCRASESVDSYVNSFMHWYQQKPG
QPPKLLIYRVSNLESGIPARFSGSGSRTDFTLTINPVEADDVATYYCQQSNEDPFTFGSGTKLEIKRT
VAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC.
Anti-DCIR_31A6.1F5_H-var2-V-hIgG4H-C (SEQ ID NO: 36):
ATGGAATGTAACTGGATACTTCCTTTTATTCTGTCGGTAATTTCAGGGGTCTACTCAGAGGTTC
AGCTCCAGCAGTCTGGGACTGTGCTGGCAAGGCCTGGGGCTTCCGTGAATATGTCCTGTAAGG
CTGCTGGCTACAGCTTTACCAGTTACTGGGTGTACTGGGTCAAACAGAGGCCTGGACAGGGTC
TGGAATGGATTGGTGCTATTTACCCTAAAAATAGTAGAACTAGCTACAACCAGAAGTTCCAGG
ACAAGGCCACACTGACTGCAGTCACATCCGCCAGCACTGCCTACATGGAGCTCAGCAGCCTGA
CAAATGAGGACTCTGCGGTCTATTACTGTACAAGACCTCACTATGATTCGTTTGGTTACTGGGG
CCAAGGGACTCTGGTCACTGTCTCTGCAGCCAAAACAaagggcccATCCGTCTTCCCCCTGGCGCC
CTGCTCCAGGAGCACCTCCGAGAGCACAGCCGCCCTGGGCTGCCTGGTCAAGGACTACTTCCC
CGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTG
GGCACGAAGACCTACACCTGCAACGTAGATCACAAGCCCAGCAACACCAAGGTGGACAAGAG
AGTTGAGTCCAAATATGGTCCCCCATGCCCACCCTGCCCAGCACCTGAGTTCGAAGGGGGACC
ATCAGTCTTCCTGTTCCCCCCAAAACCCAAGGACACTCTCATGATCTCCCGGACCCCTGAGGTC
ACGTGCGTGGTGGTGGACGTGAGCCAGGAAGACCCCGAGGTCCAGTTCAACTGGTACGTGGAT
GGCGTGGAGGTGCATAATGCCAAGACAAAGCCGCGGGAGGAGCAGTTCAACAGCACGTACCG
TGTGGTCAGCGTCCTCACCGTCCTGCACCAGGACTGGCTGAACGGCAAGGAGTACAAGTGCAA
GGTCTCCAACAAAGGCCTCCCGTCCTCCATCGAGAAAACCATCTCCAAAGCCAAAGGGCAGCC
CCGAGAGCCACAGGTGTACACCCTGCCCCCATCCCAGGAGGAGATGACCAAGAACCAGGTCA
GCCTGACCTGCCTGGTCAAAGGCTTCTACCCCAGCGACATCGCCGTGGAGTGGGAGAGCAATG
GGCAGCCGGAGAACAACTACAAGACCACGCCTCCCGTGCTGGACTCCGACGGCTCCTTCTTCC
TCTACAGCAGGCTAACCGTGGACAAGAGCAGGTGGCAGGAGGGGAATGTCTTCTCATGCTCCG
TGATGCATGAGGCTCTGCACAACCACTACACACAGAAGAGCCTCTCCCTGTCTCTGGGTAAAG
CTAGCTGA
Anti-DCIR_31A6.1F5_H-var2-V-hIgG4H-C (SEQ ID NO: 101):
MECNWILPFILSVISGVYSEVQLQQSGTVLARPGASVNMSCKAAGYSFTSYWVYWVKQRPGQGLE
WIGAIYPKNSRTSYNQKFQDKATLTAVTSASTAYMELSSLTNEDSAVYYCTRPHYDSFGYWGQGT
LVTVSAAKTKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFEGGPSVFLFPPKPK
DTLMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSL
SLGKAS
Anti-DCIR_31A6.1F5_K-var2-V-hIgGK-C (SEQ ID NO: 37):
ATGGAGACAGACACACTCCTGCTATGGGTGCTGCTGCTCTGGGTTCCAGGTTCCACAGGTGAC
ATTGTGCTGACCCAATCTCCAGCTTCTTTGGCTGTGTCTCTAGGGCAGAGGGCCACCATATCCT
GCAGAGCCAGTGAAAGTGTAGATAGTTATGGCATTAGTTTTATGCACTGGTACCAGCAGAAAC
CAGGACAGCCACCCAAACTCCTCATCTATCGTGCATCCAACCAAGAATCTGGGATCCCTGCCA
GGTTCAGTGGCAGTGGGTCTAGGACAGACTTCACCCTCACCATTAATCCTGTGGAGGCTGATG
ATGTTGCAACCTATTACTGTCAGCAAAGTAATGAGGATCCGCTCACGTTCGGTGCTGGGACCA
AGCTCGAGATCAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACG
AGAAACACAAAGTCTATGCCTGCGAAGTCACCCATCAGGGCCTGAGCTCGCCCGTCACAAAGA
GCTTCAACAGGGGAGAGTGTTAG
Anti-DCIR_31A6.1F5_K-var2-V-hIgGK-C (SEQ ID NO: 102):
METDTLLLWVLLLWVPGSTGDIVLTQSPASLAVSLGQRATISCRASESVDSYGISFMHWYQQKPGQ
PPKLLIYRASNQESGIPARFSGSGSRTDFTLTINPVEADDVATYYCQQSNEDPLTFGAGTKLEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-DCIR_3C2.2D9_H-LV-hIgG4H-C (SEQ ID NO: 38):
ATGAACAGGCTTACTTCCTCATTGCTGCTGCTGATTGTCCCTGCATATGTCCTGTCCCAGGTTAC
TCTGAAAGAGTCTGGCCCTGGGATATTGCAGCCCTCCCAGACCCTCAGTCTGACTTGTTCTTTC
TCTGGGTTTTCACTGAGCACTTCTGGTATGGGTGTGAGCTGGATTCGTCAGCCTTCAGGAAAGG
GTCTGGAGTGGCTGGCACACATTTACTGGGATGATGACAAGCGCTATAATCCATCCCTGAAGA
GCCGGCTCACAATCTTTAAGGATCCCTCCAGCAACCAGGTATTCCTCAGGATCACCAGTGTGG
ACACTGCAGATACTGCCACATACTACTGTGCTCGAAACTCCCATTACTACGGTAGTACTTACGG
GGGATACTTCGATGTCTGGGGCGCAGGGACCACGGTCACCGTCTCCTCAGCCAAAACAAAGGG
CCCATCCGTCTTCCCCCTGGCGCCCTGCTCCAGGAGCACCTCCGAGAGCACAGCCGCCCTGGG
CTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTG
GTGACCGTGCCCTCCAGCAGCTTGGGCACGAAGACCTACACCTGCAACGTAGATCACAAGCCC
AGCAACACCAAGGTGGACAAGAGAGTTGAGTCCAAATATGGTCCCCCATGCCCACCCTGCCCA
GCACCTGAGTTCGAAGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGCACCAGGACTGGCT
GAACGGCAAGGAGTACAAGTGCAAGGTCTCCAACAAAGGCCTCCCGTCCTCCATCGAGAAAA
CCATCTCCAAAGCCAAAGGGCAGCCCCGAGAGCCACAGGTGTACACCCTGCCCCCATCCCAGG
AGGAGATGACCAAGAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACCACGCCTCCCGTG
CTGGACTCCGACGGCTCCTTCTTCCTCTACAGCAGGCTAACCGTGGACAAGAGCAGGTGGCAG
GAGGGGAATGTCTTCTCATGCTCCGTGATGCATGAGGCTCTGCACAACCACTACACACAGAAG
AGCCTCTCCCTGTCTCTGGGTAAAGCTAGCTGA
Anti-DCIR_3C2.2D9_H-LV-hIgG4H-C (SEQ ID NO: 103):
NRLTSSLLLLIVPAYVLSQQVTLKESGPGILQPSQTLSLTCSFSGFSLSTSGMGVSWIRQPSGKGLEW
LAHIYWDDDKRYNPSLKSRLTIFKDPSSNQVFLRITSVDTADTATYYCARNSHYYGSTYGGYFDV
WGAGTTVTVSSAKTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQ
KSLSLSLGKAS.
Anti-DCIR_3C2.2D9_K-LV-hIgGK-C (SEQ ID NO: 39):
ATGGAGACAGACACACTCCTGCTATGGGTGCTGCTGCTCGGGGTTCCAGGTTCCACAGGTAAC
ATTGTGCTGACCCAGTCTCCAACTTCTTTCACTGTGTCTCTTGGGCAGAGGGCCACCATATCCT
GCAGAGCCAGTGAAAGTGTTCATAGTTATGGCAATAGTTTTATGCACTGGTACCAGCAGAAAC
CAGGGCAGCCACCCAAACTCCTCATCTATCTTGCATCCAACGTAGAATCTGGGGTCCCTGCCA
GGTTCAGTGGTAGTGGGTCCAGGACAGACTTCACCCTCACCATTGATCCTGTGGAGGCTGATG
ATGCTGCAACCTATTACTGTCAGCAAAATAGTGAGGATCCGTGGACGTTCGGTGGAGGCACCA
AGCTCGAGATCAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACG
AGAAACACAAAGTCTATGCCTGCGAAGTCACCCATCAGGGCCTGAGCTCGCCCGTCACAAAGA
GCTTCAACAGGGGAGAGTGTTAG
Anti-DCIR_3C2.2D9_K-LV-hIgGK-C (SEQ ID NO: 104):
METDTLLLWVLLLGVPGSTGNIVLTQSPTSFTVSLGQRATISCRASESVHSYGNSFMHWYQQKPGQ
PPKLLIYLASNVESGVPARFSGSGSRTDFTLTIDPVEADDAATYYCQQNSEDPWTFGGGTKLEIKRT
VAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-DCIR_6C8.1G9_H-V-hIgG4H-C (SEQ ID NO: 40):
ATGGAATGGACCTGGGTCTTTCTCTTCCTCCTGTCAGTAACTGCAGGTGTCCACTCCCAGGTTC
AGCTGCAGCAGTCTGGAACTGAGCTGATGAAGCCTGGGGCCTCAGTGAAGATATCCTGCAAGG
CTACTGGCTACACATTCAGTACCTACTGGATAGAGTGGGTAAAGCAGAGGCCTGGACATGGCC
TTGAGTGGATTGGAGAGATTTTACCTGGAAGTGGTAGGACTAACGACAATGAGAAGTTCAAGG
GCAAGGCCACAATCACTGCAGATACATCCTCCAAGAAAGCCTACATGCAACTCAGCAGCCTGA
CATCTGAGGACTCTGCCGTCTATTACTGTGCAAGAAGGGGTGGTTACTCCTTTGCTTTCTGGGG
CCAAGGGACTCTGGTCTCTGTCTCTGCAGCCAAAACAAAGGGCCCATCCGTCTTCCCCCTGGCG
CCCTGCTCCAGGAGCACCTCCGAGAGCACAGCCGCCCTGGGCTGCCTGGTCAAGGACTACTTC
CCCGAACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAGCAGCT
TGGGCACGAAGACCTACACCTGCAACGTAGATCACAAGCCCAGCAACACCAAGGTGGACAAG
AGAGTTGAGTCCAAATATGGTCCCCCATGCCCACCCTGCCCAGCACCTGAGTTCGAAGGGGGA
CCATCAGTCTTCCTGTTCCCCCCAAAACCCAAGGACACTCTCATGATCTCCCGGACCCCTGAGG
TCACGTGCGTGGTGGTGGACGTGAGCCAGGAAGACCCCGAGGTCCAGTTCAACTGGTACGTGG
ATGGCGTGGAGGTGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAAGGAGTACAAGTGC
AAGGTCTCCAACAAAGGCCTCCCGTCCTCCATCGAGAAAACCATCTCCAAAGCCAAAGGGCAG
CCCCGAGAGCCACAGGTGTACACCCTGCCCCCATCCCAGGAGGAGATGACCAAGAACCAGGT
CAGCCTGACCTGCCTGGTCAAAGGCTTCTACCCCAGCGACATCGCCGTGGAGTGGGAGAGCAA
TGGGCAGCCGGAGAACAACTACAAGACCACGCCTCCCGTGCTGGACTCCGACGGCTCCTTCTT
CCTCTACAGCAGGCTAACCGTGGACAAGAGCAGGTGGCAGGAGGGGAATGTCTTCTCATGCTC
CGTGATGCATGAGGCTCTGCACAACCACTACACACAGAAGAGCCTCTCCCTGTCTCTGGGTAA
AGCTAGCTGA
Anti-DCIR_6C8.1G9_H-V-hIgG4H-C (SEQ ID NO: 105):
MEWTWVFLFLLSVTAGVHSQVQLQQSGTELMKPGASVKISCKATGYTFSTYWIEWVKQRPGHGL
EWIGEILPGSGRTNDNEKFKGKATITADTSSKKAYMQLSSLTSEDSAVYYCARRGGYSFAFWGQGT
LVSVSAAKTKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFEGGPSVFLFPPKPK
DTLMISRTPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSL
SLGKAS
Anti-DCIR_6C8.1G9_K-V-hIgGK-C (SEQ ID NO: 41):
ATGACCATGTTCTCACTAGCTCTTCTCCTCAGTCTTCTTCTCCTCTGTGTCTCTGATTCTAGGGC
AGAAACAACTGTGACCCAGTCTCCAGCATCCCTGTCCATGGCTATAGGAGAAAAAGTCACCAT
CAGATGCGTAACCAGCACTGATATTGATGATGATGTGAACTGGTACCAGCAGAAGCCAGGGG
AACCTCCTAAGCTCCTTATTTCAGAAGGCAATACTCTTCGTGCTGGAGTCCCATCCCGATTCTC
CAGCAGTGGCTATGGTACAGATTTTGTTTTTACAATTGAGAACATGCTCTCAGAAGATGTTGCA
GATTACTACTGTTTGCAAAGTGGTAACTTGCCGTACACGTTCGGAGGGGGGACCAAGCTCGAG
ATCAAACGAACTGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCA
AGGACAGCACCTACAGCCTCAGCAGCACCCTGACGCTGAGCAAAGCAGACTACGAGAAACAC
AAAGTCTATGCCTGCGAAGTCACCCATCAGGGCCTGAGCTCGCCCGTCACAAAGAGCTTCAAC
AGGGGAGAGTGTTAG
Anti-DCIR_6C8.1G9_K-V-hIgGK-C (SEQ ID NO: 106):
MTMFSLALLLSLLLLCVSDSRAETTVTQSPASLSMAIGEKVTIRCVTSTDIDDDVNWYQQKPGEPPK
LLISEGNTLRAGVPSRFSSSGYGTDFVFTIENMLSEDVADYYCLQSGNLPYTFGGGTKLEIKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Anti-DCIR_9E8.1E3_H-V-hIgG4H-C (SEQ ID NO: 42):
ATGAACAGGCTTACTTCCTCATTGCTGCTGCTGATTGTCCCTGCATATGTCCTGTCCCAGGTTAC
TCTGAAAGAGTCTGGCCCTGGGATATTGCAGCCCTCCCAGACCCTCAGTCTGACTTGTTCTTTC
TCTGGGTTTTCACTGAGCACTTCTGGTATGGGTCTGAGCTGGATTCGTCAGCCTTCAGGAAAGG
GTCTGGAGTGGCTGGCACACATTTACTGGGATGATGACAAGCGCTATAACCCATCCCTGAAGA
GCCGGCTCACAATCTCCAAGGATACCTCCAGCAACCAGGTTTTCCTCAAGATCACCATTGTGG
ACACTGCAGATGCTGCCACATACTACTGTGCTCGAAGCTCCCATTACTACGGTTATGGCTACGG
GGGATACTTCGATGTCTGGGGCGCAGGGACCACGGTCACCGTCTCCTCAGCCAAAACGAAGGG
CCCATCCGTCTTCCCCCTGGCGCCCTGCTCCAGGAGCACCTCCGAGAGCACAGCCGCCCTGGG
CTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTG
GTGACCGTGCCCTCCAGCAGCTTGGGCACGAAGACCTACACCTGCAACGTAGATCACAAGCCC
AGCAACACCAAGGTGGACAAGAGAGTTGAGTCCAAATATGGTCCCCCATGCCCACCCTGCCCA
GCACCTGAGTTCGAAGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGCACCAGGACTGGCT
GAACGGCAAGGAGTACAAGTGCAAGGTCTCCAACAAAGGCCTCCCGTCCTCCATCGAGAAAA
CCATCTCCAAAGCCAAAGGGCAGCCCCGAGAGCCACAGGTGTACACCCTGCCCCCATCCCAGG
AGGAGATGACCAAGAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACCACGCCTCCCGTG
CTGGACTCCGACGGCTCCTTCTTCCTCTACAGCAGGCTAACCGTGGACAAGAGCAGGTGGCAG
GAGGGGAATGTCTTCTCATGCTCCGTGATGCATGAGGCTCTGCACAACCACTACACACAGAAG
AGCCTCTCCCTGTCTCTGGGTAAAGCTAGCTGA
Anti-DCIR_9E8.1E3_H-V-hIgG4H-C (SEQ ID NO: 107):
MNRLTSSLLLLIVPAYVLSQVTLKESGPGILQPSQTLSLTCSFSGFSLSTSGMGLSWIRQPSGKGLEW
LAHIYWDDDKRYNPSLKSRLTISKDTSSNQVFLKITIVDTADAATYYCARSSHYYGYGYGGYFDV
WGAGTTVTVSSAKTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQ
KSLSLSLGKAS
Anti-DCIR_9E8.1E3_K-LV-hIgGK-C (SEQ ID NO: 43):
ATGGAGACAGACACACTCCTGCTATGGGTGCTGCTGCTCTGGGTTCCAGGTTCCACAGGTAAC
ATTGTGCTGACCCAATCTCCAGCTTCTTTGGCTGTGTCTCTAGGGCAGAGGGCCACCATATCCT
GCAGAGCCAGTGAAAGTATTCATAGTTATGGCAATAGTTTTCTGCACTGGTACCAGCAGAAAC
CAGGACAGCCACCCAAACTCCTCATCTATCTTGCATCCAACCTAGAATCTGGGGTCCCTGCCAG
GTTCAGCGGCAGTGGGTCTAGGACAGACTTCACCCTCACCATTGATCCTGTGGAGGCTGATGA
TGCTGCAACCTATTACTGTCAGCAAAATAATGAGGATCCGTGGACGTTCGGTGGAGGCACCAA
GCTCGAGATCAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGA
GAAACACAAAGTCTATGCCTGCGAAGTCACCCATCAGGGCCTGAGCTCGCCCGTCACAAAGAG
CTTCAACAGGGGAGAGTGTTAGGCGGCCGCACTAGCGCGGGCCGCATTCGAAGAGCTCGGTAC
CCGGGGATCCTCTAGAGTCGACCTGCAGGCATGCAAGCTGGCCGCGACTCTAGATCATAATCA
GC
Anti-DCIR_9E8.1E3_K-LV-hIgGK-C (SEQ ID NO: 108):
METDTLLLWVLLLWVPGSTGNIVLTQSPASLAVSLGQRATISCRASESIHSYGNSFLHWYQQKPGQ
PPKLLIYLASNLESGVPARFSGSGSRTDFTLTIDPVEADDAATYYCQQNNEDPWTFGGGTKLEIKRT
VAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-DCIR2C9H-LV-hIgG4H-V-hIgG4H-C (SEQ ID NO: 44):
ATGAAATGCAGCTGGGTCATCTTCTTCCTGATGGCAGTGGTTACAGGGGTCAATTCAGAGGTTC
AGCTGCAGCAGTCTGGGGCTGAGCTTGTGAGGCCAGGGGCCTTAGTCAAGTTGTCCTGCAAAG
CTTCTGGCTTCAACATTAATGACTACTATATCCACTGGGTGAAGCAGCGGCCTGAACAGGGCC
TGGAGCGGATTGGATGGATTGATCCTGACAATGGTAATACTATATATGACCCGAAGTTCCAGG
GCAAGGCCAGTATAACAGCAGACACATCCCCCAACACAGCCTACCTGCAGCTCAGCAGCCTGA
CATCTGAGGACACTGCCGTCTATTACTGTGCTAGAACCCGATCTCCTATGGTTACGACGGGGTT
TGTTTACTGGGGCCAAGGGACTGTGGTCACTGTCTCTGCAGCCAAAACGAAGGGCCCATCCGT
CTTCCCCCTGGCGCCCTGCTCCAGGAGCACCTCCGAGAGCACAGCCGCCCTGGGCTGCCTGGT
CAAGGACTACTTCCCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCAGCGTGGTGACCGTG
CCCTCCAGCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGCACCTGAG
TTCGAAGGGGGACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGAGGTCCAGTTCA
ACTGGTACGTGGATGGCGTGGAGGTGCATAATGCCAAGACRAAGCCGCGGGAGGAGCAGTTC
AACAGCACGTACCGTGTGGTCAGCGTCCTCACCGTCCTGCACCAGGACTGGCTGAACGGCAAG
GAGTACAAGTGCAAGGTCTCCAACAAAGGCCTCCCGTCCTCCATCGAGAAAACCATCTCCAAA
GCCAAAGGGCAGCCCCGAGAGCCACAGGTGTACACCCTGCCCCCATCCCAGGAGGAGATGAC
CAAGAACCAGGTCAGCCTGACCTGCCTGGTCAAAGGCTTCTACCCCAGCGACATCGCCGTGGA
GTGGGAGAGCAATGGGCAGCCGGAGAACAACTACAAGACCACGCCTCCCGTGCTGGACTCCG
ACGGCTCCTTCTTCCTCTACAGCAGGCTAACCGTGGACAAGAGCAGGTGGCAGGAGGGGAATG
TCTTCTCATGCTCCGTGATGCATGAGGCTCTGCACAACCACTACACACAGAAGAGCCTCTCCCT
GTCTCTGGGTAAATGA
Anti-DCIR2C9H-LV-hIgG4H-V-hIgG4H-C (SEQ ID NO: 109):
MKCSWVIFFLMAVVTGVNSEVQLQQSGAELVRPGALVKLSCKASGFNINDYYIHWVKQRPEQGLE
RIGWIDPDNGNTIYDPKFQGKASITADTSPNTAYLQLSSLTSEDTAVYYCARTRSPMVTTGFVYWG
QGTVVTVSAAKTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGGPSVFLFPP
KPKDTLMISRTPEVTCVVVDVSQEDPEVQFNWYVDGVEVHNAKXKPREEQFNSTYRVVSVLTVL
HQDWLNGKEYKCKVSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMHEALHNHYTQKS
LSLSLGK
Anti-DC-SIGNL16E3H (SEQ ID NO: 45):
ATGGAAAGGCACTGGATCTTTCTCTTCCTGTTTTCAGTAACTGCAGGTGTCCACTCCCAGGTCC
AGCTTCAGCAGTCTGGGGCTGAGCTGGCAAAACCTGGGGCCTCAGTGAAGATGTCCTGCAAGG
CTTCTGGCTACACCTTTACTACCTACTGGATGCACTGGGTAAAACAGAGGCCTGGACAGGGTC
TGGAATGGATTGGATACATTAATCCTATCACTGGTTATACTGAGTACAATCAGAAGTTCAAGG
ACAAGGCCACCTTGACTGCAGACAAATCCTCCAGCACAGCCTACATGCAACTGAGCAGCCTGA
CATCTGAGGACTCTGCAGTCTATTACTGTGCAAGAGAGGGTTTAAGTGCTATGGACTATTGGG
GTCAGGGAACCTCAGTCACCGTCACCTCAGCCAAAACAACAGCCCCATCGGTCTATCCACTGG
CCCCTGTGTGTGGAGATACAACTGGCTCCTCGGTAACTCTAGGATGCCTGGTCAAGGGTTATTT
CCCTGAGCCAGTGACCTTGACCTGGAACTCTGGATCCCTGTCCAGTGGTGTGCACACCTTCCCA
GCTGTCCTGCAGTCTGACCTCTACACCCTCAGCAGCTCAGTGACTGTAACCTCGAGCACCTGGC
CCAGCCAGACCGTCACCTGCAGCGTTGCTCACCCAGCCAGCAGCACCACGGTGGACAAAAAAC
TTGAGCCCAGCGGGCCCATTTCAACAATCAACCCCTGTCCTCCATGCAAGGAGTGTCACAAAT
GCCCAGCTCCTAACCTCGAGGGTGGACCATCCGTCTTCATCTTCCCTCCAAATATCAAGGATGT
ACTCATGATCTCCCTGACACCCAAGGTCACGTGTGTGGTGGTGGATGTGAGCGAGGATGACCC
AGACGTCCAGATCAGCTGGTTTGTGAACAACGTGGAAGTACACACAGCTCAGACACAAACCCA
TAGAGAGGATTACAACAGTACTATCCGGGTGGTCAGCACCCTCCCCATCCAGCACCAGGACTG
GATGAGTGGCAAGGAGTTCAAATGCAAGGTCAACAACAAAGACCTCCCATCACCCATCGAGA
GAACCATCTCAAAAATTAAAGGGCTAGTCAGAGCTCCACAAGTATACATCTTGCCGCCACCAG
CAGAGCAGTTGTCCAGGAAAGATGTCAGTCTCACTTGCCTGGTCGTGGGCTTCAACCCTGGAG
ACATCAGTGTGGAGTGGACCAGCAATGGGCATACAGAGGAGAACTACAAGGACACCGCACCA
GTCCTGGACTCTGACGGTTCTTACTTCATATATAGCAAGCTCAATATGAAAACAAGCAAGTGG
GAGAAAACAGATTCCTTCTCATGCAACGTGAGACACGAGGGTCTGAAAAATTACTACCTGAAG
AAGACCATCTCCCGGTCTCCGGGTAAAGCTAGCTGA
Anti-DC-SIGNL16E3H (SEQ ID NO: 110):
MERHWIFLFLFSVTAGVHSQVQLQQSGAELAKPGASVKMSCKASGYTFTTYWMHWVKQRPGQG
LEWIGYINPITGYTEYNQKFKDKATLTADKSSSTAYMQLSSLTSEDSAVYYCAREGLSAMDYWGQ
GTSVTVTSAKTTAPSVYPLAPVCGDTTGSSVTLGCLVKGYFPEPVTLTWNSGSLSSGVHTFPAVLQ
SDLYTLSSSVTVTSSTWPSQTVTCSVAHPASSTTVDKKLEPSGPISTINPCPPCKECHKCPAPNLEGG
PSVFIFPPNIKDVLMISLTPKVTCVVVDVSEDDPDVQISWFVNNVEVHTAQTQTHREDYNSTIRVVS
TLPIQHQDWMSGKEFKCKVNNKDLPSPIERTISKIKGLVRAPQVYILPPPAEQLSRKDVSLTCLVVG
FNPGDISVEWTSNGHTEENYKDTAPVLDSDGSYFIYSKLNMKTSKWEKTDSFSCNVRHEGLKNYY
LKKTISRSPGKAS
Anti-DC-SIGNL16E3K (SEQ ID NO: 46):
ATGGGCATCAAGATGGAGTCACGGATTCAGGCATTTGTATTCGTGTTTCTCTGGTTGTCTGGTG
TTGGCGGAGACATTGTGATGACCCAGTCTCACAAATTCATGTCCACATCAGTAGGAGACAGGG
TCAGCGTCACCTGCAAGGCCAGTCAGGATGTGACTTCTGCTGTAGCCTGGTATCAACAAAAAC
CAGGGCAATCTCCTAAACTACTGATTTACTGGGCATCCACCCGGCACACTGGAGTCCCTGATC
GCTTCACAGGCAGTGGATCTGGGACAGATTATACTCTCACCATCAGCAGTGGGCAGGCTGAAG
ACCTGGCACTTTATTACTGTCACCAATATTATAGCGCTCCTCGGACGTTCGGTGGAGGCACCAA
GCTGGAAGTCAAACGGGCTGATGCTGCACCAACTGTATCCATCTTCCCACCATCCAGTGAGCA
GTTAACATCTGGAGGTGCCTCAGTCGTGTGCTTCTTGAACAACTTCTACCCCAAAGACATCAAT
GTCAAGTGGAAGATTGATGGCAGTGAACGACAAAATGGCGTCCTGAACAGTTGGACTGATCA
GGACAGCAAAGACAGCACCTACAGCATGAGCAGCACCCTCACGTTGACCAAGGACGAGTATG
AACGACATAACAGCTATACCTGTGAGGCCACTCACAAGACATCAACTTCACCCATCGTCAAGA
GCTTCAATAGGAATGAGTGTTAG
Anti-DC-SIGNL16E3K (SEQ ID NO: 111):
MESRIQAFVFVFLWLSGVGGDIVMTQSHKFMSTSVGDRVSVTCKASQDVTSAVAWYQQKPGQSP
KLLIYWASTRHTGVPDRFTGSGSGTDYTLTISSGQAEDLALYYCHQYYSAPRTFGGGTKLEVKRAD
AAPTVSIFPPSSEQLTSGGASVVCFLNNFYPKDINVKWKIDGSERQNGVLNSWTDQDSKDSTYSMS
STLTLTKDEYERHNSYTCEATHKTSTSPIVKSFNRNEC
Anti-DC-SIGNL16E7H-LV-hIgG4H-C (SEQ ID NO: 47):
ATGGAAAGGCACTGGATCTTTCTCTTCCTGTTTTCAGTAACTGCAGGTGTCCACTCCCAGGTCC
AGCTTCAGCAGTCTGGGGCTGAGCTGGCAAAACCTGGGGCCTCAGTGAAGATGTCCTGCAAGG
CTTCTGGCTACACCTTTACTACCTACTGGATGCACTGGGTAAAACAGAGGCCTGGACAGGGTC
TGGAATGGATTGGATACATTAATCCTATCACTGGTTATACTGAGTACAATCAGAAGTTCAAGG
ACAAGGCCACCTTGACTGCAGACAAATCCTCCAGCACAGCCTACATGCAACTGAGCAGCCTGA
CATCTGAGGACTCTGCAGTCTATTACTGTGCAAGAGAGGGTTTAAGTGCTATGGACTATTGGG
GTCAGGGAACCTCAGTCACCGTCACCTCAGCCAAAACAACGGGCCCATCCGTCTTCCCCCTGG
CGCCCTGCTCCAGGAGCACCTCCGAGAGCACAGCCGCCCTGGGCTGCCTGGTCAAGGACTACT
TCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGGTGGACA
AGAGAGTTGAGTCCAAATATGGTCCCCCATGCCCACCCTGCCCAGCACCTGAGTTCGAAGGGG
GACCATCAGTCTTCCTGTTCCCCCCAAAACCCAAGGACACTCTCATGATCTCCCGGACCCCTGA
GGTCACGTGCGTGGTGGTGGACGTGAGCCAGGAAGACCCCGAGGTCCAGTTCAACTGGTACGT
GGATGGCGTGGAGGTGCATAATGCCAAGACAAAGCCGCGGGAGGAGCAGTTCAACAGCACGT
ACCGTGTGGTCAGCGTCCTCACCGTCCTGCACCAGGACTGGCTGAACGGCAAGGAGTACAAGT
GCAAGGTCTCCAACAAAGGCCTCCCGTCCTCCATCGAGAAAACCATCTCCAAAGCCAAAGGGC
AGCCCCGAGAGCCACAGGTGTACACCCTGCCCCCATCCCAGGAGGAGATGACCAAGAACCAG
GTCAGCCTGACCTGCCTGGTCAAAGGCTTCTACCCCAGCGACATCGCCGTGGAGTGGGAGAGC
AATGGGCAGCCGGAGAACAACTACAAGACCACGCCTCCCGTGCTGGACTCCGACGGCTCCTTC
TTCCTCTACAGCAGGCTAACCGTGGACAAGAGCAGGTGGCAGGAGGGGAATGTCTTCTCATGC
TCCGTGATGCATGAGGCTCTGCACAACCACTACACACAGAAGAGCCTCTCCCTGTCTCTGGGT
AAAGCTAGCTGA
Anti-DC-SIGNL16E7H-LV-hIgG4H-C (SEQ ID NO: 112):
MERHWIFLFLFSVTAGVHSQVQLQQSGAELAKPGASVKMSCKASGYTFTTYWMHWVKQRPGQG
LEWIGYINPITGYTEYNQKFKDKATLTADKSSSTAYMQLSSLTSEDSAVYYCAREGLSAMDYWGQ
GTSVTVTSAKTTGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSVFLFPPKP
KDTLMISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLS
LSLGKAS.
Anti-DC-SIGNL16E7K-LV-hIgGK-C (SEQ ID NO: 48):
ATGGGCATCAAGATGGAGTCACAGATTCAGGCATTTGTATTCGTGTTTCTCTGGTTGTCTGGTG
TTGGCGGAGACATTGTGATGACCCAGTCTCACAAATTCATGTCCACATCAGTAGGAGACAGGG
TCAGCGTCACCTGCAAGGCCAGTCAGGATGTGACTTCTGCTGTAGCCTGGTATCAACAAAAAC
CAGGGCAATCTCCTAAACTACTGATTTACTGGGCATCCACCCGGCACACTGGAGTCCCTGATC
GCTTCACAGGCAGTGGATCTGGGACAGATTATACTCTCACCATCAGCAGTGGGCAGGCTGAAG
ACCTGGCACTTTATTACTGTCACCAATATTATAGCGCTCCTCGGACGTTCGGTGGAGGCACCAA
GCTCGAGATCAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGA
GAAACACAAAGTCTATGCCTGCGAAGTCACCCATCAGGGCCTGAGCTCGCCCGTCACAAAGAG
CTTCAACAGGGGAGAGTGTTAG
Anti-DC-SIGNL16E7K-LV-hIgGK-C (SEQ ID NO: 113):
MESQIQAFVFVFLWLSGVGGDIVMTQSHKFMSTSVGDRVSVTCKASQDVTSAVAWYQQKPGQSP
KLLIYWASTRHTGVPDRFTGSGSGTDYTLTISSGQAEDLALYYCHQYYSAPRTFGGGTKLEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-Dectin_1_11B6.4_H-V-hIgG4H-C (SEQ ID NO: 49):
ATGGCTGTCCTGGCACTACTCCTCTGCCTGGTGGCTTTCCCAACTTGTACCCTGTCCCAGGTGC
AACTGAAGGAGTCAGGACCTGGCCTGGTGGCGCCCTCACAGAGCCTGTCCATTACCTGCTCTG
TCTCTGGGTTCTCATTAAGCAACTATGATATAAGCTGGATTCGCCAGCCACCAGGAAAGGGTC
TGGAGTGGCTTGGAGTAATGTGGACTGGTGGAGGCGCAAATTATAATTCAGCTTTCATGTCCA
GACTGAGCATCAACAAGGACAACTCCAAGAGCCAAGTTTTTTTAAAAATGAACAATCTGCAAA
CTGATGACACAGCCATTTATTACTGTGTCAGAGATGCGGTGAGGTACTGGAACTTCGATGTCTG
GGGCGCAGGGACCACGGTCACCGTCTCCTCAGCCAAAACGAAGGGCCCATCCGTCTTCCCCCT
GGCGCCCTGCTCCAGGAGCACCTCCGAGAGCACAGCCGCCCTGGGCTGCCTGGTCAAGGACTA
CTTCCCCGAACCGGTGACGGTGTCGTGGAACTCAGGCGCCCTGACCAGCGGCGTGCACACCTT
CCCGGCTGTCCTACAGTCCTCAGGACTCTACTCCCTCAGCAGCGTGGTGACCGTGCCCTCCAGC
AGCTTGGGCACGAAGACCTACACCTGCAACGTAGATCACAAGCCCAGCAACACCAAGGTGGA
CAAGAGAGTTGAGTCCAAATATGGTCCCCCATGCCCACCCTGCCCAGCACCTGAGTTCGAAGG
GGGACCATCAGTCTTCCTGTTCCCCCCAAAACCCAAGGACACTCTCATGATCTCCCGGACCCCT
GAGGTCACGTGCGTGGTGGTGGACGTGAGCCAGGAAGACCCCGAGGTCCAGTTCAACTGGTAC
GTGGATGGCGTGGAGGTGCATAATGCCAAGACAAAGCCGCGGGAGGAGCAGTTCAACAGCAC
GTACCGTGTGGTCAGCGTCCTCACCGTCCTGCACCAGGACTGGCTGAACGGCAAGGAGTACAA
GTGCAAGGTCTCCAACAAAGGCCTCCCGTCCTCCATCGAGAAAACCATCTCCAAAGCCAAAGG
GCAGCCCCGAGAGCCACAGGTGTACACCCTGCCCCCATCCCAGGAGGAGATGACCAAGAACC
AGGTCAGCCTGACCTGCCTGGTCAAAGGCTTCTACCCCAGCGACATCGCCGTGGAGTGGGAGA
GCAATGGGCAGCCGGAGAACAACTACAAGACCACGCCTCCCGTGCTGGACTCCGACGGCTCCT
TCTTCCTCTACAGCAGGCTAACCGTGGACAAGAGCAGGTGGCAGGAGGGGAATGTCTTCTCAT
GCTCCGTGATGCATGAGGCTCTGCACAACCACTACACACAGAAGAGCCTCTCCCTGTCTCTGG
GTAAAGCTAGCTGA
Anti-Dectin_1_11B6.4_H-V-hIgG4H-C (SEQ ID NO: 114):
MAVLALLLCLVAFPTCTLSQVQLKESGPGLVAPSQSLSITCSVSGFSLSNYDISWIRQPPGKGLEWL
GVMWTGGGANYNSAFMSRLSINKDNSKSQVFLKMNNLQTDDTAIYYCVRDAVRYWNFDVWGA
GTTVTVSSAKTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSVFLFPPKP
KDTLMISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLS
LSLGKAS
Anti-Dectin_1_11B6.4_K-LV-hIgGK-C (SEQ ID NO: 50):
ATGGATTTTCAAGCGCAGATTTTCAGCTTCCTGCTAATCAGTGCTTCAGTCATAATGTCCAGAG
GACAAATTGTTCTCTCCCAGTCACCAGCAATCCTGTCTGCATCTCCAGGGGAGAAGGTCACAA
TGACTTGCAGGGCCAGCTCAAGTGTAAGTTACATACACTGGTACCAGCAGAAGCCAGGATCCT
CCCCCAAACCCTGGATTTATGCCACATCCCACCTGGCTTCTGGAGTCCCTGCTCGCTTCAGTGG
CAGTGGGTCTGGGACCTCTTACTCTCTCACAATCAGCAGAGTGGAGGCTGAAGATACTGCCAC
TTATTACTGCCAGCAGTGGAGTAGTAACCCATTCACGTTCGGCTCGGGGACAAAGCTCGAGAT
CAAACGAACTGTGGCTGCACCATCTGTCTTCATCTTCCCGCCATCTGATGAGCAGTTGAAATCT
GGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAA
GGACAGCACCTACAGCCTCAGCAGCACCCTGACGCTGAGCAAAGCAGACTACGAGAAACACA
AAGTCTATGCCTGCGAAGTCACCCATCAGGGCCTGAGCTCGCCCGTCACAAAGAGCTTCAACA
GGGGAGAGTGTTAG
Anti-Dectin_1_11B6.4_K-LV-hIgGK-C (SEQ ID NO: 115):
MDFQAQIFSFLLISASVIMSRGQIVLSQSPAILSASPGEKVTMTCRASSSVSYIHWYQQKPGSSPKPWI
YATSHLASGVPARFSGSGSGTSYSLTISRVEAEDTATYYCQQWSSNPFTFGSGTKLEIKRTVAAPSV
FIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
Anti-Dectin_1_15E2.5_H-V-hIgG4H-C (SEQ ID NO: 51):
ATGGAAAGGCACTGGATCTTTCTACTCCTGTTGTCAGTAACTGCAGGTGTCCACTCCCAGGTCC
AGCTGCAGCAGTCTGGGGCTGAACTGGCAAGACCTGGGGCCTCAGTGAAGATGTCCTGCAAGG
CTTCTGGCTACACCTTTACTACCTACACTATGCACTGGGTAAAACAGAGGCCTGGACAGGGTCT
GGAATGGATTGGATACATTAATCCTAGCAGTGGTTATACTAATTACAATCAGAAGTTCAAGGA
CAAGGCCACATTGACTGCAGACAAATCCTCCAGCACAGCCTCCATGCAACTGAGCAGCCTGAC
ATCTGAGGACTCTGCAGTCTATTACTGTGCAAGAGAGAGGGCGGTATTAGTCCCCTATGCTAT
GGACTACTGGGGTCAAGGAACCTCAGTCACCGTCTCCTCAGCCAAAACAAAGGGCCCATCCGT
CTTCCCCCTGGCGCCCTGCTCCAGGAGCACCTCCGAGAGCACAGCCGCCCTGGGCTGCCTGGT
CAAGGACTACTTCCCCGAACCGGTGACGGTGTCGTGGAACTCAGGCGCCCTGACCAGCGGCGT
GCACACCTTCCCGGCTGTCCTACAGTCCTCAGGACTCTACTCCCTCAGCAGCGTGGTGACCGTG
CCCTCCAGCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGCACCTGAG
TTCGAAGGGGGACCATCAGTCTTCCTGTTCCCCCCAAAACCCAAGGACACTCTCATGATCTCCC
GGACCCCTGAGGTCACGTGCGTGGTGGTGGACGTGAGCCAGGAAGACCCCGAGGTCCAGTTCA
ACTGGTACGTGGATGGCGTGGAGGTGCATAATGCCAAGACAAAGCCGCGGGAGGAGCAGTTC
AACAGCACGTACCGTGTGGTCAGCGTCCTCACCGTCCTGCACCAGGACTGGCTGAACGGCAAG
GAGTACAAGTGCAAGGTCTCCAACAAAGGCCTCCCGTCCTCCATCGAGAAAACCATCTCCAAA
GCCAAAGGGCAGCCCCGAGAGCCACAGGTGTACACCCTGCCCCCATCCCAGGAGGAGATGAC
CAAGAACCAGGTCAGCCTGACCTGCCTGGTCAAAGGCTTCTACCCCAGCGACATCGCCGTGGA
GTGGGAGAGCAATGGGCAGCCGGAGAACAACTACAAGACCACGCCTCCCGTGCTGGACTCCG
ACGGCTCCTTCTTCCTCTACAGCAGGCTAACCGTGGACAAGAGCAGGTGGCAGGAGGGGAATG
TCTTCTCATGCTCCGTGATGCATGAGGCTCTGCACAACCACTACACACAGAAGAGCCTCTCCCT
GTCTCTGGGTAAAGCTAGCTGA
Anti-Dectin_1_15E2.5_H-V-hIgG4H-C (SEQ ID NO: 116):
MERHWIFLLLLSVTAGVHSQVQLQQSGAELARPGASVKMSCKASGYTFTTYTMHWVKQRPGQGL
EWIGYINPSSGYTNYNQKFKDKATLTADKSSSTASMQLSSLTSEDSAVYYCARERAVLVPYAMDY
WGQGTSVTVSSAKTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQ
KSLSLSLGKAS
Anti-Dectin_1_15E2.5_K-V-hIgGK-C (SEQ ID NO: 52):
ATGCATTTTCAAGTGCAGATTTTCAGCTTCCTGCTAATCAGTGCCTCAGTCATAATGTCCAGAG
GACAAATTGTTCTCACCCAGTCTCCAGCAGTCATGTCTGCATCTCCAGGGGAGAAGGTCACCA
TAACCTGCACTGCCAGCTCAAGTTTAAGTTACATGCACTGGTTCCAGCAGAAGCCAGGCACTT
CTCCCAAACTCTGGCTTTATAGCACATCCATCCTGGCTTCTGGAGTCCCTACTCGCTTCAGTGG
CAGTGGATCTGGGACCTCTTACTCTCTCACAATCAGCCGAATGGAGGCTGAAGATGCTGCCAC
TTATTACTGCCAGCAAAGGAGTAGTTCCCCATTCACGTTCGGCTCGGGGACAAAGCTCGAGAT
CAAACGAACTGTGGCTGCACCATCTGTCTTCATCTTCCCGCCATCTGATGAGCAGTTGAAATCT
GGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAA
GGACAGCACCTACAGCCTCAGCAGCACCCTGACGCTGAGCAAAGCAGACTACGAGAAACACA
AAGTCTATGCCTGCGAAGTCACCCATCAGGGCCTGAGCTCGCCCGTCACAAAGAGCTTTCAACA
GGGGAGAGTGTTAG
Anti-Dectin_1_15E2.5_K-V-hIgGK-C (SEQ ID NO: 117):
MHFQVQIFSFLLISASVIMSRGQIVLTQSPAVMSASPGEKVTITCTASSSLSYMHWFQQKPGTSPKL
WLYSTSILASGVPTRFSGSGSGTSYSLTISRMEAEDAATYYCQQRSSSPFTFGSGTKLEIKRTVAAPS
VFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Anti-Dectin_1_2D8.2D4H-V-hIgG4H-C (SEQ ID NO: 53):
ATGGGATGGACCTGGATCTTTATTTTAATCCTGTCAGTTACTACAGGTGTCCACTCTGAGGTCC
AGCTGCAGCAGTCTGGACCTGAGCTGGAGAAGCCTGGCGCTTCAGTGAAGATATCCTGCAAGG
CTTCTGGTTACTCCTTCACTGGCTACAACATGAACTGGGTGAAACAGAGCAATGGAAAGAGCC
TTGAGTGGATTGGAAATATTGATCCTTACTATGGTGATACTAACTACAACCAGAAGTTCAAGG
GCAAGGCCACATTGACTGTAGACAAATCCTCCAGCACAGCCTACATGCACCTCAAGAGCCTGA
CATCTGAGGACTCTGCAGTCTATTACTGTGCAAGACCCTACGGTAGTGAGGCCTACTTTGCTTA
CTGGGGCCAAGGGACTCTGGTCACTGTCTCTGCAGCCAAAACGAAGGGCCCATCCGTCTTCCC
CCTGGCGCCCTGCTCCAGGAGCACCTCCGAGAGCACAGCCGCCCTGGGCTGCCTGGTCAAGGA
CTACTTCCCCGAACCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGGTGACCGTGCCCTCC
AGCAGCTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAGTTCGA
AGGGGGACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTTCAACTG
GTACGTGGATGGCGTGGAGGTGCATAATGCCAAGACAAAGCCGCGGGAGGAGCAGTTCAACA
GCACGTACCGTGTGGTCAGCGTCCTCACCGTCCTGCACCAGGACTGGCTGAACGGCAAGGAGT
ACAAGTGCAAGGTCTCCAACAAAGGCCTCCCGTCCTCCATCGAGAAAACCATCTCCAAAGCCA
AAGGGCAGCCCCGAGAGCCACAGGTGTACACCCTGCCCCCATCCCAGGAGGAGATGACCAAG
AACCAGGTCAGCCTGACCTGCCTGGTCAAAGGCTTCTACCCCAGCGACATCGCCGTGGAGTGG
GAGAGCAATGGGCAGCCGGAGAACAACTACAAGACCACGCCTCCCGTGCTGGACTCCGACGG
CTCCTTCTTCCTCTACAGCAGGCTAACCGTGGACAAGAGCAGGTGGCAGGAGGGGAATGTCTT
CTCATGCTCCGTGATGCATGAGGCTCTGCACAACCACTACACACAGAAGAGCCTCTCCCTGTCT
CTGGGTAAAGCTAGCTGA
Anti-Dectin_1_2D8.2D4H-V-hIgG4H-C (SEQ ID NO: 118):
MGWTWIFILILSVTTGVHSEVQLQQSGPELEKPGASVKISCKASGYSFTGYNMNWVKQSNGKSLE
WIGNIDPYYGDTNYNQKFKGKATLTVDKSSSTAYMHLKSLTSEDSAVYYCARPYGSEAYFAYWG
QGTLVTVSAAKTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGGPSVFLFPP
KPKDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSL
SLSLGKAS
Anti-Dectin_1_2D8.2D4K-V-hIgGK-C (SEQ ID NO: 54):
ATGGTGTCCACTTCTCAGCTCCTTGGACTTTTGCTTTTCTGGACTTCAGCCTCCAGATGTGACAT
TGTGATGACTCAGTCTCCAGCCACCCTGTCTGTGACTCCAGGAGATAGAGTCTCTCTTTCCTGC
AGGGCCAGCCAGAGTATTAGCGACTACTTACACTGGTATCAACAAAAATCACATGAGTCTCCA
AGGCTTCTCATCAAATATGCTGCCCAATCCATCTCTGGGATCCCCTCCAGGTTCAGTGGCAGTG
GATCAGGGTCAGATTTCACTCTCAGTATCAACGGTGTGGAACCTGAAGATGTTGGAGTGTATT
ACTGTCAAAATGGTCACAGCTTTCCGTACACGTTCGGAGGGGGGACCAAGCTCGAGATCAAAC
GAACTGTGGCTGCACCATCTGTCTTCATCTTCCCGCCATCTGATGAGCAGTTGAAATCTGGAAC
TGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAAACACAAAGTC
TATGCCTGCGAAGTCACCCATCAGGGCCTGAGCTCGCCCGTCACAAAGAGCTTCAACAGGGGA
GAGTGTTAG
Anti-Dectin_1_2D8.2D4K-V-hIgGK-C (SEQ ID NO: 119):
DIVMTQSPATLSVTPGDRVSLSCRASQSISDYLHWYQQKSHESPRLLIKYAAQSISGIPSRFSGSGSG
SDFTLSINGVEPEDVGVYYCQNGHSFPYTFGGGTKLEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQG
LSSPVTKSFNRGEC
Anti-Langerin15B10H-LV-hIgG4H-C (SEQ ID NO: 55):
ATGGAATGGAGGATCTTTCTCTTCATCCTGTCAGGAACTGCAGGTGTCCACTCCCAGGTTCAGC
TGCGGCAGTCTGGACCTGAGCTGGTGAAGCCTGGGGCTTCAGTGAAGATGTCCTGCAAGGCTT
CTGGATACACATTTACTGACTATGTTATAAGTTGGGTGAAGCAGAGAACTGGACAGGGCCTTG
AGTGGATTGGAGATATTTATCCTGGAAGTGGTTATTCTTTCTACAATGAGAACTTCAAGGGCAA
GGCCACACTGACTGCAGACAAATCCTCCACCACAGCCTACATGCAGCTCAGCAGCCTGACATC
TGAGGACTCTGCGGTCTATTTCTGTGCAACCTACTATAACTACCCTTTTGCTTACTGGGGCCAA
GGGACTCTGGTCACTGTCTCTGCAGCCAAAACAACGGGCCCATCCGTCTTCCCCCTGGCGCCCT
GCTCCAGGAGCACCTCCGAGAGCACAGCCGCCCTGGGCTGCCTGGTCAAGGACTACTTCCCCG
AACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGG
CACGAAGACCTACACCTGCAACGTAGATCACAAGCCCAGCAACACCAAGGTGGACAAGAGAG
TTGAGTCCAAATATGGTCCCCCATGCCCACCCTGCCCAGCACCTGAGTTCGAAGGGGGACCAT
CAGTCTTCCTGTTCCCCCCAAAACCCAAGGACACTCTCATGATCTCCCGGACCCCTGAGGTCAC
GTGCGTGGTGGTGGACGTGAGCCAGGAAGACCCCGAGGTCCAGTTCAACTGGTACGTGGATGG
CGTGGAGGTGCATAATGCCAAGACAAAGCCGCGGGAGGAGCAGTTCAACAGCACGTACCGTG
TGGTCAGCGTCCTCACCGTCCTGCACCAGGACTGGCTGAACGGCAAGGAGTACAAGTGCAAGG
TCTCCAACAAAGGCCTCCCGTCCTCCATCGAGAAAACCATCTCCAAAGCCAAAGGGCAGCCCC
GAGAGCCACAGGTGTACACCCTGCCCCCATCCCAGGAGGAGATGACCAAGAACCAGGTCAGC
CTGACCTGCCTGGTCAAAGGCTTCTACCCCAGCGACATCGCCGTGGAGTGGGAGAGCAATGGG
CAGCCGGAGAACAACTACAAGACCACGCCTCCCGTGCTGGACTCCGACGGCTCCTTCTTCCTCT
ACAGCAGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTAAAGCT
AGCTGA
Anti-Langerin15B10H-LV-hIgG4H-C (SEQ ID NO: 120):
QVQLRQSGPELVKPGASVKMSCKASGYTFTDYVISWVKQRTGQGLEWIGDIYPGSGYSFYNENFK
GKATLTADKSSTTAYMQLSSLTSEDSAVYFCATYYNYPFAYWGQGTLVTVSAAKTTGPSVFPLAP
CSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNGKEYKCKVSNKGLPSS
IEKTISKAKGQPREPQVYTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSLGKAS.
Anti-Langerin15B10K-LV-hIgGK-C (SEQ ID NO: 56):
ATGAAGTTGCCTGTTAGGCTGTTGGTGCTGATGTTCTGGATTCCTGCTTCCAGCAGTGATGTTG
TGATGACCCAAACTCCACTCTCCCTGCCTGTCCGTCTTGGAGATCAAGCCTCCATCTCTTGCAG
ATCTAGTCAGAGCCTTGTACACAGTAATGGAAACACCTATTTACATTGGTACCTGCAGAAGCC
AGGCCAGTCTCCAAAGCTCCTGATCTACAAAGTTTCCAACCGATTTTCTGGGGTCCCAGACAG
GTTCAGTGGCAGTGGATCAGGGACAAATTTCACACTCAAGATCAGCAGAGTGGAGGCTGAGG
ATCTGGGACTTTATTTCTGCTCTCAAAGTACACATGTTCCGTACACGTTCGGAGGGGGGACCAA
GCTCGAGATCAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGA
GAAACACAAAGTCTATGCCTGCGAAGTCACCCATCAGGGCCTGAGCTCGCCCGTCACAAAGAG
CTTCAACAGGGGAGAGTGTTAG
Anti-Langerin15B10K-LV-hIgGK-C (SEQ ID NO: 121):
DVVMTQTPLSLPVRLGDQASISCRSSQSLVHSNGNTYLHWYLQKPGQSPKLLIYKVSNRFSGVPDR
FSGSGSGTNFTLKISRVEAEDLGLYFCSQSTHVPYTFGGGTKLEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC
Anti-Langerin2G3H-LV-hIgG4H-C (SEQ ID NO: 57):
ATGACATTGAACATGCTGTTGGGGCTGAGGTGGGTTTTCTTTGTTGTTTTTTATCAAGGTGTGC
ATTGTGAGGTGCAGCTTGTTGAGTCTGGTGGAGGATTGGTGCAGCCTAAAGGGTCATTGAAAC
TCTCATGTGCAGCCTCTGGATTAACCTTCAATATCTACGCCATGAACTGGGTCCGCCAGGCTCC
AGGAAAGGGTTTGGAATGGGTTGCTCGCATAAGAAATAAAAGTAATAATTATGCAACATATTA
TGCCGATTCAGTGAAAGACAGGTTCACCATCTCCAGAGATGATTCACAAAGCTTGCTCTATCTG
CAAATGAACAACTTGAAAACTGAGGACACAGCCATGTATTACTGTGTGGGACGGGACTGGTTT
GATTACTGGGGCCAAGGGACTCTGGTCACTGTCTCTGCAGCCAAAACGAAGGGCCCATCCGTC
TTCCCCCTGGCGCCCTGCTCCAGGAGCACCTCCGAGAGCACAGCCGCCCTGGGCTGCCTGGTC
AAGGACTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGACCGTGC
CCTCCAGCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACCTGAGT
TCGAAGGGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCAGTTCAA
CTGGTACGTGGATGGCGTGGAGGTGCATAATGCCAAGACAAAGCCGCGGGAGGAGCAGTTCA
ACAGCACGTACCGTGTGGTCAGCGTCCTCACCGTCCTGCACCAGGACTGGCTGAACGGCAAGG
AGTACAAGTGCAAGGTCTCCAACAAAGGCCTCCCGTCCTCCATCGAGAAAACCATCTCCAAAG
CCAAAGGGCAGCCCCGAGAGCCACAGGTGTACACCCTGCCCCCATCCCAGGAGGAGATGACC
AAGAACCAGGTCAGCCTGACCTGCCTGGTCAAAGGCTTCTACCCCAGCGACATCGCCGTGGAG
TGGGAGAGCAATGGGCAGCCGGAGAACAACTACAAGACCACGCCTCCCGTGCTGGACTCCGA
CGGCTCCTTCTTCCTCTACAGCAGGCTAACCGTGGACAAGAGCAGGTGGCAGGAGGGGAATGT
CTTCTCATGCTCCGTGATGCATGAGGCTCTGCACAACCACTACACACAGAAGAGCCTCTCCCTG
TCTCTGGGTAAAGCTAGCTGA
Anti-Langerin2G3H-LV-hIgG4H-C (SEQ ID NO: 122):
MTLNMLLGLRWVFFVVFYQGVHCEVQLVESGGGLVQPKGSLKLSCAASGLTFNIYAMNWVRQAP
GKGLEWVARIRNKSNNYATYYADSVKDRFTISRDDSQSLLYLQMNNLKTEDTAMYYCVGRDWF
DYWGQGTLVTVSAAKTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNVFSCSVMHEALHNHY
TQKSLSLSLGKAS.
Anti-Langerin2G3L-LV-hIgGK-C (SEQ ID NO: 58):
ATGGCCTGGATTTCACTTATACTCTCTCTCCTGGCTCTCAGCTCAGGGGCCATTTCCCAGGCTGT
TGTGACTCAGGAATCTGCACTCACCACATCACCTGGTGAAACAGTCACACTCACTTGTCGCTCA
AGTACTGGGGCTGTTACAACTAGTAACTATGCCAACTGGGTCCAAGAAAAACCAGATCATTTA
TTCACTGGTCTAATAGGTGGTACCAACAACCGAGTTTCAGGTGTTCCTGCCAGATTCTCAGGCT
CCCTGATTGGAGACAAGGCTGCCCTCACCATCACAGGGGCACAGACTGAGGATGAGGCAATAT
ATTTCTGTGCTCTATGGTACAGCAACCATTGGGTGTTCGGTGGAGGAACCAAACTCGAGATCA
AACGAACTGTGGCTGCACCATCTGTCTTCATCTTCCCGCCATCTGATGAGCAGTTGAAATCTGG
AACTGCCTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAACACAAA
GTCTATGCCTGCGAAGTCACCCATCAGGGCCTGAGCTCGCCCGTCACAAAGAGCTTCAACAGG
GGAGAGTGTTAG
Anti-Langerin2G3L-LV-hIgGK-C (SEQ ID NO: 123):
MAWISLILSLLALSSGAISQAVVTQESALTTSPGETVTLTCRSSTGAVTTSNYANWVQEKPDHLFTG
LIGGTNNRVSGVPARFSGSLIGDKAALTITGAQTEDEAIYFCALWYSNHWVFGGGTKLEIKRTVAA
PSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Anti-Lox_1_10F9H-LV-hIgG4H-C (SEQ ID NO: 59):
ATGGAATGGACCTGGGTCTTTCTCTTCCTCCTGTCAGTAACTGCAGGTGTCCACTCCCAGGTTC
AGCTGCAGCAGTCTGGAGCTGAGCTGATGAAGCCTGGGGCCTCAGTGAAGATATCCTGCAAGG
CTACTGGCTACACATTCGGTAGCTACTGGATAGAGTGGGTAAAGCAGAGGCCTGGACATGGCC
TTGAGTGGATTGGAGAGATTTTACCTGGAAGTGGTAATACTAACTACAATGAGAACTTCAAGG
GCAAGGCCACATTCACTGCAGATACATCCTCCAACACAGCCTACATGCAACTCACCAGTCTGA
CATCTGAGGACTCTGCCGTCTATTACTGTGCTAGGGCGGGGATTTATTGGGGCCAAGGGACTCT
GGTCACTGTCTCTGCAGCCAAAACGAAGGGCCCATCCGTCTTCCCCCTGGCGCCCTGCTCCAGG
AGCACCTCCGAGAGCACAGCCGCCCTGGGCTGCCTGGTCAAGGACTACTTCCCCGAACCGGTG
ACGGTGTCGTGGAACTCAGGCGCCCTGACCAGCGGCGTGCACACCTTCCCGGCTGTCCTACAG
TCCTCAGGACTCTACTCCCTCAGCAGCGTGGTGACCGTGCCCTCCAGCAGCTTGGGCACGAAG
ACCTACACCTGCAACGTAGATCACAAGCCCAGCAACACCAAGGTGGACAAGAGAGTTGAGTC
CAAATATGGTCCCCCATGCCCACCCTGCCCAGCACCTGAGTTCGAAGGGGGACCATCAGTCTT
CCTGTTCCCCCCAAAACCCAAGGACACTCTCATGATCTCCCGGACCCCTGAGGTCACGTGCGTG
GTGGTGGACGTGAGCCAGGAAGACCCCGAGGTCCAGTTCAACTGGTACGTGGATGGCGTGGA
GGTGCATAATGCCAAGACAAAGCCGCGGGAGGAGCAGTTCAACAGCACGTACCGTGTGGTCA
GCGTCCTCACCGTCCTGCACCAGGACTGGCTGAACGGCAAGGAGTACAAGTGCAAGGTCTCCA
ACAAAGGCCTCCCGTCCTCCATCGAGAAAACCATCTCCAAAGCCAAAGGGCAGCCCCGAGAG
CCACAGGTGTACACCCTGCCCCCATCCCAGGAGGAGATGACCAAGAACCAGGTCAGCCTGACC
TGCCTGGTCAAAGGCTTCTACCCCAGCGACATCGCCGTGGAGTGGGAGAGCAATGGGCAGCCG
GAGAACAACTACAAGACCACGCCTCCCGTGCTGGACTCCGACGGCTCCTTCTTCCTCTACAGC
AGGCTAACCGTGGACAAGAGCAGGTGGCAGGAGGGGAATGTCTTCTCATGCTCCGTGATGCAT
GAGGCTCTGCACAACCACTACACACAGAAGAGCCTCTCCCTGTCTCTGGGTAAAGCTAGCTGA
Anti-Lox_1_10F9H-LV-hIgG4H-C (SEQ ID NO: 124):
MEWTWVFLFLLSVTAGVHSQVQLQQSGAELMKPGASVKISCKATGYTFGSYWIEWVKQRPGHGL
EWIGEILPGSGNTNYNENFKGKATFTADTSSNTAYMQLTSLTSEDSAVYYCARAGIYWGQGTLVT
VSAAKTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFEGGPSVFLFPPKPKDTL
MISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHYTQKSLSLSLGK
AS
Anti-Lox_1_10F9K-LV-hIgGK-C (SEQ ID NO: 60):
ATGGAGAAAGACACACTCCTGCTATGGGTCCTGCTTCTCTGGGTTCCAGGTTCCACAGGTGAC
ATTGTGCTGACCCAATCTCCAGCTTTTTTGGCTGTGTCTCTAGGGCAGAGGGCCACCATCTCCT
GCAGAGCCAGCGAAAGTGTTGATAATTATGGCATTAGTTTTATGAACTGGTTCCAACAGAAAC
CAGGACAGCCACCCAAACTCCTCATCTATGTTGCATCCAAGCAAGGATCCGGGGTCCCTGCCA
GGTTTAGTGGCAGTGGGTCTGGGACAGACTTCAGCCTCAACATCCATCCTATGGAGGAGGATG
ATACTGCAATGTATTTCTGTCAGCAAAGTAAGGAGGTTCCTCGGACGTTCGGTGGAGGCACCA
AGCTCGAGATCAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACG
AGAAACACAAAGTCTATGCCTGCGAAGTCACCCATCAGGGCCTGAGCTCGCCCGTCACAAAGA
GCTTCAACAGGGGAGAGTGTTAG
Anti-Lox_1_10F9K-LV-hIgGK-C (SEQ ID NO: 125):
MEKDTLLLWVLLLWVPGSTGDIVLTQSPAFLAVSLGQRATISCRASESVDNYGISFMNWFQQKPG
QPPKLLIYVASKQGSGVPARFSGSGSGTDFSLNIHPMEEDDTAMYFCQQSKEVPRTFGGGTKLEIKR
TVAAPSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Anti-LOX-111C8H-LV-hIgG4H-C (SEQ ID NO: 61):
ATGGAATGTAACTGGATACTTCCTTTTATTCTGTCGGTAACTTCAGGGGTCTACTCAGAGGTTC
AGCTCCAGCAGTCTGGGACTGTGCTGGCAAGGCCTGGGGCTTCAGTGAAGATGTCCTGCAAGG
CTTCTGGCTACACCTTTACCAGCTACTGGATGCACTGGGTAAAACAGAGGCCTGGACAGGGTC
TGGAATGGATTGGCGCTATTTATCCTGGAAATAGTGATACTACCTACAACCAGAAGTTCAAGG
GCAAGGCCAAACTGACTGCAGTCACATCCACCAGCACTGCCTACATGGAGCTCAGCAGCCTGA
CAAATGAGGACTCTGCGGTCTATTACTGTACACCTACTTACTACTTTGACTACTGGGGCCAAGG
CACCTCTCTCACAGTCTCCTCAGCCAAAACGAAGGGCCCATCCGTCTTCCCCCTGGCGCCCTGC
TCCAGGAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCA
CGAAGACCTACACCTGCAACGTAGATCACAAGCCCAGCAACACCAAGGTGGACAAGAGAGTT
GAGTCCAAATATGGTCCCCCATGCCCACCCTGCCCAGCACCTGAGTTCGAAGGGGGACCATCA
GTCTTCCTGTTCCCCCCAAAACCCAAGGACACTCTCATGATCTCCCGGACCCCTGAGGTCACGT
GCGTGGTGGTGGACGTGAGCCAGGAAGACCCCGAGGTCCAGTTCAACTGGTACGTGGATGGC
GTGGAGGTGCATAATGCCAAGACAAAGCCGCGGGAGGAGCAGTTCAACAGCACGTACCGTGT
GGTCAGCGTCCTCACCGTCCTGCACCAGGACTGGCTGAACGGCAAGGAGTACAAGTGCAAGGT
CTCCAACAAAGGCCTCCCGTCCTCCATCGAGAAAACCATCTCCAAAGCCAAAGGGCAGCCCCG
AGAGCCACAGGTGTACACCCTGCCCCCATCCCAGGAGGAGATGACCAAGAACCAGGTCAGCC
TGACCTGCCTGGTCAAAGGCTTCTACCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCT
ACAGCAGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTAAAGCT
AGCTGA
Anti-LOX-111C8H-LV-hIgG4H-C (SEQ ID NO: 126):
MECNWILPFILSVTSGVYSEVQLQQSGTVLARPGASVKMSCKASGYTFTSYWMHWVKQRPGQGL
EWIGAIYPGNSDTTYNQKFKGKAKLTAVTSTSTAYMELSSLTNEDSAVYYCTPTYYFDYWGQGTS
LTVSSAKTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPPCPAPEFEGGPSVFLFPPKPKD
TLMISRTPEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSL
GKAS
Anti-LOX-111C8K-LV-hIgGK-C (SEQ ID NO: 62):
ATGAGTCCTGCCCAATTCCTGTTTCTGTTAGTGCTCTGGATTCGGGAAACCAACGGTGATGTTG
TGATGACCCAGACTCCACTCACTTTGTCGGTTACCATTGGACAACCAGCCTCCATCTCTTGCAA
GTCAAGTCAGAGCCTCTTAGATAGTGATGGAAAGACATATTTGAATTGGTTCTTACAGAGGCC
AGGCCAGTCTCCAAAGCGCCTAATCTATCTGGTGTCTAAACTGGACTCTGGAGTCCCTGACAG
GTTCACTGGCAGTGGATCAGGGACAGATTTCACACTGAAAATCAGCAGAGTGGAGGCTGAGG
ATTTGGGAGTTTATTATTGCTGGCAAGGTACACATTTTCCGTGGACGTTCGGTGGAGGCACCAA
GCTCGAGATCAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAG
GACAGCAAGGACAGCACCTACAGCCTCAGCAGCACCCTGACGCTGAGCAAAGCAGACTACGA
GAAACACAAAGTCTATGCCTGCGAAGTCACCCATCAGGGCCTGAGCTCGCCCGTCACAAAGAG
CTTCAACAGGGGAGAGTGTTAG
Anti-LOX-111C8K-LV-hIgGK-C (SEQ ID NO: 127):
MSPAQFLFLLVLWIRETNGDVVMTQTPLTLSVTIGQPASISCKSSQSLLDSDGKTYLNWFLQRPGQS
PKRLIYLVSKLDSGVPDRFTGSGSGTDFTLKISRVEAEDLGVYYCWQGTHFPWTFGGGTKLEIKRT
VAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-LOX-115C4H-LV-hIgG4H-C (SEQ ID NO: 63):
ATGGGAGGGATCTGGATCTTTCTCTTCCTCCTGTCAGGAACTGCAGGTGCCCACTCTGAGATCC
AGCTGCAGCAGACTGGACCTGAGCTGGTGAAGCCTGGGGCTTCAGTGAAGATATCCTGCAAGG
CTTCTGGTTATCCATTCACTGACTACATCATGGTCTGGGTGAAGCAGAGCCATGGAAAGAGCC
TTGAGTGGATTGGAAATATTAGTCCTTACTATGGTACTACTAACTACAATCTGAAGTTCAAGGG
CAAGGCCACATTGACTGTAGACAAATCTTCCAGCACAGCCTACATGCAGCTCAACAGTCTGAC
ATCTGAGGACTCTGCAGTCTATTACTGTGCAAGATCCCCTAACTGGGACGGGGCCTGGTTTGCT
CACTGGGGCCAAGGGGCTCTGGTCACTGTCTCTGCAGCCAAAACAAAGGGCCCATCCGTCTTC
CCCCTGGCGCCCTGCTCCAGGAGCACCTCCGAGAGCACAGCCGCCCTGGGCTGCCTGGTCAAG
GACTACTTCCCCGAACCGGTGACGGTGTCGTGGAACTCAGGCGCCCTGACCAGCGGCGTGCAC
ACCTTCCCGGCTGTCCTACAGTCCTCAGGACTCTACTCCCTCAGCAGCGTGGTGACCGTGCCCT
CCAGCAGCTTGGGCACGAAGACCTACACCTGCAACGTAGATCACAAGCCCAGCAACACCAAG
GTGGACAAGAGAGTTGAGTCCAAATATGGTCCCCCATGCCCACCCTGCCCAGCACCTGAGTTC
GAAGGGGGACCATCAGTCTTCCTGTTCCCCCCAAAACCCAAGGACACTCTCATGATCTCCCGG
ACCCCTGAGGTCACGTGCGTGGTGGTGGACGTGAGCCAGGAAGACCCCGAGGTCCAGTTCAAC
TGGTACGTGGATGGCGTGGAGGTGCATAATGCCAAGACAAAGCCGCGGGAGGAGCAGTTCAA
CAGCACGTACCGTGTGGTCAGCGTCCTCACCGTCCTGCACCAGGACTGGCTGAACGGCAAGGA
GTACAAGTGCAAGGTCTCCAACAAAGGCCTCCCGTCCTCCATCGAGAAAACCATCTCCAAAGC
CAAAGGGCAGCCCCGAGAGCCACAGGTGTACACCCTGCCCCCATCCCAGGAGGAGATGACCA
AGAACCAGGTCAGCCTGACCTGCCTGGTCAAAGGCTTCTACCCCAGCGACATCGCCGTGGAGT
GGGAGAGCAATGGGCAGCCGGAGAACAACTACAAGACCACGCCTCCCGTGCTGGACTCCGAC
GGCTCCTTCTTCCTCTACAGCAGGCTAACCGTGGACAAGAGCAGGTGGCAGGAGGGGAATGTC
TTCTCATGCTCCGTGATGCATGAGGCTCTGCACAACCACTACACACAGAAGAGCCTCTCCCTGT
CTCTGGGTAAAGCTAGCTGATTAATTAA
Anti-LOX-115C4H-LV-hIgG4H-C (SEQ ID NO: 128):
MGGIWIFLFLLSGTAGAHSEIQLQQTGPELVKPGASVKISCKASGYPFTDYIMVWVKQSHGKSLEW
IGNISPYYGTTNYNLKFKGKATLTVDKSSSTAYMQLNSLTSEDSAVYYCARSPNWDGAWFAHWG
QGALVTVSAAKTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGGPSVFLFPP
KPKDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSL
SLSLGKAS
Anti-LOX-115C4K-LV-hIgGK-C (SEQ ID NO: 64):
ATGGAGACAGACACAATCCTGCTATGGGTGCTGCTGCTCTGGGTTCCAGGCTCCACTGGTGAC
ATTGTGCTGACCCAATCTCCAGCTTCTTTGGCTGTGTCTCTAGGGCAGAGGGCCACCATCTCCT
GCAAGGCCAGCCAAAGTGTTGATTATGATGGTGATAGTTATATGAACTGGTTCCAACAGAAAC
CAGGACAGCCACCCAAACTCCTCATCTATGCTGCATCCAATCTAGAATCTGGGATCCCAGCCA
GGTTTAGTGGCAGTGGGTCTGGGACAGACTTCACCCTCAACATCCATCCTGTGGAGGAGGAGG
ATGCTGCAACCTATTACTGTCAGCAAAGTAATGAGGATCCATTCACGTTCGGCTCGGGGACAA
AGCTCGAGATCAAACGAACTGTGGCTGCACCATCTGTCTTCATCTTCCCGCCATCTGATGAGCA
GTTGAAATCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCACAGAGCA
GGACAGCAAGGACAGCACCTACAGCCTCAGCAGCACCCTGACGCTGAGCAAAGCAGACTACG
AGAAACACAAAGTCTATGCCTGCGAAGTCACCCATCAGGGCCTGAGCTCGCCCGTCACAAAGA
GCTTCAACAGGGGAGAGTGTTAG
Anti-LOX-115C4K-LV-hIgGK-C (SEQ ID NO: 129):
METDTILLWVLLLWVPGSTGDIVLTQSPASLAVSLGQRATISCKASQSVDYDGDSYMNWFQQKPG
QPPKLLIYAASNLESGIPARFSGSGSGTDFTLNIHPVEEEDAATYYCQQSNEDPFTFGSGTKLEIKRT
VAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Anti-Marco_10B7.3G4H-LV-hIgG4H-C (SEQ ID NO: 65):
ATGGCTGTCCTGGGGCTGCTTCTCTGCCTGGTGACGTTCCCAAGCTGTGTCCTGTCCCAGGTGC
AGCTGAAGGAGTCAGGACCTGGCCTGGTGGCACCCTCACAGAGCCTGTCCATCACATGCACTG
TCTCTGGGTTCTCATTATCCAGATATAGTGTATTTTGGGTTCGCCAGCCTCCAGGAAAGGGTCT
GGAGTGGCTGGGATTGATATGGGGTGGTGGAAGCACAGACTATAATTCAGCTCTCAAATCCAG
ACTGAGCATCAGCAAGGACAACTCCAAGAGCCAAGTTTTCTTAAAAATGAACAGTCTGCAAAC
TGATGACACAGCCATGTACTACTGTGCCAGAATCTACTTTGATTACGACGGGGCTATGGACTA
CTGGGGTCAAGGAACCTCAGTCACCGTCTCCTCAGCCAAAACAACGGGCCCATCCGTCTTCCC
CCTGGCGCCCTGCTCCAGGAGCACCTCCGAGAGCACAGCCGCCCTGGGCTGCCTGGTCAAGGA
CTACTTCCCCGAACCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGGTGACCGTGCCCTCC
AGCAGCTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAGTTCGA
AGGGGGACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTTCAACTG
GTACGTGGATGGCGTGGAGGTGCATAATGCCAAGACAAAGCCGCGGGAGGAGCAGTTCAACA
GCACGTACCGTGTGGTCAGCGTCCTCACCGTCCTGCACCAGGACTGGCTGAACGGCAAGGAGT
ACAAGTGCAAGGTCTCCAACAAAGGCCTCCCGTCCTCCATCGAGAAAACCATCTCCAAAGCCA
AAGGGCAGCCCCGAGAGCCACAGGTGTACACCCTGCCCCCATCCCAGGAGGAGATGACCAAG
AACCAGGTCAGCCTGACCTGCCTGGTCAAAGGCTTCTACCCCAGCGACATCGCCGTGGAGTGG
GAGAGCAATGGGCAGCCGGAGAACAACTACAAGACCACGCCTCCCGTGCTGGACTCCGACGG
CTCCTTCTTCCTCTACAGCAGGCTAACCGTGGACAAGAGCAGGTGGCAGGAGGGGAATGTCTT
CTCATGCTCCGTGATGCATGAGGCTCTGCACAACCACTACACACAGAAGAGCCTCTCCCTGTCT
CTGGGTAAAGCTAGCTG
Anti-Marco_10B7.3G4H-LV-hIgG4H-C (SEQ ID NO: 130):
MAVLGLLLCLVTFPSCVLSQVQLKESGPGLVAPSQSLSITCTVSGFSLSRYSVFWVRQPPGKGLEWL
GLIWGGGSTDYNSALKSRLSISKDNSKSQVFLKMNSLQTDDTAMYYCARIYFDYDGAMDYWGQG
TSVTVSSAKTTGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFEGGPSVFLFPPKP
KDTLMISRTPEVTCVVVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLS
LSLGKAS
AntiMarco_10B7.3G4K_H-V-hIgGK-C (SEQ ID NO: 66):
ATGCATCGCACCAGCATGGGCATCAAGATGGAGTCACGGATTCAGGCATTTGTATTCGTGTTTC
TCTGGTTGTCTGGTGTTGGCGGAGACATTGTGATGACCCAGTCTCACAAATTCATGTCCACATC
AGTAGGAGACAGGGTCAGCGTCACCTGCAAGGCCAGTCAGGATGTGACTTCTGCTGTAGCCTG
GTATCAACAAAAACCAGGGCAATCTCCTAAACTACTGATTTACTGGGCATCCACCCGGCACAC
TGGAGTCCCTGATCGCTTCACAGGCAGTGGATCTGGGACAGATTATACTCTCACCATCAGCAG
TGGGCAGGCTGAAGACCTGGCACTTTATTACTGTCACCAATATTATAGCGCTCCTCGGACGTTC
GGTGGAGGCACCAAGCTCGAGATCAAACGAACTGTGGCTGCACCATCTGTCTTCATCTTCCCG
CCATCTGATGAGCAGTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGTAACTCCCAGGAG
AGTGTCACAGAGCAGGACAGCAAGGACAGCACCTACAGCCTCAGCAGCACCCTGACGCTGAG
CAAAGCAGACTACGAGAAACACAAAGTCTATGCCTGCGAAGTCACCCATCAGGGCCTGAGCTC
GCCCGTCACAAAGAGCTTCAACAGGGGAGAGTGTTAG
AntiMarco_10B7.3G4K_H-V-hIgGK-C (SEQ ID NO: 131):
MHRTSMGIKMESRIQAFVFVFLWLSGVGGDIVMTQSHKFMSTSVGDRVSVTCKASQDVTSAVAW
YQQKPGQSPKLLIYWASTRHTGVPDRFTGSGSGTDYTLTISSGQAEDLALYYCHQYYSAPRTFGGG
TKLEIKRTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
Anti-Marco_11A8.3C9_H-V-hIgG4H-C (SEQ ID NO: 67):
ATGGAATGGAACTGGGTCGTTCTCTTCCTCCTGTCATTAACTGCAGGTGTCTATGCCCAGGGTC
AGATGCAGCAGTCTGGAGCTGAGCTGGTGAAGCCTGGGGCTTCAGTGAAGCTGTCCTGCAAGA
CTTCTGGCTTCACCTTCAGCAGTAACTATATAAGTTGGTTGAAGCAAAAGCCTGGACAGAGTCT
TGAGTGGATTGCATGGATTTATGCTGGAACTGGTGGTATTACCTATAATCAGAAGTTCAGAGG
CAGGGCCCAACTGACTGTAGACACATCCTCCAGCACAGCCTACATGCAGTTCAGCAGCCTGAC
AACTGATGACTCTGCCATCTATTACTGTGCAAGACACGTGAGGGGTTACCATCCTATGGACTAC
TGGGGTCAAGGAACCTCAGTCACCGTCTCCTCAGCCAAAACGAAGGGCCCATCCGTCTTCCCC
CTGGCGCCCTGCTCCAGGAGCACCTCCGAGAGCACAGCCGCCCTGGGCTGCCTGGTCAAGGAC
TACTTCCCCGAACCGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACCAAGGTG
GACAAGAGAGTTGAGTCCAAATATGGTCCCCCATGCCCACCCTGCCCAGCACCTGAGTTCGAA
GGGGGACCATCAGTCTTCCTGTTCCCCCCAAAACCCAAGGACACTCTCATGATCTCCCGGACCC
CTGAGGTCACGTGCGTGGTGGTGGACGTGAGCCAGGAAGACCCCGAGGTCCAGTTCAACTGGT
ACGTGGATGGCGTGGAGGTGCATAATGCCAAGACAAAGCCGCGGGAGGAGCAGTTCAACAGC
ACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAA
GGGCAGCCCCGAGAGCCACAGGTGTACACCCTGCCCCCATCCCAGGAGGAGATGACCAAGAA
CCAGGTCAGCCTGACCTGCCTGGTCAAAGGCTTCTACCCCAGCGACATCGCCGTGGAGTGGGA
GAGCAATGGGCAGCCGGAGAACAACTACAAGACCACGCCTCCCGTGCTGGACTCCGACGGCT
CCTTCTTCCTCTACAGCAGGCTAACCGTGGACAAGAGCAGGTGGCAGGAGGGGAATGTCTTCT
CATGCTCCGTGATGCATGAGGCTCTGCACAACCACTACACACAGAAGAGCCTCTCCCTGTCTCT
GGGTAAAGCTAGCTGA
Anti-Marco_11A8.3C9_H-V-hIgG4H-C (SEQ ID NO: 132):
MEWNWVVLFLLSLTAGVYAQGQMQQSGAELVKPGASVKLSCKTSGFTFSSNYISWLKQKPGQSL
EWIAWIYAGTGGITYNQKFRGRAQLTVDTSSSTAYMQFSSLTTDDSAIYYCARHVRGYHPMDYW
GQGTSVTVSSAKTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFEGGPSVFLFPP
KPKDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSL
SLSLGKAS
Anti-Marco_11A8.3C9_H-V-hIgGK-C (SEQ ID NO: 68):
ATGGAGTCACAGACTCAGGTCTTTGTATACATGTTGCTGTGGTTGTCTGGTGTTGATGGAGACA
TTGTGATGACCCAGTCTCAAAAATTCATGTCCGCATCAGTAGGGGACAGGGTCAGCGTCACCT
GCAGGGCCAGTCAGAATGTGGTTACTAATGTAGGCTGGTATCAACAGAAACCAGGGCAATCTC
CTAAAGTACTGATTTACTCGGCATCCTTCCGGTACAGTGGAGTCCCTGATCGCTTCACAGGCAG
TGGATCTGGGACAGATTTCACTCTCACCATCACCAATGTGCAGTCTGAAGACTTGGCAGAGTA
TTTCTGTCAGCAATATAACAACTATCCGTACACGTTCGGAGGGGGGACCAAGCTCGAGATCAA
ACGAACTGTGGCTGCACCATCTGTCTTCATCTTCCCGCCATCTGATGAGCAGTTGAAATCTGGA
ACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGA
CAGCACCTACAGCCTCAGCAGCACCCTGACGCTGAGCAAAGCAGACTACGAGAAACACAAAG
TCTATGCCTGCGAAGTCACCCATCAGGGCCTGAGCTCGCCCGTCACAAAGAGCTTCAACAGGG
GAGAGTGTTAG
Anti-Marco_11A8.3C9_H-V-hIgGK-C (SEQ ID NO: 133):
MESQTQVFVYMLLWLSGVDGDIVMTQSQKFMSASVGDRVSVTCRASQNVVTNVGWYQQKPGQS
PKVLIYSASFRYSGVPDRFTGSGSGTDFTLTITNVQSEDLAEYFCQQYNNYPYTFGGGTKLEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Anti-Marco_3H10.1F3_H-V-hIgG4H-C (SEQ ID NO: 69):
ATGGGATGGAGCTATATCATCCTCTTTTTGGTAGCAACAGCTACAGATGTCCACTCCCAGGTCC
AACTGCAGCAGCCTGGGGCTGAACTGGTGAAGCCTGGGGCTTCAGTGAAGCTGTCCTGCAAGG
CTTCTGGCTACACCTTCACCAGCTACTGGATGCACTGGGTGAAGCAGAGGCCTGGAGAAGGCC
TTGAGTGGATTGGAGAGATTAATCCTAGCTACGGTCGTACTGACTACAATGGGAAGTTCAAGA
ACAAGGCCACACTGACTGTAGCCAAATCCTCCAGCACAGCCTACATGCAACTCAGCAGCCTGA
CATCTGAGGACTCTGCGGTCTATTACTGTGCAAGAGGAGATTACTACGGTAGTAGCTCGTTTGC
TTACTGGGGCCAAGGGACTCTGGTCACTGTCTCTGCAGCCAAAACAAAGGGCCCATCCGTCTT
CCCCCTGGCGCCCTGCTCCAGGAGCACCTCCGAGAGCACAGCCGCCCTGGGCTGCCTGGTCAA
GGACTACTTCCCCGAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCGTGGTGACCGTGCCC
TCCAGCAGCTTGGGCACGAAGACCTACACCTGCAACGTAGATCACAAGCCCAGCAACACCAA
GGTGGACAAGAGAGTTGAGTCCAAATATGGTCCCCCATGCCCACCCTGCCCAGCACCTGAGTT
CGAAGGGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCAGTTCAA
CTGGTACGTGGATGGCGTGGAGGTGCATAATGCCAAGACAAAGCCGCGGGAGGAGCAGTTCA
ACAGCACGTACCGTGTGGTCAGCGTCCTCACCGTCCTGCACCAGGACTGGCTGAACGGCAAGG
AGTACAAGTGCAAGGTCTCCAACAAAGGCCTCCCGTCCTCCATCGAGAAAACCATCTCCAAAG
CCAAAGGGCAGCCCCGAGAGCCACAGGTGTACACCCTGCCCCCATCCCAGGAGGAGATGACC
AAGAACCAGGTCAGCCTGACCTGCCTGGTCAAAGGCTTCTACCCCAGCGACATCGCCGTGGAG
TGGGAGAGCAATGGGCAGCCGGAGAACAACTACAAGACCACGCCTCCCGTGCTGGACTCCGA
CGGCTCCTTCTTCCTCTACAGCAGGCTAACCGTGGACAAGAGCAGGTGGCAGGAGGGGAATGT
CTTCTCATGCTCCGTGATGCATGAGGCTCTGCACAACCACTACACACAGAAGAGCCTCTCCCTG
TCTCTGGGTAAAGCTAGCTGA
Anti-Marco_3H10.1F3_H-V-hIgG4H-C (SEQ ID NO: 134):
MGWSYIILFLVATATDVHSQVQLQQPGAELVKPGASVKLSCKASGYTFTSYWMHWVKQRPGEGL
EWIGEINPSYGRTDYNGKFKNKATLTVAKSSSTAYMQLSSLTSEDSAVYYCARGDYYGSSSFAYW
GQGTLVTVSAAKTKGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEFEGGPSVFLFP
PKPKDTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMHEALHNHYTQKS
LSLSLGKAS
Anti-Marco_3H10.1F3_K-V-hIgGK-C (SEQ ID NO: 70):
ATGGAGTCACAGACTCAGGTCTTTGTATACATGTTGCTGTGGTTGTCTGGTGTTGATGGAGACA
TTGTGATGACCCAGTCTCAAAAATTCATGTCCACATCATTAGGAGACAGGGTCAGCGTCACCT
GCAAGGCCAGTCAGAATGTGGGTACTAATGTAGCCTGGTATCAACAGAAACCAGGGCACTCTC
CTAAAGCACTGATTTACTCGGCATCCTACCGGTACAGTGGAGTCCCTGATCGCTTCACAGGCA
GTGGATCTGGGACAGATTTCACTCTCACCATCAGCAATGTGCAGTCTGAAGACTTGGCAGAGT
TTTTCTGTCAGCAATATAACAACTATCCGTACACGTTCGGAGGGGGGACCACGCTCGAGATCA
AACGAACTGTGGCTGCACCATCTGTCTTCATCTTCCCGCCATCTGATGAGCAGTTGAAATCTGG
AACTGCCTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAACACAAA
GTCTATGCCTGCGAAGTCACCCATCAGGGCCTGAGCTCGCCCGTCACAAAGAGCTTCAACAGG
GGAGAGTGTTAG
Anti-Marco_3H10.1F3_K-V-hIgGK-C (SEQ ID NO: 135):
MESQTQVFVYMLLWLSGVDGDIVMTQSQKFMSTSLGDRVSVTCKASQNVGTNVAWYQQKPGHS
PKALIYSASYRYSGVPDRFTGSGSGTDFTLTISNVQSEDLAEFFCQQYNNYPYTFGGGTTLEIKRTV
AAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

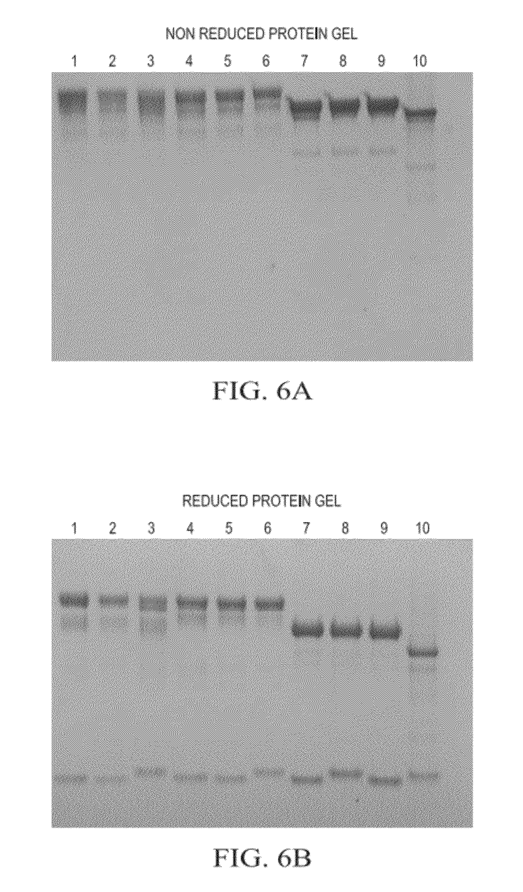

FIGS. 6A (non-reduced polyacrylamide gel) and 6B (reduced polyacrylamide gel) are PAGE gels that show the expression of the constructs listed as follows: In FIG. 6A—Lane 1. hIgG4-Flex-v1-m.tbRv1980-f4-m.tbRv0288-f2-m.tbAg85BDel41; Lane 2. manti-CD40_{—}12E12.3F3-hIgG4HC-Flex-v1m.tbRv1980-f4-m.tbRv0288-f2-m.tbAg85BDel41; Lane 3. mAnti-DCIR_{—}9E8-hIgG4-C-Flex-v1-m.tbRv1980-f4-m.tbRv0288-f2-m.tbAg85BDel41; Lane 4. hIgG4-Flex-v1-m.tbAg85BDel4142-m.tbESAT644-m.tbRv1980; Lane 5. manti-CD40_{—}12E12-hIgG4-C-Flex-v1-m.tbAg85BDel4142-m.tbESAT644-m.tbRv1980; Lane 6. hIgG4-Flex-v1-m.tbRv1980-f4-m.tbRv0288-f2-m.tbAg85BDel41; Lane 7. manti-CD40_{—}12E12.3F3-hIgG4HC-Flex-v1-m.tbRv1980-f4-m.tbRv0288-f2-m.tbAg85BDel41; Lane 8. mAnti-DCIR_{—}9E8-hIgG4-C-Flex-v1-m.tbRv1980-f4-m.tbRv0288-f2-m.tbAg85BDel41; Lane 9. hIgG4-Flex-v1-m.tbAg85BDel41-f2-m.tbESAT6-f4-m.tbRv1980; Lane 10. manti-CD40_{—}12E12-hIgG4-C-Flex-v1-m.tbAg85BDel41-f2-m.tbESAT6-f4-m.tbRv1980. In FIG. 6B—Lane 1. hIgG4-Flex-v1-m.tbRv1980-f4-m.tbRv0288-f2-m.tbAg85BDel41; Lane 2. manti-CD40_{—}12E12.3F3-hIgG4HC-Flex-v1-m.tbRv1980-f4-m.tbRv0288-f2-m.tbAg85BDel41; Lane 3. mAnti-DCIR_{—}9E8-hIgG4-C-Flex-v1-m.tbRv1980-f4-m.tbRv0288-f2-m.tbAg85BDel41; Lane 4. hIgG4-Flex-v1-m.tbAg85BDel41-f2-m.tbESAT6-f4-m.tbRv1980; Lane 5. manti-CD40_{—}12E12-hIgG4-C-Flex-v1-m.tbAg85BDel41-f2-m.tbESAT6-f4-m.tbRv1980; Lane 6. mAnti-DCIR_{—}9E8-hIgG4-C-Flex-v1-m.tbAg85BDel41-f2-m.tbESAT6-f4-m.tbRv1980; Lane 7. manti-CD40_{—}12E12-hIgG4-C-Flex-v1-m.tbCFP10-f2-m.tbESAT6; Lane 8. hIgG4-Flex-v1-m.tbCFP10-f2-m.tbESAT6; Lane 9. mAnti-DCIR_{—}9E8-hIgG4-C-Flex-v1-m.tbCFP10-f2-m.tbESAT6; Lane 10. mAnti-DCIR_{—}9E8-hIgG4-C-Flex-v1-m.tbHspX. The sequences for each of these constructs is detailed hereinbelow.

C2092: rAB-cetHS-puro [mAnti-DCIR_9E8_H-LV-hIgG4H-C-Flex-v1-m.tbAg85BDel41
(bold) - f2-m.tbESAT6 (underlined)-f4-m.tbRv1980 (italics)]
(SEQ ID NO: 136)
ATGAACAGGCTTACTTCCTCATTGCTGCTGCTGATTGTCCCTGCATATGTCCTGTCCCAGGTTAC
TCTGAAAGAGTCTGGCCCTGGGATATTGCAGCCCTCCCAGACCCTCAGTCTGACTTGTTCTTTC
TCTGGGTTTTCACTGAGCACTTCTGGTATGGGTCTGAGCTGGATTCGTCAGCCTTCAGGAAAGG
GTCTGGAGTGGCTGGCACACATTTACTGGGATGATGACAAGCGCTATAACCCATCCCTGAAGA
GCCGGCTCACAATCTCCAAGGATACCTCCAGCAACCAGGTTTTCCTCAAGATCACCATTGTGG
ACACTGCAGATGCTGCCACATACTACTGTGCTCGAAGCTCCCATTACTACGGTTATGGCTACGG
GGGATACTTCGATGTCTGGGGCGCAGGGACCACGGTCACCGTCTCCTCAGCCAAAACGAAGGG
CCCATCCGTCTTCCCCCTGGCGCCCTGCTCCAGGAGCACCTCCGAGAGCACAGCCGCCCTGGG
CTGCCTGGTCAAGGACTACTTCCCCGAACCGGTGACGGTGTCGTGGAACTCAGGCGCCCTGAC
CAGCGGCGTGCACACCTTCCCGGCTGTCCTACAGTCCTCAGGACTCTACTCCCTCAGCAGCGTG
GTGACCGTGCCCTCCAGCAGCTTGGGCACGAAGACCTACACCTGCAACGTAGATCACAAGCCC
AGCAACACCAAGGTGGACAAGAGAGTTGAGTCCAAATATGGTCCCCCATGCCCACCCTGCCCA
GCACCTGAGTTCGAAGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGCACCAGGACTGGCT
GAACGGCAAGGAGTACAAGTGCAAGGTCTCCAACAAAGGCCTCCCGTCCTCCATCGAGAAAA
CCATCTCCAAAGCCAAAGGGCAGCCCCGAGAGCCACAGGTGTACACCCTGCCCCCATCCCAGG
AGGAGATGACCAAGAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACCACGCCTCCCGTG
CTGGACTCCGACGGCTCCTTCTTCCTCTACAGCAGGCTAACCGTGGACAAGAGCAGGTGGCAG
GAGGGGAATGTCTTCTCATGCTCCGTGATGCATGAGGCTCTGCACAACCACTACACACAGAAG
AGCCTCTCCCTGTCTCTGGGTAAAGCTAGTCAGACCCCCACCAACACCATCAGCGTGACCCCC
ACCAACAACAGCACCCCCACCAACAACAGCAACCCCAAGCCCAACCCCGCTAGCTCCCGGCCG
GGGCTGCCGGTCGAGTACCTGCAGGTGCCGTCGCCGTCGATGGGCCGCGACATCAAGGTTCAG
TTCCAGAGCGGTGGGAACAACTCACCTGCGGTTTATCTGCTCGACGGCCTGCGCGCCCAAGAC
GACTACAACGGCTGGGATATCAACACCCCGGCGTTCGAGTGGTACTACCAGTCGGGACTGTCG
ATAGTCATGCCGGTCGGCGGGCAGTCCAGCTTCTACAGCGACTGGTACAGCCCGGCCTGCGGT
AAGGCTGGCTGCCAGACTTACAAGTGGGAAACCTTCCTGACCAGCGAGCTGCCGCAATGGTTG
TCCGCCAACAGGGCCGTGAAGCCCACCGGCAGCGCTGCAATCGGCTTGTCGATGGCCGGCTCG
TCGGCAATGATCTTGGCCGCCTACCACCCCCAGCAGTTCATCTACGCCGGCTCGCTGTCGGCCC
TGCTGGACCCCTCTCAGGGGATGGGGCCTAGCCTGATCGGCCTCGCGATGGGTGACGCCGGCG
GTTACAAGGCCGCAGACATGTGGGGTCCCTCGAGTGACCCGGCATGGGAGCGCAACGACCCTA
CGCAGCAGATCCCCAAGCTGGTCGCAAACAACACCCGGCTATGGGTTTATTGCGGGAACGGCA
CCCCGAACGAGTTGGGCGGTGCCAACATACCCGCCGAGTTCTTGGAGAACTTCGTTCGTAGCA
GCAACCTGAAGTTCCAGGATGCGTACAACGCCGCGGGCGGGCACAACGCCGTGTTCAACTTCC
CGCCCAACGGCACGCACAGCTGGGAGTACTGGGGCGCTCAGCTCAACGCCATGAAGGGTGAC
CTGCAGAGTTCGTTAGGCGCCGGCGAATTCCCCACCAGCACCCCCGCCGACAGCAGCACCATC
ACCCCCACCGCCACCCCCACCGCCACCCCCACCATCAAGGGCACTAGCATGACAGAGCAGCAG
TGGAATTTCGCGGGTATCGAGGCCGCGGCAAGCGCAATCCAGGGAAATGTCACGTCCATTCAT
TCCCTCCTTGACGAGGGGAAGCAGTCCCTGACCAAGCTCGCAGCGGCCTGGGGCGGTAGCGGT
TCGGAGGCGTACCAGGGTGTCCAGCAAAAATGGGACGCCACGGCTACCGAGCTGAACAACGC
GCTGCAGAACCTGGCGCGGACGATCAGCGAAGCCGGTCAGGCAATGGCTTCGACCGAAGGCA
ACGTCACTGGGATGTTCGCAAAGCTTGGCGGCGCGCCTACCAACGGCAGCATCACCGTGGCCG
CCACCGCCCCCACCGTGACCCCCACCGTGAACGCCACCCCCAGCGCCGCCTCTAGCGCCGCGC
CCAAGACCTACTGCGAGGAGTTGAAAGGCACCGATACCGGCCAGGCGTGCCAGATTCAAATGT
CCGACCCGGCCTACAACATCAACATCAGCCTGCCCAGTTACTACCCCGACCAGAAGTCGCTGG
AAAATTACATCGCCCAGACGCGCGACAAGTTCCTCAGCGCGGCCACATCGTCCACTCCACGCG
AAGCCCCCTACGAATTGAATATCACCTCGGCCACATACCAGTCCGCGATACCGCCGCGTGGTA
CGCAGGCCGTGGTGCTCAAGGTCTACCAGAACGCCGGCGGCACGCACCCAACGACCACGTAC
AAGGCCTTCGATTGGGACCAGGCCTATCGCAAGCCAATCACCTATGACACGCTGTGGCAGGCT
GACACCGATCCGCTGCCAGTCGTCTTCCCCATTGTGCAAGGTGAACTGAGCAAGCAGACCGGA
CAACAGGTATCGATAGCGCCGAATGCCGGCTTGGACCCGGTGAATTATCAGAACTTCGCAGTC
ACGAACGACGGGGTGATTTTCTTCTTCAACCCGGGGGAGTTGCTGCCCGAAGCAGCCGGCCCA
ACCCAGGTATTGGTCCCACGTTCCGCGATCGACTCGATGCTGGCCGAATTCTAGGCGGCCG
Protein sequence PAB2008 - rAB-cetHS-puro [mAnti-DCIR_9E8_H-LV-hIgG4H-C-Flex-v1-
m.tbAg85BDel41 (bold)-f2-m.tbESAT6 (underlined)-f4-m.tbRv1980 (italics)]
(SEQ ID NO: 137)
QVTLKESGPGILQPSQTLSLTCSFSGFSLSTSGMGLSWIRQPSGKGLEWLAHIYWDDDKRYNPSLKS
RLTISKDTSSNQVFLKITIVDTADAATYYCARSSHYYGYGYGGYFDVWGAGTTVTVSSAKTKGPS
VFPLAPCSRSTSESTAALGCLVKDYFPEPVTVSWNSGALTSGVHTFPAVLQSSGLYSLSSVVTVPSS
SLGTKTYTCNVDHKPSNTKVDKRVESKYGPPCPPCPAPEFEGGPSVFLFPPKPKDTLMISRTPEVTC
VVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSLSLSLGKASQTPTNTIS
VTPTNNSTPTNNSNPKPNPASSRPGLPVEYLQVPSPSMGRDIKVQFQSGGNNSPAVYLLDGLRA
QDDYNGWDINTPAFEWYYQSGLSIVMPVGGQSSFYSDWYSPACGKAGCQTYKWETFLTSEL
PQWLSANRAVKPTGSAAIGLSMAGSSAMILAAYHPQQFIYAGSLSALLDPSQGMGPSLIGLAM
GDAGGYKAADMWGPSSDPAWERNDPTQQIPKLVANNTRLWVYCGNGTPNELGGANIPAEFL
ENFVRSSNLKFQDAYNAAGGHNAVFNFPPNGTHSWEYWGAQLNAMKGDLQSSLGAGEFPTS
TPADSSTITPTATPTATPTIKGTSMTEQQWNFAGIEAAASAIQGNVTSIHSLLDEGKQSLTKLAAA
WGGSGSEAYQGVQQKWDATATELNNALQNLARTISEAGQAMASTEGNVTGMFAKLGGAPT
NGSITVAATAPTVTPTVNATPSAASS

[symbol]

[symbol]

[symbol]

[symbol]

[symbol]

[symbol]

[symbol]

[symbol]

[symbol]

EF
C2091: rAB-cetHS-puro[mAnti-DCIR_9E8_H-LV-hIgG4H-C-Flex-v1-m.tbRv1980 (M. tb.
antigen - bold)-f4-m.tbRv0288 (M. tb. - antigen - underlined)-f2-m.tbAg85BDel41
(M. tb. Antigen - italics]
(SEQ ID NO: 138)
ATGAACAGGCTTACTTCCTCATTGCTGCTGCTGATTGTCCCTGCATATGTCCTGTCCCAG
GTTACTCTGAAAGAGTCTGGCCCTGGGATATTGCAGCCCTCCCAGACCCTCAGTCTGACTTGTT
CTTTCTCTGGGTTTTCACTGAGCACTTCTGGTATGGGTCTGAGCTGGATTCGTCAGCCTTCAGG
AAAGGGTCTGGAGTGGCTGGCACACATTTACTGGGATGATGACAAGCGCTATAACCCATCCCT
GAAGAGCCGGCTCACAATCTCCAAGGATACCTCCAGCAACCAGGTTTTCCTCAAGATCACCAT
TGTGGACACTGCAGATGCTGCCACATACTACTGTGCTCGAAGCTCCCATTACTACGGTTATGGC
TACGGGGGATACTTCGATGTCTGGGGCGCAGGGACCACGGTCACCGTCTCCTCAGCCAAAACG
AAGGGCCCATCCGTCTTCCCCCTGGCGCCCTGCTCCAGGAGCACCTCCGAGAGCACAGCCGCC
CTGGGCTGCCTGGTCAAGGACTACTTCCCCGAACCGGTGACGGTGTCGTGGAACTCAGGCGCC
CTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCT
GCCCAGCACCTGAGTTCGAAGGGGGACCATCAGTCTTCCTGTTCCCCCCAAAACCCAAGGACA
CTCTCATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCCCATCCC
AGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTACACACAG
AAGAGCCTCTCCCTGTCTCTGGGTAAAGCTAGTCAGACCCCCACCAACACCATCAGCGTGACC
CCCACCAACAACAGCACCCCCACCAACAACAGCAACCCCAAGCCCAACCCCGCTAGCGCCGC
GCCCAAGACCTACTGCGAGGAGTTGAAAGGCACCGATACCGGCCAGGCGTGCCAGATTCAAA
TGTCCGACCCGGCCTACAACATCAACATCAGCCTGCCCAGTTACTACCCCGACCAGAAGTCGC
TGGAAAATTACATCGCCCAGACGCGCGACAAGTTCCTCAGCGCGGCCACATCGTCCACTCCAC
GCGAAGCCCCCTACGAATTGAATATCACCTCGGCCACATACCAGTCCGCGATACCGCCGCGTG
GTACGCAGGCCGTGGTGCTCAAGGTCTACCAGAACGCCGGCGGCACGCACCCAACGACCACGT
ACAAGGCCTTCGATTGGGACCAGGCCTATCGCAAGCCAATCACCTATGACACGCTGTGGCAGG
CTGACACCGATCCGCTGCCAGTCGTCTTCCCCATTGTGCAAGGTGAACTGAGCAAGCAGACCG
GACAACAGGTATCGATAGCGCCGAATGCCGGCTTGGACCCGGTGAATTATCAGAACTTCGCAG
TCACGAACGACGGGGTGATTTTCTTCTTCAACCCGGGGGAGTTGCTGCCCGAAGCAGCCGGCC
CAACCCAGGTATTGGTCCCACGTTCCGCGATCGACTCGATGCTGGCCAAGCTTGGCGGCGCGC
CTACCAACGGCAGCATCACCGTGGCCGCCACCGCCCCCACCGTGACCCCCACCGTGAACGCCA
CCCCCAGCGCCGCCTCTAGCATGTCGCAAATCATGTACAACTACCCCGCGATGTTGGGTCACG
CCGGGGATATGGCCGGATATGCCGGCACGCTGCAGAGCTTGGGTGCCGAGATCGCCGTGGAGC
AGGCCGCGTTGCAGAGTGCGTGGCAGGGCGATACCGGGATCACGTATCAGGCGTGGCAGGCA
CAGTGGAACCAGGCCATGGAAGATTTGGTGCGGGCCTATCATGCGATGTCCAGCACCCATGAA
GCCAACACCATGGCGATGATGGCCCGCGACACGGCCGAAGCCGCCAAATGGGGCGGCGAATT
CCCCACCAGCACCCCCGCCGACAGCAGCACCATCACCCCCACCGCCACCCCCACCGCCACCCC
CACCATCAAGGGCACTAGCTCCCGGCCGGGGCTGCCGGTCGAGTACCTGCAGGTGCCGTCGCC
GTCGATGGGCCGCGACATCAAGGTTCAGTTCCAGAGCGGTGGGAACAACTCACCTGCGGTTTA
TCTGCTCGACGGCCTGCGCGCCCAAGACGACTACAACGGCTGGGATATCAACACCCCGGCGTT
CGAGTGGTACTACCAGTCGGGACTGTCGATAGTCATGCCGGTCGGCGGGCAGTCCAGCTTCTA
CAGCGACTGGTACAGCCCGGCCTGCGGTAAGGCTGGCTGCCAGACTTACAAGTGGGAAACCTT
CCTGACCAGCGAGCTGCCGCAATGGTTGTCCGCCAACAGGGCCGTGAAGCCCACCGGCAGCGC
TGCAATCGGCTTGTCGATGGCCGGCTCGTCGGCAATGATCTTGGCCGCCTACCACCCCCAGCA
GTTCATCTACGCCGGCTCGCTGTCGGCCCTGCTGGACCCCTCTCAGGGGATGGGGCCTAGCCTG
ATCGGCCTCGCGATGGGTGACGCCGGCGGTTACAAGGCCGCAGACATGTGGGGTCCCTCGAGT
GACCCGGCATGGGAGCGCAACGACCCTACGCAGCAGATCCCCAAGCTGGTCGCAAACAACAC
CCGGCTATGGGTTTATTGCGGGAACGGCACCCCGAACGAGTTGGGCGGTGCCAACATACCCGC
CGAGTTCTTGGAGAACTTCGTTCGTAGCAGCAACCTGAAGTTCCAGGATGCGTACAACGCCGC
GGGCGGGCACAACGCCGTGTTCAACTTCCCGCCCAACGGCACGCACAGCTGGGAGTACTGGGG
CGCTCAGCTCAACGCCATGAAGGGTGACCTGCAGAGTTCGTTAGGCGCCGGCTGAGCGGCCG
Protein sequence PAB2059 - rAB-cetHS-puro[mAnti-DCIR_9E8_H-LV-hIgG4H-C-Flex-v1-
m.tbRv1980 (M. tb. antigen - bold)-f4-m.tbRv0288 (M. tb. - antigen - underlined)-
f2-m.tbAg85BDel41 (M. tb. Antigen - italics]
(SEQ ID NO: 139)
QVTLKESGPGILQPSQTLSLTCSFSGFSLSTSGMGLSWIRQPSGKGLEWLAHIYWDDDKRY
NPSLKSRLTISKDTSSNQVFLKITIVDTADAATYYCARSSHYYGYGYGGYFDVWGAGTTVTVSSAK
TKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFEGGPSVFLFPPKPKDTLMISRTP
EVTCVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSLSLSLGKASQTPT
NTISVTPTNNSTPTNNSNPKPNPASAAPKTYCEELKGTDTGQACQIQMSDPAYNINISLPSYYPD
QKSLENYIAQTRDKFLSAATSSTPREAPYELNITSATYQSAIPPRGTQAVVLKVYQNAGGTHPT
TTYKAFDWDQAYRKPITYDTLWQADTDPLPVVFPIVQGELSKQTGQQVSIAPNAGLDPVNYQ
NFAVTNDGVIFFFNPGELLPEAAGPTQVLVPRSAIDSMLAKLGGAPTNGSITVAATAPTVTPTVN
ATPSAASSMSQIMYNYPAMLGHAGDMAGYAGTLQSLGAEIAVEQAALQSAWQGDTGITYQAWQ
AQWNQAMEDLVRAYHAMSSTHEANTMAMMARDTAEAAKWGGEFPTSTPADSSTITPTATPTATP
TIKGTSSRPGLPVEYLQVPSPSMGRDIKVQFQSGGNNSPAVYLLDGLRAQDDYNGWDINTPAFEWYYQS
GLSIVMPVGGQSSFYSDWYSPACGKAGCQTYKWETFLTSELPQWLSANRAVKPTGSAAIGLSMAGSSAMI
LAAYHPQQFIYAGSLSALLDPSQGMGPSLIGLAMGDAGGYKAADMWGPSSDPAWERNDPTQQIPKLVA
NNTRLWVYCGNGTPNELGGANIPAEFLENFVRSSNLKFQDAYNAAGGHNAVFNFPPNGTHSWEYWGAQ
LNAMKGDLQSSLGAG
C2088: rAB-cetHS-puro[hIgG4H-Flex-v1-m.tbAg85BDel41 (M. tb. Antigen - bold) -f2-
m.tbESAT6 (M. tb. Antigen - underlined)-f4-m.tbRv1980 (M. tb. Antigen - italics)]
(SEQ ID NO: 140)
ATGGACCTCCTGTGCAAGAACATGAAGCACCTGTGGTTCTTCCTCCTGCTGGTGGCGGC
TCCCAGATGGGTCCTGTCCCGGCTGCAGCTGCAGGAGTCGGGCCCAGGCCTGCTGAAGCCTTC
GGTGACCCTGTCCCTCACCTGCACTGTCTCGGGTGACTCCGTCGCCAGTAGTTCTTATTACTGG
GGCTGGGTCCGTCAGCCCCCAGGGAAGGGACTCGAGTGGATAGGGACTATCAATTTTAGTGGC
AATATGTATTATAGTCCGTCCCTCAGGAGTCGAGTGACCATGTCGGCAGACATGTCCGAGAAC
TCCTTCTATCTGAAATTGGACTCTGTGACCGCAGCAGACACGGCCGTCTATTATTGTGCGGCAG
GACACCTCGTTATGGGATTTGGGGCCCACTGGGGACAGGGAAAACTGGTCTCCGTCTCTCCAG
CTTCCACCAAGGGCCCATCCGTCTTCCCCCTGGCGCCCTGCTCCAGGAGCACCTCCGAGAGCAC
AGCCGCCCTGGGCTGCCTGGTCAAGGACTACTTCCCCGAACCGGTGACGGTGTCGTGGAACTC
AGGCGCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACGAAGACCTACACCTGCAACGTA
GATCACAAGCCCAGCAACACCAAGGTGGACAAGAGAGTTGAGTCCAAATATGGTCCCCCATG
CCCACCCTGCCCAGCACCTGAGTTCGAAGGGGGACCATCAGTCTTCCTGTTCCCCCCAAAACCC
AAGGACACTCTCATGATCTCCCGGACCCCTGAGGTCACGTGCGTGGTGGTGGACGTGAGCCAG
GAAGACCCCGAGGTCCAGTTCAACTGGTACGTGGATGGCGTGGAGGTGCATAATGCCAAGAC
AAAGCCGCGGGAGGAGCAGTTCAACAGCACGTACCGTGTGGTCAGCGTCCTCACCGTCCTGCA
CCAGGACTGGCTGAACGGCAAGGAGTACAAGTGCAAGGTCTCCAACAAAGGCCTCCCGTCCTC
CATCGAGAAAACCATCTCCAAAGCCAAAGGGCAGCCCCGAGAGCCACAGGTGTACACCCTGC
CCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACAACCACT
ACACACAGAAGAGCCTCTCCCTGTCTCTGGGTAAAGCTAGTCAGACCCCCACCAACACCATCA
GCGTGACCCCCACCAACAACAGCACCCCCACCAACAACAGCAACCCCAAGCCCAACCCCGCTA
GCTCCCGGCCGGGGCTGCCGGTCGAGTACCTGCAGGTGCCGTCGCCGTCGATGGGCCGCGACA
TCAAGGTTCAGTTCCAGAGCGGTGGGAACAACTCACCTGCGGTTTATCTGCTCGACGGCCTGC
GCGCCCAAGACGACTACAACGGCTGGGATATCAACACCCCGGCGTTCGAGTGGTACTACCAGT
CGGGACTGTCGATAGTCATGCCGGTCGGCGGGCAGTCCAGCTTCTACAGCGACTGGTACAGCC
CGGCCTGCGGTAAGGCTGGCTGCCAGACTTACAAGTGGGAAACCTTCCTGACCAGCGAGCTGC
CGCAATGGTTGTCCGCCAACAGGGCCGTGAAGCCCACCGGCAGCGCTGCAATCGGCTTGTCGA
TGGCCGGCTCGTCGGCAATGATCTTGGCCGCCTACCACCCCCAGCAGTTCATCTACGCCGGCTC
GCTGTCGGCCCTGCTGGACCCCTCTCAGGGGATGGGGCCTAGCCTGATCGGCCTCGCGATGGG
TGACGCCGGCGGTTACAAGGCCGCAGACATGTGGGGTCCCTCGAGTGACCCGGCATGGGAGC
GCAACGACCCTACGCAGCAGATCCCCAAGCTGGTCGCAAACAACACCCGGCTATGGGTTTATT
GCGGGAACGGCACCCCGAACGAGTTGGGCGGTGCCAACATACCCGCCGAGTTCTTGGAGAACT
TCGTTCGTAGCAGCAACCTGAAGTTCCAGGATGCGTACAACGCCGCGGGCGGGCACAACGCCG
TGTTCAACTTCCCGCCCAACGGCACGCACAGCTGGGAGTACTGGGGCGCTCAGCTCAACGCCA
TGAAGGGTGACCTGCAGAGTTCGTTAGGCGCCGGCGAATTCCCCACCAGCACCCCCGCCGACA
GCAGCACCATCACCCCCACCGCCACCCCCACCGCCACCCCCACCATCAAGGGCACTAGCATGA
CAGAGCAGCAGTGGAATTTCGCGGGTATCGAGGCCGCGGCAAGCGCAATCCAGGGAAATGTC
ACGTCCATTCATTCCCTCCTTGACGAGGGGAAGCAGTCCCTGACCAAGCTCGCAGCGGCCTGG
GGCGGTAGCGGTTCGGAGGCGTACCAGGGTGTCCAGCAAAAATGGGACGCCACGGCTACCGA
GCTGAACAACGCGCTGCAGAACCTGGCGCGGACGATCAGCGAAGCCGGTCAGGCAATGGCTT
CGACCGAAGGCAACGTCACTGGGATGTTCGCAAAGCTTGGCGGCGCGCCTACCAACGGCAGC
ATCACCGTGGCCGCCACCGCCCCCACCGTGACCCCCACCGTGAACGCCACCCCCAGCGCCGCC
TCTAGCGCCGCGCCCAAGACCTACTGCGAGGAGTTGAAAGGCACCGATACCGGCCAGGCGTGC
CAGATTCAAATGTCCGACCCGGCCTACAACATCAACATCAGCCTGCCCAGTTACTACCCCGAC
CAGAAGTCGCTGGAAAATTACATCGCCCAGACGCGCGACAAGTTCCTCAGCGCGGCCACATCG
TCCACTCCACGCGAAGCCCCCTACGAATTGAATATCACCTCGGCCACATACCAGTCCGCGATA
CCGCCGCGTGGTACGCAGGCCGTGGTGCTCAAGGTCTACCAGAACGCCGGCGGCACGCACCCA
ACGACCACGTACAAGGCCTTCGATTGGGACCAGGCCTATCGCAAGCCAATCACCTATGACACG
CTGTGGCAGGCTGACACCGATCCGCTGCCAGTCGTCTTCCCCATTGTGCAAGGTGAACTGAGC
AAGCAGACCGGACAACAGGTATCGATAGCGCCGAATGCCGGCTTGGACCCGGTGAATTATCA
GAACTTCGCAGTCACGAACGACGGGGTGATTTTCTTCTTCAACCCGGGGGAGTTGCTGCCCGA
AGCAGCCGGCCCAACCCAGGTATTGGTCCCACGTTCCGCGATCGACTCGATGCTGGCCGAATT
CTAGGCGGCCGCG
Protein sequence PAB 2032- rAB-cetHS-puro[hIgG4H-Flex-v1-m.tbAg85BDel41 (M. tb.
Antigen - bold) -f2-m.tbESAT6 (M. tb. Antigen - underlined) -f4-m.tbRv1980
(M. tb. Antigen - italics)]
(SEQ ID NO: 141)
RLQLQESGPGLLKPSVTLSLTCTVSGDSVASSSYYWGWVRQPPGKGLEWIGTINFSGNMYY
SPSLRSRVTMSADMSENSFYLKLDSVTAADTAVYYCAAGHLVMGFGAHWGQGKLVSVSPASTKG
PSVFPLAPCSRSTSESTAALGCLVKDYFPEPVTVSWNSGALTSGVHTFPAVLQSSGLYSLSSVVTVP
SSSLGTKTYTCNVDHKPSNTKVDKRVESKYGPPCPPCPAPEFEGGPSVFLFPPKPKDTLMISRTPEVT
CVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKASQTPTNTI
SVTPTNNSTPTNNSNPKPNPASSRPGLPVEYLQVPSPSMGRDIKVQFQSGGNNSPAVYLLDGLR
AQDDYNGWDINTPAFEWYYQSGLSIVMPVGGQSSFYSDWYSPACGKAGCQTYKWETFLTSE
LPQWLSANRAVKPTGSAAIGLSMAGSSAMILAAYHPQQFIYAGSLSALLDPSQGMGPSLIGLA
MGDAGGYKAADMWGPSSDPAWERNDPTQQIPKLVANNTRLWVYCGNGTPNELGGANIPAE
FLENFVRSSNLKFQDAYNAAGGHNAVFNFPPNGTHSWEYWGAQLNAMKGDLQSSLGAGEFP
TSTPADSSTITPTATPTATPTIKGTSMTEQQWNFAGIEAAASAIQGNVTSIHSLLDEGKQSLTKLAAA
WGGSGSEAYQGVQQKWDATATELNNALQNLARTISEAGQAMASTEGNVTGMFAKLGGAPTNGSI
TVAATAPTVTPTVNATPSAASSAAPKTYCEELKGTDTGQACQIQMSDPAYNINISLPSYYPDQKSLENYI
AQTRDKFLSAATSSTPREAPYELNITSATYQSAIPPRGTQAVVLKVYQNAGGTHPTTTYKAFDWDQAYRKP
ITYDTLWQADTDPLPVVFPIVQGELSKQTGQQVSIAPNAGLDPVNYQNFAVTNDGVIFFFNPGELLPEAA
GPTQVLVPRSAIDSMLAEF
C2087: rAB-cetHS-puro[hIgG4H-Flex-v1-m.tbRv1980 (M. tb. antigen - bold)-f4-
m.tbRv0288 (M. tb. antigen - underlined)-f2-m.tbAg85BDel41 (M. tb. antigen -
italics)]
(SEQ ID NO: 142)
ATGGACCTCCTGTGCAAGAACATGAAGCACCTGTGGTTCTTCCTCCTGCTGGTGGCGGC
TCCCAGATGGGTCCTGTCCCGGCTGCAGCTGCAGGAGTCGGGCCCAGGCCTGCTGAAGCCTTC
GGTGACCCTGTCCCTCACCTGCACTGTCTCGGGTGACTCCGTCGCCAGTAGTTCTTATTACTGG
GGCTGGGTCCGTCAGCCCCCAGGGAAGGGACTCGAGTGGATAGGGACTATCAATTTTAGTGGC
AATATGTATTATAGTCCGTCCCTCAGGAGTCGAGTGACCATGTCGGCAGACATGTCCGAGAAC
TCCTTCTATCTGAAATTGGACTCTGTGACCGCAGCAGACACGGCCGTCTATTATTGTGCGGCAG
GACACCTCGTTATGGGATTTGGGGCCCACTGGGGACAGGGAAAACTGGTCTCCGTCTCTCCAG
CTTCCACCAAGGGCCCATCCGTCTTCCCCCTGGCGCCCTGCTCCAGGAGCACCTCCGAGAGCAC
AGCCGCCCTGGGCTGCCTGGTCAAGGACTACTTCCCCGAACCGGTGACGGTGTCGTGGAACTC
AGGCGCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACGAAGACCTACACCTGCAACGTA
GATCACAAGCCCAGCAACACCAAGGTGGACAAGAGAGTTGAGTCCAAATATGGTCCCCCATG
CCCACCCTGCCCAGCACCTGAGTTCGAAGGGGGACCATCAGTCTTCCTGTTCCCCCCAAAACCC
AAGGACACTCTCATGATCTCCCGGACCCCTGAGGTCACGTGCGTGGTGGTGGACGTGAGCCAG
GAAGACCCCGAGGTCCAGTTCAACTGGTACGTGGATGGCGTGGAGGTGCATAATGCCAAGAC
AAAGCCGCGGGAGGAGCAGTTCAACAGCACGTACCGTGTGGTCAGCGTCCTCACCGTCCTGCA
CCAGGACTGGCTGAACGGCAAGGAGTACAAGTGCAAGGTCTCCAACAAAGGCCTCCCGTCCTC
CATCGAGAAAACCATCTCCAAAGCCAAAGGGCAGCCCCGAGAGCCACAGGTGTACACCCTGC
CCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACAACCACT
ACACACAGAAGAGCCTCTCCCTGTCTCTGGGTAAAGCTAGTCAGACCCCCACCAACACCATCA
GCGTGACCCCCACCAACAACAGCACCCCCACCAACAACAGCAACCCCAAGCCCAACCCCGCTA
GCGCCGCGCCCAAGACCTACTGCGAGGAGTTGAAAGGCACCGATACCGGCCAGGCGTGCCAG
ATTCAAATGTCCGACCCGGCCTACAACATCAACATCAGCCTGCCCAGTTACTACCCCGACCAG
AAGTCGCTGGAAAATTACATCGCCCAGACGCGCGACAAGTTCCTCAGCGCGGCCACATCGTCC
ACTCCACGCGAAGCCCCCTACGAATTGAATATCACCTCGGCCACATACCAGTCCGCGATACCG
CCGCGTGGTACGCAGGCCGTGGTGCTCAAGGTCTACCAGAACGCCGGCGGCACGCACCCAACG
ACCACGTACAAGGCCTTCGATTGGGACCAGGCCTATCGCAAGCCAATCACCTATGACACGCTG
TGGCAGGCTGACACCGATCCGCTGCCAGTCGTCTTCCCCATTGTGCAAGGTGAACTGAGCAAG
CAGACCGGACAACAGGTATCGATAGCGCCGAATGCCGGCTTGGACCCGGTGAATTATCAGAAC
TTCGCAGTCACGAACGACGGGGTGATTTTCTTCTTCAACCCGGGGGAGTTGCTGCCCGAAGCA
GCCGGCCCAACCCAGGTATTGGTCCCACGTTCCGCGATCGACTCGATGCTGGCCAAGCTTGGC
GGCGCGCCTACCAACGGCAGCATCACCGTGGCCGCCACCGCCCCCACCGTGACCCCCACCGTG
AACGCCACCCCCAGCGCCGCCTCTAGCATGTCGCAAATCATGTACAACTACCCCGCGATGTTG
GGTCACGCCGGGGATATGGCCGGATATGCCGGCACGCTGCAGAGCTTGGGTGCCGAGATCGCC
GTGGAGCAGGCCGCGTTGCAGAGTGCGTGGCAGGGCGATACCGGGATCACGTATCAGGCGTG
GCAGGCACAGTGGAACCAGGCCATGGAAGATTTGGTGCGGGCCTATCATGCGATGTCCAGCAC
CCATGAAGCCAACACCATGGCGATGATGGCCCGCGACACGGCCGAAGCCGCCAAATGGGGCG
GCGAATTCCCCACCAGCACCCCCGCCGACAGCAGCACCATCACCCCCACCGCCACCCCCACCG
CCACCCCCACCATCAAGGGCACTAGCTCCCGGCCGGGGCTGCCGGTCGAGTACCTGCAGGTGC
CGTCGCCGTCGATGGGCCGCGACATCAAGGTTCAGTTCCAGAGCGGTGGGAACAACTCACCTG
CGGTTTATCTGCTCGACGGCCTGCGCGCCCAAGACGACTACAACGGCTGGGATATCAACACCC
CGGCGTTCGAGTGGTACTACCAGTCGGGACTGTCGATAGTCATGCCGGTCGGCGGGCAGTCCA
GCTTCTACAGCGACTGGTACAGCCCGGCCTGCGGTAAGGCTGGCTGCCAGACTTACAAGTGGG
AAACCTTCCTGACCAGCGAGCTGCCGCAATGGTTGTCCGCCAACAGGGCCGTGAAGCCCACCG
GCAGCGCTGCAATCGGCTTGTCGATGGCCGGCTCGTCGGCAATGATCTTGGCCGCCTACCACC
CCCAGCAGTTCATCTACGCCGGCTCGCTGTCGGCCCTGCTGGACCCCTCTCAGGGGATGGGGC
CTAGCCTGATCGGCCTCGCGATGGGTGACGCCGGCGGTTACAAGGCCGCAGACATGTGGGGTC
CCTCGAGTGACCCGGCATGGGAGCGCAACGACCCTACGCAGCAGATCCCCAAGCTGGTCGCAA
ACAACACCCGGCTATGGGTTTATTGCGGGAACGGCACCCCGAACGAGTTGGGCGGTGCCAACA
TACCCGCCGAGTTCTTGGAGAACTTCGTTCGTAGCAGCAACCTGAAGTTCCAGGATGCGTACA
ACGCCGCGGGCGGGCACAACGCCGTGTTCAACTTCCCGCCCAACGGCACGCACAGCTGGGAGT
ACTGGGGCGCTCAGCTCAACGCCATGAAGGGTGACCTGCAGAGTTCGTTAGGCGCCGGCTGAG
CGGCCG
Protein sequence PAB2007 - rAB-cetHS-puro[hIgG4H-Flex-v1-m.tbRv1980 (M. tb.
antigen - bold)-f4-m.tbRv0288 (M. tb. antigen - underlined)-f2-m.tbAg85BDel41
(M. tb. antigen - italics)]
(SEQ ID NO: 143)
RLQLQESGPGLLKPSVTLSLTCTVSGDSVASSSYYWGWVRQPPGKGLEWIGTINFSGNMYY
SPSLRSRVTMSADMSENSFYLKLDSVTAADTAVYYCAAGHLVMGFGAHWGQGKLVSVSPASTKG
PSVFPLAPCSRSTSESTAALGCLVKDYFPEPVTVSWNSGALTSGVHTFPAVLQSSGLYSLSSVVTVP
SSSLGTKTYTCNVDHKPSNTKVDKRVESKYGPPCPPCPAPEFEGGPSVFLFPPKPKDTLMISRTPEVT
CVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKASQTPTNTI
SVTPTNNSTPTNNSNPKPNPASAAPKTYCEELKGTDTGQACQIQMSDPAYNINISLPSYYPDQKS
LENYIAQTRDKFLSAATSSTPREAPYELNITSATYQSAIPPRGTQAVVLKVYQNAGGTHPTTTY
KAFDWDQAYRKPITYDTLWQADTDPLPVVFPIVQGELSKQTGQQVSIAPNAGLDPVNYQNFA
VTNDGVIFFFNPGELLPEAAGPTQVLVPRSAIDSMLAKLGGAPTNGSITVAATAPTVTPTVNATP
SAASSMSQIMYNYPAMLGHAGDMAGYAGTLQSLGAEIAVEQAALQSAWQGDTGITYQAWQAQ
WNQAMEDLVRAYHAMSSTHEANTMAMMARDTAEAAKWGGEFPTSTPADSSTITPTATPTATPTI
KGTSSRPGLPVEYLQVPSPSMGRDIKVQFQSGGNNSPAVYLLDGLRAQDDYNGWDINTPAFEWYYQSGL
SIVMPVGGQSSFYSDWYSPACGKAGCQTYKWETFLTSELPQWLSANRAVKPTGSAAIGLSMAGSSAMILA
AYHPQQFIYAGSLSALLDPSQGMGPSLIGLAMGDAGGYKAADMWGPSSDPAWERNDPTQQIPKLVANN
TRLWVYCGNGTPNELGGANIPAEFLENFVRSSNLKFQDAYNAAGGHNAVFNFPPNGTHSWEYWGAQLN
AMKGDLQSSLGAG
C2084: rAB-cetHS-puro[manti-CD40_12E12.3F3_H-V-SLAML-hIgG4H-C-Flex-v1-
m.tbAg85BDel41 (M. tb. antigen - bold)-f2-m.tbESAT6 (M. tb. antigen -
underlined)- f4-m.tbRv1980(M. tb. antigen-italics)]
(SEQ ID NO: 144)
ATGGACCCCAAAGGCTCCCTTTCCTGGAGAATACTTCTGTTTCTCTCCCTGGCTTTTGAG
TTGTCGTACGGAGAAGTGAAGCTGGTGGAGTCTGGGGGAGGCTTAGTGCAGCCTGGAGGGTCC
CTGAAACTCTCCTGTGCAACCTCTGGATTCACTTTCAGTGACTATTACATGTATTGGGTTCGCC
AGACTCCAGAGAAGAGGCTGGAGTGGGTCGCATACATTAATTCTGGTGGTGGTAGCACCTATT
ATCCAGACACTGTAAAGGGCCGATTCACCATCTCCAGAGACAATGCCAAGAACACCCTGTACC
TGCAAATGAGCCGGCTGAAGTCTGAGGACACAGCCATGTATTACTGTGCAAGACGGGGGTTAC
CGTTCCATGCTATGGACTATTGGGGTCAAGGAACCTCAGTCACCGTCTCCTCAGCCAAAACGA
AGGGCCCATCCGTCTTCCCCCTGGCGCCCTGCTCCAGGAGCACCTCCGAGAGCACAGCCGCCC
TGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAG
CGTGGTGACCGTGCCCTCCAGCAGCTTGGGCACGAAGACCTACACCTGCAACGTAGATCACAA
GCCCAGCAACACCAAGGTGGACAAGAGAGTTGAGTCCAAATATGGTCCCCCATGCCCACCCTG
CCCAGCACCTGAGTTCGAAGGGGGACCATCAGTCTTCCTGTTCCCCCCAAAACCCAAGGACAC
TCTCATGATCTCCCGGACCCCTGAGGTCACGTGCGTGGTGGTGGACGTGAGCCAGGAAGACCC
CGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCCCATCCC
AGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTACACACAG
AAGAGCCTCTCCCTGTCTCTGGGTAAAGCTAGTCAGACCCCCACCAACACCATCAGCGTGACC
CCCACCAACAACAGCACCCCCACCAACAACAGCAACCCCAAGCCCAACCCCGCTAGCTCCCGG
CCGGGGCTGCCGGTCGAGTACCTGCAGGTGCCGTCGCCGTCGATGGGCCGCGACATCAAGGTT
CAGTTCCAGAGCGGTGGGAACAACTCACCTGCGGTTTATCTGCTCGACGGCCTGCGCGCCCAA
GACGACTACAACGGCTGGGATATCAACACCCCGGCGTTCGAGTGGTACTACCAGTCGGGACTG
TCGATAGTCATGCCGGTCGGCGGGCAGTCCAGCTTCTACAGCGACTGGTACAGCCCGGCCTGC
GGTAAGGCTGGCTGCCAGACTTACAAGTGGGAAACCTTCCTGACCAGCGAGCTGCCGCAATGG
TTGTCCGCCAACAGGGCCGTGAAGCCCACCGGCAGCGCTGCAATCGGCTTGTCGATGGCCGGC
TCGTCGGCAATGATCTTGGCCGCCTACCACCCCCAGCAGTTCATCTACGCCGGCTCGCTGTCGG
CCCTGCTGGACCCCTCTCAGGGGATGGGGCCTAGCCTGATCGGCCTCGCGATGGGTGACGCCG
GCGGTTACAAGGCCGCAGACATGTGGGGTCCCTCGAGTGACCCGGCATGGGAGCGCAACGAC
CCTACGCAGCAGATCCCCAAGCTGGTCGCAAACAACACCCGGCTATGGGTTTATTGCGGGAAC
GGCACCCCGAACGAGTTGGGCGGTGCCAACATACCCGCCGAGTTCTTGGAGAACTTCGTTCGT
AGCAGCAACCTGAAGTTCCAGGATGCGTACAACGCCGCGGGCGGGCACAACGCCGTGTTCAA
CTTCCCGCCCAACGGCACGCACAGCTGGGAGTACTGGGGCGCTCAGCTCAACGCCATGAAGGG
TGACCTGCAGAGTTCGTTAGGCGCCGGCGAATTCCCCACCAGCACCCCCGCCGACAGCAGCAC
CATCACCCCCACCGCCACCCCCACCGCCACCCCCACCATCAAGGGCACTAGCATGACAGAGCA
GCAGTGGAATTTCGCGGGTATCGAGGCCGCGGCAAGCGCAATCCAGGGAAATGTCACGTCCAT
TCATTCCCTCCTTGACGAGGGGAAGCAGTCCCTGACCAAGCTCGCAGCGGCCTGGGGCGGTAG
CGGTTCGGAGGCGTACCAGGGTGTCCAGCAAAAATGGGACGCCACGGCTACCGAGCTGAACA
ACGCGCTGCAGAACCTGGCGCGGACGATCAGCGAAGCCGGTCAGGCAATGGCTTCGACCGAA
GGCAACGTCACTGGGATGTTCGCAAAGCTTGGCGGCGCGCCTACCAACGGCAGCATCACCGTG
GCCGCCACCGCCCCCACCGTGACCCCCACCGTGAACGCCACCCCCAGCGCCGCCTCTAGCGCC
GCGCCCAAGACCTACTGCGAGGAGTTGAAAGGCACCGATACCGGCCAGGCGTGCCAGATTCA
AATGTCCGACCCGGCCTACAACATCAACATCAGCCTGCCCAGTTACTACCCCGACCAGAAGTC
GCTGGAAAATTACATCGCCCAGACGCGCGACAAGTTCCTCAGCGCGGCCACATCGTCCACTCC
ACGCGAAGCCCCCTACGAATTGAATATCACCTCGGCCACATACCAGTCCGCGATACCGCCGCG
TGGTACGCAGGCCGTGGTGCTCAAGGTCTACCAGAACGCCGGCGGCACGCACCCAACGACCAC
GTACAAGGCCTTCGATTGGGACCAGGCCTATCGCAAGCCAATCACCTATGACACGCTGTGGCA
GGCTGACACCGATCCGCTGCCAGTCGTCTTCCCCATTGTGCAAGGTGAACTGAGCAAGCAGAC
CGGACAACAGGTATCGATAGCGCCGAATGCCGGCTTGGACCCGGTGAATTATCAGAACTTCGC
AGTCACGAACGACGGGGTGATTTTCTTCTTCAACCCGGGGGAGTTGCTGCCCGAAGCAGCCGG
CCCAACCCAGGTATTGGTCCCACGTTCCGCGATCGACTCGATGCTGGCCGAATTCTAGGCG
Protein sequence PAB2058 - rAB-cetHS-puro[manti-CD40_12E12.3F3_H-V-SLAML-hIgG4H-C-
Flex-v1-m.tbAg85BDel41 (M. tb. antigen - bold)-f2-m.tbESAT6 (M. tb. antigen -
underlined)-f4-m.tbRv1980 (M. tb. antigen - italics)].
(SEQ ID NO: 145)
EVKLVESGGGLVQPGGSLKLSCATSGFTFSDYYMYWVRQTPEKRLEWVAYINSGGGSTYY
PDTVKGRFTISRDNAKNTLYLQMSRLKSEDTAMYYCARRGLPFHAMDYWGQGTSVTVSSAKTKG
PSVFPLAPCSRSTSESTAALGCLVKDYFPEPVTVSWNSGALTSGVHTFPAVLQSSGLYSLSSVVTVP
SSSLGTKTYTCNVDHKPSNTKVDKRVESKYGPPCPPCPAPEFEGGPSVFLFPPKPKDTLMISRTPEVT
CVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKASQTPTNTI
SVTPTNNSTPTNNSNPKPNPASSRPGLPVEYLQVPSPSMGRDIKVQFQSGGNNSPAVYLLDGLR
AQDDYNGWDINTPAFEWYYQSGLSIVMPVGGQSSFYSDWYSPACGKAGCQTYKWETFLTSE
LPQWLSANRAVKPTGSAAIGLSMAGSSAMILAAYHPQQFIYAGSLSALLDPSQGMGPSLIGLA
MGDAGGYKAADMWGPSSDPAWERNDPTQQIPKLVANNTRLWVYCGNGTPNELGGANIPAE
FLENFVRSSNLKFQDAYNAAGGHNAVFNFPPNGTHSWEYWGAQLNAMKGDLQSSLGAGEFP
TSTPADSSTITPTATPTATPTIKGTSMTEQQWNFAGIEAAASAIQGNVTSIHSLLDEGKQSLTKLAAA
WGGSGSEAYQGVQQKWDATATELNNALQNLARTISEAGQAMASTEGNVTGMFAKLGGAPTNGSI
TVAATAPTVTPTVNATPSAASSAAPKTYCEELKGTDTGQACQIQMSDPAYNINISLPSYYPDQKSLENYI
AQTRDKFLSAATSSTPREAPYELNITSATYQSAIPPRGTQAVVLKVYQNAGGTHPTTTYKAFDWDQAYRKP
ITYDTLWQADTDPLPVVFPIVQGELSKQTGQQVSIAPNAGLDPVNYQNFAVTNVDGVIFFFNPGELLPEAA
GPTQVLVPRSAIDSMLAEF
C2083: rAB-cetHS-puro[manti-CD40_12E12.3F3_H-V-SLAML-hIgG4H-C-Flex-v1-m.tbRv1980
(M. tb. antigen - bold)-f4-m.tbRv0288 (M. tb. antigen - underlined)-f2-
m.tbAg85BDel41(M. tb. antigen - italics)]
(SEQ ID NO: 146)
ATGGACCCCAAAGGCTCCCTTTCCTGGAGAATACTTCTGTTTCTCTCCCTGGCTTTTGAG
TTGTCGTACGGAGAAGTGAAGCTGGTGGAGTCTGGGGGAGGCTTAGTGCAGCCTGGAGGGTCC
CTGAAACTCTCCTGTGCAACCTCTGGATTCACTTTCAGTGACTATTACATGTATTGGGTTCGCC
AGACTCCAGAGAAGAGGCTGGAGTGGGTCGCATACATTAATTCTGGTGGTGGTAGCACCTATT
ATCCAGACACTGTAAAGGGCCGATTCACCATCTCCAGAGACAATGCCAAGAACACCCTGTACC
TGCAAATGAGCCGGCTGAAGTCTGAGGACACAGCCATGTATTACTGTGCAAGACGGGGGTTAC
CGTTCCATGCTATGGACTATTGGGGTCAAGGAACCTCAGTCACCGTCTCCTCAGCCAAAACGA
AGGGCCCATCCGTCTTCCCCCTGGCGCCCTGCTCCAGGAGCACCTCCGAGAGCACAGCCGCCC
TGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAG
CGTGGTGACCGTGCCCTCCAGCAGCTTGGGCACGAAGACCTACACCTGCAACGTAGATCACAA
GCCCAGCAACACCAAGGTGGACAAGAGAGTTGAGTCCAAATATGGTCCCCCATGCCCACCCTG
CCCAGCACCTGAGTTCGAAGGGGGACCATCAGTCTTCCTGTTCCCCCCAAAACCCAAGGACAC
TCTCATGATCTCCCGGACCCCTGAGGTCACGTGCGTGGTGGTGGACGTGAGCCAGGAAGACCC
CGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCCCATCCC
AGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTACACACAG
AAGAGCCTCTCCCTGTCTCTGGGTAAAGCTAGTCAGACCCCCACCAACACCATCAGCGTGACC
CCCACCAACAACAGCACCCCCACCAACAACAGCAACCCCAAGCCCAACCCCGCTAGCGCCGC
GCCCAAGACCTACTGCGAGGAGTTGAAAGGCACCGATACCGGCCAGGCGTGCCAGATTCAAA
TGTCCGACCCGGCCTACAACATCAACATCAGCCTGCCCAGTTACTACCCCGACCAGAAGTCGC
TGGAAAATTACATCGCCCAGACGCGCGACAAGTTCCTCAGCGCGGCCACATCGTCCACTCCAC
GCGAAGCCCCCTACGAATTGAATATCACCTCGGCCACATACCAGTCCGCGATACCGCCGCGTG
GTACGCAGGCCGTGGTGCTCAAGGTCTACCAGAACGCCGGCGGCACGCACCCAACGACCACGT
ACAAGGCCTTCGATTGGGACCAGGCCTATCGCAAGCCAATCACCTATGACACGCTGTGGCAGG
CTGACACCGATCCGCTGCCAGTCGTCTTCCCCATTGTGCAAGGTGAACTGAGCAAGCAGACCG
GACAACAGGTATCGATAGCGCCGAATGCCGGCTTGGACCCGGTGAATTATCAGAACTTCGCAG
TCACGAACGACGGGGTGATTTTCTTCTTCAACCCGGGGGAGTTGCTGCCCGAAGCAGCCGGCC
CAACCCAGGTATTGGTCCCACGTTCCGCGATCGACTCGATGCTGGCCAAGCTTGGCGGCGCGC
CTACCAACGGCAGCATCACCGTGGCCGCCACCGCCCCCACCGTGACCCCCACCGTGAACGCCA
CCCCCAGCGCCGCCTCTAGCATGTCGCAAATCATGTACAACTACCCCGCGATGTTGGGTCACG
CCGGGGATATGGCCGGATATGCCGGCACGCTGCAGAGCTTGGGTGCCGAGATCGCCGTGGAGC
AGGCCGCGTTGCAGAGTGCGTGGCAGGGCGATACCGGGATCACGTATCAGGCGTGGCAGGCA
CAGTGGAACCAGGCCATGGAAGATTTGGTGCGGGCCTATCATGCGATGTCCAGCACCCATGAA
GCCAACACCATGGCGATGATGGCCCGCGACACGGCCGAAGCCGCCAAATGGGGCGGCGAATT
CCCCACCAGCACCCCCGCCGACAGCAGCACCATCACCCCCACCGCCACCCCCACCGCCACCCC
CACCATCAAGGGCACTAGCTCCCGGCCGGGGCTGCCGGTCGAGTACCTGCAGGTGCCGTCGCC
GTCGATGGGCCGCGACATCAAGGTTCAGTTCCAGAGCGGTGGGAACAACTCACCTGCGGTTTA
TCTGCTCGACGGCCTGCGCGCCCAAGACGACTACAACGGCTGGGATATCAACACCCCGGCGTT
CGAGTGGTACTACCAGTCGGGACTGTCGATAGTCATGCCGGTCGGCGGGCAGTCCAGCTTCTA
CAGCGACTGGTACAGCCCGGCCTGCGGTAAGGCTGGCTGCCAGACTTACAAGTGGGAAACCTT
CCTGACCAGCGAGCTGCCGCAATGGTTGTCCGCCAACAGGGCCGTGAAGCCCACCGGCAGCGC
TGCAATCGGCTTGTCGATGGCCGGCTCGTCGGCAATGATCTTGGCCGCCTACCACCCCCAGCA
GTTCATCTACGCCGGCTCGCTGTCGGCCCTGCTGGACCCCTCTCAGGGGATGGGGCCTAGCCTG
ATCGGCCTCGCGATGGGTGACGCCGGCGGTTACAAGGCCGCAGACATGTGGGGTCCCTCGAGT
GACCCGGCATGGGAGCGCAACGACCCTACGCAGCAGATCCCCAAGCTGGTCGCAAACAACAC
CCGGCTATGGGTTTATTGCGGGAACGGCACCCCGAACGAGTTGGGCGGTGCCAACATACCCGC
CGAGTTCTTGGAGAACTTCGTTCGTAGCAGCAACCTGAAGTTCCAGGATGCGTACAACGCCGC
GGGCGGGCACAACGCCGTGTTCAACTTCCCGCCCAACGGCACGCACAGCTGGGAGTACTGGGG
CGCTCAGCTCAACGCCATGAAGGGTGACCTGCAGAGTTCGTTAGGCGCCGGCTGAGCGGCC
Protein sequence PAB2122 - rAB-cetHS-puro[manti-CD40_12E12.3F3_H-V-SLAML-hIgG4H-C-
Flex-v1-m.tbRv1980 (M. tb. antigen - bold)-f4-m.tbRv0288 (M. tb. antigen -
underlined)-f2-m.tbAg85BDel41 (M. tb. antigen - italics)].
(SEQ ID NO: 147)
EVKLVESGGGLVQPGGSLKLSCATSGFTFSDYYMYWVRQTPEKRLEWVAYINSGGGSTYY
PDTVKGRFTISRDNAKNTLYLQMSRLKSEDTAMYYCARRGLPFHAMDYWGQGTSVTVSSAKTKG
PSVFPLAPCSRSTSESTAALGCLVKDYFPEPVTVSWNSGALTSGVHTFPAVLQSSGLYSLSSVVTVP
SSSLGTKTYTCNVDHKPSNTKVDKRVESKYGPPCPPCPAPEFEGGPSVFLFPPKPKDTLMISRTPEVT
CVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKASQTPTNTI
SVTPTNNSTPTNNSNPKPNPASAAPKTYCEELKGTDTGQACQIQMSDPAYNINISLPSYYPDQKS
LENYIAQTRDKFLSAATSSTPREAPYELNITSATYQSAIPPRGTQAVVLKVYQNAGGTHPTTTY
KAFDWDQAYRKPITYDTLWQADTDPLPVVFPIVQGELSKQTGQQVSIAPNAGLDPVNYQNFA
VTNDGVIFFFNPGELLPEAAGPTQVLVPRSAIDSMLAKLGGAPTNGSITVAATAPTVTPTVNATP
SAASSMSQIMYNYPAMLGHAGDMAGYAGTLQSLGAEIAVEQAALQSAWQGDTGITYQAWQAQ
WNQAMEDLVRAYHAMSSTHEANTMAMMARDTAEAAKWGGEFPTSTPADSSTITPTATPTATPTI
KGTSSRPGLPVEYLQVPSPSMGRDIKVQFQSGGNNSPAVYLLDGLRAQDDYNGWDINTPAFEWYYQSGL
SIVMPVGGQSSFYSDWYSPACGKAGCQTYKWETFLTSELPQWLSANRAVKPTGSAAIGLSMAGSSAMILA
AYHPQQFIYAGSLSALLDPSQGMGPSLIGLAMGDAGGYKAADMWGPSSDPAWERNDPTQQIPKLVANN
TRLWVYCGNGTPNELGGANIPAEFLENFVRSSNLKFQDAYNAAGGHNAVFNFPPNGTHSWEYWGAQLN
AMKGDLQSSLGAG
C1590: rAB-cetHS-puro[mAnti-DCIR_9E8_H-LV-hIgG4H-C-Flex-v1-m.tbCFP10 (M. tb.
antigen - bold)-f2-m.tbESAT6 (M. tb. antigen - underlined)].
(SEQ ID NO: 148)
ATGAACAGGCTTACTTCCTCATTGCTGCTGCTGATTGTCCCTGCATATGTCCTGTCCCAG
GTTACTCTGAAAGAGTCTGGCCCTGGGATATTGCAGCCCTCCCAGACCCTCAGTCTGACTTGTT
CTTTCTCTGGGTTTTCACTGAGCACTTCTGGTATGGGTCTGAGCTGGATTCGTCAGCCTTCAGG
AAAGGGTCTGGAGTGGCTGGCACACATTTACTGGGATGATGACAAGCGCTATAACCCATCCCT
GAAGAGCCGGCTCACAATCTCCAAGGATACCTCCAGCAACCAGGTTTTCCTCAAGATCACCAT
TGTGGACACTGCAGATGCTGCCACATACTACTGTGCTCGAAGCTCCCATTACTACGGTTATGGC
TACGGGGGATACTTCGATGTCTGGGGCGCAGGGACCACGGTCACCGTCTCCTCAGCCAAAACG
AAGGGCCCATCCGTCTTCCCCCTGGCGCCCTGCTCCAGGAGCACCTCCGAGAGCACAGCCGCC
CTGGGCTGCCTGGTCAAGGACTACTTCCCCGAACCGGTGACGGTGTCGTGGAACTCAGGCGCC
CTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCT
GCCCAGCACCTGAGTTCGAAGGGGGACCATCAGTCTTCCTGTTCCCCCCAAAACCCAAGGACA
CTCTCATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCCCATCCC
AGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTACACACAG
AAGAGCCTCTCCCTGTCTCTGGGTAAAGCTAGCATGGCAGAGATGAAGACCGATGCCGCTACC
CTCGCGCAGGAGGCAGGTAATTTCGAGCGGATCTCCGGCGACCTGAAAACCCAGATCGACCAG
GTGGAGTCGACGGCAGGTTCGTTGCAGGGCCAGTGGCGCGGCGCGGCGGGGACGGCCGCCCA
GGCCGCGGTGGTGCGCTTCCAAGAAGCAGCCAATAAGCAGAAGCAGGAACTCGACGAGATCT
CGACGAATATTCGTCAGGCCGGCGTCCAATACTCGAGGGCCGACGAGGAGCAGCAGCAGGCG
CTGTCCTCGCAAATGGGCTTCGAATTCCCCACCAGCACCCCCGCCGACAGCAGCACCATCACC
CCCACCGCCACCCCCACCGCCACCCCCACCATCAAGGGCACTAGCATGACAGAGCAGCAGTGG
AATTTCGCGGGTATCGAGGCCGCGGCAAGCGCAATCCAGGGAAATGTCACGTCCATTCATTCC
CTCCTTGACGAGGGGAAGCAGTCCCTGACCAAGCTCGCAGCGGCCTGGGGCGGTAGCGGTTCG
GAGGCGTACCAGGGTGTCCAGCAAAAATGGGACGCCACGGCTACCGAGCTGAACAACGCGCT
GCAGAACCTGGCGCGGACGATCAGCGAAGCCGGTCAGGCAATGGCTTCGACCGAAGGCAACG
TCACTGGGATGTTCGCATAGGCGGCC
Protein sequence PAB1686 - rAB-cetHS-puro[mAnti-DCIR_9E8_H-LV-hIgG4H-C-Flex-v1-
m.tbCFP10 (M. tb. antigen - bold)-f2-m.tbESAT6 (M. tb. antigen - underlined)].
(SEQ ID NO: 149)
QVTLKESGPGILQPSQTLSLTCSFSGFSLSTSGMGLSWIRQPSGKGLEWLAHIYWDDDKRY
NPSLKSRLTISKDTSSNQVFLKITIVDTADAATYYCARSSHYYGYGYGGYFDVWGAGTTVTVSSAK
TKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFEGGPSVFLFPPKPKDTLMISRTP
EVTCVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSLSLSLGKASMAE
MKTDAATLAQEAGNFERISGDLKTQIDQVESTAGSLQGQWRGAAGTAAQAAVVRFQEAANK
QKQELDEISTNIRQAGVQYSRADEEQQQALSSQMGFEFPTSTPADSSTITPTATPTATPTIKGTSM
TEQQWNFAGIEAAASAIQGNVTSIHSLLDEGKQSLTKLAAAWGGSGSEAYQGVQQKWDATATEL
NNALQNLARTISEAGQAMASTEGNVTGMFA
C1589: rAB-cetHS-puro[hIgG4H-Flex-v1-m.tbCFP10 (M. tb. antigen - bold)-
f2-m.tbESAT6 (M. tb. antigen - underlined)].
(SEQ ID NO: 150)
ATGGACCTCCTGTGCAAGAACATGAAGCACCTGTGGTTCTTCCTCCTGCTGGTGGCGGC
TCCCAGATGGGTCCTGTCCCGGCTGCAGCTGCAGGAGTCGGGCCCAGGCCTGCTGAAGCCTTC
GGTGACCCTGTCCCTCACCTGCACTGTCTCGGGTGACTCCGTCGCCAGTAGTTCTTATTACTGG
GGCTGGGTCCGTCAGCCCCCAGGGAAGGGACTCGAGTGGATAGGGACTATCAATTTTAGTGGC
AATATGTATTATAGTCCGTCCCTCAGGAGTCGAGTGACCATGTCGGCAGACATGTCCGAGAAC
TCCTTCTATCTGAAATTGGACTCTGTGACCGCAGCAGACACGGCCGTCTATTATTGTGCGGCAG
GACACCTCGTTATGGGATTTGGGGCCCACTGGGGACAGGGAAAACTGGTCTCCGTCTCTCCAG
CTTCCACCAAGGGCCCATCCGTCTTCCCCCTGGCGCCCTGCTCCAGGAGCACCTCCGAGAGCAC
AGCCGCCCTGGGCTGCCTGGTCAAGGACTACTTCCCCGAACCGGTGACGGTGTCGTGGAACTC
AGGCGCCCTGACCAGCGGCGTGCACACCTTCCCGGCTGTCCTACAGTCCTCAGGACTCTACTCC
CTCAGCAGCGTGGTGACCGTGCCCTCCAGCAGCTTGGGCACGAAGACCTACACCTGCAACGTA
GATCACAAGCCCAGCAACACCAAGGTGGACAAGAGAGTTGAGTCCAAATATGGTCCCCCATG
CCCACCCTGCCCAGCACCTGAGTTCGAAGGGGGACCATCAGTCTTCCTGTTCCCCCCAAAACCC
AAGGACACTCTCATGATCTCCCGGACCCCTGAGGTCACGTGCGTGGTGGTGGACGTGAGCCAG
GAAGACCCCGAGGTCCAGTTCAACTGGTACGTGGATGGCGTGGAGGTGCATAATGCCAAGAC
AAAGCCGCGGGAGGAGCAGTTCAACAGCACGTACCGTGTGGTCAGCGTCCTCACCGTCCTGCA
CCAGGACTGGCTGAACGGCAAGGAGTACAAGTGCAAGGTCTCCAACAAAGGCCTCCCGTCCTC
CATCGAGAAAACCATCTCCAAAGCCAAAGGGCAGCCCCGAGAGCCACAGGTGTACACCCTGC
CCCCATCCCAGGAGGAGATGACCAAGAACCAGGTCAGCCTGACCTGCCTGGTCAAAGGCTTCT
ACCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACAACCACT
ACACACAGAAGAGCCTCTCCCTGTCTCTGGGTAAAGCTAGCATGGCAGAGATGAAGACCGATG
CCGCTACCCTCGCGCAGGAGGCAGGTAATTTCGAGCGGATCTCCGGCGACCTGAAAACCCAGA
TCGACCAGGTGGAGTCGACGGCAGGTTCGTTGCAGGGCCAGTGGCGCGGCGCGGCGGGGACG
GCCGCCCAGGCCGCGGTGGTGCGCTTCCAAGAAGCAGCCAATAAGCAGAAGCAGGAACTCGA
CGAGATCTCGACGAATATTCGTCAGGCCGGCGTCCAATACTCGAGGGCCGACGAGGAGCAGC
AGCAGGCGCTGTCCTCGCAAATGGGCTTCGAATTCCCCACCAGCACCCCCGCCGACAGCAGCA
CCATCACCCCCACCGCCACCCCCACCGCCACCCCCACCATCAAGGGCACTAGCATGACAGAGC
AGCAGTGGAATTTCGCGGGTATCGAGGCCGCGGCAAGCGCAATCCAGGGAAATGTCACGTCC
ATTCATTCCCTCCTTGACGAGGGGAAGCAGTCCCTGACCAAGCTCGCAGCGGCCTGGGGCGGT
AGCGGTTCGGAGGCGTACCAGGGTGTCCAGCAAAAATGGGACGCCACGGCTACCGAGCTGAA
CAACGCGCTGCAGAACCTGGCGCGGACGATCAGCGAAGCCGGTCAGGCAATGGCTTCGACCG
AAGGCAACGTCACTGGGATGTTCGCATAGGCGGCC
Protein sequence PAB1687 - rAB-cetHS-puro[hIgG4H-Flex-v1-m.tbCFP10 (M. tb.
antigen - bold)-f2-m.tbESAT6 (M. tb. antigen - underlined)].
(SEQ ID NO: 151)
RLQLQESGPGLLKPSVTLSLTCTVSGDSVASSSYYWGWVRQPPGKGLEWIGTINFSGNMYY
SPSLRSRVTMSADMSENSFYLKLDSVTAADTAVYYCAAGHLVMGFGAHWGQGKLVSVSPASTKG
PSVFPLAPCSRSTSESTAALGCLVKDYFPEPVTVSWNSGALTSGVHTFPAVLQSSGLYSLSSVVTVP
SSSLGTKTYTCNVDHKPSNTKVDKRVESKYGPPCPPCPAPEFEGGPSVFLFPPKPKDTLMISRTPEVT
CVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKASMAEMK
TDAATLAQEAGNFERISGDLKTQIDQVESTAGSLQGQWRGAAGTAAQAAVVRFQEAANKQK
QELDEISTNIRQAGVQYSRADEEQQQALSSQMGFEFPTSTPADSSTITPTATPTATPTIKGTSMTE
QQWNFAGIEAAASAIQGNVTSIHSLLDEGKQSLTKLAAAWGGSGSEAYQGVQQKWDATATELNN
ALQNLARTISEAGQAMASTEGNVTGMFA
C1588: rAB-cetHS-puro[manti-CD40_12E12.3F3_H-V-SLAML-hIgG4H-C-Flex-v1-m.tbCFP10
(M. tb. antigen - bold)-f2-m.tbESAT6 (M. tb. antigen - underlined)].
(SEQ ID NO: 152)
ATGGACCCCAAAGGCTCCCTTTCCTGGAGAATACTTCTGTTTCTCTCCCTGGCTTTTGAG
TTGTCGTACGGAGAAGTGAAGCTGGTGGAGTCTGGGGGAGGCTTAGTGCAGCCTGGAGGGTCC
CTGAAACTCTCCTGTGCAACCTCTGGATTCACTTTCAGTGACTATTACATGTATTGGGTTCGCC
AGACTCCAGAGAAGAGGCTGGAGTGGGTCGCATACATTAATTCTGGTGGTGGTAGCACCTATT
ATCCAGACACTGTAAAGGGCCGATTCACCATCTCCAGAGACAATGCCAAGAACACCCTGTACC
TGCAAATGAGCCGGCTGAAGTCTGAGGACACAGCCATGTATTACTGTGCAAGACGGGGGTTAC
CGTTCCATGCTATGGACTATTGGGGTCAAGGAACCTCAGTCACCGTCTCCTCAGCCAAAACGA
AGGGCCCATCCGTCTTCCCCCTGGCGCCCTGCTCCAGGAGCACCTCCGAGAGCACAGCCGCCC
TGGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGCAG
CGTGGTGACCGTGCCCTCCAGCAGCTTGGGCACGAAGACCTACACCTGCAACGTAGATCACAA
GCCCAGCAACACCAAGGTGGACAAGAGAGTTGAGTCCAAATATGGTCCCCCATGCCCACCCTG
CCCAGCACCTGAGTTCGAAGGGGGACCATCAGTCTTCCTGTTCCCCCCAAAACCCAAGGACAC
TCTCATGATCTCCCGGACCCCTGAGGTCACGTGCGTGGTGGTGGACGTGAGCCAGGAAGACCC
CGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCCCATCCC
AGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTACACACAG
AAGAGCCTCTCCCTGTCTCTGGGTAAAGCTAGCATGGCAGAGATGAAGACCGATGCCGCTACC
CTCGCGCAGGAGGCAGGTAATTTCGAGCGGATCTCCGGCGACCTGAAAACCCAGATCGACCAG
GTGGAGTCGACGGCAGGTTCGTTGCAGGGCCAGTGGCGCGGCGCGGCGGGGACGGCCGCCCA
GGCCGCGGTGGTGCGCTTCCAAGAAGCAGCCAATAAGCAGAAGCAGGAACTCGACGAGATCT
CGACGAATATTCGTCAGGCCGGCGTCCAATACTCGAGGGCCGACGAGGAGCAGCAGCAGGCG
CTGTCCTCGCAAATGGGCTTCGAATTCCCCACCAGCACCCCCGCCGACAGCAGCACCATCACC
CCCACCGCCACCCCCACCGCCACCCCCACCATCAAGGGCACTAGCATGACAGAGCAGCAGTGG
AATTTCGCGGGTATCGAGGCCGCGGCAAGCGCAATCCAGGGAAATGTCACGTCCATTCATTCC
CTCCTTGACGAGGGGAAGCAGTCCCTGACCAAGCTCGCAGCGGCCTGGGGCGGTAGCGGTTCG
GAGGCGTACCAGGGTGTCCAGCAAAAATGGGACGCCACGGCTACCGAGCTGAACAACGCGCT
GCAGAACCTGGCGCGGACGATCAGCGAAGCCGGTCAGGCAATGGCTTCGACCGAAGGCAACG
TCACTGGGATGTTCGCATAGGCGGCC
Protein sequence PAB1685 - rAB-cetHS-puro[manti-CD40_12E12.3F3_H-V-SLAML-hIgG4H-C-
Flex-v1-m.tbCFP10 (M. tb. antigen - bold)-f2-m.tbESAT6 (M. tb. antigen -
underlined)].
(SEQ ID NO: 153)
EVKLVESGGGLVQPGGSLKLSCATSGFTFSDYYMYWVRQTPEKRLEWVAYINSGGGSTYY
PDTVKGRFTISRDNAKNTLYLQMSRLKSEDTAMYYCARRGLPFHAMDYWGQGTSVTVSSAKTKG
PSVFPLAPCSRSTSESTAALGCLVKDYFPEPVTVSWNSGALTSGVHTFPAVLQSSGLYSLSSVVTVP
SSSLGTKTYTCNVDHKPSNTKVDKRVESKYGPPCPPCPAPEFEGGPSVFLFPPKPKDTLMISRTPEVT
CVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGKASMAEMK
TDAATLAQEAGNFERISGDLKTQIDQVESTAGSLQGQWRGAAGTAAQAAVVRFQEAANKQK
QELDEISTNIRQAGVQYSRADEEQQQALSSQMGFEFPTSTPADSSTITPTATPTATPTIKGTSMTE
QQWNFAGIEAAASAIQGNVTSIHSLLDEGKQSLTKLAAAWGGSGSEAYQGVQQKWDATATELNN
ALQNLARTISEAGQAMASTEGNVTGMFA
C1261: rAB-pIRES2[mAnti-DCIR_9E8_H-LV-hIgG4H-C-Flex-v1-m.tbHspX (M. tb. antigen -
bold)].
(SEQ ID NO: 154)
ATGAACAGGCTTACTTCCTCATTGCTGCTGCTGATTGTCCCTGCATATGTCCTGTCCCAG
GTTACTCTGAAAGAGTCTGGCCCTGGGATATTGCAGCCCTCCCAGACCCTCAGTCTGACTTGTT
CTTTCTCTGGGTTTTCACTGAGCACTTCTGGTATGGGTCTGAGCTGGATTCGTCAGCCTTCAGG
AAAGGGTCTGGAGTGGCTGGCACACATTTACTGGGATGATGACAAGCGCTATAACCCATCCCT
GAAGAGCCGGCTCACAATCTCCAAGGATACCTCCAGCAACCAGGTTTTCCTCAAGATCACCAT
TGTGGACACTGCAGATGCTGCCACATACTACTGTGCTCGAAGCTCCCATTACTACGGTTATGGC
TACGGGGGATACTTCGATGTCTGGGGCGCAGGGACCACGGTCACCGTCTCCTCAGCCAAAACG
AAGGGCCCATCCGTCTTCCCCCTGGCGCCCTGCTCCAGGAGCACCTCCGAGAGCACAGCCGCC
CTGGGCTGCCTGGTCAAGGACTACTTCCCCGAACCGGTGACGGTGTCGTGGAACTCAGGCGCC
CTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACGAAGACCTACACCTGCAACGTAGATCACA
AGCCCAGCAACACCAAGGTGGACAAGAGAGTTGAGTCCAAATATGGTCCCCCATGCCCACCCT
GCCCAGCACCTGAGTTCGAAGGGGGACCATCAGTCTTCCTGTTCCCCCCAAAACCCAAGGACA
CTCTCATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCAGGACT
GGCTGAACGGCAAGGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACCCTGCCCCCATCCC
AGGAGGAGATGACCAAGAACCAGGTCAGCCTGACCTGCCTGGTCAAAGGCTTCTACCCCAGCG
ACATCGCCGTGGAGTGGGAGAGCAATGGGCAGCCGGAGAACAACTACAAGACCACGCCTCCC
GTGCTGGACTCCGACGGCTCCTTCTTCCTCTACAGCAGGCTAACCGTGGACAAGAGCAGGTGG
CAGGAGGGGAATGTCTTCTCATGCTCCGTGATGCATGAGGCTCTGCACAACCACTACACACAG
AAGAGCCTCTCCCTGTCTCTGGGTAAAGCTAGTCAGACCCCCACCAACACCATCAGCGTGACC
CCCACCAACAACAGCACCCCCACCAACAACAGCAACCCCAAGCCCAACCCCGCTAGCATGGCC
ACCACCCTTCCCGTTCAGCGCCACCCGCGGTCCCTCTTCCCCGAGTTTTCTGAGCTGTTCGCGG
CCTTCCCGTCATTCGCCGGACTCCGGCCCACCTTCGACACCCGGTTGATGCGGCTGGAAGACG
AGATGAAAGAGGGGCGCTACGAGGTACGCGCGGAGCTTCCCGGGGTCGACCCCGACAAGGAC
GTCGACATTATGGTCCGCGATGGTCAGCTGACCATCAAGGCCGAGCGCACCGAGCAGAAGGA
CTTCGACGGTCGCTCGGAATTCGCGTACGGTTCCTTCGTTCGCACGGTGTCGCTGCCGGTAGGT
GCTGACGAGGACGACATTAAGGCCACCTACGACAAGGGCATTCTTACTGTGTCGGTGGCGGTT
TCGGAAGGGAAGCCAACCGAAAAGCACATTCAGATCCGGTCCACCAACTGA
Protein sequence PAB1360-rAB-pIRES2[mAnti-DCIR_9E8_H-LV-hIgG4H-C-Flex-v1-m.tbHspX
(M. tb. antigen - bold)].
(SEQ ID NO: 155)
QVTLKESGPGILQPSQTLSLTCSFSGFSLSTSGMGLSWIRQPSGKGLEWLAHIYWDDDKRY
NPSLKSRLTISKDTSSNQVFLKITIVDTADAATYYCARSSHYYGYGYGGYFDVWGAGTTVTVSSAK
TKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFEGGPSVFLFPPKPKDTLMISRTP
EVTCVVVDVSQEDPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSLSLSLGKASQTPT
NTISVTPTNNSTPTNNSNPKPNPASMATTLPVQRHPRSLFPEFSELFAAFPSFAGLRPTFDTRLM
RLEDEMKEGRYEVRAELPGVDPDKDVDIMVRDGQLTIKAERTEQKDFDGRSEFAYGSFVR
Rv1626: C1256: Ecoli-pET28[6xHis-Cohesin (underlined)-m.tbRv1626 (M. tb.
Antigen - bold)].
(SEQ ID NO: 156)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGACCGGCCCCACCACCGACGCCGATGCCGCTGTCCCACG
TCGGGTCTTGATCGCGGAAGATGAAGCGCTCATCCGCATGGACCTGGCCGAGATGTTGCGAGA
GGAGGGATATGAAATTGTCGGCGAGGCCGGCGACGGCCAGGAAGCCGTCGAGCTGGCCGAGC
TGCACAAGCCCGACCTGGTGATCATGGACGTGAAGATGCCGCGCCGGGACGGGATCGACGCC
GCATCCGAAATCGCCAGCAAACGTATTGCCCCGATCGTGGTGCTGACCGCGTTCAGCCAGCGT
GATCTGGTCGAACGTGCGCGTGATGCCGGGGCGATGGCATACCTGGTAAAGCCTTTCAGCATC
AGCGACCTGATTCCAGCGATTGAATTGGCGGTCAGCCGGTTCAGGGAGATCACCGCGTTGGAA
GGCGAGGTGGCGACGCTATCTGAACGGTTGGAAACCCGCAAGCTGGTGGAACGAGCAAAAGG
CCTGCTGCAGACCAAACATGGGATGACCGAGCCGGACGCTTTCAAGTGGATTCAACGTGCCGC
CATGGATCGGCGCACCACCATGAAGCGGGTGGCCGAAGTCGTGCTGGAAACCCTCGGAACAC
CCAAAGACACCTGAGCGGCCGCACTCGAGCACCACCACCACCACCACTGAGATCCGGCTGCTA
ACAAAGCCCGAAAGGAAGCTGAGTTGGCTGCTGCCACCGCTGAGCAATAACTAGCATAACCCC
TTGGGGCCTCTAAACGGGTCTTGAGGGGTTTTTTGCTGAAAGGAGGAACTATATCCGGAT
Protein sequence - C1256: Ecoli-pET28[6xHis-Cohesin (underlined)-m.tbRv1626
(M. tb. Antigen - bold)]
(SEQ ID NO: 157)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MTGPTTDADAAVPRRVLIAEDEALIRMDLAEMLREEGYEIVGEAGDGQEAVELAELHKPDLV
IMDVKMPRRDGIDAASEIASKRIAPIVVLTAFSQRDLVERARDAGAMAYLVKPFSISDLIPAIEL
AVSRFREITALEGEVATLSERLETRKLVERAKGLLQTKHGMTEPDAFKWIQRAAMDRRTTM
KRVAEVVLETLGTPKDT
Rv3044: C1312: Ecoli-pET28[6xHis-Cohesin (underlined)-m.tbRv3044 (M. tb.
Antigen in bold)]
(SEQ ID NO: 158)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGCGATCCACTGTTGCTGTCGCCGTAGCGGCAGCCGT
GATCGCAGCGTCCAGTGGTTGCGGCTCCGATCAACCGGCCCATAAGGCGTCACAATCGA
TGATCACGCCCACCACCCAGATCGCCGGCGCCGGGGTGCTGGGAAACGACAGAAAGCCG
GATGAGTCGTGCGCGCGTGCGGCGGCCGCGGCCGATCCGGGGCCACCGACCCGACCAG
CGCACAATGCGGCGGGAGTCAGCCCGGAGATGGTGCAGGTGCCGGCGGAGGCGCAGCG
CATCGTGGTGCTCTCCGGTGACCAGCTCGACGCGCTGTGCGCGCTGGGCCTGCAATCGC
GGATCGTCGCCGCCGCGTTGCCGAACAGCTCCTCAAGTCAACCTTCCTATCTGGGCACG
ACCGTGCATGATCTGCCCGGTGTCGGTACTCGCAGCGCCCCCGACCTGCGCGCCATTGC
GGCGGCTCACCCGGATCTGATCCTGGGTTCGCAGGGTTTGACGCCGCAGTTGTATCCGC
AGCTGGCGGCGATCGCCCCGACGGTGTTTACCGCGGCACCGGGCGCGGACTGGGAAAAT
AACCTGCGTGGTGTCGGTGCCGCCACGGCCCGTATCGCCGCGGTGGACGCGCTGATCAC
CGGGTTCGCCGAACACGCCACCCAGGTCGGGACCAAGCATGACGCGACCCACTTCCAAG
CGTCGATCGTGCAGCTGACCGCCAACACCATGCGGGTATACGGCGCCAACAACTTCCCG
GCCAGCGTGCTGAGCGCGGTCGGCGTCGACCGACCGCCGTCTCAACGGTTCACCGACAA
GGCCTACATCGAGATCGGCACCACGGCCGCCGACCTGGCGAAATCACCGGACTTCTCGG
CGGCCGACGCCGATATCGTCTACCTGTCGTGCGCGTCGGAAGCAGCCGCGGAACGCGCG
GCCGTCATCCTGGATAGCGACCCATGGCGCAAGCTGTCCGCCAACCGTGACAACCGGGT
CTTCGTCGTCAACGACCAGGTATGGCAGACCGGCGAGGGTATGGTCGCTGCCCGCGGCA
TTGTCGATGATCTGCGCTGGGTCGACGCGCCGATCAACTAGAAGCTTGCGGCCGCACTCG
AGCACCACCACCACCACCACTGAGATCCGGCTGCTAACAAAGCCCGAAAGGAAGCTGAGTTG
GCTGCTGCCACCGCTGAGCAATAACTAGCATAACCCCTTGGGGCCTCTAAACGGGTCTTGAGG
GGTTTTTTGCTGAAAGGAGGAACTATATCCGGAT
Protein sequence - C1312: Ecoli-pET28[6xHis-Cohesin (underlined)-m.tbRv3044
(M. tb. Antigen in bold)]
(SEQ ID NO: 159)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MRSTVAVAVAAAVIAASSGCGSDQPAHKASQSMITPTTQIAGAGVLGNDRKPDESCARAAAA
ADPGPPTRPAHNAAGVSPEMVQVPAEAQRIVVLSGDQLDALCALGLQSRIVAAALPNSSSSQP
SYLGTTVHDLPGVGTRSAPDLRAIAAAHPDLILGSQGLTPQLYPQLAAIAPTVFTAAPGADWE
NNLRGVGAATARIAAVDALITGFAEHATQVGTKHDATHFQASIVQLTANTMRVYGANNFPAS
VLSAVGVDRPPSQRFTDKAYIEIGTTAADLAKSPDFSAADADIVYLSCASEAAAERAAVILDSD
PWRKLSANRDNRVFVVNDQVWQTGEGMVAARGIVDDLRWVDAPIN
Ag85 A: C1241: Ecoli-pET28[6xHis-Cohesin (underlined)-m.tbAg85A (M. tb antigen -
bold)]
(SEQ ID NO: 160)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGCAGCTTGTTGACAGGGTTCGTGGCGCCGTCACGGG
TATGTCGCGTCGACTCGTGGTCGGGGCCGTCGGCGCGGCCCTAGTGTCGGGTCTGGTCG
GCGCCGTCGGTGGCACGGCGACCGCGGGGGCATTTTCCCGGCCGGGCTTGCCGGTGGA
GTACCTGCAGGTGCCGTCGCCGTCGATGGGCCGTGACATCAAGGTCCAATTCCAAAGTG
GTGGTGCCAACTCGCCCGCCCTGTACCTGCTCGACGGCCTGCGCGCGCAGGACGACTTC
AGCGGCTGGGACATCAACACCCCGGCGTTCGAGTGGTACGACCAGTCGGGCCTGTCGGT
GGTCATGCCGGTGGGTGGCCAGTCAAGCTTCTACTCCGACTGGTACCAGCCCGCCTGCG
GCAAGGCCGGTTGCCAGACTTACAAGTGGGAGACCTTCCTGACCAGCGAGCTGCCGGGG
TGGCTGCAGGCCAACAGGCACGTCAAGCCCACCGGAAGCGCCGTCGTCGGTCTTTCGAT
GGCTGCTTCTTCGGCGCTGACGCTGGCGATCTATCACCCCCAGCAGTTCGTCTACGCGG
GAGCGATGTCGGGCCTGTTGGACCCCTCCCAGGCGATGGGTCCCACCCTGATCGGCCTG
GCGATGGGTGACGCTGGCGGCTACAAGGCCTCCGACATGTGGGGCCCGAAGGAGGACC
CGGCGTGGCAGCGCAACGACCCGCTGTTGAACGTCGGGAAGCTGATCGCCAACAACACC
CGCGTCTGGGTGTACTGCGGCAACGGCAAGCCGTCGGATCTGGGTGGCAACAACCTGCC
GGCCAAGTTCCTCGAGGGCTTCGTGCGGACCAGCAACATCAAGTTCCAAGACGCCTACA
ACGCCGGTGGCGGCCACAACGGCGTGTTCGACTTCCCGGACAGCGGTACGCACAGCTGG
GAGTACTGGGGCGCGCAGCTCAACGCTATGAAGCCCGACCTGCAACGGGCACTGGGTGC
CACGCCCAACACCGGGCCCGCGCCCCAGGGCGCCTAGGCGGCCGCACTCGAGCACCACCA
CCACCACCACTGAGATCCGGCTGCTAACAAAGCCCGAAAGGAAGCTGAGTTGGCTGCTGCCAC
CGCTGAGCAATAACTAGCATAACCCCTTGGGGCCTCTAAACGGGTCTTGAGGGGTTTTTTGCTG
AAAGGAGGAACTATATCCGGAT
Protein sequence - Ecoli-pET28[6xHis-Cohesin (underlined)-m.tbAg85A (M. tb
antigen - bold)].
(SEQ ID NO: 161)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MQLVDRVRGAVTGMSRRLVVGAVGAALVSGLVGAVGGTATAGAFSRPGLPVEYLQVPSPSM
GRDIKVQFQSGGANSPALYLLDGLRAQDDFSGWDINTPAFEWYDQSGLSVVMPVGGQSSFYS
DWYQPACGKAGCQTYKWETFLTSELPGWLQANRHVKPTGSAVVGLSMAASSALTLAIYHPQ
QFVYAGAMSGLLDPSQAMGPTLIGLAMGDAGGYKASDMWGPKEDPAWQRNDPLLNVGKLI
ANNTRVWVYCGNGKPSDLGGNNLPAKFLEGFVRTSNIKFQDAYNAGGGHNGVFDFPDSGTH
SWEYWGAQLNAMKPDLQRALGATPNTGPAPQGA
Rv1860: C2342: Ecoli-pET28[6xHis-Cohesin (bold)-m.tbRv1860 (m. tb.Antigen - bold)].
(SEQ ID NO: 162)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGC [symbol] CAAGGGACG
ATTGGCGGCACTGGCTATCGCGGCGATGGCCAGCGCCAGCCTGGTGACCGTTGCGGTGC
CCGCGACCGCCAACGCCGATCCGGAGCCAGCGCCCCCGGTACCCACAACGGCCGCCTCG
CCGCCGTCGACCGCTGCAGCGCCACCCGCACCGGCGACACCTGTTGCCCCCCCACCACC
GGCCGCCGCCAACACGCCGAATGCCCAGCCGGGCGATCCCAACGCAGCACCTCCGCCGG
CCGACCCGAACGCACCGCCGCCACCTGTCATTGCCCCAAACGCACCCCAACCTGTCCGG
ATCGACAACCCGGTTGGAGGATTCAGCTTCGCGCTGCCTGCTGGCTGGGTGGAGTCTGA
CGCCGCCCACTTCGACTACGGTTCAGCACTCCTCAGCAAAACCACCGGGGACCCGCCAT
TTCCCGGACAGCCGCCGCCGGTGGCCAATGACACCCGTATCGTGCTCGGCCGGCTAGAC
CAAAAGCTTTACGCCAGCGCCGAAGCCACCGACTCCAAGGCCGCGGCCCGGTTGGGCTC
GGACATGGGTGAGTTCTATATGCCCTACCCGGGCACCCGGATCAACCAGGAAACCGTCT
CGCTCGACGCCAACGGGGTGTCTGGAAGCGCGTCGTATTACGAAGTCAAGTTCAGCGAT
CCGAGTAAGCCGAACGGCCAGATCTGGACGGGCGTAATCGGCTCGCCCGCGGCGAACGC
ACCGGACGCCGGGCCCCCTCAGCGCTGGTTTGTGGTATGGCTCGGGACCGCCAACAACC
CGGTGGACAAGGGCGCGGCCAAGGCGCTGGCCGAATCGATCCGGCCTTTGGTCGCCCCG
CCGCCGGCGCCGGCACCGGCTCCTGCAGAGCCCGCTCCGGCGCCGGCGCCGGCCGGGG
AAGTCGCTCCTACCCCGACGACACCGACACCGCAGCGGACCTTACCGGCCGAATTCTGAG
CGGCCGCACTCGAGCACCACCACCACCACCACTGAGATCCGGCTGCTAACAAAGCCCGAAAG
GAAGCTGAGTTGGCTGCTGCCACCGCTGAGCAATAACTAGCATAACCCCTTGGGGCCTCTAAA
CGGGTCTTGAGGGGTTTTTTGCTGAAAGGAGGAACTATATCCGGAT
Protein sequence - C2342: Ecoli-pET28[6xHis-Cohesin (bold) -m.tbRv1860 (m. tb.
Antigen - bold)].
(SEQ ID NO: 163)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MHQVDPNLTRRKGRLAALAIAAMASASLVTVAVPATANADPEPAPPVPTTAASPPSTAAAPPA
PATPVAPPPPAAANTPNAQPGDPNAAPPPADPNAPPPPVIAPNAPQPVRIDNPVGGFSFALPAG
WVESDAAHFDYGSALLSKTTGDPPFPGQPPPVANDTRIVLGRLDQKLYASAEATDSKAAARL
GSDMGEFYMPYPGTRINQETVSLDANGVSGSASYYEVKFSDPSKPNGQIWTGVIGSPAANAPD
AGPPQRWFVVWLGTANNPVDKGAAKALAESIRPLVAPPPAPAPAPAEPAPAPAPAGEVAPTP
TTPTPQRTLPA
Rv3812: C1255: Ecoli-pET28[6xHis-Cohesin (underlined)-m.tbRv3812 (M. tb.
Antigen - bold)].
(SEQ ID NO: 164)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGTGTGTCGTTCGTGGTCACAGTGCCGGAGGCCGTGGCGGCTGC
GGCGGGGGATTTGGCGGCCATCGGCTCGACGCTTCGGGAAGCGACCGCTGCGGCGGCGGGCC
CCACGACCGGGCTGGCGGCCGCGGCCGCCGACGACGTGTCGATCGCTGTCTCGCAGCTGTTCG
GCAGGTACGGCCAGGAATTTCAAACCGTGAGCAACCAACTGGCCGCGTTTCATACCGAGTTCG
TACGCACGTTGAACCGCGGCGCGGCGGCGTATCTCAACACCGAAAGCGCTAACGGCGGGCAG
CTGTTCGGTCAGATCGAGGCGGGACAGCGCGCCGTTTCCGCGGCCGCGGCCGCCGCTCCGGGC
GGCGCATACGGCCAACTCGTTGCCAACACGGCCACCAACCTGGAATCCCTCTACGGCGCATGG
TCGGCCAACCCGTTCCCATTCCTCCGCCAGATCATCGCCAACCAGCAGGTTTACTGGCAGCAG
ATCGCCGCGGCGCTCGCCAACGCCGTCCAGAACTTCCCCGCCCTGGTGGCGAATTTGCCAGCG
GCCATCGACGCGGCCGTCCAGCAATTCCTGGCCTTCAACGCGGCGTACTACATCCAACAGATT
ATTAGCTCGCAGATCGGCTTCGCCCAGCTATTCGCCACGACGGTCGGTCAGGGGGTCACCAGC
GTCATTGCCGGGTGGCCCAACCTTGCGGCGGAGCTTCAGCTAGCGTTTCAACAGCTTCTGGTGG
GTGACTACAACGCCGCGGTGGCGAACCTGGGTAAGGCCATGACAAACCTTCTGGTCACCGGGT
TCGACACCAGCGACGTGACGATCGGCACAATGGGCACCACCATTAGTGTCACCGCGAAACCCA
AGCTGCTGGGCCCGCTGGGAGATCTGTTCACCATCATGACCATCCCGGCACAAGAGGCGCAGT
ACTTCACCAACCTGATGCCCCCCTCCATCCTGCGAGACATGTCGCAGAACTTCACCAACGTG
CTCACGACGCTCTCCAACCCGAACATCCAGGCGGTCGCTTCGTTCGATATCGCAACCACC
GCCGGGACTTTGAGCACCTTCTTCGGGGTGCCATTGGTGCTCACTTACGCCACATTGGGT
GCGCCGTTCGCGTCACTGAACGCGATTGCGACGAGCGCGGAAACCATCGAGCAGGCCCT
GTTGGCCGGCAACTACCTAGGGGCGGTGGGTGCGCTTATCGACGCCCCGGCCCACGCGT
TAGACGGCTTCCTCAACAGCGCAACCGTGTTGGATACGCCGATCCTGGTGCCCACGGGG
CTCCCGTCCCCTCTGCCCCCGACGGTCGGGATCACGCTGCACTTGCCTTTCGACGGGATT
CTCGTGCCGCCGCATCCCGTCACCGCGACGATCAGCTTCCCGGGTGCTCCGGTTCCTATT
CCCGGTTTCCCAACCACCGTAACCGTTTTCGGCACACCCTTCATGGGAATGGCTCCGCTG
CTGATCAACTACATTCCCCAACAGCTCGCCCTGGCAATCAAACCGGCGGCTTAGCTCGAG
CACCACCACCACCACCACTGAGATCCGGCTGCTAACAAAGCCCGAAAGGAAGCTGAGTTGGCT
GCTGCCACCGCTGAGCAATAACTAGCATAACCCCTTGGGGCCTCTAAACGGGTCTTGAGGGGT
TTTTTGCTGAAAGGAGGAACTATATCCGGAT
Protein sequence - Ecoli-pET28[6xHis-Cohesin (underlined)-m.tbRv3812 (M. tb.
Antigen - bold)].
(SEQ ID NO: 165)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
VSFVVTVPEAVAAAAGDLAAIGSTLREATAAAAGPTTGLAAAAADDVSIAVSQLFGRYGQEFQ
TVSNQLAAFHTEFVRTLNRGAAAYLNTESANGGQLFGQIEAGQRAVSAAAAAAPGGAYGQL
VANTATNLESLYGAWSANPFPFLRQIIANQQVYWQQIAAALANAVQNFPALVANLPAAIDAA
VQQFLAFNAAYYIQQIISSQIGFAQLFATTVGQGVTSVIAGWPNLAAELQLAFQQLLVGDYNA
AVANLGKAMTNLLVTGFDTSDVTIGTMGTTISVTAKPKLLGPLGDLFTIMTIPAQEAQYFTNL
MPPSILRDMSQNFTNVLTTLSNPNIQAVASFDIATTAGTLSTFFGVPLVLTYATLGAPFASLNAI
ATSAETIEQALLAGNYLGAVGALIDAPAHALDGFLNSATVLDTPILVPTGLPSPLPPTVGITLH
LPFDGILVPPHPVTATISFPGAPVPIPGFPTTVTVFGTPFMGMAPLLINYIPQQLALAIKPAA
Rv3878: C2343: Ecoli-pET28[6xHis-Cohesin (underlined)-m.tbRv3878 (M. tb.
Antigen - bold)].
(SEQ ID NO: 166)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGGCTGAACCGTTGGCCGTCGATCCCACCGGCTTGAG
CGCAGCGGCCGCGAAATTGGCCGGCCTCGTTTTTCCGCAGCCTCCGGCGCCGATCGCGG
TCAGCGGAACGGATTCGGTGGTAGCAGCAATCAACGAGACCATGCCAAGCATCGAATCG
CTGGTCAGTGACGGGCTGCCCGGCGTGAAAGCCGCCCTGACTCGAACAGCATCCAACAT
GAACGCGGCGGCGGACGTCTATGCGAAGACCGATCAGTCACTGGGAACCAGTTTGAGCC
AGTATGCATTCGGCTCGTCGGGCGAAGGCCTGGCTGGCGTCGCCTCGGTCGGTGGTCAG
CCAAGTCAGGCTACCCAGCTGCTGAGCACACCCGTGTCACAGGTCACGACCCAGCTCGG
CGAGACGGCCGCTGAGCTGGCACCCCGTGTTGTTGCGACGGTGCCGCAACTCGTTCAGC
TGGCTCCGCACGCCGTTCAGATGTCGCAAAACGCATCCCCCATCGCTCAGACGATCAGTC
AAACCGCCCAACAGGCCGCCCAGAGCGCGCAGGGCGGCAGCGGCCCAATGCCCGCACA
GCTTGCCAGCGCTGAAAAACCGGCCACCGAGCAAGCGGAGCCGGTCCACGAAGTGACAA
ACGACGATCAGGGCGACCAGGGCGACGTGCAGCCGGCCGAGGTCGTTGCCGCGGCACG
TGACGAAGGCGCCGGCGCATCACCGGGCCAGCAGCCCGGCGGGGGCGTTCCCGCGCAA
GCCATGGATACCGGAGCCGGTGCCCGCCCAGCGGCGAGTCCGCTGGCGGCCCCCGTCG
ATCCGTCGACTCCGGCACCCTCAACAACCACAACGTTGGAATTCTAAAAGCTTGCGGCCGC
ACTCGAGCACCACCACCACCACCACTGAGATCCGGCTGCTAACAAAGCCCGAAAGGAAGCTG
AGTTGGCTGCTGCCACCGCTGAGCAATAACTAGCATAACCCCTTGGGGCCTCTAAACGGGTCT
TGAGGGGTTTTTTGCTGAAAGGAGGAACTATATCCGGAT
Protein sequence - Ecoli-pET28[6xHis-Cohesin (underlined) -m.tbRv3878 (M. tb.
Antigen - bold)].
(SEQ ID NO: 167)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MAEPLAVDPTGLSAAAAKLAGLVFPQPPAPIAVSGTDSVVAAINETMPSIESLVSDGLPGVKAA
LTRTASNMNAAADVYAKTDQSLGTSLSQYAFGSSGEGLAGVASVGGQPSQATQLLSTPVSQV
TTQLGETAAELAPRVVATVPQLVQLAPHAVQMSQNASPIAQTISQTAQQAAQSAQGGSGPMP
AQLASAEKPATEQAEPVHEVTNDDQGDQGDVQPAEVVAAARDEGAGASPGQQPGGGVPAQ
AMDTGAGARPAASPLAAPVDPSTPAPSTTTTLEF
Rv1837: C2341: Ecoli-pET28[6xHis-Cohesin (underlined) -m.tbRv1837 (M. tb.
antigen - bold)].
(SEQ ID NO: 168)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGACAGATCGCGTGTCGGTGGGCAACTTGCGCATCGC
TCGGGTGCTCTACGACTTCGTGAACAATGAAGCCCTGCCTGGCACCGATATCGACCCGG
ACAGCTTCTGGGCGGGCGTCGACAAGGTCGTCGCCGACCTGACCCCGCAGAACCAAGCT
CTGTTGAACGCCCGCGACGAGCTGCAGGCGCAGATCGACAAGTGGCACCGGCGTCGGGT
GATCGAGCCCATCGACATGGATGCCTACCGCCAGTTCCTCACCGAGATCGGCTACCTGCT
TCCCGAACCTGATGACTTCACCATCACCACGTCCGGTGTCGACGCTGAGATCACCACGAC
CGCCGGCCCCCAGCTGGTGGTGCCGGTGCTCAACGCGCGGTTTGCTCTGAACGCGGCCA
ACGCTCGCTGGGGCTCCCTCTACGACGCCTTGTATGGCACCGATGTCATCCCCGAGACC
GACGGCGCCGAAAAAGGCCCCACGTACAACAAGGTTCGTGGCGACAAGGTGATCGCGTA
TGCCCGCAAGTTCCTCGACGACAGTGTTCCGCTGTCGTCGGGTTCCTTTGGCGACGCCAC
CGGTTTCACAGTGCAGGATGGCCAGCTCGTGGTTGCCTTGCCGGATAAGTCCACCGGCC
TGGCCAACCCCGGCCAGTTCGCCGGCTACACCGGCGCAGCCGAGTCGCCGACATCGGTG
CTGCTAATCAATCACGGTTTGCACATCGAGATCCTGATCGATCCGGAGTCGCAGGTCGGC
ACCACCGACCGGGCCGGCGTCAAGGACGTGATCCTGGAATCCGCGATCACCACGATCAT
GGACTTCGAGGACTCGGTGGCCGCCGTGGACGCCGCCGACAAGGTGCTGGGTTATCGGA
ACTGGCTCGGCCTGAACAAGGGCGACCTGGCAGCAGCGGTAGACAAGGACGGCACCGCT
TTCCTGCGGGTGCTCAATAGGGACCGGAACTACACCGCACCCGGCGGTGGCCAGTTCAC
GCTGCCTGGACGCAGCCTCATGTTCGTCCGCAACGTCGGTCACTTGATGACGAATGACG
CCATCGTCGACACTGACGGCAGCGAGGTGTTCGAAGGCATCATGGATGCCCTATTCACC
GGCCTGATCGCCATCCACGGGCTAAAGGCCAGCGACGTCAACGGGCCGCTGATCAACAG
CCGCACCGGCTCCATCTACATCGTCAAGCCGAAGATGCACGGTCCGGCCGAGGTGGCGT
TTACCTGCGAACTGTTCAGCCGGGTTGAAGATGTGCTGGGGTTGCCGCAAAACACCATG
AAGATCGGCATCATGGACGAGGAACGCCGGACCACGGTCAACCTCAAGGCGTGCATCAA
AGCTGCCGCGGACCGCGTGGTGTTCATCAACACCGGGTTCCTGGACCGCACCGGCGATG
AAATCCACACCTCGATGGAGGCCGGCCCGATGGTGCGCAAGGGCACCATGAAGAGCCAG
CCGTGGATCTTGGCCTACGAGGACCACAACGTCGATGCCGGCCTGGCCGCCGGGTTCAG
CGGCCGAGCCCAGGTCGGCAAGGGCATGTGGACAATGACCGAGCTGATGGCCGACATG
GTCGAGACAAAAATCGCCCAGCCGCGCGCCGGGGCCAGCACCGCCTGGGTTCCCTCTCC
CACTGCGGCCACCCTGCATGCGCTGCACTACCACCAGGTCGACGTCGCCGCGGTGCAAC
AAGGACTGGCGGGGAAGCGTCGCGCCACCATCGAACAATTGCTGACCATTCCGCTGGCC
AAGGAATTGGCCTGGGCTCCCGACGAGATCCGCGAAGAGGTCGACAACAACTGTCAATC
CATCCTCGGCTACGTGGTTCGCTGGGTTGATCAAGGTGTCGGCTGCTCGAAGGTGCCCG
ACATCCACGACGTCGCGCTCATGGAGGACCGGGCCACGCTGCGAATCTCCAGCCAATTG
TTGGCCAACTGGCTGCGCCACGGTGTGATCACCAGCGCGGATGTGCGGGCCAGCTTGGA
GCGGATGGCGCCGTTGGTCGATCGACAAAACGCGGGCGACGTGGCATACCGACCGATGG
CACCCAACTTCGACGACAGTATCGCCTTCCTGGCCGCGCAGGAGCTGATCTTGTCCGGG
GCCCAGCAGCCCAACGGCTACACCGAGCCGATCCTGCACCGACGTCGTCGGGAGTTTAA
GGCCCGGGCCGCTGAGAAGCCGGCCCCATCGGACAGGGCCGGTGACGATGCGGCCCGC
GAATTCTAGGCGGCCGCACTCGAGCACCACCACCACCACCACTGAGATCCGGCTGCTAACAAA
GCCCGAAAGGAAGCTGAGTTGGCTGCTGCCACCGCTGAGCAATAACTAGCATAACCCCTTGGG
GCCTCTAAACGGGTCTTGAGGGGTTTTTTGCTGAAAGGAGGAACTATATCCGGAT
Protein sequence - Ecoli-pET28[6xHis-Cohesin (underlined) -m.tbRv1837 (M. tb.
antigen - bold)]
(SEQ ID NO: 169)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MTDRVSVGNLRIARVLYDFVNNEALPGTDIDPDSFWAGVDKVVADLTPQNQALLNARDELQA
QIDKWHRRRVIEPIDMDAYRQFLTEIGYLLPEPDDFTITTSGVDAEITTTAGPQLVVPVLNARF
ALNAANARWGSLYDALYGTDVIPETDGAEKGPTYNKVRGDKVIAYARKFLDDSVPLSSGSFG
DATGFTVQDGQLVVALPDKSTGLANPGQFAGYTGAAESPTSVLLINHGLHIEILIDPESQVGTT
DRAGVKDVILESAITTIMDFEDSVAAVDAADKVLGYRNWLGLNKGDLAAAVDKDGTAFLRV
LNRDRNYTAPGGGQFTLPGRSLMFVRNVGHLMTNDAIVDTDGSEVFEGIMDALFTGLIAIHG
LKASDVNGPLINSRTGSIYIVKPKMHGPAEVAFTCELFSRVEDVLGLPQNTMKIGIMDEERRT
TVNLKACIKAAADRVVFINTGFLDRTGDEIHTSMEAGPMVRKGTMKSQPWILAYEDHNVDA
GLAAGFSGRAQVGKGMWTMTELMADMVETKIAQPRAGASTAWVPSPTAATLHALHYHQV
DVAAVQQGLAGKRRATIEQLLTIPLAKELAWAPDEIREEVDNNCQSILGYVVRWVDQGVGCS
KVPDIHDVALMEDRATLRISSQLLANWLRHGVITSADVRASLERMAPLVDRQNAGDVAYRP
MAPNFDDSIAFLAAQELILSGAQQPNGYTEPILHRRRREFKARAAEKPAPSDRAGDDAAR
Rv3803: C2357: Ecoli-pET28 [6xHis-CthermoCohesin (underlined) -m.tbRv3803 (M. tb.
antigen - bold)].
(SEQ ID NO: 170)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCGCGGAACCCACCGCCAAGGCCGCCCCATACGAGAACCTGAT
GGTGCCGTCGCCCTCGATGGGCCGGGACATCCCGGTGGCCTTCCTAGCCGGTGGGCCGCACGC
GGTGTATCTGCTGGACGCCTTCAACGCCGGCCCGGATGTCAGTAACTGGGTCACCGCGGGTAA
CGCGATGAACACGTTGGCGGGCAAGGGGATTTCGGTGGTGGCACCGGCCGGTGGTGCGTACA
GCATGTACACCAACTGGGAGCAGGATGGCAGCAAGCAGTGGGACACCTTCTTGTCCGCTGAGC
TGCCCGACTGGCTGGCCGCTAACCGGGGCTTGGCCCCCGGTGGCCATGCGGCCGTTGGCGCCG
CTCAGGGCGGTTACGGGGCGATGGCGCTGGCGGCCTTCCACCCCGACCGCTTCGGCTTCGCTG
GCTCGATGTCGGGCTTTTTGTACCCGTCGAACACCACCACCAACGGTGCGATCGCGGCGGGCA
TGCAGCAATTCGGCGGTGTGGACACCAACGGAATGTGGGGAGCACCACAGCTGGGTCGGTGG
AAGTGGCACGACCCGTGGGTGCATGCCAGCCTGCTGGCGCAAAACAACACCCGGGTGTGGGT
GTGGAGCCCGACCAACCCGGGAGCCAGCGATCCCGCCGCCATGATCGGCCAAGCCGCCGAGG
CGATGGGTAACAGCCGCATGTTCTACAACCAGTATCGCAGCGTCGGCGGGCACAACGGACACT
TCGACTTCCCAGCCAGCGGTGACAACGGCTGGGGCTCGTGGGCGCCCCAGCTGGGCGCTATGT
CGGGCGATATCGTCGGTGCGATCCGCGAATTCTAAGCGGCCGCACTCGAGCACCACCACCACC
ACCACTGAGATCCGGCTGCTAACAAAGCCCGAAAGGAAGCTGAGTTGGCTGCTGCCACCGCTG
AGCAATAACTAGCATAACCCCTTGGGGCCTCTAAACGGGTCTTGAGGGGTTTTTTGCTGAAAG
GAGGAACTATATCCGGAT
Protein sequence - Ecoli-pET28 [6xHis-CthermoCohesin (underlined) -m.tbRv3803 (M.
tb. antigen - bold)].
(SEQ ID NO: 171)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
AEPTAKAAPYENLMVPSPSMGRDIPVAFLAGGPHAVYLLDAFNAGPDVSNWVTAGNAMNTL
AGKGISVVAPAGGAYSMYTNWEQDGSKQWDTFLSAELPDWLAANAIAAGMQQFGGVDTNG
MWGAPQLGRWKWHDPWVHASLLAQNNTRVWVWSPTNPGASDPAAMIGQAAEAMGNSRM
FYNQYRSVGGHNGHFDFPASGDNGWGSWAPQLGAMSGDIVGAIREF
HspX: C1253: Ecoli-pET28[6xHis-Cohesin (underlined) -m.tbHspX (M. tb. antigen -
bold)].
(SEQ ID NO: 172)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGGCCACCACCCTTCCCGTTCAGCGCCACCCGCGGTCCCTC
TTCCCCGAGTTTTCTGAGCTGTTCGCGGCCTCCCGTCATTCGCCGGACTCCGGCCCACCTTCG
ACACCCGGTTGATGCGGCTGGAAGACGAGATGAAAGAGGGGCGCTACGAGGTACGCGCGGAG
CTTCCCGGGGTCGACCCCGACAAGGACGTCGACATTATGGTCCGCGATGGTCAGCTGACCATC
AAGGCCGAGCGCACCGAGCAGAAGGACTTCGACGGTCGCTCGGAATTCGCGTACGGTTCCTTC
GTTCGCACGGTGTCGCTGCCGGTAGGTGCTGACGAGGACGACATTAAGGCCACCTACGACAAG
GGCATTCTTACTGTGTCGGTGGCGGTTTCGGAAGGGAAGCCAACCGAAAAGCACATTCAGATC
CGGTCCACCAACTGAGCGGCCGCACTCGAGCACCACCACCACCACCACTGAGATCCGGCTGCT
AACAAAGCCCGAAAGGAAGCTGAGTTGGCTGCTGCCACCGCTGAGCAATAACTAGCATAACC
CCTTGGGGCCTCTAAACGGGTCTTGAGGGGTTTTTTGCTGAAAGGAGGAACTATATCCGGAT
Protein sequence - Ecoli - pET28[6xHis-Cohesin (underlined) -m.tbHspX (M. tb.
antigen - bold)].
(SEQ ID NO: 173)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MATTLPVQRHPRSLFPEFSELFAAFPSFAGLRPTFDTRLMRLEDEMKEGRYEVRAELPGVDP
DKDVDIMVRDGQLTIKAERTEQKDFDGRSEFAYGSFVRTVSLPVGADEDDIKATYDKGILTVS
VAVSEGKPTEKHIQIRSTN
Rv2875: C1310: Ecoli-pET28[6xHis-Cohesin (underlined) -m.tbRv2875 (M. tb.
antigen - bold)].
(SEQ ID NO: 174)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGAAGGTAAAGAACACAATTGCGGCAACCAGTTTCGCGGC
GGCCGGCCTGGCGGCTCTGGCGGTGGCTGTCTCACCGCCGGCGGCCGCAGGCGATCTGGTGGG
CCCGGGCTGCGCGGAATACGCGGCAGCCAATCCCACTGGGCCGGCCTCGGTGCAGGGAATGTC
GCAGGACCCGGTCGCGGTGGCGGCCTCGAACAATCCGGAGTTGACAACGCTGACGGCTGCACT
GTCGGGCCAGCTCAATCCGCAAGTAAACCTGGTGGACACCCTCAACAGCGGTCAGTACACGGT
GTTCGCACCGACCAACGCGGCATTTAGCAAGCTGCCGGCATCCACGATCGACGAGCTCAAGAC
CAATTCGTCACTGCTGACCAGCATCCTGACCTACCACGTAGTGGCCGGCCAAACCAGCCCGGC
CAACGTCGTCGGCACCCGTCAGACCCTCCAGGGCGCCAGCGTGACGGTGACCGGTCAGGGTAA
CAGCCTCAAGGTCGGTAACGCCGACGTCGTCTGTGGTGGGGTGTCTACCGCCAACGCGACGGT
GTACATGATTGACAGCGTGCTAATGCCTCCGGCGTAAAAGCTTGCGGCCGCACTCGAGCACCA
CCACCACCACCACTGAGATCCGGCTGCTAACAAAGCCCGAAAGGAAGCTGAGTTGGCTGCTGC
CACCGCTGAGCAATAACTAGCATAACCCCTTGGGGCCTCTAAACGGGTCTTGAGGGGTTTTTTG
CTGAAAGGAGGAACTATATCCGGAT
Protein sequence - Ecoli-pET28[6xHis-Cohesin (underlined) -m.tbRv2875 (M. tb.
antigen - bold)]..
(SEQ ID NO: 175)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MKVKNTIAATSFAAAGLAALAVAVSPPAAAGDLVGPGCAEYAAANPTGPASVQGMSQDPVA
VAASNNPELTTLTAALSGQLNPQVNLVDTLNSGQYTVFAPTNAAFSKLPASTIDELKTNSSLLT
SILTYHVVAGQTSPANVVGTRQTLQGASVTVTGQGNSLKVGNADVVCGGVSTANATVYMIDS
VLMPPA
Rv0125: C1334: Ecoli-pET28[6xHis-Cohesin (underlined) -m.tbRv0125 (M. tb.
antigen - bold)].
(SEQ ID NO: 176)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGAGCAATTCGCGCCGCCGCTCACTCAGGTGGTCATGGTT
GCTGAGCGTGCTGGCTGCCGTCGGGCTGGGCCTGGCCACGGCGCCGGCCCAGGCGGCCCCGCC
GGCCTTGTCGCAGGACCGGTTCGCCGACTTCCCCGCGCTGCCCCTCGACCCGTCCGCGATGGTC
GCCCAAGTGGGGCCACAGGTGGTCAACATCAACACCAAACTGGGCTACAACAACGCCGTGGG
CGCCGGGACCGGCATCGTCATCGATCCCAACGGTGTCGTGCTGACCAACAACCACGTGATCGC
GGGCGCCACCGACATCAATGCGTTCAGCGTCGGCTCCGGCCAAACCTACGGCGTCGATGTGGT
CGGGTATGACCGCACCCAGGATGTCGCGGTGCTGCAGCTGCGCGGTGCCGGTGGCCTGCCGTC
GGCGGCGATCGGTGGCGGCGTCGCGGTTGGTGAGCCCGTCGTCGCGATGGGCAACAGCGGTG
GGCAGGGCGGAACGCCCCGTGCGGTGCCTGGCAGGGTGGTCGCGCTCGGCCAAACCGTGCAG
GCGTCGGATTCGCTGACCGGTGCCGAAGAGACATTGAACGGGTTGATCCAGTTCGATGCCGCG
ATCCAGCCCGGTGATTCGGGCGGGCCCGTCGTCAACGGCCTAGGACAGGTGGTCGGTATGAAC
ACGGCCGCGTCCGATAACTTCCAGCTGTCCCAGGGTGGGCAGGGATTCGCCATTCCGATCGGG
CAGGCGATGGCGATCGCGGGCCAGATCCGATCGGGTGGGGGGTCACCCACCGTTCATATCGGG
CCTACCGCCTTCCTCGGCTTGGGTGTTGTCGACAACAACGGCAACGGCGCACGAGTCCAACGC
GTGGTCGGGAGCGCTCCGGCGGCAAGTCTCGGCATCTCCACCGGCGACGTGATCACCGCGGTC
GACGGCGCTCCGATCAACTCGGCCACCGCGATGGCGGACGCGCTTAACGGGCATCATCCCGGT
GACGTCATCTCGGTGACCTGGCAAACCAAGTCGGGCGGCACGCGTACAGGGAACGTGACATTG
GCCGAGGGACCCCCGGCCTGAGCGGCCGCACTCGAGCACCACCACCACCACCACTGAGATCCG
GCTGCTAACAAAGCCCGAAAGGAAGCTGAGTTGGCTGCTGCCACCGCTGAGCAATAACTAGCA
TAACCCCTTGGGGCCTCTAAACGGGTCTTGAGGGGTTTTTTGCTGAAAGGAGGAACTATATCC
GGAT
Protein sequence - Ecoli-pET28[6xHis-Cohesin (underlined) -m.tbRv0125 (M. tb.
antigen - bold)].
(SEQ ID NO: 177)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MSNSRRRSLRWSWLLSVLAAVGLGLATAPAQAAPPALSQDRFADFPALPLDPSAMVAQVGPQ
VVNINTKLGYNNAVGAGTGIVIDPNGVVLTNNHVIAGATDINAFSVGSGQTYGVDVVGYDRT
QDVAVLQLRGAGGLPSAAIGGGVAVGEPVVAMGNSGGQGGTPRAVPGRVVALGQTVQASD
SLTGAEETLNGLIQFDAAIQPGDSGGPVVNGLGQVVGMNTAASDNFQLSQGGQGFAIPIGQA
MAIAGQIRSGGGSPTVHIGPTAFLGLGVVDNNGNGARVQRVVGSAPAASLGISTGDVITAVDG
APINSATAMADALNGHHPGDVISVTWQTKSGGTRTGNVTLAEGPPA
Rv0570: C1500: Ecoli-pET28[6xHis-Cohesin (underlined)-m.tbRv0570 (M. tb. antigen-
bold)].
(SEQ ID NO: 178)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGAGACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCGTGGGTGTCAGCTGGCCGGCGAAAGTCCGGCGCCGGGATG
GAACGCTGGTGCCGTTCGACATCGCGCGGATCGAAGCAGCGGTGACGCGGGCAGCGCGCGAG
GTGGCTTGCGACGACCCCGATATGCCGGGCACCGTAGCGAAAGCCGTCGCCGACGCACTCGGG
CGCGGTATCGCTCCCGTTGAGGACATTCAGGACTGCGTGGAGGCCCGGCTGGGGGAAGCCGGT
CTGGATGACGTGGCCCGTGTTTACATCATCTACCGGCAGCGGCGCGCCGAGCTGCGGACGGCT
AAGGCCTTGCTCGGCGTGCGGGACGAGTTAAAGCTGAGCTTGGCGGCCGTGACGGTACTGCGC
GAGCGCTATCTGCTGCACGACGAGCAGGGCCGGCCGGCCGAGTCGACCGGCGAGCTGATGGA
CCGATCGGCGCGCTGTGTCGCGGCGGCCGAGGACCAGTATGAGCCGGGCTCGTCGAGGCGGTG
GGCCGAGCGGTTCGCCACGCTATTACGCAACCTGGAATTCCTGCCGAATTCGCCCACGTTGAT
GAACTCTGGCACCGACCTGGGACTGCTCGCCGGCTGTTTTGTTCTGCCGATTGAGGATTCGCTG
CAATCGATCTTTGCGACGCTGGGACAGGCCGCCGAGCTGCAGCGGGCTGGAGGCGGCACCGG
ATATGCGTTCAGCCACCTGCGACCCGCCGGGGATCGGGTGGCCTCCACGGGCGGCACGGCCAG
CGGACCGGTGTCGTTTCTACGGCTGTATGACAGTGCCGCGGGTGTGGTCTCCATGGGCGGTCG
CCGGCGTGGCGCCTGTATGGCTGTGCTTGATGTGTCGCACCCGGATATCTGTGATTTCGTCACC
GCCAAGGCCGAATCCCCCAGCGAGCTCCCGCATTTCAACCTATCGGTTGGTGTGACCGACGCG
TTCCTGCGGGCCGTCGAACGCAACGGCCTACACCGGCTGGTCAATCCGCGAACCGGCAAGATC
GTCGCGCGGATGCCCGCCGCCGAGCTGTTCGACGCCATCTGCAAAGCCGCGCACGCCGGTGGC
GATCCCGGGCTGGTGTTTCTCGACACGATCAATAGGGCAAACCCGGTGCCGGGGAGAGGCCGC
ATCGAGGCGACCAACCCGTGCGGGGAGGTCCCACTGCTGCCTTACGAGTCATGTAATCTCGGC
TCGATCAACCTCGCCCGGATGCTCGCCGACGGTCGCGTCGACTGGGACCGGCTCGAGGAGGTC
GCCGGTGTGGCGGTGCGGTTCCTTGATGACGTCATCGATGTCAGCCGCTACCCCTTCCCCGAAC
TGGGTGAGGCGGCCCGCGCCACCCGCAAGATCGGGCTGGGAGTCATGGGTTTGGCGGAACTGC
TTGCCGCACTGGGTATTCCGTACGACAGTGAAGAAGCCGTGCGGTTAGCCACCCGGCTCATGC
GTCGCATACAGCAGGCGGCGCACACGGCATCGCGGAGGCTGGCCGAAGAGCGGGGCGCATTC
CCGGCGTTCACCGATAGCCGGTTCGCGCGGTCGGGCCCGAGGCGCAACGCACAGGTCACCTCC
GTCGCTCCGACGGGCACCATCTCACTGATCGCCGGAACCACCGCGGGCATCGAGCCGATGTTC
GCTATCGCGTTCACCCGCGCCATCGTCGGCCGGCATCTGCTGGAGGTCAATCCGTGCTTCGACC
GACTGGCCCGCGATCGGGGCTTTTATCGTGACGAGCTGATCGCCGAGATCGCTCAGCGTGGCG
GAGTCCGTGGCTATCCGCGGCTGCCTGCTGAGGTGCGGGCCGCGTTCCCGACCGCGGCGGAGA
TCGCGCCGCAGTGGCATCTGCGCATGCAGGCCGCGGTGCAGCGCCACGTCGAGGCCGCCGTGT
CCAAGACGGTCAACTTGCCCGCCACGGCGACGGTCGATGACGTCCGCGCCATCTATGTGGCCG
CCTGGAAGGCAAAGGTCAAGGGCATCACGGTGTATCGCTACGGCAGCCGGGAAGGACAGGTA
CTGTCCTACGCCGCGCCGAAACCGCTACTGGCGCAGGCTGACACGGAGTTCAGCGGCGGCTGT
GCGGGCCGCTCCTGCGAGTTCTGAGCGGCCGCACTCGAGCACCACCACCACCACCACTGAGAT
CCGGCTGCTAACAAAGCCCGAAAGGAAGCTGAGTTGGCTGCTGCCACCGCTGAGCAATAACTA
GCATAACCCCTTGGGGCCTCTAAACGGGTCTTGAGGGGTTTTTTGCTGAAAGGAGGAACTATA
TCCGGAT
Protein sequence - Ecoli-pET28[6xHis-Cohesin (underlined) -m.tbRv0570 (M. tb.
antigen - bold)].
(SEQ ID NO: 179)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
VGVSWPAKVRRRDGTLVPFDIARIEAAVTRAAREVACDDPDMPGTVAKAVADALGRGIAPVE
DIQDCVEARLGEAGLDDVARVYIIYRQRRAELRTAKALLGVRDELKLSLAAVTVLRERYLLH
DEQGRPAESTGELMDRSARCVAAAEDQYEPGSSRRWAERFATLLRNLEFLPNSPTLMNSGTD
LGLLAGCFVLPIEDSLQSIFATLGQAAELQRAGGGTGYAFSHLRPAGDRVASTGGTASGPVSF
LRLYDSAAGVVSMGGRRRGACMAVLDVSHPDICDFVTAKAESPSELPHFNLSVGVTDAFLRA
VERNGLHRLVNPRTGKIVARMPAAELFDAICKAAHAGGDPGLVFLDTINRANPVPGRGRIEA
TNPCGEVPLLPYESCNLGSINLARMLADGRVDWDRLEEVAGVAVRFLDDVIDVSRYPFPELG
EAARATRKIGLGVMGLAELLAALGIPYDSEEAVRLATRLMRRIQQAAHTASRRLAEERGAFP
AFTDSRFARSGPRRNAQVTSVAPTGTISLIAGTTAGIEPMFAIAFTRAIVGRHLLEVNPCFDRL
ARDRGFYRDELIAEIAQRGGVRGYPRLPAEVRAAFPTAAEIAPQWHLRMQAAVQRHVEAAV
SKTVNLPATATVDDVRAIYVAAWKAKVKGITVYRYGSREGQVLSYAAPKPLLAQADTEFSGG
CAGRSCEF
Rv1980: C2197: Ecoli-pET28[6xHis-CthermoCohesin (underlined) -m.tbRv1980 (M. tb.
antigen - bold)].
(SEQ ID NO: 180)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCGCCGCGCCCAAGACCTACTGCGAGGAGTTGAAAGGCACCG
ATACCGGCCAGGCGTGCCAGATTCAAATGTCCGACCCGGCCTACAACATCAACATCAGCCTGC
CCAGTTACTACCCCGACCAGAAGTCGCTGGAAAATTACATCGCCCAGACGCGCGACAAGTTCC
TCAGCGCGGCCACATCGTCCACTCCACGCGAAGCCCCCTACGAATTGAATATCACCTCGGCCA
CATACCAGTCCGCGATACCGCCGCGTGGTACGCAGGCCGTGGTGCTCAAGGTCTACCAGAACG
CCGGCGGCACGCACCCAACGACCACGTACAAGGCCTTCGATTGGGACCAGGCCTATCGCAAGC
CAATCACCTATGACACGCTGTGGCAGGCTGACACCGATCCGCTGCCAGTCGTCTTCCCCATTGT
GCAAGGTGAACTGAGCAAGCAGACCGGACAACAGGTATCGATAGCGCCGAATGCCGGCTTGG
ACCCGGTGAATTATCAGAACTTCGCAGTCACGAACGACGGGGTGATTTTCTTCTTCAACCCGG
GGGAGTTGCTGCCCGAAGCAGCCGGCCCAACCCAGGTATTGGTCCCACGTTCCGCGATCGACT
CGATGCTGGCCGAATTCTAGGCGGCCGCACTCGAGCACCACCACCACCACCACTGAGATCCGG
CTGCTAACAAAGCCCGAAAGGAAGCTGAGTTGGCTGCTGCCACCGCTGAGCAATAACTAGCAT
AACCCCTTGGGGCCTCTAAACGGGTCTTGAGGGGTTTTTTGCTGAAAGGAGGAACTATATCCG
GAT
Protein sequence - Ecoli-pET28[6xHis-CthermoCohesin (underlined) -m.tbRv1980 (M.
tb. antigen - bold)].
(SEQ ID NO: 181)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
AAPKTYCEELKGTDTGQACQIQMSDPAYNINISLPSYYPDQKSLENYIAQTRDKFLSAATSSTP
REAPYELNITSATYQSAIPPRGTQAVVLKVYQNAGGTHPTTTYKAFDWDQAYRKPITYDTLW
QADTDPLPVVFPIVQGELSKQTGQQVSIAPNAGLDPVNYQNFAVTNDGVIFFFNPGELLPEAA
GPTQVLVPRSAIDSMLAEF
Rv0475: C2199: Ecoli-pET28[6xHis-CthermoCohesin (underlined) -m.tbRv0475 (M. tb.
antigen - bold)].
(SEQ ID NO: 182)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCTGCAGCTGTATGGCTGAAAACTCGAACATTGATGACATCAA
GGCTCCGTTGCTTGCCGCGCTTGGAGCGGCCGACCTGGCCTTGGCCACTGTCAACGAGTTGATC
ACGAACCTGCGTGAGCGTGCGGAGGAGACTCGTACGGACACCCGCAGCCGGGTCGAGGAGAG
CCGTGCTCGCCTGACCAAGCTGCAGGAAGATCTGCCCGAGCAGCTCACCGAGCTGCGTGAGAA
GTTCACCGCCGAGGAGCTGCGTAAGGCCGCCGAGGGCTACCTCGAGGCCGCGACTAGCCGGTA
CAACGAGCTGGTCGAGCGCGGTGAGGCCGCTCTAGAGCGGCTGCGCAGCCAGCAGAGCTTCG
AGGAAGTGTCGGCGCGCGCCGAAGGCTACGTGGACCAGGCGGTGGAGTTGACCCAGGAGGCG
TTGGGTACGGTCGCATCGCAGACCCGCGCGGTCGGTGAGCGTGCCGCCAAGCTGGTCGGCATC
GAGCTGCCTAAGAAGGCTGCTCCGGCCAAGAAGGCCGCTCCGGCCAAGAAGGCCGCTCCGGC
CAAGAAGGCGGCGGCCAAGAAGGCGCCCGCGAAGAAGGCGGCGGCCAAGAAGGTCACCCAG
AAGTGCAGCTGTTAGGCGGCCGCACTCGAGCACCACCACCACCACCACTGAGATCCGGCTGCT
AACAAAGCCCGAAAGGAAGCTGAGTTGGCTGCTGCCACCGCTGAGCAATAACTAGCATAACC
CCTTGGGGCCTCTAAACGGGTCTTGAGGGGTTTTTTGCTGAAAGGAGGAACTATATCCGGAT
Protein sequence - Ecoli-pET28[6xHis-CthermoCohesin (underlined) -m.tbRv0475 (M.
tb. antigen - bold)].
(SEQ ID NO: 183)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
CSCMAENSNIDDIKAPLLAALGAADLALATVNELITNLRERAEETRTDTRSRVEESRARLTKL
QEDLPEQLTELREKFTAEELRKAAEGYLEAATSRYNELVERGEAALERLRSQQSFEEVSARA
EGYVDQAVELTQEALGTVASQTRAVGERAAKLVGIELPKKAAPAKKAAPAKKAAPAKKAAA
KKAPAKKAAAKKVTQKCSC
Rv0288: C2202: Ecoli-pET28[6xHis-CthermoCohesin (underlined) -m.tbRv0288 (M. tb.
antigen - bold)].
(SEQ ID NO: 184)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGTCGCAAATCATGTACAACTACCCCGCGATGTTGGGTCA
CGCCGGGGATATGGCCGGATATGCCGGCACGCTGCAGAGCTTGGGTGCCGAGATCGCCGTGGA
GCAGGCCGCGTTGCAGAGTGCGTGGCAGGGCGATACCGGGATCACGTATCAGGCGTGGCAGG
CACAGTGGAACCAGGCCATGGAAGATTTGGTGCGGGCCTATCATGCGATGTCCAGCACCCATG
AAGCCAACACCATGGCGATGATGGCCCGCGACACGGCCGAAGCCGCCAAATGGGGCGGCTAG
GCGGCCGCACTCGAGCACCACCACCACCACCACTGAGATCCGGCTGCTAACAAAGCCCGAAA
GGAAGCTGAGTTGGCTGCTGCCACCGCTGAGCAATAACTAGCATAACCCCTTGGGGCCTCTAA
ACGGGTCTTGAGGGGTTTTTTGCTGAAAGGAGGAACTATATCCGGAT
Protein sequence- Ecoli-pET28[6xHis-CthermoCohesin (underlined) -m.tbRv0288
(M. tb. antigen - bold)].
(SEQ ID NO: 185)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MSQIMYNYPAMLGHAGDMAGYAGTLQSLGAEIAVEQAALQSAWQGDTGITYQAWQAQWN
QAMEDLVRAYHAMSSTHEANTMAMMARDTAEAAKWGG
Ag85BΔ41: C2200: Ecoli-pET28[6xHis-CthermoCohesin (underlined) -m.tbAg85BDel41
(M. tb. antigen)].
(SEQ ID NO: 186)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCTCCCGGCCGGGGCTGCCGGTCGAGTACCTGCAGGTGCCGTC
GCCGTCGATGGGCCGCGACATCAAGGTTCAGTTCCAGAGCGGTGGGAACAACTCACCTGCGGT
TTATCTGCTCGACGGCCTGCGCGCCCAAGACGACTACAACGGCTGGGATATCAACACCCCGGC
GTTCGAGTGGTACTACCAGTCGGGACTGTCGATAGTCATGCCGGTCGGCGGGCAGTCCAGCTT
CTACAGCGACTGGTACAGCCCGGCCTGCGGTAAGGCTGGCTGCCAGACTTACAAGTGGGAAAC
CTTCCTGACCAGCGAGCTGCCGCAATGGTTGTCCGCCAACAGGGCCGTGAAGCCCACCGGCAG
CGCTGCAATCGGCTTGTCGATGGCCGGCTCGTCGGCAATGATCTTGGCCGCCTACCACCCCCAG
CAGTTCATCTACGCCGGCTCGCTGTCGGCCCTGCTGGACCCCTCTCAGGGGATGGGGCCTAGCC
TGATCGGCCTCGCGATGGGTGACGCCGGCGGTTACAAGGCCGCAGACATGTGGGGTCCCTCGA
GTGACCCGGCATGGGAGCGCAACGACCCTACGCAGCAGATCCCCAAGCTGGTCGCAAACAAC
ACCCGGCTATGGGTTTATTGCGGGAACGGCACCCCGAACGAGTTGGGCGGTGCCAACATACCC
GCCGAGTTCTTGGAGAACTTCGTTCGTAGCAGCAACCTGAAGTTCCAGGATGCGTACAACGCC
GCGGGCGGGCACAACGCCGTGTTCAACTTCCCGCCCAACGGCACGCACAGCTGGGAGTACTGG
GGCGCTCAGCTCAACGCCATGAAGGGTGACCTGCAGAGTTCGTTAGGCGCCGGCTGAGCGGCC
GCACTCGAGCACCACCACCACCACCACTGAGATCCGGCTGCTAACAAAGCCCGAAAGGAAGC
TGAGTTGGCTGCTGCCACCGCTGAGCAATAACTAGCATAACCCCTTGGGGCCTCTAAACGGGT
CTTGAGGGGTTTTTTGCTGAAAGGAGGAACTATATCCGGAT
Protein sequence - Ecoli-pET28[6xHis-CthermoCohesin (underlined) -m.tbAg85BDel41
(M. tb. antigen - bold)].
(SEQ ID NO: 187)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
SRPGLPVEYLQVPSPSMGRDIKVQFQSGGNNSPAVYLLDGLRAQDDYNGWDINTPAFEWYYQ
SGLSIVMPVGGQSSFYSDWYSPACGKAGCQTYKWETFLTSELPQWLSANRAVKPTGSAAIGL
SMAGSSAMILAAYHPQQFIYAGSLSALLDPSQGMGPSLIGLAMGDAGGYKAADMWGPSSDPA
WERNDPTQQIPKLVANNTRLWVYCGNGTPNELGGANIPAEFLENFVRSSNLKCFQDAYNAAG
GHNAVFNFPPNGTHSWEYWGAQLNAMKGDLQSSLGAG
CFP10: C1254: Ecoli-pET28 [6xHis-Cohesin (underlined) -m.tbCFP10
(M. tb. antigen)].
(SEQ ID NO: 188)
TGGCGAATGGGACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGAATTTTAA
CAAAATATTAACGTTTACAATTTCAGGTGGCACTTTTCGGGGAAATGTGCGCGGAACCCCTATT
TGTTTATTTTTCTAAATACATTCAAATATGTATCCGCTCATGAATTAATTCTTAGAAAAACTCAT
CGAGCATCAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGATCCTGGTA
TCGGTCTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAAGTTTA
TGCATTTCTTTCCAGACTTGTTCAACAGGCCAGCCATTACGCTCGTCATCAAAATCACTCGCAT
CAACCAAACCGTTATTCATTCGTGATTGCGCCTGAGCGAGACGAAATACGCGATCGCTGTTAA
AAGGACAATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGGATCGCAG
TGGTGAGTAACCATGCATCATCAGGAGTACGGATAAAATGCTTGATGGTCGGAAGAGGCATAA
ATTCCGTCAGCCAGTTTAGTCTGACCATCTCATCTGTAACATCATTGGCAACGCTACCTTTGCC
ATGTTTCAGAAACAACTCTGGCGCATCGGGCTTCCCATACAATCGATAGATTGTCGCACCTGAT
TGCCCGACATTATCGCGAGCCCATTTATACCCATATAAATCAGCATCCATGTTGGAATTTAATC
GCGGCCTAGAGCAAGACGTTTCCCGTTGAATATGGCTCATAACACCCCTTGTATTACTGTTTAT
GTAAGCAGACAGTTTTATTGTTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGC
TGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGT
AGCCGTAGTTAGGCCACCACTTCAAGAACTCTGTAGCACCGCCTACATACCTCGCTCTGCTAAT
CCTGTTACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGCCCAGCTT
GGAGCGAACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGGCAGAGATGAAGACCGATGCCGCTACCCTCGCGCA
GGAGGCAGGTAATTTCGAGCGGATCTCCGGCGACCTGAAAACCCAGATCGACCAGGTGG
AGTCGACGGCAGGTTCGTTGCAGGGCCAGTGGCGCGGCGCGGCGGGGACGGCCGCCCA
GGCCGCGGTGGTGCGCTTCCAAGAAGCAGCCAATAAGCAGAAGCAGGAACTCGACGAGA
TCTCGACGAATATTCGTCAGGCCGGCGTCCAATACTCGAGGGCCGACGAGGAGCAGCAG
CAGGCGCTGTCCTCGCAAATGGGCTTCTGAGCGGCCGCACTCGAGCACCACCACCACCA
CCACTGAGATCCGGCTGCTAACAAAGCCCGAAAGGAAGCTGAGTTGGCTGCTGCCACCGCTG
AGCAATAACTAGCATAACCCCTTGGGGCCTCTAAACGGGTCTTGAGGGGTTTTTTGCTGAAAG
GAGGAACTATATCCGGAT
Protein sequence - Ecoli-pET28[6xHis-Cohesin (underlined) -m.tbCFP10 (M. tb.
antigen - bold)].
(SEQ ID NO: 189)
MGSSHHHHHHSSGLVPRGSHMASMDLDAVRIKVDTVNAKPGDTVNIPVRFSGIPSKGIANC
DFVYSYDPNVLEIIEIKPGELIVDPNPTKSFDTAVYPDRKMIVFLFAEDSGTGAYAITKDGVFATIVA
KVKEGAPNGLSVIKFVEVGGFANNDLVEQKTQFFDGGVNVGDTTEPATPTTPVTTPTTTDDLDAAS
MAEMKTDAATLAQEAGNFERISGDLKTQIDQVESTAGSLQGQWRGAAGTAAQAAVVRFQE
AANKQKQELDEISTNIRQAGVQYSRADEEQQQALSSQMGF
ESAT6 - C1251 Ecoli-pET28[6xHis-Cohesin (underlined)-m.tbESAT6 (bold)];
(SEQ ID NO: 190)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGACAGAGCAGCAGTGGAATTTCGCGGGTATCGAGGC
CGCGGCAAGCGCAATCCAGGGAAATGTCACGTCCATTCATTCCCTCCTTGACGAGGGGA
AGCAGTCCCTGACCAAGCTCGCAGCGGCCTGGGGCGGTAGCGGTTCGGAGGCGTACCAG
GGTGTCCAGCAAAAATGGGACGCCACGGCTACCGAGCTGAACAACGCGCTGCAGAACCT
GGCGCGGACGATCAGCGAAGCCGGTCAGGCAATGGCTTCGACCGAAGGCAACGTCACTG
GGATGTTCGCATAGGCGGCCGCACTCGAGCACCACCACCACCACCACTGAGATCCGGCTGCT
AACAAAGCCCGAAAGGAAGCTGAGTTGGCTGCTGCCACCGCTGAGCAATAACTAGCATAACC
CCTTGGGGCCTCTAAACGGGTCTTGAGGGGTTTTTTGCTGAAAGGAGGAACTATATCCGGAT
Protein sequence ESAT6 - C1251 Ecoli-pET28[6xHis-Cohesin (underlined)-m.tbESAT6]
(SEQ ID NO: 191)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMTEQ
QWNFAGIEAAASAIQGNVTSIHSLLDEGKQSLTKLAAAWGGSGSEAYQGVQQKWDATATEL
NNALQNLARTISEAGQAMASTEGNVTGMFA
Rv3478 - C1308 Ecoli-pET28[6xHis-Cohesin (underlined)-m.tbRv3478 (bold)];
(SEQ ID NO: 192)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCGTGGTGGATTTCGGGGCGTTACCACCGGAGATCAACTC
CGCGAGGATGTACGCCGGCCCGGGTTCGGCCTCGCTGGTGGCCGCCGCGAAGATGTGG
GACAGCGTGGCGAGTGACCTGTTTTCGGCCGCGTCGGCGTTTCAGTCGGTGGTCTGGGG
TCTGACGGTGGGGTCGTGGATAGGTTCGTCGGCGGGTCTGATGGCGGCGGCGGCCTCGC
CGTATGTGGCGTGGATGAGCGTCACCGCGGGGCAGGCCCAGCTGACCGCCGCCCAGGTC
CGGGTTGCTGCGGCGGCCTACGAGACAGCGTATAGGCTGACGGTGCCCCCGCCGGTGAT
CGCCGAGAACCGTACCGAACTGATGACGCTGACCGCGACCAACCTCTTGGGGCAAAACA
CGCCGGCGATCGAGGCCAATCAGGCCGCATACAGCCAGATGTGGGGCCAAGACGCGGA
GGCGATGTATGGCTACGCCGCCACGGCGGCGACGGCGACCGAGGCGTTGCTGCCGTTCG
AGGACGCCCCACTGATCACCAACCCCGGCGGGCTCCTTGAGCAGGCCGTCGCGGTCGAG
GAGGCCATCGACACCGCCGCGGCGAACCAGTTGATGAACAATGTGCCCCAAGCGCTGCA
ACAGCTGGCCCAGCCAGCGCAGGGCGTCGTACCTTCTTCCAAGCTGGGTGGGCTGTGGA
CGGCGGTCTCGCCGCATCTGTCGCCGCTCAGCAACGTCAGTTCGATAGCCAACAACCAC
ATGTCGATGATGGGCACGGGTGTGTCGATGACCAACACCTTGCACTCGATGTTGAAGGG
CTTAGCTCCGGCGGCGGCTCAGGCCGTGGAAACCGCGGCGGAAAACGGGGTCTGGGCG
ATGAGCTCGCTGGGCAGCCAGCTGGGTTCGTCGCTGGGTTCTTCGGGTCTGGGCGCTGG
GGTGGCCGCCAACTTGGGTCGGGCGGCCTCGGTCGGTTCGTTGTCGGTGCCGCCAGCAT
GGGCCGCGGCCAACCAGGCGGTCACCCCGGCGGCGCGGGCGCTGCCGCTGACCAGCCT
GACCAGCGCCGCCCAAACCGCCCCCGGACACATGCTGGGCGGGCTACCGCTGGGGCACT
CGGTCAACGCCGGCAGCGGTATCAACAATGCGCTGCGGGTGCCGGCACGGGCCTACGCG
ATACCCCGCACACCGGCCGCCGGATAGGCGGCCGCACTCGAGCACCACCACCACCACCACT
GAGATCCGGCTGCTAACAAAGCCCGAAAGGAAGCTGAGTTGGCTGCTGCCACCGCTGAGCAAT
AACTAGCATAACCCCTTGGGGCCTCTAAACGGGTCTTGAGGGGTTTTTTGCTGAAAGGAGGAA
CTATATCCGGAT
Protein sequence - Rv3478 - C1308 Ecoli-pET28[6xHis-Cohesin (underlined)-
m.tbRv3478 (bold)];
(SEQ ID NO: 193)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASVVDF
GALPPEINSARMYAGPGSASLVAAAKMWDSVASDLFSAASAFQSVVWGLTVGSWIGSSAGLM
AAAASPYVAWMSVTAGQAQLTAAQVRVAAAAYETAYRLTVPPPVIAENRTELMTLTATNLL
GQNTPAIEANQAAYSQMWGQDAEAMYGYAATAATATEALLPFEDAPLITNPGGLLEQAVAV
EEAIDTAAANQLMNNVPQALQQLAQPAQGVVPSSKLGGLWTAVSPHLSPLSNVSSIANNHMS
MMGTGVSMTNTLHSMLKGLAPAAAQAVETAAENGVWAMSSLGSQLGSSLGSSGLGAGVAA
NLGRAASVGSLSVPPAWAAANQAVTPAARALPLTSLTSAAQTAPGHMLGGLPLGHSVNAGSG
INNALRVPARAYAIPRTPAAG
Rv0934- C2390 Ecoli-pET28[6xHis-CthermoCohesin (underlined)-m.tbRv0934 (bold)]
Additional modifications are in italics.
(SEQ ID NO: 194)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGAAAAACGCCAGCAA
CGCGGCCTTTTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAGAGCGCCTGATGCGGTATTTTCT
CCTTACGCATCTGTGCGGTATTTCACACCGCATATATGGTGCACTCTCAGTACAATCTGCTCTG
ATGCCGCATAGTTAAGCCAGTATACACTCCGCTATCGCTACGTGACTGGGTCATGGCTGCGCCC
CGACACCCGCCAACACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCACCGTCATCACCGAAA
CGCGCGAGGCAGCTGCGGTAAAGCTCATCAGCGTGGTCGTGAAGCGATTCACAGATGTCTGCC
TGTTCATCCGCGTCCAGCTCGTTGAGTTTCTCCAGAAGCGTTAATGTCTGGCTTCTGATAAAGC
GGGCCATGTTAAGGGCGGTTTTTTCCTGTTTGGTCACTGATGCCTCCGTGTAAGGGGGATTTCT
GTTCATGGGGGTAATGATACCGATGAAACGAGAGAGGATGCTCACGATACGGGTTACTGATGA
TGAACATGCCCGGTTACTGGAACGTTGTGAGGGTAAACAACTGGCGGTATGGATGCGGCGGGA
CCAGAGAAAAATCACTCAGGGTCAATGCCAGCGCTTCGTTAATACAGATGTAGGTGTTCCACA
GGGTAGCCAGCAGCATCCTGCGATGCAGATCCGGAACATAATGGTGCAGGGCGCTGACTTCCG
CGTTTCCAGACTTTACGAAACACGGAAACCGAAGACCATTCATGTTGTTGCTCAGGTCGCAGA
CGTTTTGCAGCAGCAGTCGCTTCACGTTCGCTCGCGTATCGGTGATTCATTCTGCTAACCAGTA
AGGCAACCCCGCCAGCCTAGCCGGGTCCTCAACGACAGGAGCACGATCATGCGCACCCGTGG
GGCCGCCATGCCGGCGATAATGGCCTGCTTCTCGCCGAAACGTTTGGTGGCGGGACCAGTGAC
GAAGGCTTGAGCGAGGGCGTGCAAGATTCCGAATACCGCAAGCGACAGGCCGATCATCGTCG
CGCTCCAGCGAAAGCGGTCCTCGCCGAAAATGACCCAGAGCGCTGCCGGCACCTGTCCTACGA
GTTGCATGATAAAGAAGACAGTCATAAGTGCGGCGACGATAGTCATGCCCCGCGCCCACCGGA
AGGAGCTGACTGGGTTGAAGGCTCTCAAGGGCATCGGTCGAGATCCCGGTGCCTAATGAGTGA
GCTAACTTACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCGCCAGGGTGG
TTTTTCTTTTCACCAGTGAGACGGGCAACAGCTGATTGCCCTTCACCGCCTGGCCCTGAGAGAG
TTGCAGCAAGCGGTCCACGCTGGTTTGCCCCAGCAGGCGAAAATCCTGTTTGATGGTGGTTAA
CGGCGGGATATAACATGAGCTGTCTTCGGTATCGTCGTATCCCACTACCGAGATATCCGCACC
AACGCGCAGCCCGGACTCGGTAATGGCGCGCATTGCGCCCAGCGCCATCTGATCGTTGGCAAC
CAGCATCGCAGTGGGAACGATGCCCTCATTCAGCATTTGCATGGTTTGTTGAAAACCGGACAT
GGCACTCCAGTCGCCTTCCCGTTCCGCTATCGGCTGAATTTGATTGCGAGTGAGATATTTATGC
CAGCCAGCCAGACGCAGACGCGCCGAGACAGAACTTAATGGGCCCGCTAACAGCGCGATTTG
CTGGTGACCCAATGCGACCAGATGCTCCACGCCCAGTCGCGTACCGTCTTCATGGGAGAAAAT
AATACTGTTGATGGGTGTCTGGTCAGAGACATCAAGAAATAACGCCGGAACATTAGTGCAGGC
AGCTTCCACAGCAATGGCATCCTGGTCATCCAGCGGATAGTTAATGATCAGCCCACTGACGCG
TTGCGCGAGAAGATTGTGCACCGCCGCTTTACAGGCTTCGACGCCGCTTCGTTCTACCATCGAC
ACCACCACGCTGGCACCCAGTTGATCGGCGCGAGATTTAATCGCCGCGACAATTTGCGACGGC
GCGTGCAGGGCCAGACTGGAGGTGGCAACGCCAATCAGCAACGACTGTTTGCCCGCCAGTTGT
TGTGCCACGCGGTTGGGAATGTAATTCAGCTCCGCCATCGCCGCTTCCACTTTTTCCCGCGTTTT
CGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCGTGAAAATTCGTTTGCATACGCTGTTGGCCGTGTTGACC
GCTGCGCCGCTGCTGCTAGCAGCGGCGGGCTGTGGCTCGAAACCACCGAGCGGTTCGCC
TGAAACGGGCGCCGGCGCCGGTACTGTCGCGACTACCCCCGCGTCGTCGCCGGTGACGT
TGGCGGAGACCGGTAGCACGCTGCTCTACCCGCTGTTCAACCTGTGGGGTCCGGCCTTT
CACGAGAGGTATCCGAACGTCACGATCACCGCTCAGGGCACCGGTTCTGGTGCCGGGAT
CGCGCAGGCCGCCGCCGGGACGGTCAACATTGGGGCCTCCGACGCCTATCTGTCGGAAG
GTGATATGGCCGCGCACAAGGGGCTGATGAACATCGCGCTAGCCATCTCCGCTCAGCAG
GTCAACTACAACCTGCCCGGAGTGAGCGAGCACCTCAAGCTGAACGGAAAAGTCCTGGC
GGCCATGTACCAGGGCACCATCAAAACCTGGGACGACCCGCAGATCGCTGCGCTCAACC
CCGGCGTGAACCTGCCCGGCACCGCGGTAGTTCCGCTGCACCGCTCCGACGGGTCCGGT
GACACCTTCTTGTTCACCCAGTACCTGTCCAAGCAAGATCCCGAGGGCTGGGGCAAGTC
GCCCGGCTTCGGCACCACCGTCGACTTCCCGGCGGTGCCGGGTGCGCTGGGTGAGAACG
GCAACGGCGGCATGGTGACGGTTGCGCCGAGACACCGGGCTGCGTGGCCTATATCGGCA
TCAGCTTCCTCGACCAGGCCAGTCAACGGGGACTCGGCGAGGCCCAACTAGGCAATAGC
TCTGGCAATTTCTTGTTGCCCGACGCGCAAAGCATTCAGGCCGCGGCGGCTGGCTTCGC
ATCGAAAACCCCGGCGAACCAGGCGATTTCGATGATCGACGGGCCCGCCCCGGACGGCT
ACCCGATCATCAACTACGAGTACGCCATCGTCAACAACCGGCAAAAGGACGCCGCCACC
GCGCAGACCTTGCAGGCATTTCTGCACTGGGCGATCACCGACGGCAACAAGGCCTCGTT
CCTCGACCAGGTTCATTTCCAGCCGCTGCCGCCCGCGGTGGTGAAGTTGTCTGACGCGT
TGATCGCGACGATTTCCAGCTAG [symbol] CGGCCGCACTCGAGCACCACCACCACCACCACTGAGAT
CCGGCTGCTAACAAAGCCCGAAAGGAAGCTGAGTTGGCTGCTGCCACCGCTGAGCAATAACTA
GCATAACCCCTTGGGGCCTCTAAACGGGTCTTGAGGGGTTTTTTGCTGAAAGGAGGAACTATA
TCCGGAT
Protein sequence - Rv0934- C2390 Ecoli-pET28[6xHis-CthermoCohesin (underlined)-
m.tbRv0934 (bold)];
(SEQ ID NO: 195)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASGCGS
KPPSGSPETGAGAGTVATTPASSPVTLAETGSTLLYPLFNLWGPAFHERYPNVTITAQGTGSG
AGIAQAAAGTVNIGASDAYLSEGDMAAHKGLMNIALAISAQQVNYNLPGVSEHLKLNGKVLA
AMYQGTIKTWDDPQIAALNPGVNLPGTAVVPLHRSDGSGDTFLFTQYLSKQDPEGWGKSPG
FGTTVDFPAVPGALGENGNGGMVTGCAETPGCVAYIGISFLDQASQRGLGEAQLGNSSGNFL
LPDAQSIQAAAAGFASKTPANQAISMIDGPAPDGYPIINYEYAIVNNRQKDAATAQTLQAFLH
WAITDGNKASFLDQVHFQPLPPAVVKLSDALIATISSEF
Rv2608 - C1259 Ecoli-pET28[6xHis-Cohesin (underlined)-m.tbRv2608 (bold)]
(SEQ ID NO: 196)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGTATGAATTTCGCCGTTTTGCCGCCGGAGGTGAATTCGGCGCG
CATATTCGCCGGTGCGGGCCTGGGCCCAATGCTGGCGGCGGCGTCGGCCTGGGACGGGTTGGC
CGAGGAGTTGCATGCCGCGGCGGGCTCGTTCGCGTCGGTGACCACCGGGTTGGCGGGCGACGC
GTGGCATGGTCCGGCGTCGCTGGCGATGACCCGCGCGGCCAGCCCGTATGTGGGGTGGT
TGAACACGGCGGCGGGTCAGGCCGCGCAGGCGGCCGGCCAGGCGCGGCTAGCGGCGAG
CGCGTTCGAGGCGACGCTGGCGGCCACCGTGTCTCCAGCGATGGTCGCGGCCAACCGGA
CACGGCTGGCGTCGCTGGTGGCAGCCAACTTGCTGGGCCAGAACGCCCCGGCGATCGCG
GCCGCGGAGGCTGAATACGAGCAGATATGGGCCCAGGACGTGGCCGCGATGTTCGGCTA
TCACTCCGCCGCGTCGGCGGTGGCCACGCAGCTGGCGCCTATTCAAGAGGGTTTGCAGC
AGCAGCTGCAAAACGTGCTGGCCCAGTTGGCTAGCGGGAACCTGGGCAGCGGAAATGTG
GGCGTCGGCAACATCGGCAACGACAACATTGGCAACGCAAACATCGGCTTCGGAAATCG
AGGCGACGCCAACATCGGCATCGGGAATATCGGCGACAGAAACCTCGGCATTGGGAACA
CCGGCAATTGGAATATCGGCATCGGCATCACCGGCAACGGACAAATCGGCTTCGGCAAG
CCTGCCAACCCCGACGTCTTGGTGGTGGGCAACGGCGGCCCGGGAGTAACCGCGTTGGT
CATGGGCGGCACCGACAGCCTACTGCCGCTGCCCAACATCCCCTTACTCGAGTACGCTG
CGCGGTTCATCACCCCCGTGCATCCCGGATACACCGCTACGTTCCTGGAAACGCCATCGC
AGTTTTTCCCATTCACCGGGCTGAATAGCCTGACCTATGACGTCTCCGTGGCCCAGGGCG
TAACGAATCTGCACACCGCGATCATGGCGCAACTCGCGGCGGGAAACGAAGTCGTCGTC
TTCGGCACCTCCCAAAGCGCCACGATAGCCACCTTCGAAATGCGCTATCTGCAATCCCTG
CCAGCACACCTGCGTCCGGGTCTCGACGAATTGTCCTTTACGTTGACCGGCAATCCCAAC
CGGCCCGACGGTGGCATTCTTACGCGTTTTGGCTTCTCCATACCGCAGTTGGGTTTCACA
TTGTCCGGCGCGACGCCCGCCGACGCCTACCCCACCGTCGATTACGCGTTCCAGTACGA
CGGCGTCAACGACTTCCCCAAATACCCGCTGAATGTCTTCGCGACCGCCAACGCGATCG
CGGGCATCCTTTTCCTGCACTCCGGGTTGATTGCGTTGCCGCCCGATCTTGCCTCGGGCG
TGGTTCAACCGGTGTCCTCACCGGACGTCCTGACCACCTACATCCTGCTGCCCAGCCAAG
ATCTGCCGCTGCTGGTCCCGCTGCGTGCTATCCCCCTGCTGGGAAACCCGCTTGCCGAC
CTCATCCAGCCGGACTTGCGGGTGCTCGTCGAGTTGGGTTATGACCGCACCGCCCACCA
GGACGTGCCCAGCCCGTTCGGACTGTTTCCGGACGTCGATTGGGCCGAGGTGGCCGCGG
ACCTGCAGCAAGGCGCCGTGCAAGGCGTCAACGACGCCCTGTCCGGACTGGGGCTGCCG
CCGCCGTGGCAGCCGGCGCTACCCCGACTTTTCTAAAAGCTTGCGGCCGCACTCGAGCA
CCACCACCACCACCACTGAGATCCGGCTGCTAACAAAGCCCGAAAGGAAGCTGAGTTGGCTG
CTGCCACCGCTGAGCAATAACTAGCATAACCCCTTGGGGCCTCTAAACGGGTCTTGAGGGGTT
TTTTGCTGAAAGGAGGAACTATATCCGGAT
Protein sequence Rv2608 - C1259 Ecoli-pET28[6xHis-Cohesin (underlined)-m.tbRv2608
(bold)]
(SEQ ID NO: 197)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMNFA
VLPPEVNSARIFAGAGLGPMLAAASAWDGLAEELHAAAGSFASVTTGLAGDAWHGPASLAM
TRAASPYVGWLNTAAGQAAQAAGQARLAASAFEATLAATVSPAMVAANRTRLASLVAANLL
GQNAPAIAAAEAEYEQIWAQDVAAMFGYHSAASAVATQLAPIQEGLQQQLQNVLAQLASGN
LGSGNVGVGNIGNDNIGNANIGFGNRGDANIGIGNIGDRNLGIGNTGNWNIGIGITGNGQIGFG
KPANPDVLVVGNGGPGVTALVMGGTDSLLPLPNIPLLEYAARFITPVHPGYTATFLETPSQFF
PFTGLNSLTYDVSVAQGVTNLHTAIMAQLAAGNEVVVFGTSQSATIATFEMRYLQSLPAHLRP
GLDELSFTLTGNPNRPDGGILTRFGFSIPQLGFTLSGATPADAYPTVDYAFQYDGVNDFPKYPL
NVFATANAIAGILFLHSGLIALPPDLASGVVQPVSSPDVLTTYILLPSQDLPLLVPLRAIPLLGNP
LADLIQPDLRVLVELGYDRTAHQDVPSPFGLFPDVDWAEVAADLQQGAVQGVNDALSGLGL
PPPWQPALPRLF
Rv3864 - C2363 Ecoli-pET28[6xHis-CthermoCohesin (underlined)-m.tbRv3864 (bold)];
Additional modifications are in italics.
(SEQ ID NO: 198)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGGCATCGGGTAGCGGTCTTTGCAAGACGACGAGTAA
CTTTATTTGGGGCCAGTTACTCTTGCTTGGAGAGGGAATCCCCGACCCAGGCGACATTTT
CAACACCGGTTCGTCGCTGTTCAAACAAATCAGCGACAAAATGGGACTCGCCATTCCGG
GCACCAACTGGATCGGCCAAGCGGCGGAAGCTTACCTAAACCAGAACATCGCGCAACAA
CTTCGCGCACAGGTGATGGGCGATCTCGACAAATTAACCGGCAACATGATCTCGAATCA
GGCCAAATACGTCTCCGATACGCGCGACGTCCTGCGGGCCATGAAGAAGATGATTGACG
GTGTCTACAAGGTTTGTAAGGGCCTCGAAAAGATTCCGCTGCTCGGCCACTTGTGGTCGT
GGGAGCTCGCAATCCCTATGTCCGGCATCGCGATGGCCGTTGTCGGCGGCGCATTGCTC
TATCTAACGATTATGACGCTGATGAATGCGACCAACCTGAGGGGAATTCTCGGCAGGCT
GATCGAGATGTTGACGACCTTGCCAAAGTTCCCCGGCCTGCCCGGGTTGCCCAGCCTGC
CCGACATCATCGACGGCCTCTGGCCGCCGAAGTTGCCCGACATTCCGATCCCCGGCCTG
CCCGACATCCCGGGCCTACCCGACTTCAAATGGCCGCCCACCCCCGGCAGCCCGTTGTT
CCCCGACCTCCCGTCGTTCCCAGGGTTCCCCGGGTTCCCGGAGTTCCCCGCCATCCCCG
GGTTCCCCGCACTGCCCGGGTTGCCCAGCATTCCCAACTTGTTCCCCGGCTTGCCGGGTC
TGGGCGACCTGCTGCCCGGCGTAGGCGATTTGGGCAAGTTACCCACCTGGACTGAGCTG
GCCGCTTTGCCTGACTTCTTGGGCGGCTTCGCCGGCCTGCCCAGCTTGGGTTTTGGCAAT
CTGCTCAGCTTTGCCAGTTTGCCCACCGTGGGTCAGGTGACCGCCACCATGGGTCAGCT
GCAACAGCTCGTGGCGGCCGGCGGTGGCCCCAGCCAACTGGCCAGCATGGGCAGCCAA
CAAGCGCAACTGATCTCGTCGCAGGCCCAGCAAGGAGGCCAGCAGCACGCCACCCTCGT
GAGCGACAAGAAGGAAGACGAGGAAGGCGTGGCCGAGGCGGAGCGTGCACCCATCGAC
GCTGGCACCGCGGCCAGCCAACGGGGGCAGGAGGGGACCGTCCTTTGACTCGAGTAAGC
GGCCGCACTCGAGCACCACCACCACCACCACTGAGATCCGGCTGCTAACAAAGCCCGAAAGGA
AGCTGAGTTGGCTGCTGCCACCGCTGAGCAATAACTAGCATAACCCCTTGGGGCCTCTAAACG
GGTCTTGAGGGGTTTTTTGCTGAAAGGAGGAACTATATCCGGAT
Protein sequence - Rv3864 - C2363 Ecoli-pET28[6xHis-CthermoCohesin (underlined)-
m.tbRv3864 (bold)];
(SEQ ID NO: 199)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMASG
SGLCKTTSNFIWGQLLLLGEGIPDPGDIFNTGSSLFKQISDKMGLAIPGTNWIGQAAEAYLNQ
NIAQQLRAQVMGDLDKLTGNMISNQAKYVSDTRDVLRAMKKMIDGVYKVCKGLEKIPLLG
HLWSWELAIPMSGIAMAVVGGALLYLTIMTLMNATNLRGILGRLIEMLTTLPKFPGLPGLPS
LPDIIDGLWPPKLPDIPIPGLPDIPGLPDFKWPPTPGSPLFPDLPSFPGFPGFPEFPAIPGFPALPG
LPSIPNLFPGLPGLGDLLPGVGDLGKLPTWTELAALPDFLGGFAGLPSLGFGNLLSFASLPTV
GQVTATMGQLQQLVAAGGGPSQLASMGSQQAQLISSQAQQGGQQHATLVSDKKEDEEGVA
EAERAPIDAGTAASQRGQEGTVLLE
Rv3881 - C2365 Ecoli-pET28[6xHis-CthermoCohesin (underlined)-m.tbRv3881 (bold)]
Additional modifications are in italics.
(SEQ ID NO: 200)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGC [symbol] AGCAAGAGAT
TTTGAACAGGGCCAACGAGGTGGAGGCCCCGATGGCGGACCCACCGACTGATGTCCCCA
TCACACCGTGCGAACTCACGGCGGCTAAAAACGCCGCCCAACAGCTGGTATTGTCCGCC
GACAACATGCGGGAATACCTGGCGGCCGGTGCCAAAGAGCGGCAGCGTCTGGCGACCTC
GCTGCGCAACGCGGCCAAGGCGTATGGCGAGGTTGATGAGGAGGCTGCGACCGCGCTG
GACAACGACGGCGAAGGAACTGTGCAGGCAGAATCGGCCGGGGCCGTCGGAGGGGACA
GTTCGGCCGAACTAACCGATACGCCGAGGGTGGCCACGGCCGGTGAACCCAACTTCATG
GATCTCAAAGAAGCGGCAAGGAAGCTCGAAACGGGCGACCAAGGCGCATCGCTCGCGCA
CTTTGCGGATGGGTGGAACACTTTCAACCTGACGCTGCAAGGCGACGTCAAGCGGTTCC
GGGGGTTTGACAACTGGGAAGGCGATGCGGCTACCGCTTGCGAGGCTTCGCTCGATCAA
CAACGGCAATGGATACTCCACATGGCCAAATTGAGCGCTGCGATGGCCAAGCAGGCTCA
ATATGTCGCGCAGCTGCACGTGTGGGCTAGGCGGGAACATCCGACTTATGAAGACATAG
TCGGGCTCGAACGGCTTTACGCGGAAAACCCTTCGGCCCGCGACCAAATTCTCCCGGTG
TACGCGGAGTATCAGCAGAGGTCGGAGAAGGTGCTGACCGAATACAACAACAAGGCAGC
CCTGGAACCGGTAAACCCGCCGAAGCCTCCCCCCGCCATCAAGATCGACCCGCCCCCGC
CTCCGCAAGAGCAGGGATTGATCCCTGGCTTCCTGATGCCGCCGTCTGACGGCTCCGGT
GTGACTCCCGGTACCGGGATGCCAGCCGCACCGATGGTTCCGCCTACCGGATCGCCGGG
TGGTGGCCTCCCGGCTGACACGGCGGCGCAGCTGACGTCGGCTGGGCGGGAAGCCGCA
GCGCTGTCGGGCGACGTGGCGGTCAAAGCGGCATCGCTCGGTGGCGGTGGAGGCGGCG
GGGTGCCGTCGGCGCCGTTGGGATCCGCGATCGGGGGCGCCGAATCGGTGCGGCCCGC
TGGCGCTGGTGACATTGCCGGCTTAGGCCAGGGAAGGGCCGGCGGCGGCGCCGCGCTG
GGCGGCGGTGGCATGGGAATGCCGATGGGTGCCGCGCATCAGGGACAAGGGGGCGCCA
AGTCCAAGGGTTCTCAGCAGGAAGACGAGGCGCTCTACACCGAGGATCGGGCATGGACC
GAGGCCGTCATTGGTAAC [symbol] GAATTCTGAGCGGCC
GCACTCGAGCACCACCACCACCACCACTGAGATCCGGCTGCTAACAAAGCCCGAAAGGAAGCT
GAGTTGGCTGCTGCCACCGCTGAGCAATAACTAGCATAACCCCTTGGGGCCTCTAAACGGGTC
TTGAGGGGTTTTTTGCTGAAAGGAGGAACTATATCCGGAT
Protein sequence - Rv3881 - C2365 Ecoli-pET28[6xHis-CthermoCohesin (underlined)-
m.tbRv3881 (bold)]
(SEQ ID NO: 201)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMTQS
QTVTVDQQEILNRANEVEAPMADPPTDVPITPCELTAAKNAAQQLVLSADNMREYLAAGAKE
RQRLATSLRNAAKAYGEVDEEAATALDNDGEGTVQAESAGAVGGDSSAELTDTPRVATAGE
PNFMDLKEAARKLETGDQGASLAHFADGWNTFNLTLQGDVKRFRGFDNWEGDAATACEAS
LDQQRQWILHMAKLSAAMAKQAQYVAQLHVWARREHPTYEDIVGLERLYAENPSARDQILP
VYAEYQQRSEKVLTEYNNKAALEPVNPPKPPPAIKIDPPPPPQEQGLIPGFLMPPSDGSGVTPG
TGMPAAPMVPPTGSPGGGLPADTAAQLTSAGREAAALSGDVAVKAASLGGGGGGGVPSAPL
GSAIGGAESVRPAGAGDIAGLGQGRAGGGAALGGGGMGMPMGAAHQGQGGAKSKGSQQE
DEALYTEDRAWTEAVIGNRRRQDSKESK
Rv2660 - C2366 Ecoli-pET28[6xHis-CthermoCohesin (underlined)-m.tbRv2660 (bold)]
(SEQ ID NO: 202)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGTGTGATAGCGGGCGTCGACCAGGCGCTTGCAGCAACAGG
CCAGGCTAGCCAGCGGGCGGCAGGCGCATCTGGTGGGGTCACCGTCGGTGTCGGCGTG
GGCACGGAACAGAGGAACCTTTCGGTGGTTGCACCGAGTCAGTTCACATTTAGTTCACG
CAGCCCAGATTTTGTGGATGAAACCGCAGGTCAATCGTGGTGCGCGATACTGGGATTGA
ACCAGTTTCACGAATTCTAGGCGGCCGCACTCGAGCACCACCACCACCACCACTGAGATCCG
GCTGCTAACAAAGCCCGAAAGGAAGCTGAGTTGGCTGCTGCCACCGCTGAGCAATAACTAGCA
TAACCCCTTGGGGCCTCTAAACGGGTCTTGAGGGGTTTTTTGCTGAAAGGAGGAACTATATCC
GGAT
Protein sequence - Rv2660 - C2366 Ecoli-pET28[6xHis-CthermoCohesin (underlined)-
m.tbRv2660 (bold)]
(SEQ ID NO: 203)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASVIAGV
DQALAATGQASQRAAGASGGVTVGVGVGTEQRNLSVVAPSQFTFSSRSPDFVDETAGQSWCA
ILGLNQFHEF
Rv2873- C2362 Ecoli-pET28[6xHis-CthermoCohesin (underlined)-m.tbRv2873 (bold)]
Additional modifications are in italics.
(SEQ ID NO: 204)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCAAACCCGTGTCGCAAGACACCAGCCCGAAACCGGCGAC
CAGCCCGGCGGCGCCCGTTACCACGGCGGCAATGGCTGACCCCGCAGCGGACCTGATTG
GTCGTGGGTGCGCGCAATACGCGGCGCAAAATCCCACCGGTCCCGGATCGGTGGCCGGA
ATGGCGCAAGACCCGGTCGCTACCGCGGCTTCCAACAACCCGATGCTCAGTACCCTGAC
CTCGGCTCTGTCGGGCAAGCTGAACCCGGATGTGAATCTGGTCGACACCCTCAACGGCG
GCGAGTACACCGTTTTCGCCCCCACCAACGCCGCATTCGACAAGCTGCCGGCGGCCACT
ATCGATCAACTCAAGACTGACGCCAAGCTGCTCAGCAGCATCCTGACCTACCACGTGATA
GCCGGCCAGGCGAGTCCGAGCAGGATCGACGGCACCCATCAGACCCTGCAAGGTGCCGA
CCTGACGGTGATAGGCGCCCGCGACGACCTCATGGTCAACAACGCCGGTTTGGTATGTG
GCGGAGTTCACACCGCCAACGCGACGGTGTACATGATCGATACGGTGCTGATGCCCCCG
GCACAGGAATTCTAAGCGGCCGCACTCGAGCACCACCACCACCACCACTGAGATCCGGCTGCT
AACAAAGCCCGAAAGGAAGCTGAGTTGGCTGCTGCCACCGCTGAGCAATAACTAGCATAACC
CCTTGGGGCCTCTAAACGGGTCTTGAGGGGTTTTTTGCTGAAAGGAGGAACTATATCCGGAT
Protein sequence - Rv2873- C2362 Ecoli-pET28[6xHis-CthermoCohesin (underlined)-
m.tbRv2873 (bold)]
(SEQ ID NO: 205)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASKPVSQ
DTSPKPATSPAAPVTTAAMADPAADLIGRGCAQYAAQNPTGPGSVAGMAQDPVATAASNNP
MLSTLTSALSGKLNPDVNLVDTLNGGEYTVFAPTNAAFDKLPAATIDQLKTDAKLLSSILTYH
VIAGQASPSRIDGTHQTLQGADLTVIGARDDLMVNNAGLVCGGVHTANATVYMIDTVLMPP
AQEF
Rv0831 - C2356 Ecoli-pET28[6xHis-CthermoCohesin (underlined)-m.tbRv0831(bold)]
Additional modifications are in italics.
(SEQ ID NO: 206)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGC [symbol] AGCCCAACGC
ACCCGTTGCCCTGGTGACGGTGGAAATCCGTCACCCGACAACGGATTCGCTCACCGAAT
CAGCGAACCGGGAGCTCAAACACCTGCTTATCAATGATCTACCGATCGAACGCCAGGCG
CAGGACGTCAGCTGGGGGATGACGGCGCCCGGTGGAGCCCCCACCCCGGTCGCGGATC
GTTTCGTTCGTTATGTCAATCGCGATAACACCACCGCCGCTTCACTGAAGAACCAGGCGA
TAGTCGTGGAGACCACCGCCTACCGCAGCTTTGAGGCCTTTACCGACGTTGTGATGCGG
GTCGTGGATGCTCGCGCGCAGGTCTCGTCAATCGTTGGGTTGGAGCGTATCGGTCTTCG
CTTTGTTCTGGAGATCCGCGTCCCCGCGGGTGTCGACGGCCGGATCACGTGGAGCAACT
GGATCGACGAGCAGCTGCTCGGGCCGCAGCGTTTCACTCCCGGCGGCCTGGTCCTGACC
GAGTGGCAGGGTGCCGCAGTCTACCGTGAGCTACAACCAGGCAAATCGCTCATCGTGCG
CTACGGCCCGGGTATGGGCCAAGCGCTTGATCCCAATTACCATCTGCGCCGAATAACAC
CCGCCCAAACCGGACCATTCTTCCTGCTGGACATCGATAGCTTTTGGACTCCCAGTGGCG
GCTCCATTCCCGAGTACAACAGGGACGCCTTAGTGTCGACATTCCAGGACCTGTACGGTC
CGGCCCAGGTCGTGTTTCAGGAGATGAT [symbol]
GAATTCTAAGCGGCCGCACTCGAGCACCACCACCACCACCACTGAGATCCGGCTGCTAACAAAG
CCCGAAAGGAAGCTGAGTTGGCTGCTGCCACCGCTGAGCAATAACTAGCATAACCCCTTGGGG
CCTCTAAACGGGTCTTGAGGGGTTTTTTGCTGAAAGGAGGAACTATATCCGGAT
Protein sequence - Rv0831-C2356 Ecoli-pET28[6xHis-CthermoCohesin (underlined)-
m.tbRv0831(bold)]
(SEQ ID NO: 207)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMLPE
TNQDEVQPNAPVALVTVEIRHPTTDSLTESANRELKHLLINDLPIERQAQDVSWGMTAPGGAP
TPVADRFVRYVNRDNTTAASLKNQAIVVETTAYRSFEAFTDVVMRVVDARAQVSSIVGLERIG
LRFVLEIRVPAGVDGRITWSNWIDEQLLGPQRFTPGGLVLTEWQGAAVYRELQPGKSLIVRY
GPGMGQALDPNYHLRRITPAQTGPFFLLDIDSFWTPSGGSIPEYNRDALVSTFQDLYGPAQVV
FQEMITSRLKDELLRQ
Rv0632 - C2355 Ecoli-pET28[6xHis-CthermoCohesin (underlined) -m.tbRv0632 (bold)]
Additional modifications are in italics.
(SEQ ID NO: 208)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCATGAGCGACCCGGTCAGCTATACCCGCAAGGATTCCATC
GCCGTCATCAGTATGGACGACGGCAAGGTCAACGCACTGGGCCCGGCGATGCAACAAGC
CCTCAATGCAGCGATCGACAACGCGGACCGTGATGATGTTGGGGCGCTGGTGATCACCG
GTAATGGCCGGGTATTCAGCGGAGGCTTCGACCTGAAGATCCTCACCTCCGGTGAAGTG
CAGCCCGCGATCGACATGCTCAGGGGCGGCTTCGAGCTGGCGTATCGCCTCTTGTCCTA
CCCCAAACCGGTGGTGATGGCGTGCACCGGTCACGCCATCGCCATGGGCGCGTTTCTGT
TGTCCTGCGGCGATCATCGGGTGGCGGCCCACGCATACAACATCCAGGCCAATGAGGTC
GCGATCGGCATGACCATTCCGTACGCGGCGTTAGAGATCATGAAGCTGCGACTGACCCG
GTCGGCATACCAGCAGGCAACCGGGCTGGCCAAGACGTTCTTCGGGGAAACCGCGCTGG
CCGCCGGGTTTATCGACGAGATCGCCCTGCCGGAGGTGGTGGTCAGCCGCGCCGAGGAA
GCCGCACGAGAGTTCGCCGGTCTCAACCAACACGCCCATGCCGCGACCAAGTTGCGCTC
CCGCGCCGACGCGCTCACTGCGATTCGGGCCGGGATCGACGGGATAGCAGCCGAGTTCG
GGCTGGAATTCTAAGCGGCCGCACTCGAGCACCACCACCACCACCACTGAGATCCGGCTGCTA
ACAAAGCCCGAAAGGAAGCTGAGTTGGCTGCTGCCACCGCTGAGCAATAACTAGCATAACCCC
TTGGGGCCTCTAAACGGGTCTTGAGGGGTTTTTTGCTGAAAGGAGGAACTATATCCGGAT
Protein sequence-Rv0632 - C2355 Ecoli-pET28[6xHis-CthermoCohesin (underlined) -
m.tbRv0632 (bold)]
(SEQ ID NO: 209)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMSDP
VSYTRKDSIAVISMDDGKVNALGPAMQQALNAAIDNADRDDVGALVITGNGRVFSGGFDLKI
LTSGEVQPAIDMLRGGFELAYRLLSYPKPVVMACTGHAIAMGAFLLSCGDHRVAAHAYNIQA
NEVAIGMTIPYAALEIMKLRLTRSAYQQATGLAKTFFGETALAAGFIDEIALPEVVVSRAEEA
AREFAGLNQHAHAATKLRSRADALTAIRAGIDGIAAEFGLEF
Ag85C - C2391 Ecoli-pET28[6xHis-CthermoCohesin (underlined)-m.tbAg85C (bold)]
Additional modifications are in italics.
(SEQ ID NO: 210)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGCTGCAGCTGT [symbol] [symbol]
CGCAGCGACAACCCTGCCGCGCCGGCTGGCTATCGCGGCTATGGGGGCTGTCCTGGT
TTACGGTCTGGTCGGTACCTTCGGCGGGCCGGCCACCGCGGGCGCATTCTCTAGGCCCG
GTCTTCCAGTGGAATATCTGCAGGTGCCATCCGCGTCGATGGGCCGCGACATCAAGGTC
CAGTTCCAGGGCGGCGGACCGCACGCGGTCTACCTGCTCGACGGTCTGCGGGCCCAGGA
TGACTACAACGGCTGGGACATCAACACCCCGGCCTTCGAGGAGTACTACCAGTCAGGGT
TGTCGGTGATCATGCCCGTGGGCGGCCAATCCAGTTTCTACACCGACTGGTATCAGCCCT
CGCAGAGCAACGGCCAGAACTACACCTACAAGTGGGAGACCTTCCTTACCAGAGAGATG
CCCGCCTGGCTACAGGCCAACAAGGGCGTGTCCCCGACAGGCAACGCGGCGGTGGGTCT
TTCGATGTCGGGCGGTTCCGCGCTGATCCTGGCCGCGTACTACCCGCAGCAGTTCCCGT
ACGCCGCGTCGTTGTCGGGCTTCCTCAACCCGTCCGAGGGCTGGTGGCCGACGCTGATC
GGCCTGGCGATGAACGACTCGGGCGGTTACAACGCCAACAGCATGTGGGGTCCGTCCAG
CGACCCGGCCTGGAAGCGCAACGACCCAATGGTTCAGATTCCCCGCCTGGTCGCCAACA
ACACCCGGATCTGGGTGTACTGCGGTAACGGCACACCCAGCGACCTCGGCGGCGACAAC
ATACCGGCGAAGTTCCTGGAAGGCCTCACCCTGCGCACCAACCAGACCTTCCGGGACAC
CTACGCGGCCGACGGTGGACGCAACGGGGTGTTTAACTTCCCGCCCAACGGAACACACT
CGTGGCCCTACTGGAACGAGCAGCTGGTCGCCATGAAGGCCGATATCCAGCATGTGCTC
AACGGCGCG [symbol] GCAGCTGTTGAGCGGCCGCA
CTCGAGCACCACCACCACCACCACTGAGATCCGGCTGCTAACAAAGCCCGAAAGGAAGCTGAG
TTGGCTGCTGCCACCGCTGAGCAATAACTAGCATAACCCCTTGGGGCCTCTAAACGGGTCTTG
AGGGGTTTTTTGCTGAAAGGAGGAACTATATCCGGAT
Protein sequence-Ag85C-C2391 Ecoli-pET28[6xHis-CthermoCohesin (underlined)-
m.tbAg85C (bold)]
(SEQ ID NO: 211)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASCSCMT
FFEQVRRLRSAATTLPRRLAIAAMGAVLVYGLVGTFGGPATAGAFSRPGLPVEYLQVPSASM
GRDIKVQFQGGGPHAVYLLDGLRAQDDYNGWDINTPAFEEYYQSGLSVIMPVGGQSSFYTD
WYQPSQSNGQNYTYKWETFLTREMPAWLQANKGVSPTGNAAVGLSMSGGSALILAAYYPQ
QFPYAASLSGFLNPSEGWWPTLIGLAMNDSGGYNANSMWGPSSDPAWKRNDPMVQIPRLVA
NNTRIWVYCGNGTPSDLGGDNIPAKFLEGLTLRTNQTFRDTYAADGGRNGVFNFPPNGTHSW
PYWNEQLVAMKADIQHVLNGATPPAAPAAPAACSC
Rv2658-C2361 Ecoli-pET28[6xHis-CthermoCohesin (underlined)-m.tbRv2658 (bold)]
Additional modifications are in italics.
(SEQ ID NO: 212)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGAAAAACGCCAGCAA
CGCGGCCTTTTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGCCGCAGCCGA
ACGACCGAGCGCAGCGAGTCAGTGAGCGAGGAAGCGGAAGAGCGCCTGATGCGGTATTTTCT
CCTTACGCATCTGTGCGGTATTTCACACCGCATATATGGTGCACTCTCAGTACAATCTGCTCTG
ATGCCGCATAGTTAAGCCAGTATACACTCCGCTATCGCTACGTGACTGGGTCATGGCTGCGCCC
CGACACCCGCCAACACCCGCTGACGCGCCCTGACGGGCTTGTCTGCTCCCGGCATCCGCTTAC
AGACAAGCTGTGACCGTCTCCGGGAGCTGCATGTGTCAGAGGTTTTCACCGTCATCACCGAAA
CGCGCGAGGCAGCTGCGGTAAAGCTCATCAGCGTGGTCGTGAAGCGATTCACAGATGTCTGCC
TGTTCATCCGCGTCCAGCTCGTTGAGTTTCTCCAGAAGCGTTAATGTCTGGCTTCTGATAAAGC
GTTCATGGGGGTAATGATACCGATGAAACGAGAGAGGATGCTCACGATACGGGTTACTGATGA
TGAACATGCCCGGTTACTGGAACGTTGTGAGGGTAAACAACTGGCGGTATGGATGCGGCGGGA
CCAGAGAAAAATCACTCAGGGTCAATGCCAGCGCTTCGTTAATACAGATGTAGGTGTTCCACA
GGGTAGCCAGCAGCATCCTGCGATGCAGATCCGGAACATAATGGTGCAGGGCGCTGACTTCCG
CGTTTCCAGACTTTACGAAACACGGAAACCGAAGACCATTCATGTTGTTGCTCAGGTCGCAGA
CGTTTTGCAGCAGCAGTCGCTTCACGTTCGCTCGCGTATCGGTGATTCATTCTGCTAACCAGTA
AGGCAACCCCGCCAGCCTAGCCGGGTCCTCAACGACAGGAGCACGATCATGCGCACCCGTGG
GGCCGCCATGCCGGCGATAATGGCCTGCTTCTCGCCGAAACGTTTGGTGGCGGGACCAGTGAC
GAAGGCTTGAGCGAGGGCGTGCAAGATTCCGAATACCGCAAGCGACAGGCCGATCATCGTCG
CGCTCCAGCGAAAGCGGTCCTCGCCGAAAATGACCCAGAGCGCTGCCGGCACCTGTCCTACGA
GTTGCATGATAAAGAAGACAGTCATAAGTGCGGCGACGATAGTCATGCCCCGCGCCCACCGGA
AGGAGCTGACTGGGTTGAAGGCTCTCAAGGGCATCGGTCGAGATCCCGGTGCCTAATGAGTGA
GCTAACTTACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCGCCAGGGTGG
TTTTTCTTTTCACCAGTGAGACGGGCAACAGCTGATTGCCCTTCACCGCCTGGCCCTGAGAGAG
TTGCAGCAAGCGGTCCACGCTGGTTTGCCCCAGCAGGCGAAAATCCTGTTTGATGGTGGTTAA
CGGCGGGATATAACATGAGCTGTCTTCGGTATCGTCGTATCCCACTACCGAGATATCCGCACC
AACGCGCAGCCCGGACTCGGTAATGGCGCGCATTGCGCCCAGCGCCATCTGATCGTTGGCAAC
CAGCATCGCAGTGGGAACGATGCCCTCATTCAGCATTTGCATGGTTTGTTGAAAACCGGACAT
GGCACTCCAGTCGCCTTCCCGTTCCGCTATCGGCTGAATTTGATTGCGAGTGAGATATTTATGC
CAGCCAGCCAGACGCAGACGCGCCGAGACAGAACTTAATGGGCCCGCTAACAGCGCGATTTG
CTGGTGACCCAATGCGACCAGATGCTCCACGCCCAGTCGCGTACCGTCTTCATGGGAGAAAAT
AATACTGTTGATGGGTGTCTGGTCAGAGACATCAAGAAATAACGCCGGAACATTAGTGCAGGC
AGCTTCCACAGCAATGGCATCCTGGTCATCCAGCGGATAGTTAATGATCAGCCCACTGACGCG
TTGCGCGAGAAGATTGTGCACCGCCGCTTTACAGGCTTCGACGCCGCTTCGTTCTACCATCGAC
ACCACCACGCTGGCACCCAGTTGATCGGCGCGAGATTTAATCGCCGCGACAATTTGCGACGGC
GCGTGCAGGGCCAGACTGGAGGTGGCAACGCCAATCAGCAACGACTGTTTGCCCGCCAGTTGT
TGTGCCACGCGGTTGGGAATGTAATTCAGCTCCGCCATCGCCGCTTCCACTTTTTCCCGCGTTTT
CGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGC [symbol] TGCAACTGCGA
CGACGAACCGGGAGCGCTCATCATCGCCTGGATCGACGACGAACGACCCGCCGGCGGGC
ACATACAGATGCGGTCGAACACCCGCTTCACCGAAACACAGTGGGGCCGCCATATCGAG
TGGAAACTCGAATGCCGGGCATGCCGAAAGTATGCGCCGATATCCGAGATGACCGCCGC
GGCGATCCTCGACGGTTTCGGGGCGAAGCTTCACGAGCTGAGAACGTCGACCATCCCCG
ACGCTGACGATCCATCAATAGCAGAGGCGCGACACGTAATTCCGTTCAGC [symbol] [symbol]
GAATTCTAAGCGGCCGCACTCGAGCACCACCACCACCACCAC
TGAGATCCGGCTGCTAACAAAGCCCGAAAGGAAGCTGAGTTGGCTGCTGCCACCGCTGAGCAA
TAACTAGCATAACCCCTTGGGGCCTCTAAACGGGTCTTGAGGGGTTTTTTGCTGAAAGGAGGA
ACTATATCCGGAT
Protein sequence-Rv2658-C2361 Ecoli-pET28[6xHis-CthermoCohesin (underlined)-
m.tbRv2658 (bold)]
(SEQ ID NO: 213)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMADA
VKYVVMCNCDDEPGALIIAWIDDERPAGGHIQMRSNTRFTETQWGRHIEWKLECRACRKYA
PISEMTAAAILDGFGAKLHELRTSTIPDADDPSIAEARHVIPFSALCLRLSQLGGEF
Rv2659-C2388 Ecoli-pET28[6xHis-CthermoCohesin (underlined)-m.tbRv2659 (bold)]
Additional modifications are in italics.
(SEQ ID NO: 214)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGC [symbol] AATTCGGTCG
CATCCGACAGTTCAACTCCGGCCGCTGGCAAGCCAGCTACACCGGCCCCGACGGCCGCG
TGTACATCGCCCCCAAAACCTTCAACGCCAAGATCGACGCCGAAGCATGGCTCACCGAC
CGCCGCCGCGAAATCGACCGACAACTATGGTCCCCGGCATCGGGTCAGGAAGACCGCCC
CGGAGCCCCATTCGGTGAGTACGCCGAAGGATGGCTGAAGCAGCGTGGAATCAAGGACC
GCACCCGCGCCCACTATCGCAAACTGCTGGACAACCACATCCTGGCCACCTTCGCTGACA
CCGACCTACGCGACATCACCCCGGCCGCCGTGCGCCGCTGGTACGCCACCACCGCCGTG
GGCACACCGACCATGCGGGCACACTCCTACAGCTTGCTGCGCGCAATCATGCAGACCGC
CTTGGCCGACGACCTGATCGACTCCAACCCCTGCCGCATCTCAGGCGCGTCCACCGCCC
GCCGCGTCCACAAGATCAGGCCCGCCACCCTCGACGAGCTGGAAACCATCACCAAAGCC
ATGCCCGACCCCTACCAGGCGTTCGTGCTGATGGCGGCATGGCTGGCCATGCGCTACGG
CGAGCTGACCGAATTACGCCGCAAAGACATCGACCTGCACGGCGAGGTTGCGCGGGTGC
GGCGGGCTGTCGTTCGGGTGGGCGAAGGCTTCAAGGTGACGACACCGAAAAGCGATGC
GGGAGTGCGCGACATAAGTATCCCGCCACATCTGATACCCGCCATCGAAGACCACCTTC
ACAAACACGTCAACCCCGGCCGGGAGTCCCTGCTGTTCCCATCGGTCAACGACCCCAAC
CGTCACCTAGCACCCTCGGCGCTGTACCGCATGTTCTACAAGGCCCGAAAAGCCGCCGG
CCGACCAGACTTACGGGTGCACGACCTTCGACACTCCGGCGCCGTGTTGGCTGCATCCA
CCGGCGCCACACTGGCCGAACTGATGCAGCGGCTAGGACACAGCACAGCCGGCGCCGCA
CTCCGCTACCAGCACGCCGCCAAGGGCCGGGACCGCGAAATCGCCGCACTGTTAAGCAA
ACTGGCCGAGAACCAGGAGATGGAATTCTAAGCGGCCGCACTCGAGCACCACCACCACCAC
CACTGAGATCCGGCTGCTAACAAAGCCCGAAAGGAAGCTGAGTTGGCTGCTGCCACCGCTGAG
CAATAACTAGCATAACCCCTTGGGGCCTCTAAACGGGTCTTGAGGGGTTTTTTGCTGAAAGGA
GGAACTATATCCGGAT
Protein sequence-Rv2659-C2388 Ecoli-pET28[6xHis-CthermoCohesin (underlined)-
m.tbRv2659 (bold)]
(SEQ ID NO: 215)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASVTQT
GKRQRRKFGRIRQFNSGRWQASYTGPDGRVYIAPKTFNAKIDAEAWLTDRRREIDRQLWSPA
SGQEDRPGAPFGEYAEGWLKQRGIKDRTRAHYRKLLDNHILATFADTDLRDITPAAVRRWY
ATTAVGTPTMRAHSYSLLRAIMQTALADDLIDSNPCRISGASTARRVHKIRPATLDELETITKA
MPDPYQAFVLMAAWLAMRYGELTELRRKDIDLHGEVARVRRAVVRVGEGFKVTTPKSDAG
VRDISIPPHLIPAIEDHLHKHVNPGRESLLFPSVNDPNRHLAPSALYRMFYKARKAAGRPDLRV
HDLRHSGAVLAASTGATLAELMQRLGHSTAGAALRYQHAAKGRDREIAALLSKLAENQEMEF
Rv0577-C2411 Ecoli-pET28[6xHis-CthermoCohesin (underlined)-m.tbRv0577 (bold)]
Additional modifications are in italics.
(SEQ ID NO: 216)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGT [symbol] CCGAACTG
GGTCGACCTTCAGACCACCGATCAGTCCGCCGCCAAAAAGTTCTACACATCGTTGTTCGG
CTGGGGTTACGACGACAACCCGGTCCCCGGAGGCGGTGGGGTCTATTCCATGGCCACGC
TGAACGGCGAAGCCGTGGCCGCCATCGCACCGATGCCCCCGGGTGCACCGGAGGGGAT
GCCGCCGATCTGGAACACCTATATCGCGGTGGACGACGTCGATGCGGTGGTGGACAAGG
TGGTGCCCGGGGGCGGGCAGGTGATGATGCCGGCCTTCGACATCGGCGATGCCGGCCG
GATGTCGTTCATCACCGATCCGACCGGCGCTGCCGTGGGCCTATGGCAGGCCAATCGGC
ACATCGGAGCGACGTTGGTCAACGAGACGGGCACGCTCATCTGGAACGAACTGCTCACG
GACAAGCCGGATTTGGCGCTAGCGTTCTACGAGGCTGTGGTTGGCCTCACCCACTCGAG
CATGGAGATAGCTGCGGGCCAGAACTATCGGGTGCTCAAGGCCGGCGACGCGGAAGTCG
GCGGCTGTATGGAACCGCCGATGCCCGGCGTGCCGAATCATTGGCACGTCTACTTTGCG
GTGGATGACGCCGACGCCACGGCGGCCAAAGCCGCCGCAGCGGGCGGCCAGGTCATTG
CGGAACCGGCTGACATTCCGTCGGTGGGCCGGTTCGCCGTGTTGTCCGATCCGCAGGGC
GCG [symbol] GCGGCCGCACTCGAGCACCACCACC
ACCACCACTGAGATCCGGCTGCTAACAAAGCCCGAAAGGAAGCTGAGTTGGCTGCTGCCACCG
CTGAGCAATAACTAGCATAACCCCTTGGGGCCTCTAAACGGGTCTTGAGGGGTTTTTTGCTGAA
AGGAGGAACTATATCCGGAT
Protein sequence- Rv0577- C2411 Ecoli-pET28[6xHis-CthermoCohesin (underlined)-
m.tbRv0577 (bold)]
(SEQ ID NO: 217)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMPKR
SEYRQGTPNWVDLQTTDQSAAKKFYTSLFGWGYDDNPVPGGGGVYSMATLNGEAVAAIAP
MPPGAPEGMPPIWNTYIAVDDVDAVVDKVVPGGGQVMMPAFDIGDAGRMSFITDPTGAAVG
LWQANRHIGATLVNETGTLIWNELLTDKPDLALAFYEAVVGLTHSSMEIAAGQNYRVLKAG
DAEVGGCMEPPMPGVPNHWHVYFAVDDADATAAKAAAAGGQVIAEPADIPSVGRFAVLSDP
QGAIFSVLKPAPQQ
Mtb72f - C2236 Ecoli-pET28[6xHis-CthermoCohesin (underlined)-m.tbMtb72f (bold)]
Additional modifications are in italics.
(SEQ ID NO: 218)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGC [symbol] GGGTGGGCA
GGGATTCGCCATTCCGATCGGGCAGGCGATGGCGATCGCGGGCCAGATCCGATCGGGTG
GGGGGTCACCCACCGTTCATATCGGGCCTACCGCCTTCCTCGGCTTGGGTGTTGTCGAC
AACAACGGCAACGGCGCACGAGTCCAACGCGTGGTCGGGAGCGCTCCGGCGGCAAGTCT
CGGCATCTCCACCGGCGACGTGATCACCGCGGTCGACGGCGCTCCGATCAACTCGGCCA
CCGCGATGGCGGACGCGCTTAACGGGCATCATCCCGGTGACGTCATCTCGGTGACCTGG
CAAACCAAGTCGGGCGGCACGCGTACAGG [symbol] G
AATTC [symbol] AACTCCGCGAGGATGTACGCCGGC
CCGGGTTCGGCCTCGCTGGTGGCCGCGGCTCAGATGTGGGACAGCGTGGCGAGTGACCT
GTTTTCGGCCGCGTCGGCGTTTCAGTCGGTGGTCTGGGGTCTGACGGTGGGGTCGTGGA
TAGGTTCGTCGGCGGGTCTGATGGTGGCGGCGGCCTCGCCGTATGTGGCGTGGATGAGC
GTCACCGCGGGGCAGGCCGAGCTGACCGCCGCCCAGGTCCGGGTTGCTGCGGCGGCCT
ACGAGACGGCGTATGGGCTGACGGTGCCCCCGCCGGTGATCGCCGAGAACCGTGCTGAA
CTGATGATTCTGATAGCGACCAACCTCTTGGGGCAAAACACCCCGGCGATCGCGGTCAA
CGAGGCCGAATACGGCGAGATGTGGGCCCAAGACGCCGCCGCGATGTTTGGCTACGCCG
CGGCGACGGCGACGGCGACGGCGACGTTGCTGCCGTTCGAGGAGGCGCCGGAGATGAC
CAGCGCGGGTGGGCTCCTCGAGCAGGCCGCCGCGGTCGAGGAGGCCTCCGACACCGCC
GCGGCGAACCAGTTGATGAACAATGTGCCCCAGGCGCTGCAACAGCTGGCCCAGCCCAC
GCAGGGCACCACGCCTTCTTCCAAGCTGGGTGGCCTGTGGAAGACGGTCTCGCCGCATC
GGTCGCCGATCAGCAACATGGTGTCGATGGCCAACAACCACATGTCGATGACCAACTCG
GGTGTGTCGATGACCAACACCTTGAGCTCGATGTTGAAGGGCTTTGCTCCGGCGGCGGC
CGCCCAGGCCGTGCAAACCGCGGCGCAAAACGGGGTCCGGGCGATGAGCTCGCTGGGC
AGCTCGCTGGGTTCTTCGGGTCTGGGCGGTGGGGTGGCCGCCAACTTGGGTCGGGCGGC
CTCGGTCGGTTCGTTGTCGGTGCCGCAGGCCTGGGCCGCGGCCAACCAGGCAGTCACCC
CGGCGGCGCGGGCGCTGCCGCTGACCAGCCTGACCAGCGCCGCGGAAAGAGGGCCCGG
GCAGATGCTGGGCGGGCTGCCGGTGGGGCAGATGGGCGCCAGGGCCGGTGGTGGGCTC
AGTGGTGTGCTGCGTGTTCCGCCGCG [symbol] [symbol] [symbol]
CCGACTTCCCCGCGCTGCCCCTCGACC
CGTCCGCGATGGTCGCCCAAGTGGGGCCACAGGTGGTCAACATCAACACCAAACTGGGC
TACAACAACGCCGTGGGCGCCGGGACCGGCATCGTCATCGATCCCAACGGTGTCGTGCT
GACCAACAACCACGTGATCGCGGGCGCCACCGACATCAATGCGTTCAGCGTCGGCTCCG
GCCAAACCTACGGCGTCGATGTGGTCGGGTATGACCGCACCCAGGATGTCGCGGTGCTG
CAGCTGCGCGGTGCCGGTGGCCTGCCGTCGGCGGCGATCGGTGGCGGCGTCGCGGTTG
GTGAGCCCGTCGTCGCGATGGGCAACAGCGGTGGGCAGGGCGGAACGCCCCGTGCGGT
GCCTGGCAGGGTGGTCGCGCTCGGCCAAACCGTGCAGGCGTCGGATTCGCTGACCGGTG
CCGAAGAGACATTGAACGGGTTGATCCAGTTCGATGCCGCGATCCAGCCCGGTGATTCG
GGCGGGCCCGTCGTCAACGGCCT [symbol] [symbol]
GCACTCGAGCACCACCACCACCACCAC [symbol] GATCCGGCTGCTAACAAAGCC
CGAAAGGAAGCTGAGTTGGCTGCTGCCACCGCTGAGCAATAACTAGCATAACCCCTTGGGGCC
TCTAAACGGGTCTTGAGGGGTTTTTTGCTGAAAGGAGGAACTATATCCGGAT
Protein sequence - Mtb72f - C2236 Ecoli-pET28[6xHis-CthermoCohesin (underlined)-
m.tbMtb72f (bold)]
(SEQ ID NO: 219)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASTAASD
NFQLSQGGQGFAIPIGQAMAIAGQIRSGGGSPTVHIGPTAFLGLGVVDNNGNGARVQRVVGS
APAASLGISTGDVITAVDGAPINSATAMADALNGHHPGDVISVTWQTKSGGTRTGNVTLAEGP
PAEFMVDFGALPPEINSARMYAGPGSASLVAAAQMWDSVASDLFSAASAFQSVVWGLTVGS
WIGSSAGLMVAAASPYVAWMSVTAGQAELTAAQVRVAAAAYETAYGLTVPPPVIAENRAEL
MILIATNLLGQNTPAIAVNEAEYGEMWAQDAAAMFGYAAATATATATLLPFEEAPEMTSAG
GLLEQAAAVEEASDTAAANQLMNNVPQALQQLAQPTQGTTPSSKLGGLWKTVSPHRSPISN
MVSMANNHMSMTNSGVSMTNTLSSMLKGFAPAAAAQAVQTAAQNGVRAMSSLGSSLGSSG
LGGGVAANLGRAASVGSLSVPQAWAAANQAVTPAARALPLTSLTSAAERGPGQMLGGLPVG
QMGARAGGGLSGVLRVPPRPYVMPHSPAAGDIAPPALSQDRFADFPALPLDPSAMVAQVGPQ
VVNINTKLGYNNAVGAGTGIVIDPNGVVLTNNHVIAGATDINAFSVGSGQTYGVDVVGYDRT
QDVAVLQLRGAGGLPSAAIGGGVAVGEPVVAMGNSGGQGGTPRAVPGRVVALGQTVQASD
SLTGAEETLNGLIQFDAAIQPGDSGGPVVNGLGQVVGMNTAAS
Rv1411-LPTEG - C2198 Ecoli-pET28[6xHis-CthermoCohesin (underlined)-m.tbRv1411-
LPETGsite-6xHis (bold)] Additional modifications are in italics.
(SEQ ID NO: 220)
TGGCGAATGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCCTTTCTCG
CCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGT
TCCAAACTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTTTAACAA
AATATTAACGTTTACAATTTCAGGTGGCACTTTTCGGGGAAATGTGCGCGGAACCCCTATTTGT
TTATTTTTCTAAATACATTCAAATATGTATCCGCTCATGAATTAATTCTTAGAAAAACTCATCG
AGCATCAAATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCCTGGTATC
GGTCTGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGTTTATG
CATTTCTTTCCAGACTTGTTCAACAGGCCAGCCATTACGCTCGTCATCAAAATCACTCGCATCA
ACCAAACCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATCAACAAT
ATTTTCACCTGAATCAGGATATTCTTCTAATACCTGGAATGCTGTTTTCCCGGGGATCGCAGTG
GTGAGTAACCATGCATCATCAGGAGTACGGATAAAATGCTTGATGGTCGGAAGAGGCATAAAT
TCCGTCAGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAATCGC
GGCCTAGAGCAAGACGTTTCCCGTTGAATATGGCTCATAACACCCCTTGTATTACTGTTTATGT
AAGCAGACAGTTTTATTGTTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTCCTTCTAGTGTAG
CCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGC
ACGAGGGAGCTTCCAGGGGGAAACGCCTGGTATCTTTATAGTCCTGTCGGGTTTCGCCACCTCT
GACTTGAGCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCTGATGCGGTATTTT
CTCCTTACGCATCTGTGCGGTATTTCACACCGCATATATGGTGCACTCTCAGTACAATCTGCTC
TGATGCCGCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAA
ACGCGCGAGGCAGCTGCGGTAAAGCTCATCAGCGTGGTCGTGAAGCGATTCACAGATGTCTGC
CTGTTCATCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGGGGATTTC
TGTTCATGGGGGTAATGATACCGATGAAACGAGAGAGGATGCTCACGATACGGGTTACTGATG
ATGAACATGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGTGTTCCAC
AGGGTAGCCAGCAGCATCCTGCGATGCAGATCCGGAACATAATGGTGCAGGGCGCTGACTTCC
GCGTTTCCAGACTTTACGAAACACGGAAACCGAAGACCATTCATGTTGTTGCTCAGGTCGCAG
ACGTTTTGCAGCAGCAGTCGCTTCACGTTCGCTCGCGTATCGGTGATTCATTCTGCTAACCAGT
AAGGCAACCCCGCCAGCCTAGCCGGGTCCTCAACGACAGGAGCACGATCATGCGCACCCGTG
GGGCCGCCATGCCGGCGATAATGGCCTGCTTCTCGCCGAAACGTTTGGTGGCGGGACCAGTGA
CGAAGGCTTGAGCGAGGGCGTGCAAGATTCCGAATACCGCAAGCGACAGGCCGATCATCGTC
GCGCTCCAGCGAAAGCGGTCCTCGCCGAAAATGACCCAGAGCGCTGCCGGCACCTGTCCTACG
AGTTGCATGATAAAGAAGACAGTCATAAGTGCGGCGACGATAGTCATGCCCCGCGCCCACCGG
AAGGAGCTGACTGGGTTGAAGGCTCTCAAGGGCATCGGTCGAGATCCCGGTGCCTAATGAGTG
AGCTAACTTACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCCAGGGTG
GTTTTTCTTTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATATCCGCAC
CAACGCGCAGCCCGGACTCGGTAATGGCGCGCATTGCGCCCAGCGCCATCTGATCGTTGGCAA
CCAGCATCGCAGTGGGAACGATGCCCTCATTCAGCATTTGCATGGTTTGTTGAAAACCGGACA
TGGCACTCCAGTCGCCTTCCCGTTCCGCTATCGGCTGAATTTGATTGCGAGTGAGATATTTATG
CCAGCCAGCCAGACGCAGACGCGCCGAGACAGAACTTAATGGGCCCGCTAACAGCGCGATTT
GCTGGTGACCCAATGCGACCAGATGCTCCACGCCCAGTCGCGTACCGTCTTCATGGGAGAAAA
TAATACTGTTGATGGGTGTCTGGTCAGAGACATCAAGAAATAACGCCGGAACATTAGTGCAGG
CAGCTTCCACAGCAATGGCATCCTGGTCATCCAGCGGATAGTTAATGATCAGCCCACTGACGC
GTTGCGCGAGAAGATTGTGCACCGCCGCTTTACAGGCTTCGACGCCGCTTCGTTCTACCATCGA
CACCACCACGCTGGCACCCAGTTGATCGGCGCGAGATTTAATCGCCGCGACAATTTGCGACGG
CGCGTGCAGGGCCAGACTGGAGGTGGCAACGCCAATCAGCAACGACTGTTTGCCCGCCAGTTG
TTGTGCCACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACACCGGCAT
ACTCTGCGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTCGACGCTC
TCCCTTATGCGACTCCTGCATTAGGAAGCAGCCCAGTAGTAGGTTGAGGCCGTTGAGCACCGC
CGCCGCAAGGAATGGTGCATGCAAGGAGATGGCGCCCAACAGTCCCCCGGCCACGGGGCCTG
CCACCATACCCACGCCGAAACAAGCGCTCATGAGCCCGAAGTGGCGAGCCCGATCTTCCCCAT
CGGTGATGTCGGCGATATAGGCGCCAGCAACCGCACCTGTGGCGCCGGTGATGCCGGCCACGA
TGCGTCCGGCGTAGAGGATCGAGATCTCGATCCCGCGAAATTAATACGACTCACTATAGGGGA
ATTGTGAGCGGATAACAATTCCCCTCTAGAAATAATTTTGTTTAACTTTAAGAAGGAGATATAC
CATGGGCAGCAGCCATCATCATCATCATCACAGCAGCGGCCTGGTGCCGCGCGGCAGCCATAT
GGCTAGTATGGATCTGGATGCAGTAAGGATTAAAGTGGACACAGTAAATGCAAAACCGGGAG
ACACAGTAAATATACCTGTAAGATTCAGTGGTATACCATCCAAGGGAATAGCAAACTGTGACT
TTGTATACAGCTATGACCCGAATGTACTTGAGATAATAGAGATAAAACCGGGAGAATTGATAG
TTGACCCGAATCCTACCAAGAGCTTTGATACTGCAGTATATCCTGACAGAAAGATGATAGTAT
TCCTGTTTGCGGAAGACAGCGGAACAGGAGCGTATGCAATAACTAAAGACGGAGTATTTGCTA
CGATAGTAGCGAAAGTAAAAGAAGGAGCACCTAACGGGCTCAGTGTAATCAAATTTGTAGAA
GTAGGCGGATTTGCGAACAATGACCTTGTAGAACAGAAGACACAGTTCTTTGACGGTGGAGTA
AATGTTGGAGATACAACAGAACCTGCAACACCTACAACACCTGTAACAACACCGACAACAAC
AGATGATCTAGATGCAGCTAGC [symbol] TCGCCGTCCT
CGCCGCCGTTAGCATCGCCGCCACTGTCGTTGCCGGCTGCTCGTCGGGCTCGAAGCCAA
GCGGCGGACCACTTCCGGACGCGAAGCCGCTGGTCGAGGAGGCCACCGCGCAGACCAA
GGCTCTCAAGAGCGCGCACATGGTGCTGACGGTCAACGGCAAGATCCCGGGACTGTCTC
TGAAGACGCTGAGCGGCGATCTCACCACCAACCCCACCGCCGCGACGGGAAACGTCAAG
CTCACGCTGGGTGGGTCTGATATCGATGCCGACTTCGTGGTGTTCGACGGGATCCTGTA
CGCCACCCTGACGCCCAACCAGTGGAGCGATTTCGGTCCCGCCGCCGACATCTACGACC
CCGCCCAGGTGCTGAATCCGGATACCGGCCTGGCCAACGTGCTGGCGAATTTCGCCGAC
GCAAAAGCCGAAGGGCGGGATACCATCAACGGCCAGAACACCATCCGCATCAGCGGGAA
GGTATCGGCACAGGCGGTGAACCAGATAGCGCCGCCGTTCAACGCGACGCAGCCGGTGC
CGGCGACCGTCTGGATTCAGGAGACCGGCGATCATCAACTGGCACAGGCCCAGTTGGAC
CGCGGCTCGGGCAATTCCGTCCAGATGACCTTGTCGAAATGGGGCGAGA [symbol] [symbol]
CTGCCTGAGACTGGTGAAGCACATCACCATCACCATCACTGAGCG
GCCGCACTCGAGCACCACCACCACCACCACTGAGATCCGGCTGCTAACAAAGCCCGAAAGGAA
GCTGAGTTGGCTGCTGCCACCGCTGAGCAATAACTAGCATAACCCCTTGGGGCCTCTAAACGG
GTCTTGAGGGGTTTTTTGCTGAAAGGAGGAACTATATCCGGAT
Protein sequence - Rv1411-LPTEG - C2198 Ecoli-pET28[6xHis-CthermoCohesin
(underlined)- m.tbRv1411-LPETGsite-6xHis (bold)]
(SEQ ID NO: 221)
MGSSHHHHHHSSGLVPRGSHMASMDLDAVRIKVDTVNAKPGDTVNIPVRFSGIPSKGIANCDFVYS
YDPNVLEIIEIKPGELIVDPNPTKSFDTAVYPDRKMIVFLFAEDSGTGAYAITKDGVFATIVAKVKEG
APNGLSVIKFVEVGGFANNDLVEQKTQFFDGGVNVGDTTEPATPTTPVTTPTTTDDLDAASMRTP
RRHCRRIAVLAAVSIAATVVAGCSSGSKPSGGPLPDAKPLVEEATAQTKALKSAHMVLTVNG
KIPGLSLKTLSGDLTTNPTAATGNVKLTLGGSDIDADFVVFDGILYATLTPNQWSDFGPAADIY
DPAQVLNPDTGLANVLANFADAKAEGRDTINGQNTIRISGKVSAQAVNQIAPPFNATQPVPAT
VWIQETGDHQLAQAQLDRGSGNSVQMTLSKWGEKVQVTKPPVSLPETGEAHHHHHH

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application No. 20040146948: Compositions and Methods for Targeting Antigen-Presenting Cells with Antibody Single-Chain Variable Region Fragments.
U.S. Pat. No. 7,560,534: Molecular Conjugates Comprising Human Monoclonal Antibodies to Dendritic Cells.
U.S. Pat. No. 7,261,897: Fusion Proteins of Mycobacterium Tuberculosis.
1. Dye, C., Scheele, S., Dolin, P., Pathania, V., and Raviglione, M. C. (1999) Jama 282, 677-686.
2. Aziz, M. A., Wright, A., Laszlo, A., De Muynck, A., Portaels, F., Van Deun, A., Wells, C., Nunn, P., Blanc, L., and Raviglione, M. (2006) Lancet 368, 2142-2154.
3. Colditz, G. A., Berkey, C. S., Mosteller, F., Brewer, T. F., Wilson, M. E., Burdick, E., and Fineberg, H. V. (1995) Pediatrics 96, 29-35.
4. van Vliet, S. J., Garcia-Vallejo, J. J., and van Kooyk, Y. (2008) Immunol Cell Biol 86, 580-587.
5. Banchereau, J., Briere, F., Caux, C., Davoust, J., Lebecque, S., Liu, Y. J., Pulendran, B., and Palucka, K. (2000) Annu Rev Immunol 18, 767-811.
6. Cella, M., Engering, A., Pinet, V., Pieters, J., and Lanzavecchia, A. (1997) Nature 388, 782-787.
7. Rescigno, M., Citterio, S., Thery, C., Rittig, M., Medaglini, D., Pozzi, G., Amigorena, S., and Ricciardi-Castagnoli, P. (1998) Proc Natl Acad Sci USA 95, 5229-5234.
8. Austyn, J. M., Kupiec-Weglinski, J. W., Hankins, D. F., and Morris, P. J. (1988) J Exp Med 167, 646-651.
9. Sallusto, F., and Lanzavecchia, A. (2000) Immunol Rev 177, 134-140.
10. Locht, C., Rouanet, C., Hougardy, J. M., and Mascart, F. (2007) Expert Opin Biol Ther 7, 1665-1677.
11. Hougardy, J. M., Schepers, K., Place, S., Drowart, A., Lechevin, V., Verscheure, V., Debrie, A. S., Doherty, T. M., Van Vooren, J. P., Locht, C., and Mascart, F. (2007) PLoS ONE 2, e926.
12. Parida, S. K., and Kaufmann, S. H. (2010) Curr Opin Immunol 22, 374-384.

What is claimed is:

1. A method for increasing the effectiveness of antigen presentation by an antigen presenting cell (APC) comprising the steps of:
isolating and purifying a dendritic cell (DC)-specific antibody or fragment thereof to which one or more antigens or recombinant antigens are attached or conjugated to form an antibody-antigen complex, and
contacting the APC with the antibody-antigen complex under conditions wherein the antibody-antigen complex is processed and presented for T cell recognition of a Mycobacterium tuberculosis antigen.

2. The method of claim 1, wherein the APC comprises an isolated dendritic cell (DC), a peripheral blood mononuclear cell (PBMC), a monocyte, a B cell, a myeloid dendritic cell and combinations thereof that have been cultured in vitro with GM-CSF and IL-4, GM-CSF and interferon alpha, and antigen.

3. The method of claim 1, which the one or more antigens or recombinant antigens comprise M. tuberculosis antigens comprises at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211.

4. The method of claim 1, wherein the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR.

5. The method of claim 1, wherein a nucleotide sequence for the DC-specific antibody or the antigen is selected from SEQ ID NOS: 6 to 70, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, or 220.

6. An immunostimulatory composition for generating an immune response, for prophylaxis, for therapy or any combination thereof against tuberculosis in a human or animal subject comprising:
an anti-dendritic cell (DC)-specific antibody or fragment thereof fused or conjugated to at least a portion of one or more M. tuberculosis antigens or antigenic peptides; and
a pharmaceutically acceptable carrier, wherein the conjugate is comprised in an amount effective to generate the immune response against tuberculosis.

7. The composition of claim 6, which the one or more antigens or recombinant antigens comprise M. tuberculosis antigens comprises at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211.

8. The composition of claim 6, wherein the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR.

9. The composition of claim 6, wherein the DC-specific antibody or fragment comprises αDCIR, αLangerin, αLOX-1 or αCD40.

10. The composition of claim 6, wherein the DC-specific antibody is humanized.

11. The composition of claim 6, wherein the composition is administered to the human or animal subject by an oral route, a nasal route, topically or as an injection.

12. The composition of claim 11, wherein the injection is selected from the group consisting of subcutaneous, intravenous, intraperitoneal, intramuscular, and intravenous.

13. A vaccine composition comprising:
an anti-dendritic cell (DC)-specific antibody or fragment thereof fused or conjugated to at least a portion of one or more recombinant or natural M. tuberculosis antigens or antigenic peptides, wherein the antigenic peptides are representative of one or more epitopes of the one or more antigens implicated in tuberculosis; and
an optional pharmaceutically acceptable carrier or an adjuvant.

14. The composition of claim 13, further comprising a flexible linker between the DC-specific antibody or fragment thereof and the one or more antigens.

15. The composition of claim 13, wherein the one or more antigens or antigenic peptides are conjugated to a heavy-chain, a light-chain or both of the anti-dendritic cell (DC)-specific antibody.

16. The composition of claim 13, which the one or more antigens or recombinant antigens comprise M. tuberculosis antigens comprises at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211.

17. The composition of claim 13, wherein the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR.

18. The composition of claim 13, wherein the DC-specific antibody or fragment comprises αDCIR, αLangerin, αLOX-1 or αCD40.

19. The composition of claim 13, wherein the DC-specific antibody is humanized.

20. The composition of claim 13, wherein the composition is administered to the human or animal subject by an oral route, a nasal route, topically or as an injection.

21. The composition of claim 20, wherein the injection is selected from the group consisting of subcutaneous, intravenous, intraperitoneal, intramuscular, and intravenous.

22. A tuberculosis vaccine comprising:
a dendritic cell (DC)-specific antibody or fragment thereof comprising αDCIR, αLangerin, αLOX-1 or αCD40 fused to one or more M. tuberculosis antigens selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, wherein the antigens or the antigenic peptides are conjugated to a heavy chain of the DC-specific antibody via one or more flexible linkers or by at least one half of a cohesin-dockerin pair; and
an optional at least one of a pharmaceutically acceptable carrier or an adjuvant.

23. A method for increasing effectiveness of one or more dendritic cells (DCs) comprising the steps of:
isolating the DCs from a patient;
exposing the DCs to activating amounts of a vaccine comprising a dendritic cell (DC)-specific antibody or fragment thereof loaded, fused or conjugated to one or more antigens or antigenic M. tuberculosis antigens selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211, wherein the antigens or the antigenic peptides are conjugated to a heavy chain, a light chain or both of the DC-specific antibody via one or more flexible linkers; and an optional pharmaceutically acceptable carrier or an adjuvant wherein the DC-specific antibody or fragment comprises αDCIR, αLangerin, αLOX-1 or αCD40; and
reintroducing the antigen-loaded and activated DCs into the patient.

24. A method for a treatment, a prophylaxis or a combination thereof against tuberculosis in a human subject comprising the steps of:
identifying the human subject in need of the treatment, the prophylaxis or a combination thereof against tuberculosis; and
administering a vaccine composition comprising:
an anti-dendritic cell (DC)-specific antibody or fragment thereof fused or conjugated to at least a portion of one or more recombinant or natural M. tuberculosis antigens or antigenic peptides, wherein the antigenic peptides are representative of one or more epitopes of the one or more antigens implicated in tuberculosis and an optional pharmaceutically acceptable carrier or an adjuvant.

25. The method of claim 24, further comprising a flexible linker between the DC-specific antibody or fragment thereof and the one or more antigens.

26. The method of claim 24, wherein the one or more antigens or antigenic peptides are conjugated to a heavy-chain, a light-chain or both of the anti-dendritic cell (DC)-specific antibody.

27. The method of claim 24, wherein the one or more M. tuberculosis antigens are selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211.

28. The method of claim 24, wherein the DC-specific antibody or fragment is selected from an anti-DCIR, αDCIR, MHC class I, MHC class II, CD1, CD2, CD3, CD4, CD8, CD11b, CD14, CD15, CD16, CD19, CD20, CD29, CD31, αCD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, αLangerin, DECTIN-1, B7-1, B7-2, IFN-γ receptor and IL-2 receptor, ICAM-1, Fcγ receptor, αLOX-1 or ASPGR.

29. The method of claim 24, wherein the DC-specific antibody or fragment comprises αDCIR, αLangerin, αLOX-1 or αCD40.

30. The method of claim 24, wherein the DC-specific antibody is humanized.

31. The method of claim 24, wherein the composition is administered to the human or animal subject by an oral route, a nasal route, topically or as an injection.

32. The method of claim 31, wherein the injection is selected from the group consisting of subcutaneous, intravenous, intraperitoneal, intramuscular, and intravenous.

33. An immunostimulatory composition for generating an immune response, for a prophylaxis, a therapy or any combination thereof against tuberculosis in a human or animal subject comprising:
an anti-dendritic cell (DC)-specific antibody or fragment thereof fused or conjugated to at least a portion of one or more M. tuberculosis antigens;
at least one Toll-Like Receptor (TLR) agonist which is selected from the group consisting of TLR1, TLR2, TLR3, TLR4, TLR5, TLR6, TLR7, and TLR8 agonists; and
a pharmaceutically acceptable carrier, wherein the conjugate is comprised in an amount effective to generate the immune response against tuberculosis.

34. The composition of claim 33, wherein the one or more M. tuberculosis antigens are selected from at least one of SEQ ID NOS: 1, 2, 3, 4, 5, 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211.

35. The composition of claim 33, wherein the DC-specific antibody or fragment comprises αDCIR, αLangerin, αLOX-1 or αCD40.

36. A tuberculosis vaccine comprising:
an anti-dendritic cell (DC)-specific antibody or fragment thereof fused or conjugated to at least a portion of one or more recombinant or natural M. tuberculosis antigens or antigenic peptides, wherein the antigenic peptides are representative of one or more epitopes of the one or more antigens implicated in tuberculosis;
at least one Toll-Like Receptor (TLR) agonist which is selected from the group consisting of TLR1, TLR2, TLR3, TLR4, TLR5, TLR6, TLR7, and TLR8 agonists; and
one or more optional pharmaceutically acceptable carriers and adjuvants.

37. The composition of claim 36, wherein the DC-specific antibody is humanized.

38. The composition of claim 36, wherein the antigen is selected from at least one of SEQ ID NOS: 71 to 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, or 211.

39. A method for a treatment, a prophylaxis or a combination thereof against tuberculosis in a human subject comprising the steps of:
identifying the human subject in need of the treatment, the prophylaxis or a combination thereof against tuberculosis; and
administering a vaccine composition comprising:
an anti-dendritic cell (DC)-specific antibody or fragment thereof fused or conjugated to at least a portion of one or more recombinant or natural M. tuberculosis antigens or antigenic peptides, wherein the antigenic peptides are representative of one or more epitopes of the one or more antigens implicated in tuberculosis; and
at least one Toll-Like Receptor (TLR) agonist which is selected from the group consisting of TLR1, TLR2, TLR3, TLR4, TLR5, TLR6, TLR7, and TLR8 agonists; and
one or more optional pharmaceutically acceptable carriers and adjuvants.

40. The method of claim 39, further comprising a flexible linker between the DC-specific antibody or fragment thereof and the one or more antigens.

41. The method of claim 39, wherein the one or more antigens or antigenic peptides are conjugated to a heavy-chain, a light-chain or both of the anti-dendritic cell (DC)-specific antibody.

42. The method of claim 39, wherein the one or more M. tuberculosis antigens are selected from the group consisting of SEQ ID NO: 1, SEQ ID NO: 2, SEQ ID NO: 3, SEQ ID NO: 4, SEQ ID NO: 5, and any combinations thereof.

43. The method of claim 39, wherein the DC-specific antibody or fragment comprises αDCIR, αLangerin, αLOX-1 or αCD40.

44. The method of claim 39, wherein the DC-specific antibody is humanized.
